(19) United States
(12) Patent Application Publication
(10) Pub. No.: US 2023/0020161 A1
(43) Pub. Date: Jan. 19, 2023

(54) Tricyclic Modulators of PP2A

(71) Applicant: Rappta Therapeutics Oy, Helsinki (FI)

(72) Inventor: George L TRAINOR, Wilmington, DE (US)

(73) Assignee: Rappta Therapeutics Oy, Helsinki (FI)

(21) Appl. No.: 17/907,949

(22) PCT Filed: Feb. 26, 2021
(86) PCT No.: PCT/FI2021/050144

§ 371 Date: Aug. 29, 2022

(30) Foreign Application Priority Data
Feb. 28, 2020 (FI) 20205211
Oct. 29, 2020 (FI) 20206080

(51) Int. Cl. C07D 223/28; C07D 265/38; C07D 413/04; C07D 405/04; A61P 31/14

(52) U.S. Cl. CPC C07D 223/28; C07D 265/38; C07D 413/04; C07D 405/04; A61P 31/14

(57) ABSTRACT

Chemical modulators of PP2A, comprising tricyclic sulfonimidamides are disclosed. The compounds are useful in preventing or treating cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease and cardiac hypertrophy. The compounds are of formula (I)

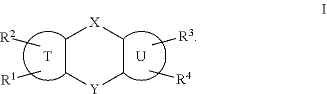

### TECHNICAL FIELD

The present disclosure relates to chemical modulators of PP2A, comprising tricyclic sulfonimidamides.

### BACKGROUND

Protein phosphatase 2A is one of the four major serine threonine phosphatases and is implicated in the negative control of cell growth and division. Protein phosphatase 2A holoenzymes are heterotrimeric proteins composed of a structural subunit A, a catalytic subunit C, and a regulatory subunit B. The PP2A heterotrimeric protein phosphatase is a ubiquitous and conserved phosphatase with broad substrate specificity and diverse cellular functions.

PP2A function may be implicated in a variety of pathologies and indications.

The compounds described herein exhibit anti-proliferative effects.

### SUMMARY

A compound is disclosed. The compound is of formula (I):

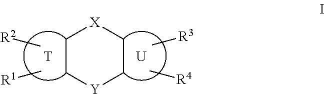

wherein:

X is absent, direct bond, —S—, —(CH₂CH₂)—, —CH═CH—; —O—, —CH₂O—, —OCH₂—, —C(═O)NR^{D}—, or —N(R^{D})C(═O)—;

R^{D} is selected from hydrogen and (C₁-C₆)alkyl;

Y is selected from the group consisting of:
N(R⁵)—, —N(R⁵)—S(O)₂—, —CH₂—N(R⁵)—CH₂—, —C(R¹⁴)(R⁵)—, —C(═R^{5a})— and —C(R^{A}R^{B})—,
R^{A} and R^{B} together with the C atom to which they are attached form a three to six membered aliphatic carbocycle or a heterocycle which is substituted with Z and attached at the C atom as a spiro ring, and the carbocycle or the heterocycle which is substituted with Z is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;
T is a benzene ring or a five- or six-membered heteroaromatic ring;
U is a benzene ring or a five- or six-membered heteroaromatic ring;
R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, (C₁-C₆)haloalkylthio, (C₁-C₆)haloalkylthio, —CHC(═O)O(C₁-C₆)alkyl;
R⁵ is —(CR¹⁵R¹⁶)ₚ-Q_{q}-(CR¹⁵R¹⁶)ₙ₋ₚ—Z or;

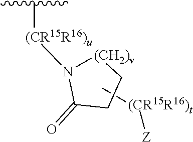

R^{5a} is ═CR¹⁴(CR¹⁵R¹⁶)ₚ-Q_{q}-(CR¹⁵R¹⁶)ₘ₋ₚ—Z;

Q is selected from —O—, —NR^{14′}—, —C(O)—, —S—, —S(O)— and —S(O)₂—;

R⁶ and R⁷ are independently selected from the group consisting of: H and (C₁-C₆)alkyl;

R¹⁴ is hydrogen or (C₁-C₆)alkyl;

R^{14′} is hydrogen or optionally substituted (C₁-C₆)alkyl, (C₃-C₇) cycloalkyl, aryl, or heteroaryl; —SO₂R⁸; —SO₂NR⁸R⁹; —C(═O) R¹⁰; —C(═O)OR¹⁰; or —C(═O)NR⁸R⁹; wherein said substituents on the (C₁-C₆)alkyl, (C₃-C₇)cycloalkyl, aryl, or heteroaryl are selected from the group consisting of hydroxy, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₃-C₇) cycloalkyl, (C₁-C₆)haloalkyl, (C₁-C₆)haloalkoxy, and (C₁-C₆)alkoxy,

R⁸ and R⁹ are independently selected in each instance from hydrogen, (C₁-C₆)alkyl, aryl, and arylalkyl, wherein said aryl or the aryl of the arylalkyl is optionally substituted with hydroxy, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, or (C₁-C₆)alkoxy;

R¹⁰ is selected from hydrogen, optionally substituted (C₁-C₆)alkyl, or optionally substituted aryl, wherein said optional substituents are selected from the group consisting of (C₁-C₆)alkyl, OR⁶, NH₂, NHMe, N(Me)₂, and heterocycle;

R¹⁵ and R¹⁶ in each occurrence are selected independently from the group consisting of from H, OH, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, or, taken together, two of R¹⁴, R^{14′}, R¹⁵ and R¹⁶ may form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;

m is an integer from 1 to 3;

n is an integer from 2 to 4;

p is zero, 1 or 2;

q is zero or 1:

with the proviso that when p is 2, m is not 1;

t is zero, 1 or 2;

u is zero, 1 or 2;

v is 1, 2 or 3;

Z is —NHS((O)(NR¹⁸))R¹⁷;

R¹⁷ is selected from phenyl and monocyclic heteroaryl, said phenyl and monocyclic heteroaryl optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O)H, (C₁-C₆)hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl;

R¹⁸ is H or (C₁-C₆)alkyl, wherein said alkyl is optionally substituted with (C₃-C₇)cycloalkyl group;

or an enantiomer, a diastereomer, a tautomer or a pharmaceutically acceptable salt thereof.

The compound may be considered to be a tricyclic sulfonimidamide.

Further, a pharmaceutical composition is disclosed. The pharmaceutical composition may comprise a compound according to one or more embodiments described in this specification, for example a compound of formula (I), an enantiomer, a diastereomer, a tautomer or a pharmaceutically acceptable salt thereof, and pharmaceutically acceptable carrier.

Further, a compound according to one or more embodiments described in this specification, for example a compound of formula (I), an enantiomer, a diastereomer, a tautomer or a pharmaceutically acceptable salt thereof, for use as a medicament is disclosed.

Further, a compound according to one or more embodiments described in this specification, for example a compound of formula (I), an enantiomer, a diastereomer, a tautomer or a pharmaceutically acceptable salt thereof, for use in preventing or treating a disease or condition ameliorated by the modulating of PP2A is disclosed.

Further, a method of preventing or treating a disease or condition is disclosed. The method may comprise administering to a patient a therapeutically effective amount of a compound according to one or more embodiments described in this specification, for example a compound of formula (I), an enantiomer, a diastereomer, a tautomer or a pharmaceutically acceptable salt thereof.

### BRIEF DESCRIPTION OF THE DRAWINGS

The accompanying drawings, which are included to provide a further understanding of the embodiments and constitute a part of this specification, illustrate various embodiments. In the drawings:

FIG. 1A illustrates Western blots of Methyl-PP2A-C, total PP2A-C, S62-MYC, total MYC, and Vinculin in LNCaP cells treated with vehicle control or 30 μM Target-2 and harvested at 1 hour.

FIG. 1B illustrates cell viability of LNCaP cells treated with increasing doses of Target-2.

FIG. 2A illustrates Western blots of Methyl-PP2A-C, total PP2A-C, S62-MYC, total MYC, Vinculin and cleaved PARP in LNCaP cells treated with vehicle control or 30 μM Target-3 and harvested at 1 hour or 12 hours.

FIG. 2B illustrates cell viability of LNCaP cells treated with increasing doses of Target-3.

FIG. 2C illustrates Colony formation assay of LNCaP cells treated with 5, 7.5, 10, and 20 μM Target-3 for 3 weeks.

FIG. 3A illustrates Western blots of Methyl-PP2A-C, total PP2A-C, S62-MYC, total MYC, and Vinculin in LNCaP cells treated with vehicle control or 30 μM Target-5 and harvested at 1 hour.

FIG. 3B illustrates cell viability of LNCaP cells treated with increasing doses of Target-5.

FIG. 4A illustrates Western blots of Methyl-PP2A-C, total PP2A-C, S62-MYC, total MYC, Vinculin and cleaved PARP in LNCaP cells treated with vehicle control or 30 μM Target-6 and harvested at 1 hour or 12 hours.

FIG. 4B illustrates cell viability of LNCaP cells treated with increasing doses of Target-6.

FIG. 4C illustrates Colony formation assay of LNCaP cells treated with 5, 7.5, 10, and 20 μM Target-6 for 3 weeks.

### DETAILED DESCRIPTION

The present disclosure relates to a compound of formula (I)

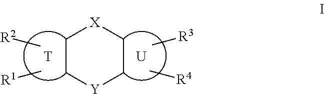

X is absent, direct bond, —S—, —(CH₂CH₂)—, —CH═CH—; —O—, —CH₂O—, —OCH₂—, —C(═O)NR^{D}—, or —N(R^{D})C(═O)—;

R^{D} is selected from hydrogen and (C₁-C₆)alkyl;

Y is selected from the group consisting of:
N(R⁵)—, —N(R⁵)—S(O)₂—, —CH₂—N(R⁵)—CH₂—, —C(R¹⁴)(R⁵)—, —C(═R^{5a})— and —C(R^{A}R^{B}),

R^{A} and R^{B} together with the C atom to which they are attached form a three to six membered aliphatic carbocycle or a heterocycle which is substituted with Z and attached at the C atom as a spiro ring, and the carbocycle or the heterocycle which is substituted with Z is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;

T is a benzene ring or a five- or six-membered heteroaromatic ring;

U is a benzene ring or a five- or six-membered heteroaromatic ring;

R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, (C₁-C₆)haloalkylthio, —CHC(═O)O(C₁-C₆)alkyl;

R⁵ is —(CR¹⁵R¹⁶)ₚ-Q_{q}-(CR¹⁵R¹⁶)ₙ₋ₚ—Z or;

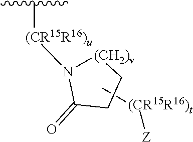

R^{5a} is ═CR¹⁴(CR¹⁵R¹⁶)ₚ-Q_{q}-(CR¹⁵R¹⁶)ₘ₋ₚ—Z;

Q is selected from —O—, —NR^{14′}—, —C(O)—, —S—, —S(O)— and —S(O)₂—;

R⁶ and R⁷ is independently selected from the group consisting of: H and (C₁-C₆)alkyl;

R¹⁴ is hydrogen or (C₁-C₆)alkyl;

R^{14′} is hydrogen or optionally substituted (C₁-C₆)alkyl, (C₃-C₇) cycloalkyl, aryl, or heteroaryl; —SO₂R⁸; —SO₂NR⁸R⁹; —C(═O) R¹⁰; —C(═O)OR¹⁰; or —C(═O)NR⁸R⁹; wherein said substituents on the (C₁-C₆)alkyl, (C₃-C₇)cycloalkyl, aryl, or heteroaryl are selected from the group consisting of hydroxy, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₃-C₇) cycloalkyl, (C₁-C₆)haloalkyl, (C₁-C₆)haloalkoxy, and (C₁-C₆)alkoxy,

R⁸ and R⁹ are independently selected in each instance from hydrogen, (C₁-C₆)alkyl, aryl, and arylalkyl, wherein said aryl or the aryl of the arylalkyl is optionally substituted with hydroxy, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, or (C₁-C₆)alkoxy;

R¹⁰ is selected from hydrogen, optionally substituted (C₁-C₆)alkyl, or optionally substituted aryl, wherein said optional substituents are selected from the group consisting of (C₁-C₆)alkyl, OR⁶, NH₂, NHMe, N(Me)₂, and heterocycle;

R¹⁵ and R¹⁶ in each occurrence are selected independently from the group consisting of from H, OH, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, or, taken together, two of R¹⁴, R^{14′}, R¹⁵ and R¹⁶ may form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;

m is an integer from 1 to 3;

n is an integer from 2 to 4;

p is zero, 1 or 2;

q is zero or 1:

with the proviso that when p is 2, m is not 1;

t is zero, 1 or 2;

u is zero, 1 or 2;

v is 1, 2 or 3;

Z is —NHS((O)(NR¹⁸))R¹⁷;

R¹⁷ is selected from phenyl and monocyclic heteroaryl, said phenyl and monocyclic heteroaryl optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl;

R¹⁸ is H or (C₁-C₆)alkyl, wherein said alkyl is optionally substituted with (C₃-C₇)cycloalkyl group;

or an enantiomer, a diastereomer, a tautomer or a pharmaceutically acceptable salt thereof.

In an embodiment, the present disclosure relates to a compound of formula (I):

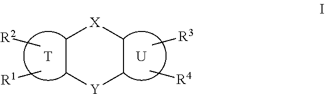

X is absent, direct bond, —S—, —(CH₂CH₂)—, —CH═CH—; —O—, —CH₂O—, —OCH₂—, —C(═O)NR^{D}—, or —N(R^{D})C(═O)—;

R^{D} is selected from hydrogen and (C₁-C₆)alkyl;

Y is selected from the group consisting of:
N(R⁵)—, —N(R⁵)—S(O)₂—, —CH₂—N(R⁵)—CH₂—, —C(R¹⁴)(R⁵)—, —C(═R^{5a})— and —C(R^{A}R^{B}),

R^{A} and R^{B} together with the C atom to which they are attached form a three to six membered aliphatic carbocycle or a heterocycle which is substituted with Z and attached at the C atom as a spiro ring, and the carbocycle or the heterocycle which is substituted with Z is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;

T is a benzene ring or a five- or six-membered heteroaromatic ring;

U is a benzene ring or a five- or six-membered heteroaromatic ring;

R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, (C₁-C₆)haloalkylthio, —CHC(═O)O(C₁-C₆)alkyl;

R⁵ is —(CR¹⁵R¹⁶)ₚ-Q_{q}-(CR¹⁵R¹⁶)ₙ₋ₚ—Z or;

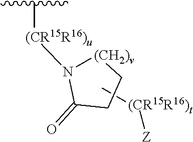

R^{5a} is ═CR¹⁴(CR¹⁵R¹⁶)ₚ-Q_{q}-(CR¹⁵R¹⁶)ₘ₋ₚ—Z;

Q is selected from —O—, —NR^{14′}—, —C(O)—, —S—, —S(O)— and —S(O)₂—;

R⁶ and R⁷ is independently selected from the group consisting of: H and (C₁-C₆)alkyl;

R¹⁴ is hydrogen or (C₁-C₆)alkyl;

R^{14′} is hydrogen or optionally substituted (C₁-C₆)alkyl, (C₃-C₇) cycloalkyl, aryl, or heteroaryl; —SO₂R⁸; —SO₂NR⁸R⁹; —C(═O) R¹⁰; —C(═O)OR¹⁰; or —C(═O)NR⁸R⁹; wherein said substituents on the (C₁-C₆)alkyl, (C₃-C₇)cycloalkyl, aryl, or heteroaryl are selected from the group consisting of hydroxy, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₃-C₇) cycloalkyl, (C₁-C₆)haloalkyl, (C₁-C₆)haloalkoxy, and (C₁-C₆)alkoxy,

R⁸ and R⁹ are independently selected in each instance from hydrogen, (C₁-C₆)alkyl, aryl, and arylalkyl, wherein said aryl or the aryl of the arylalkyl is optionally substituted with hydroxy, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, or (C₁-C₆)alkoxy;

R¹⁰ is selected from hydrogen, optionally substituted (C₁-C₆)alkyl, or optionally substituted aryl, wherein said optional substituents are selected from the group consisting of (C₁-C₆)alkyl, OR⁶, NH₂, NHMe, N(Me)₂, and heterocycle;

R¹⁵ and R¹⁶ in each occurrence are selected independently from the group consisting of from H, OH, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, or, taken together, two of R¹⁴, R^{14′}, R¹⁵ and R¹⁶ may form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;

m is an integer from 1 to 3;

n is an integer from 2 to 4;

p is zero, 1 or 2;

q is zero or 1:

with the proviso that when p is 2, m is not 1;

t is zero, 1 or 2;

u is zero, 1 or 2;

v is 1, 2 or 3;

Z is —NHS((O)(NR¹⁸))R¹⁷;

R¹⁷ is selected from phenyl and monocyclic heteroaryl, said phenyl and monocyclic heteroaryl optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl;

R¹⁸ is H, methyl, propyl, methylcyclopropyl, or (C₁-C₆)alkyl, wherein said alkyl is optionally substituted with (C₃-C₇)cycloalkyl group;

or an enantiomer, a diastereomer, a tautomer or a pharmaceutically acceptable salt thereof.

In the context of the present specification, the substituents may further comprise certain chemical structures as described in the following examples.

The term “sulfonimidamide”, may be understood as referring to a group —NHS((O)(NR¹⁸))—, i.e.

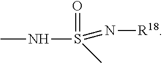

In an embodiment, the term “alkyl” means linear, branched, or cyclic hydrocarbon structures and combinations thereof, and which may be saturated or unsaturated (e.g. partially unsaturated, fully unsaturated). Thus, the term “alkyl” includes the sub-classes alkenyl, alkynyl, cycloalkyl, and the like. Alkyl groups may be optionally substituted as defined herein.

Examples of saturated straight-chain alkyl groups include methyl, ethyl, n-propyl, n-butyl, n-pentyl, n-hexyl, n-heptyl, noctyl, n-nonyl, and n-decyl and branched-chain alkyl groups include isopropyl, tert-butyl, isobutyl, sec-butyl, and neopentyl. In an embodiment, alkyl is saturated alkyl having from 2 to 6 carbon atoms. In an embodiment, a straight chain or branched chain alkyl has 6 or fewer carbon atoms in its backbone (e.g., C₁-C₆ for straight chain, C₃-C₆ for branched chain). The term (C₁-C₆)alkyl may be understood as referring to alkyl groups containing 1 to 6 carbon atoms.

In an embodiment, the term “alkenyl” means an alkyl group having one or more carbon-carbon double bonds. In an embodiment, the term “C₂₋₆ alkenyl” means an alkenyl moiety having from 2 to 6 carbon atoms.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl, pentenyl, and hexenyl.

In an embodiment, the term “alkynyl” means an alkyl group having one or more carbon-carbon triple bonds. In an embodiment, the term “C₂₋₆ alkynyl” means an alkynyl moiety having from 2 to 6 carbon atoms.

Examples of unsaturated alkynyl groups include, but are not limited to, ethynyl (ethinyl, —C═CH) and 2-propynyl (propargyl, —CH₂—C═CH).

The term “alkylene” means a straight or branched chain saturated hydrocarbon attached at two or more positions, such as methylene (—CH₂—). Unless otherwise specified, the term “alkyl” may include “alkylene” groups.

The term “cycloalkyl” or alternatively, “carbocycle”, alone or in combination, means a saturated or partially saturated monocyclic, bicyclic or tricyclic alkyl group wherein each cyclic moiety may contain from 3 to 12 carbon atom ring members and which may optionally be a benzo fused ring system which is optionally substituted as defined herein. In an embodiment, cycloalkyl comprise from 3 to 7 carbon atoms or from 3 to 6 carbon atoms.

Examples of saturated monocyclic cycloalkyl groups include, but are not limited to, cyclopropyl, cyclobutyl, cyclopentyl, cyclohexyl, cycloheptyl, methylcyclopropyl, dimethylcyclopropyl, methylcyclobutyl, dimethylcyclobutyl), methylcyclopentyl, dimethylcyclopentyl and methylcyclohexyl.

Examples of saturated monocyclic cycloalkyl groups include, but are not limited to, cyclopropenyl, cyclobutenyl, cyclopentenyl, cyclohexenyl, methylcyclopropenyl, dimethylcyclopropenyl, methylcyclobutenyl, dimethylcyclobutenyl, methylcyclopentenyl, dimethylcyclopentenyl and methylcyclohexenyl.

Examples of bicyclic cycloalkyl groups include, but are not limited to, tetrahydronapthyl, indanyl, octahydronaphthyl, 2,3-dihydro-1H-indenyl, decalinyl and the like. “Bicyclic” and “tricyclic” as used together with “cycloalkyl” are intended to include both fused ring systems, such as decahydronaphthalene, octahydronaphthalene as well as the multicyclic (multicentered) saturated or partially unsaturated type, including spiro-ring fused systems. Examples of bicyclic and tricyclic types of isomer are bicyclo[1,1,1]pentane, norbornane, camphor, adamantane, bicyclo[3,2,1]octane, and [4,4.1]-bicyclononane.

The term “heterocycle” and, interchangeably, “heterocycloalkyl” means a cycloaliphatic or aryl carbocycle residue in which from one to four carbons is replaced by a heteroatom selected from the group consisting of N, O and S. The nitrogen and sulfur heteroatoms may optionally be oxidized, and the nitrogen heteroatom may optionally be quaternized. In an embodiment, the heterocycle is non-aromatic. In a further embodiment, the heterocycle is aromatic.

Examples of heterocycles include, but are not limited to, aziridine, azetidine pyrrolidine, pyrazole, pyrrole, indole, quinoline, isoquinoline, tetrahydroisoquinoline, benzofuran, benzodioxan, benzodioxole, tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues include, but are not limited to, piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl (also historically called thiophenyl), benzothienyl, thiamorpholinyl, oxadiazolyl, triazolyl and tetrahydroquinolinyl. It is to be noted that heteroaryl is a subset of heterocycle in which the heterocycle is aromatic. An oxygen heterocycle is a heterocycle containing at least one oxygen in the ring; it may contain additional oxygens, as well as other heteroatoms. A sulphur heterocycle is a heterocycle containing at least one sulphur in the ring; it may contain additional sulphurs, as other heteroatoms. Oxygen heteroaryl is a subset of oxygen as other heteroatoms. Oxygen heteroaryl is a subset of oxygen heterocycle; examples include furan and oxazole. Sulphur heteroaryl is a subset of sulphur heterocycle; examples include, but are not limited to, thiophene and thiazine. A nitrogen heterocycle is a heterocycle containing at least one nitrogen in the ring; it may contain additional nitrogens, as well as other heteroatoms. Examples include, but are not limited to, piperidine, piperazine, morpholine, pyrrolidine and thiomorpholine. Nitrogen heteroaryl is a subset of nitrogen heterocycle; examples include, but are not limited to, pyridine, pyrrole and thiazole. The heterocycle groups may be optionally substituted unless specifically prohibited.

Aryl and heteroaryl mean (i) a phenyl group (or benzene) or a monocyclic 5- or 6-membered heteroaromatic ring containing 1-4 heteroatoms selected from O, N, or S as defined for heterocycles; (ii) a bi cyclic 9- or 10-membered aromatic or heteroaromatic ring system containing 0-4 heteroatoms selected from O, N, or S as defined for carbocycles or heterocycles; or (iii) a tricyclic 13- or 14-membered aromatic or heteroaromatic ring system containing 0-5 heteroatoms selected from O, N, or S as defined for carbocycles or heterocycles. The aromatic 6- to 14-membered carbocyclic rings include, but are not limited to, benzene, naphthalene, anthracene, indane, tetralin, and fluorene and the 5- to 10-membered aromatic heterocyclic rings include, but are not limited to, imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

Arylalkyl refers to a substituent in which an aryl residue is attached to the parent structure through alkyl. Examples of arylalkyl include, but are not limited to, benzyl, phenethyl and the like. Heteroarylalkyl refers to a substituent in which a heteroaryl residue is attached to the parent structure through alkyl. In one embodiment, the alkyl group of an arylalkyl or a heteroarylalkyl is an alkyl group of from 1 to 6 carbons. Examples include, e.g., pyridinylmethyl, pyrimidinylethyl and the like.

The term “optionally substituted” may be used interchangeably with “unsubstituted or substituted”. The term “substituted” means the replacement of one or more hydrogen atoms in a specified group with a specified radical. In an embodiment, 1, 2 or 3 hydrogen atoms are replaced with a specified radical. In the case of alkyl and cycloalkyl, more than three hydrogen atoms can be replaced by fluorine. In an embodiment, all available hydrogen atoms may be replaced by fluorine. Two substituents may be joined together to form a form a three to seven membered non-aromatic carbocycle or heterocycle consisting of zero to three heteroatoms, for example forming methylenedioxy or ethylenedioxy. In an embodiment, the formed carbocyclic or heterocyclic ring is fused ring or spiro ring.

The above groups, whether alone or part of another substituent, may themselves optionally be substituted with one or more groups selected from themselves and the additional substituents listed below. Further, the substituents listed below may themselves be substituents.

Alkoxy or alkoxyl means a group of from 1 to 6 carbon atoms of a straight, branched or cyclic configuration and combinations thereof attached to the parent structure through an oxygen.

Examples of alkoxy include, but are not limited to, methoxy, ethoxy, propoxy, isopropoxy, cyclopropyloxy, sec-butoxy, isobutoxy, tert-butoxy, cyclohexyloxy and cycloheptyyli.

“Oxy” or “oxa” means —O—.

Hydroxy means —OH.

Halo or halogen means, alone or in combination, fluorine, chlorine, bromine, or iodine. In an embodiment, halo may be fluorine or chlorine.

The term “haloalkyl” means an alkyl radical having the meaning as defined above wherein one or more hydrogens are replaced with a halogen. in an embodiment haloalkyl is monohaloalkyl, dihaloalkyl and polyhaloalkyl group. Examples of haloalkyl radicals include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” means a haloalkyl group attached at two or more positions. Examples include, but are not limited to, fluoromethylene (—CFH—), difluoromethylene (—CF₂—) and chloromethylene (—CHCl—).

The term “haloalkoxy” means a haloalkyl group attached to the parent molecular moiety through an oxygen atom.

The terms “alkylcarbonyl” and “alkoxycarbonyl” mean —C(═O)alkyl or —C(═O)alkoxy, respectively.

“Carbonyl” is a —C(O)— group and includes formyl (—C(O)H).

“Carboxyl” or “carboxy,” refers to —C(O)OH or the corresponding “carboxylate” anion, such as is in a carboxylic acid salt.

Ester (carboxylate, carboxylic acid ester, oxycarbonyl) means —C(═O)Oalkyl, wherein alkyl is an ester substituent defined for alkyl above. Examples of ester groups include, but are not limited to, —C(═O)OCH₃, —C(═O)OCH₂CH₃, —C(═O)OC(CH₃)₃, and —C(═O)OPh.

The double bonded oxygen (═O), when referred to as a substituent itself is called “oxo”.

The term “alkylamino” means an alkyl group attached to the parent molecular moiety through an amino group. Alkylamino groups may be mono- or dialkylated, forming groups such as, for example, N-methylamino, N-ethylamino, N,N-dimethylamino, N,N-ethylmethylamino and the like.

The term “amino” means —NH₂.

The term “alkylthio” means an alkyl thioether (alkyl-S—) group wherein the term alkyl is as defined for alkyl groups and wherein the sulfur may be singly or doubly oxidized. Examples of alkyl thioether groups include, but are not limited to, methylthio, ethylthio, n-propylthio, isopropylthio, n-butylthio, iso-butylthio, sec-butylthio, tert-butylthio, methanesulfonyl, ethanesulfinyl, and the like.

Amido (carbamoyl, carbamyl, aminocarbonyl, carboxamide) means a group such as —C(═O)NH₂, —C(═O)NHalkyl, or —C(═O)N(alkyl)₂, wherein alkyl groups are independently amino substituents, as defined for alkyl groups.

The term “acyl” means a carbonyl attached to an alkenyl, alkyl, aryl, cycloalkyl, heteroaryl, heterocycle, or any other moiety were the atom attached to the carbonyl is carbon. Examples of acyl groups include formyl, alkanoyl and aroyl. An “acetyl” group refers to a —C(O)CH₃ group. One or more carbons in the acyl residue may be replaced by nitrogen, oxygen or sulfur as long as the point of attachment to the parent molecular moiety remains at the carbonyl. Examples include acetyl, benzoyl, propionyl, isobutyryl, t-butoxycarbonyl, benzyloxycarbonyl and the like.

The term “acylamino” as used herein, alone or in combination, embraces an acyl group attached to the parent moiety through an amino group. An example of an “acylamino” group is acetylamino (CH₃C(O)NH—).

“Carbamate” refers to an ester of carbamic acid (—NHCOO—) which may be attached to the parent molecular moiety from either the nitrogen or acid end, and which may be optionally substituted as defined herein.

Nitro means —NO₂.

Azido means —N₃.

Cyano (nitrile, carbonitrile) means —CN.

Arylalkyl refers to a substituent in which an aryl residue is attached to the parent molecular moiety through alkyl. Examples are benzyl, phenethyl and the like. Heteroarylalkyl refers to a substituent in which a heteroaryl residue is attached to the parent molecular moiety through alkyl. In one embodiment, the alkyl group of an arylalkyl or a heteroarylalkyl is an alkyl group of from 1 to 6 carbons. Examples include, e.g., pyridinylmethyl, pyrimidinylethyl and the like.

“Imino” means ═N—.

“Thia” and “thio” mean a —S— group or an ether wherein the oxygen is replaced with sulfur. The oxidized derivatives of the thio group, namely sulfinyl and sulfonyl, are included in the definition of thia and thio.

“Sulfonate,” “sulfonic acid,” and “sulfonic,” mean the —SO₃H group and its anion as the sulfonic acid is used in salt formation.

“Sulfanyl” means —S—.

“Sulfinyl” means —S(O)—.

“Sulfonyl” means —S(O)₂—.

“Sulfonamido” (sulfinamoyl; sulfonic acid amide; sulfonamide) means —S(═O)₂NH₂, —S(═O)₂NH(alkyl), —S(═O)₂N(alkyl)₂, wherein alkyl are independently amino substituents, as defined for alkylamino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃) 2, and —S(═O)₂NHPh.

The term “pharmaceutically acceptable salt” may refer to salts prepared from pharmaceutically acceptable non-toxic acids or bases including inorganic acids and bases and organic acids and bases. When the compounds disclosed in this specification are basic, salts may be prepared from pharmaceutically acceptable non-toxic acids including inorganic and organic acids. Suitable pharmaceutically acceptable acid addition salts for the compounds disclosed in this specificationinclude acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

Unless otherwise stated or depicted, structures of compounds according to one or more embodiments disclosed in this specification are also meant to include all stereoisomeric (e.g., enantiomeric, diastereomeric, and cis-trans isomeric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and cis-trans isomeric (or conformational) mixtures of the present compounds are within the scope of the present disclosure. The configuration of any carbon-carbon double bond appearing herein is selected for convenience only and is not intended to designate a particular configuration; thus a carbon-carbon double bond depicted arbitrarily herein as trans may be cis, trans, or a mixture of the two in any proportion. Unless otherwise stated, all tautomeric forms of the compounds according to one or more embodiments disclosed in this specification are within the scope of the present disclosure. Additionally, the compounds of formula (I) can exist in unsolvated as well as solvated forms with pharmaceutically acceptable solvents such as water, ethanol, and the like. In general, the solvated forms are considered equivalent to the unsolvated forms.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

“Diastereomers” are stereoisomers that have at least two asymmetric atoms, but which are not mirror-images of each other.

The term “tautomer” or “tautomeric form” refers to structural isomers of different energies which are interconvertible via a low energy barrier. Some non-limiting examples of pro-ton tautomers (also known as prototropic tautomers) include interconversions via migration of a proton, such as keto-enol and imine-enamine isomerizations. Valence tautomers include intercon-versions by reorganization of some of the bonding electrons. Unless otherwise stated, all tauto-meric forms of the compounds disclosed herein are within the scope of the invention.

The compounds of formula (I) comprising following five or six membered rings may contain three or more asymmetric centers and may thus give rise to enantiomers, diastereomers and other diastereometric forms which may be defined in terms of absolute stereochemistry as (R)— or (S)—.

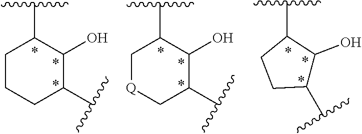

The asymmetric center is marked with * in the structures depicted above. A ring having three asymmetric centers may comprise 12 different stereoisomers such as (1R,2R,3R), (1R,2R,3S), (1R,2S,3S), (1R,2S,3R), (1S,2R,3R), (1S,2R,3S), (1R,2S,3R), (1S,2S,3R) and (1S,2S,3S), if the position of an asymmetric carbon atom having OH-substituent is defined as a 2-position.

The absolute stereochemistry may be depicted using wedge bonds (bold or parallel lines). Examples of diastereoisomers and enantiomers are shown in table 1.

The compounds according to one or more embodiments disclosed in this specification can exist in radiolabeled form, i.e., the compounds may contain one or more atoms containing an atomic mass or mass number different from the atomic mass or mass number usually found in nature. Radioisotopes of hydrogen, carbon, phosphorous, fluorine, and chlorine include ²H, ³H, ¹³C, ¹⁴C, ¹⁵N, ³⁵S, ¹⁸F, and ³⁶Cl, respectively. In an embodiment, radioisotopes are tritiated, i.e. ³H, and carbon-14, i.e., ¹⁴C, radioisotopes which are easy to prepare and detect. Compounds of formula (I) that contain isotopes ¹¹C, ¹³N, ¹⁵O and ¹⁸F are suited for positron emission tomography. Radiolabeled compounds according to one or more embodiments disclosed in this specification and prodrugs thereof can generally be prepared by methods well known to those skilled in the art. In an embodiment, such radio labeled compounds may be prepared by carrying out the procedures disclosed in the Examples by substituting a readily available radiolabeled reagent for a non-radiolabeled reagent.

In an embodiment of the compound of formula I, T is a benzene ring, U is a benzene ring, and Y is —N(R⁵)—, i.e.

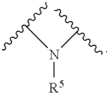

In one embodiment the compound is of formula (IA)

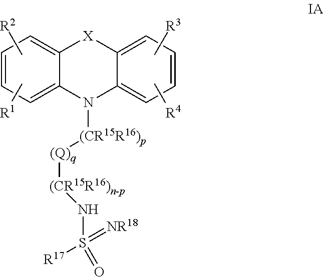

wherein X, R^{D}, R¹, R², R³, R⁴, R⁶, R⁷, R¹⁵, R¹⁶, R¹⁷, R¹⁸, Q, R^{14′}, R⁸, R⁹, R¹⁰, n, p, and q are as defined for the compound of formula (I).

In one embodiment, the compound is of formula (IA), wherein X, R^{D}, R¹, R², R³, R⁴, R⁶, R⁷, R¹⁵, R¹⁶, R¹⁷, R¹⁸, Q, R^{14′} R⁸, R⁹, R¹⁰ n, p, and q are as defined for the compound of formula (I),

provided that when q is 1 and Q is —C(O)—, n-p is not 0.

In an embodiment, the compound is of formula (IA), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇)cycloalkyl, (C₁-C₆)alkenyl, (C₁-C₆)alkynyl or aryl,
and X, R^{D}, R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, (C₁-C₆)haloalkylthio, —CHC(═O)O(C₁-C₆)alkyl,
and X, R^{D}, R¹⁵, R¹⁶, R¹⁸, Q, R^{14′}, R⁸, R⁹, R¹⁰, n, p, and q are as defined for compound of formula I, R¹⁷ is as defined for the compound of formula (IA).
In an embodiment, the compound is of formula (IA), wherein X is absent, R¹⁷ is as defined for compound of formula IA and R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein X is direct bond, R¹⁷ is as defined for compound of formula IA and R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein X is —S—, R¹⁷ is as defined for compound of formula (IA) and R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein X is —(CH₂CH₂)—, R¹⁷ is as defined for compound of formula IA and R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein X is —CH═CH—, R¹⁷ is as defined for compound of formula IA and R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein X is —O—, R¹⁷ is as defined for compound of formula IA and R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein X is —CH₂O—, R¹⁷ is as defined for compound of formula IA and R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein X is —OCH₂—, R¹⁷ is as defined for compound of formula IA and R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein X is —C(═O)NR^{D}—, R¹⁷ is as defined for compound of formula IA and R^{D}, R¹, R², R³, R⁴, R⁶, R⁷, R¹⁵, R¹⁶ and R¹⁸, Q, R^{14′}, R⁸, R⁹, R¹⁰, n, p, and q are as defined for the compound of formula (I).
In an embodiment, the compound is of formula (IA), wherein R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, (C₁-C₆)haloalkylthio, —CHC(═O)O(C₁-C₆)alkyl;
Q is —C(O)—,
q is 0 or 1
n is an integer from 2 to 4,
p is zero, 1 or 2;
R¹⁵ and R¹⁶ in each occurrence are independently H or OH,
R¹⁷ is as defined for compound of formula (IA) and X and
R¹⁸ are as defined for compound of formula (I).

In an embodiment, the compound is of formula (IA), wherein X is —(CH₂CH₂)—,

R¹, R², R³, and R⁴ are independently selected from the group consisting of: H, halo, amino, azido,

n is 2 or 3; p is 0 or 1;

Q is selected from —C(O)—,

q is 0 or 1,

R¹⁵ and R¹⁶ in each occurrence are independently H or OH

R¹⁷ is selected from phenyl, pyridinyl, thiazolyl, furanyl, thiophenyl, pyrrolyl, thienyl, each optionally substituted with one or two substituents selected independently from the group consisting of phenyl optionally substituted with one or two substituents selected independently from the group consisting of C₁-C₆)alkyl, halogen, cyano, nitro, amino, (C₁-C₆)haloalkoxy, (C₁-C₆)haloalkyl, (C₁-C₆)alkylsulfonyl, —NR⁶C(O)OR^{6′} and (C₁-C₆)alkylamino,

R^{6′} is (C₁-C₆)alkyl,

R¹⁸ is hydrogen or (C₁-C₆)alkyl, wherein said alkyl is optionally substituted with (C₃-C₇)cycloalkyl group.

In an embodiment, the compound is of formula (IA), wherein X is —S—,

R¹, R², R³, and R⁴ are independently selected from the group consisting of: H, halo, amino, azide,

n is 2 or 3,

p is 0,

q is 0,

R¹⁵ and R¹⁶ in each occurrence are independently H or OH

R¹⁷ selected from phenyl, pyridinyl, thiazolyl, furanyl, thiophenyl, pyrrolyl, thienyl, each optionally substituted with one or two substituents selected independently from the group consisting of phenyl optionally substituted with one or two substituents selected independently from the group consisting of C₁-C₆)alkyl, halogen, cyano, nitro, amino, (C₁-C₆)haloalkoxy, (C₁-C₆)haloalkyl, (C₁-C₆)alkylsulfonyl, —NR⁶C(O)OR^{6′} and (C₁-C₆)alkylamino,

R^{6′} is (C₁-C₆)alkyl, and

R¹⁸ is hydrogen or (C₁-C₆)alkyl, wherein said alkyl is optionally substituted with (C₃-C₇)cycloalkyl group.
In an embodiment, the compound is of formula (IA), wherein R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, (C₁-C₆)haloalkylthio, —CHC(═O)O(C₁-C₆)alkyl;
Q is —C(O)—,
q is 0 or 1
n is an integer from 2 to 4,
p is zero, 1 or 2;
R¹⁵ and R¹⁶ in each occurrence are independently H or OH,
R¹⁷ is as defined for compound of formula (IA) and X and R¹⁸ are as defined for compound of formula (I).

In an embodiment, the compound is of formula (IA), wherein X is —(CH₂CH₂)—,

R¹, R², R³, and R⁴ are independently selected from the group consisting of: H, halo, amino, azido,

n is 2 or 3; p is 0 or 1;

Q is selected from —C(O)—,

q is 0 or 1,

R¹⁵ and R¹⁶ in each occurrence are independently H or OH

R¹⁷ is selected from phenyl, pyridinyl, thiazolyl, furanyl, thiophenyl, pyrrolyl, thienyl, each optionally substituted with one or two substituents selected independently from the group consisting of phenyl optionally substituted with one or two substituents selected independently from the group consisting of C₁-C₆)alkyl, halogen, cyano, nitro, amino, (C₁-C₆)haloalkoxy, (C₁-C₆)haloalkyl, (C₁-C₆)alkylsulfonyl, —NR⁶C(O)OR^{6′} and (C₁-C₆)alkylamino,

R^{6′} is (C₁-C₆)alkyl,

R¹⁸ is hydrogen, methyl, propyl, methylcyclopropyl, or (C₁-C₆)alkyl, wherein said alkyl is optionally substituted with (C₃-C₇) cycloalkyl group.

In an embodiment, the compound is of formula (IA), wherein X is —S—,

R¹, R², R³, and R⁴ are independently selected from the group consisting of: H, halo, amino, azide,

n is 2 or 3,

p is 0,

q is 0,

R¹⁵ and R¹⁶ in each occurrence are independently H or OH

R¹⁷ selected from phenyl, pyridinyl, thiazolyl, furanyl, thiophenyl, pyrrolyl, thienyl, each optionally substituted with one or two substituents selected independently from the group consisting of phenyl optionally substituted with one or two substituents selected independently from the group consisting of C₁-C₆)alkyl, halogen, cyano, nitro, amino, (C₁-C₆)haloalkoxy, (C₁-C₆)haloalkyl, (C₁-C₆)alkylsulfonyl, —NR⁶C(O)OR^{6′} and (C₁-C₆)alkylamino,

R^{6′} is (C₁-C₆)alkyl, and

R¹⁸ is hydrogen, methyl, propyl, methylcyclopropyl, or (C₁-C₆)alkyl, wherein said alkyl is optionally substituted with (C₃-C₇) cycloalkyl group.

In an embodiment of compound of Formula IA, R¹⁵ and R¹⁶ in each occurrence are selected independently from the group consisting of from H, OH, cyano, amino, (C₁-C₃)alkylamino, (C₁-C₃)dialkylamino, (C₁-C₃)alkyl, (C₁-C₃)haloalkyl, (C₁-C₃)haloalkoxy, and (C₁-C₃)alkoxy, or, taken together, two of R¹⁵ and R¹⁶ form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₃)alkylamino, (C₁-C₃)dialkylamino, (C₁-C₃)alkyl, (C₁-C₃)haloalkyl, (C₁-C₃)haloalkoxy, and (C₁-C₃)alkoxy.

In an embodiment of compound of Formula IA, two R¹⁵ and/or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy. In an embodiment, two R¹⁵ and/or R¹⁶ groups from different —(CR¹⁵R¹⁶)— groups form a direct bond or an alkylene —(CH₂)_{n′}—, wherein n′ is 1, 2, 3, 4 or 5, thus forming a three to seven membered non-aromatic carbocycle or heterocycle B, wherein H atoms of alkylene are optionally replaced with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy.

In an embodiment, said compound is of formula (IA-1) or (IA-2):

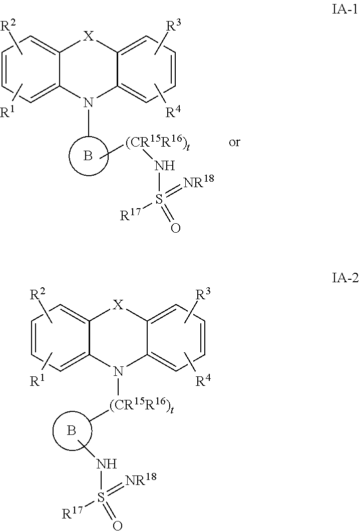

wherein
t is zero, 1 or 2,
B is as defined for compound of formula (IA) above,
and X, R^{D}, R¹, R², R³, R⁴, R⁶, R⁷, R¹⁵, R¹⁶, R¹⁷ and R¹⁸ are as defined for compound of formula (I). In an embodiment, the compound is of formula (IA-1) or (IA-2), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O)H, (C₁-C₆)hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,
and X, B, R^{D}, R¹, R², R³, R⁴, R⁶, R⁷, R¹⁵, R¹⁶ and R¹⁸, and t are as defined for compound of formula (I).
In an embodiment, the compound is of formula (IA-1) or (IA-2), wherein R¹, R², R³, and R⁴ are independently selected from the group consisting of: H, halo, (C₁-C₆)alkyl, (C₁-C₆)haloalkyl and —CN;
and X, B, R^{D}, R¹⁵, R¹⁶, R¹⁸ and t are as defined for compound of formula I, R¹⁷ is as defined for compound of formula (IA-1) or (IA-2) above.

In an embodiment, the compound is of formula (IA-1) or (IA-2), wherein B is three to seven membered non-aromatic carbocycle or heterocycle, optionally substituted with one or two substituents selected independently from the group consisting of OH and (C₁-C₆)alkyl;

and X, R^{D}, R¹⁵, R¹⁶, R¹⁸ and t are as defined for compound of formula I, and R¹⁷, R¹, R², R³, and R⁴ are as defined for compound of formula (IA-1) or (IA-2) above.

In an embodiment, the compound is of formula (IA-1) or (IA-2), wherein t is 0;

and X, R^{D}, and R¹⁸ are as defined for compound of formula I, and B, R¹⁷, R¹, R², R³, and R⁴ are as defined for compound of formula (IA-1) or (IA-2) above.

In an embodiment, the compound is of formula (IA-1) or (IA-2), wherein X is absent, R¹⁷ is as defined for compound of formula (IA-1) or (IA-2) and R¹, R², R³, R⁴, R⁶, R⁷, R¹⁵, R¹⁶ and R¹⁸ and t are as defined for compound of formula (I).

In an embodiment, the compound is of formula (IA-1) or (IA-2), wherein X is direct bond, R¹⁷ is as defined for compound of formula (IA-1) or (IA-2) and R¹, R², R³, R⁴, R⁶, R⁷, R¹⁵, R¹⁶, R¹⁸ and t are as defined for compound of formula (I).

In an embodiment, the compound is of formula (IA-1) or (IA-2), wherein X is —S—, R¹⁷ is as defined for compound of formula (IA-1) or (IA-2) and R¹, R², R³, R⁴, R⁶, R⁷, R¹⁵, R¹⁶, R¹⁸ and t are as defined for compound of formula (I).

In an embodiment, the compound is of formula (IA-1) or (IA-2), wherein X is —S—;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

B is five to six membered non-aromatic carbocycle or heterocycle containing one heteroatom selected from 0 and N, optionally substituted with one substituent selected independently from the group consisting of OH and (C₁-C₆)alkyl;

R¹⁸ is as defined for compound of formula (I).

In an embodiment, the compound is of formula (IA-1) or (IA-2), wherein X is —(CH₂CH₂)—;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

B is five to six membered non-aromatic carbocycle or heterocycle containing one heteroatom selected from 0 and N, optionally substituted with one substituent selected independently from the group consisting of OH and (C₁-C₆)alkyl;

R¹⁸ is as defined for compound of formula (I).

In an embodiment, the compound is of formula (IA-1) or (IA-2), wherein X is —CH═CH—;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

B is five to six membered non-aromatic carbocycle or heterocycle containing one heteroatom selected from 0 and N, optionally substituted with one substituent selected independently from the group consisting of OH and (C₁-C₆)alkyl;

R¹⁸ is as defined for compound of formula (I).

In an embodiment, the compound is of formula (IA-1) or (IA-2), wherein X is —O—;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

B is five to six membered non-aromatic carbocycle or heterocycle containing one heteroatom selected from 0 and N, optionally substituted with one substituent selected independently from the group consisting of OH and (C₁-C₆)alkyl;

R¹⁸ is as defined for compound of formula (I).

In an embodiment, the compound is of formula (IA-1) or (IA-2), wherein X is —CH₂O—;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃; t is 0 or 1;

B is five to six membered non-aromatic carbocycle or heterocycle containing one heteroatom selected from 0 and N, optionally substituted with one substituent selected independently from the group consisting of OH and (C₁-C₆)alkyl;

R¹⁸ is as defined for compound of formula (I).

In an embodiment, the compound is of formula (IA-1) or (IA-2), wherein X is —OCH₂—:

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

B is five to six membered non-aromatic carbocycle or heterocycle containing one heteroatom selected from 0 and N, optionally substituted with one substituent selected independently from the group consisting of OH and (C₁-C₆)alkyl;

R¹⁸ is as defined for compound of formula (I).

In an embodiment, the compound is of formula (IA-1) or (IA-2), wherein X is —C(═O)NR^{D}— and R^{D} is hydrogen or (C₁-C₆)alkyl;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

B is five to six membered non-aromatic carbocycle or heterocycle containing one heteroatom selected from 0 and N, optionally substituted with one substituent selected independently from the group consisting of OH and (C₁-C₆)alkyl;

R¹⁸ is as defined for compound of formula (I).

In an embodiment, the compound is of formula (IA-1) or (IA-2), wherein X is absent;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

B is five to six membered non-aromatic carbocycle or heterocycle containing one heteroatom selected from 0 and N, optionally substituted with one substituent selected independently from the group consisting of OH and (C₁-C₆)alkyl;

R¹⁸ is as defined for compound of formula (I).

In an embodiment, the compound is of formula (IA-1) or (IA-2), wherein X is direct bond;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

B is five to six membered non-aromatic carbocycle or heterocycle containing one heteroatom selected from 0 and N, optionally substituted with one substituent selected independently from the group consisting of OH and (C₁-C₆)alkyl;

R¹⁸ is as defined for compound of formula (I).

In an embodiment, the compound is of formula (IA-1), wherein X is —(CH₂CH₂)—;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0;

B is six membered non-aromatic carbocycle or heterocycle containing one heteroatom 0, optionally substituted with one substituent selected independently from the group consisting of OH and (C₁-C₆)alkyl;

R¹⁸ is as defined for compound of formula (I).

In an embodiment, the compound is of formula (IA-1), wherein X is —O—;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0;

B is six membered non-aromatic carbocycle or heterocycle containing one heteroatom 0, optionally substituted with one substituent selected independently from the group consisting of OH and (C₁-C₆)alkyl;

R¹⁸ is as defined for compound of formula (I).

In an embodiment, the compound is of formula (IA-1), wherein X is direct bond;

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0;

B is six membered non-aromatic carbocycle or heterocycle containing one heteroatom 0, optionally substituted with one substituent selected independently from the group consisting of OH and (C₁-C₆)alkyl;

R¹⁸ is as defined for compound of formula (I).

In an embodiment of compound of formula (IA-1) or (IA-2), B is a five-membered ring as set forth in formula:

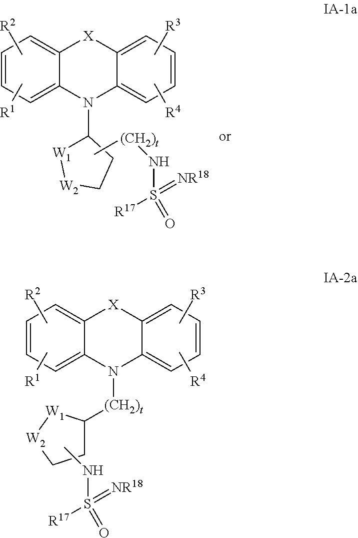

wherein:
W₁ and W₂ are both —CH₂—; or
one of W₁ and W₂ is selected from a group consisting of —O—, —NR^{14′}—, —C(O)—, —S—, —S(O)— or —S(O)₂ and the other is —CH₂—; or

one of W₁ and W₂ is —CH(OH)— and the other is —CH₂—; and

X, R^{D}, R¹, R², R³, R⁴, R⁶, R⁷, R¹⁴1, R, R⁹, R¹⁰, R⁷ and R¹⁸ and t are as defined for compound of formula (I).

In an embodiment of compound of formula (IA-1) or (IA-2), B is a five-membered ring

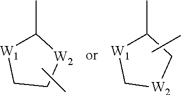

wherein
W₁ and W₂ are as defined for compound of formula of (IA-1a) or (IA-2a) above.

In an embodiment, the compound is of formula (IA-1a) or (IA-2a), wherein

R¹, R², R³, and R⁴ are independently selected from the group consisting of: H, halo, (C₁-C₆)alkyl, (C₁-C₆)haloalkyl and —CN;
R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;
t is 0 or 1;
X, R^{D}, R^{14′}, R⁸, R⁹, R¹⁰, R¹⁸ are as defined for compound of formula (I).

In an embodiment, the compound is of formula (IA-1a) or (IA-2a), wherein

t is 0;

X is —CH₂CH₂—;

t is 0;

R¹, R², R³, R⁴ and R¹⁷ are as defined for compound of formula IA-1a or IA-2a above

R^{14′} and R¹⁸ are as defined for compound of formula I.

In an embodiment of compound of formula (IA-1) or (IA-2), B is a six-membered ring as set forth in formula:

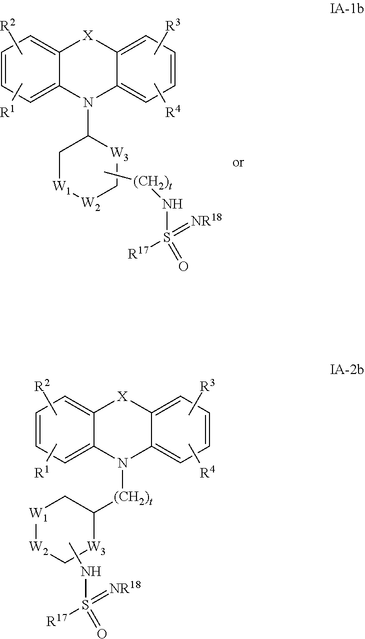

wherein:
all of W₁, W₂ and W₃ are —CH₂—; or

one of W₁, W₂ and W₃ is —O—, —NR^{14′}—, —C(O)—, —S—, —S(O)— or —S(O)₂ and the other two are —CH₂—; or

one of W₁, W₂ and W₃ is —O—, —NR^{14′}—, —C(O)—, —S—, —S(O)— or —S(O)₂ and the other two are —CH₂— and —CH(OH)—; or

one of W₁, W₂ and W₃ is —CH(OH)— and the other two are —CH₂; and

X, R^{D}, R¹, R², R³, R⁴, R⁶, R⁷, R^{14′}, R⁸, R⁹, R¹⁰, R¹⁷ and R¹⁸, and t are as defined for compound of formula (I).

In an embodiment of compound of formula (IA-1) or (IA-2), B is a six-membered ring

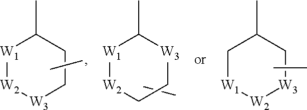

wherein
W₁, W₂ and W₃ are as defined for compound of formula of (IA-1b) or (IA-2b) above.

In an embodiment, the compound is of formula (IA-1b) or (IA-2b), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl, and X, R^{D}, R¹, R², R³, R⁴, R⁶, R⁷, R⁸, R⁹, R¹⁰, R^{14′} and R¹⁸ and t are as defined for compound of formula (I), and W₁, W₂, W₃ are as defined for compound of formula (IA-1b) or (IA-2b) above.

In an embodiment, the compound is of formula (IA-1b) or (IA-2b), wherein R¹, R², R³, and R⁴ are independently selected from the group consisting of: H, halo, (C₁-C₆)alkyl, (C₁-C₆)haloalkyl and —CN;

and X, R^{D}, R⁸, R⁹, R¹⁰, R^{14′}, R¹⁸ and t are as defined for compound of formula (I), and R¹⁷, W₁, W₂, W₃ are as defined for compound of formula (IA-1b) or (IA-2b) above.

In an embodiment, the compound is of formula (IA-1b) or (IA-2b), wherein

R¹, R², R³, R⁴, R¹⁷, W₁, W₂ and W₃ are as defined for compound of formula IA-1b or IA-2b above,

and X, R^{D}, R⁶, and R¹⁸ are as defined for compound of formula (I),

t is 0 or 1,

R^{14′}, when present, is hydrogen or optionally substituted (C₁-C₆)alkyl, (C₃-C₇) cycloalkyl, aryl, or heteroaryl; —SO₂R⁸; —SO₂NR⁸R⁹; —C(═O) R¹⁰; —C(═O)OR¹⁰; or —C(═O)NR⁸R⁹; wherein said substituents on the (C₁-C₆)alkyl, (C₃-C₇) cycloalkyl, aryl, or heteroaryl are selected from the group consisting of hydroxy, halogen, (C₁-C₆)alkyl,

R⁸ and R⁹ are independently selected in each instance from hydrogen, (C₁-C₆)alkyl, aryl, and arylalkyl, wherein said aryl or the aryl of the arylalkyl is optionally substituted with hydroxy, halogen, cyano, nitro, amino, (C₁-C₆)alkylamino, (C₁-C₆)dialkylamino, (C₁-C₆)alkyl, (C₁-C₆)haloalkyl, (C₁-C₆)haloalkoxy, or (C₁-C₆)alkoxy;

R¹⁰ is selected from hydrogen, optionally substituted (C₁-C₆)alkyl, or optionally substituted aryl, wherein said optional substituents are selected from the group consisting of (C₁-C₆)alkyl, OR⁶, NH₂, NHMe, N(Me)₂, and heterocycle.

In an embodiment, the compound is of formula IA-1b or IA-2b, wherein X is absent,

t is 0,

R¹, R², R³, R⁴, R¹⁷, W₁, W₂, W₃, R^{14′}, R⁸, R⁹ and R¹⁰ are as defined for compound of formula (IA-1b) or (IA-2b) above,

and R⁶ and R¹⁸ are as defined for compound of formula (I).

In an embodiment, the compound is of formula (IA-1b) or (IA-2b), wherein X is direct bond,

t is 0,

R¹, R², R³, R⁴, R¹⁷, W₁, W₂, W₃, R^{14′}, R⁸, R⁹ and R¹⁰ are as defined for compound of formula (IA-1b) or (IA-2b) above,

and R⁶ and R¹⁸ are as defined for compound of formula (I).

In an embodiment, the compound is of formula (IA-1b) or (IA-2b), wherein X is —S—,

t is 0,

R¹, R², R³, R⁴, R¹⁷, W₁, W₂, W₃, R^{14′}, R⁸, R⁹ and R¹⁰ are as defined for compound of formula (IA-1b) or (IA-2b) above,

and R⁶ and R¹⁸ are as defined for compound of formula (I).

In an embodiment, the compound is of formula (IA-1b) or (IA-2b), wherein X is —(CH₂CH₂)—,

t is 0,
R¹, R², R³, R⁴, R¹⁷, W₁, W₂, W₃, R^{14′}, R⁸, R⁹ and R¹⁰ are as defined for compound of formula (IA-1b) or (IA-2b) above, and R⁶ and R¹⁸ are as defined for compound of formula (I).

In an embodiment, the compound is of formula (IA-1b) or (IA-2b), wherein X is —CH═CH—;

t is 0,

R¹, R², R³, R⁴, R¹⁷, W₁, W₂, W₃, R^{14′}, R⁸, R⁹ and R¹⁰ are as defined for compound of formula (IA-1b) or (IA-2b) above,

and R⁶ and R¹⁸ are as defined for compound of formula (I).

In an embodiment, the compound is of formula (IA-1b) or (IA-2b), wherein X is —O—,

t is 0,

R¹, R², R³, R⁴, R¹⁷, W₁, W₂, W₃, R^{14′}, R⁸, R⁹ and R¹⁰ are as defined for compound of formula (IA-1b) or (IA-2b) above,

and R⁶ and R¹⁸ are as defined for compound of formula (I).

In an embodiment, the compound is of formula (IA-1b) or (IA-2b), wherein X is —CH₂O—,

t is 0,

R¹, R², R³, R⁴, R¹⁷, W₁, W₂, W₃, R^{14′}, R⁸, R⁹ and R¹⁰ are as defined for compound of formula (IA-1b) or (IA-2b) above,

and R⁶ and R¹⁸ are as defined for compound of formula (I).

In an embodiment, the compound is of formula (IA-1b) or (IA-2b), wherein X is —OCH₂—,

t is 0,

R¹, R², R³, R⁴, R¹⁷, W₁, W₂, W₃, R^{14′}, R⁸, R⁹ and R¹⁰ are as defined for compound of formula (IA-1b) or (IA-2b) above,

and R⁶ and R¹⁸ are as defined for compound of formula (I).

In an embodiment, the compound is of formula (IA-1b) or (IA-2b), wherein X is —C(═O)NR^{D}—, R^{D} is hydrogen or (C₁-C₆)alkyl;

t is 0,

R¹, R², R³, R⁴, R¹⁷, W₁, W₂, W₃, R^{14′}, R⁸, R⁹ and R¹⁰ are as defined for compound of formula (IA-1b) or (IA-2b) above,

and R⁶ and R¹⁸ are as defined for compound of formula (I).

In an embodiment of compound of Formula (IA), p and q are both zero, R¹⁵ is H and R¹⁶ is selected from H or OH. In an embodiment the compound is of formula (IA-3) or (IA-3′):

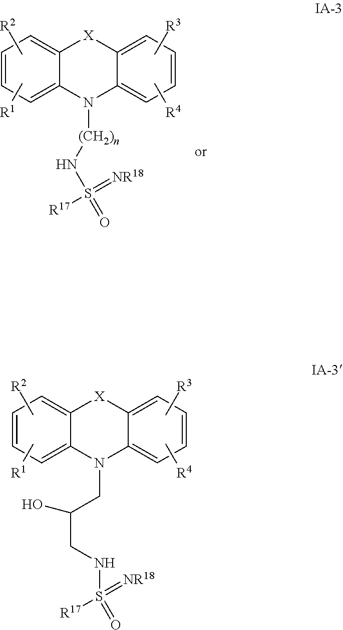

wherein
n is 2, 3 or 4,
R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇)cycloalkyl, (C₁-C₆)alkenyl, (C₁-C₆)alkynyl or aryl, R¹, R², R³, and R⁴ are independently selected from the group consisting of: H, halo, (C₁-C₆)alkyl, (C₁-C₆)haloalkyl and —CN;
and X and R¹⁸ are as defined for compound of formula (I).

In an embodiment of the compound of formula I, T is a benzene ring, U is a benzene ring, and Y is —N(R⁵)—, and R⁵ is

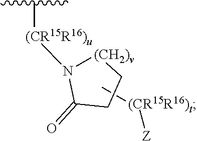

wherein X, R^{D}, R¹, R², R³, R⁴, R⁶, R⁷, R¹⁵, R¹⁶, Z, R¹⁷ and R¹⁸, u, v and t are as defined for compound of formula (I).

In an embodiment, R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,

and X, R^{D}, R¹, R², R³, R⁴, R⁶, R⁷, R¹⁵, R¹⁶, Z, and R¹⁸, u, v and t are as defined for compound of formula (I).

In an embodiment, R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, —CC(═O)O(C₁-C₆)alkyl,

and X, R^{D}, R⁶, R⁷, R¹⁵, R¹⁶, Z, R¹⁷ and R¹⁸, u, v and t are as defined for compound of formula (I).

In an embodiment of the compound of formula I, T is a benzene ring, U is a benzene ring, and Y is —N(R⁵)—S(O)₂—, and X, R^{D}, R¹, R², R³, R⁴, R⁶, R⁷, R¹⁵, R¹⁶, R¹⁷, R¹⁸, Q, R^{14′}, R⁸, R⁹, R¹⁰, n, p, and q are as defined for the compound of formula (I).

In an embodiment of the compound of formula I, T is a benzene ring, U is a benzene ring, and Y is —CH₂—N(R⁵)—CH₂—, and X, R^{D}, R¹, R², R³, R⁴, R⁶, R⁷, R¹⁵, R¹⁶, R¹⁷, R¹⁸, Q, R^{14′}, R⁸, R⁹, R¹⁰, n, p, and q are as defined for the compound of formula (I).

In an embodiment of the compound of formula (I), T is a benzene ring, U is a benzene ring, and Y is —C(R¹⁴)(R⁵)—, i.e.

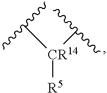

R¹⁴ is H. In an embodiment, the compound is of formula (IB)

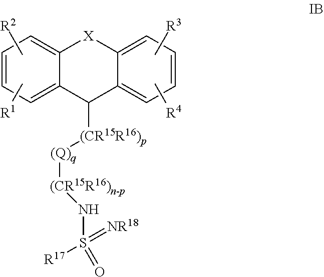

wherein X, R^{D}, R¹, R², R³, R⁴, R⁶, R⁷, R^{14′} R⁸, R⁹, R¹⁰, R¹⁵, R¹⁶, R¹⁷ and R¹⁸, Q, n, p, and q are as defined for the compound of formula (i).

In one embodiment, the compound is of formula (IB), wherein X, R^{D}, R¹, R², R³, R⁴, R⁶, R⁷, R^{14′}, R⁸, R⁹, R¹⁰, R¹⁵, R¹⁶, R¹⁷ and R¹⁸, Q, n, p, and q are as defined for the compound of formula (I),

provided that when q is 1 and Q is —C(O)—, n-p is not 0.

In an embodiment, the compound is of formula (IB), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,

and X, R^{D}, R¹, R², R³, R⁴, R⁶, R⁷, R^{14′}, R⁸, R⁹, R¹⁰, R¹⁵, R¹⁶ and R¹⁸, Q, n, p, and q are as defined for compound of formula (i).

In an embodiment, the compound is of formula (IB), wherein

R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, —CC(═O)O(C₁-C₆)alkyl,

and R¹⁷ is as defined for compound of formula IB,

and X, R^{D}, R^{14′}, R⁸, R⁹, R¹⁰, R¹⁵, R¹⁶ and R¹⁸, Q, n, p, and q are as defined for compound of formula (i).

In an embodiment, the compound is of formula (IB), wherein

p is 0,

Q is NH, q is 1 or 0,

and R¹⁷, R¹, R², R³, R⁴, R⁶ and R⁷, are as defined for compound of formula IB,

and X, R^{D}, R⁸, R⁹, R¹⁰, R¹⁵, R¹⁶ and R¹⁸ and n are as defined for compound of formula (i).

In an embodiment of compound of Formula IB, R¹⁵ and R¹⁶ in each occurrence are selected independently from the group consisting of from H, OH, cyano, amino, (C₁-C₃)alkylamino, (C₁-C₃)dialkylamino, (C₁-C₃)alkyl, (C₁-C₃)haloalkyl, (C₁-C₃)haloalkoxy, and (C₁-C₃)alkoxy, or, taken together, two R¹⁵ and R¹⁶ form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₃)alkylamino, (C₁-C₃)dialkylamino, (C₁-C₃)alkyl, (C₁-C₃)haloalkyl, (C₁-C₃)haloalkoxy, and (C₁-C₃)alkoxy.

In an embodiment of compound of Formula IB, two R¹⁵ and/or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy.

In an embodiment, two R¹⁵ and/or R¹⁶ groups from different —(CR¹⁵R¹⁶)— groups form a direct bond or an alkylene —(CH₂)_{n′}—, wherein n′ is 1, 2, 3, 4 or 5, thus forming a three to seven membered non-aromatic carbocycle or heterocycle B, wherein H atoms of alkylene are optionally replaced with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy.

In an embodiment, said compound is of formula (IB-1) or (IB-2):

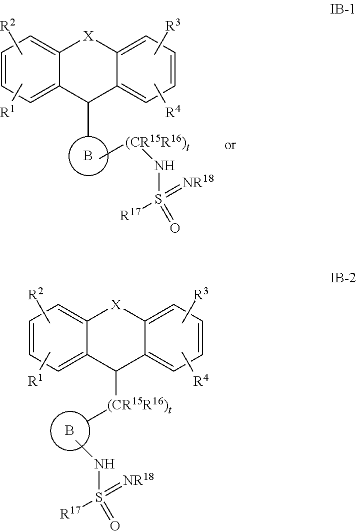

wherein

t is zero, 1 or 2,

B is as defined for compound of formula (IB) above,

and X, R^{D}, R¹, R², R³, R⁴, R⁶, R⁷, R¹⁵, R¹⁶, R¹⁷ and R¹⁸ are as defined for compound of formula (I). In an embodiment, the compound is of formula (IB-1) or (IB-2), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,

and X, B, R^{D}, R¹, R², R³, R⁴, R⁶, R⁷, R¹⁵, R¹⁶ and R¹⁸, and t are as defined for compound of formula (I),

B is as defined for compound of formula (IB-1) or (IB-2) above.

In an embodiment, the compound is of formula (IB-1) or (IB-2), wherein R¹, R², R³, and R⁴ are independently selected from the group consisting of: H, halo, (C₁-C₆)alkyl, (C₁-C₆)haloalkyl and —CN;

and X, B, R^{D}, R¹⁵, R¹⁶, R¹⁸ and t are as defined for compound of formula I, B and R¹⁷ are as defined for compound of formula (IB-1) or (IB-2) above.

In an embodiment, the compound is of formula (IB-1) or (IB-2), wherein B is three to seven membered non-aromatic carbocycle or heterocycle, optionally substituted with one or two substituents selected independently from the group consisting of OH and (C₁-C₆)alkyl;

and X, R^{D}, R¹⁵, R¹⁶, R¹⁸ and t are as defined for compound of formula I, and R¹⁷, R¹, R², R³, and R⁴ are as defined for compound of formula (IB-1) or (IB-2) above.

In an embodiment, the compound is of formula (IB-1) or (IB-2), wherein t is 0;

and X, R^{D}, and R¹⁸ are as defined for compound of formula I, and B, R¹⁷, R¹, R², R³, and R⁴ are as defined for compound of formula (IB-1) or (IB-2) above.

In an embodiment of compound of formula (IB-1) or (IB-2), B is a five-membered ring as set forth in formula:

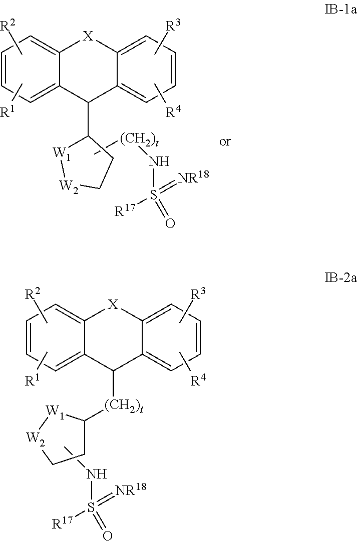

wherein:
all of W₁, W₂ and W₃ are —CH₂—; or
one of W₁, W₂ and W₃ is —O—, —NR^{14′}—, —C(O)—, —S—, —S(O)— or —S(O)₂ and the other two are —CH₂—; or
one of W₁, W₂ and W₃ is —O—, —NR^{14′}—, —C(O)—, —S—, —S(O)— or —S(O)₂,
and the other two are —CH₂— and —CH(OH)—; or
one of W₁, W₂ and W₃ is —CH(OH)— and the other two are —CH₂,
and

X, R^{D}, R¹, R², R³, R⁴, R⁶, R⁷, R⁸, R⁹, R¹⁰, R^{14′}, R¹⁷ and R¹⁸, and t are as defined for compound of formula (I).

In an embodiment of compound of formula (IB-1) or (IB-2), B is a five-membered ring or

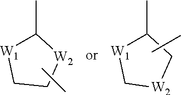

wherein
W₁ and W₂ are as defined for compound of formula of (IB-1a) or (IB-2a) above.

In an embodiment, the compound is of formula (IB-1) or (IB-2), wherein

R¹, R², R³, and R⁴ are independently selected from the group consisting of: H, halo, (C₁-C₆)alkyl, (C₁-C₆)haloalkyl and —CN;

R¹⁷ is selected from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

X, R^{D}, R^{14′}, R⁸ are as defined for compound of formula (I).

In an embodiment, the compound is of formula IB-1a or IB-2a, wherein

t is 0;

X is —CH₂CH₂—;

R¹, R², R³, R⁴ and R¹⁷ are as defined for compound of formula (IB-1a) or (IB-2a) above,

R⁸, R⁹, R¹⁰, R^{14′}, R¹⁸ are as defined for compound of formula (I).

In an embodiment of compound of formula (IB-1) or (IB-2), B is a six-membered ring as set forth in formula:

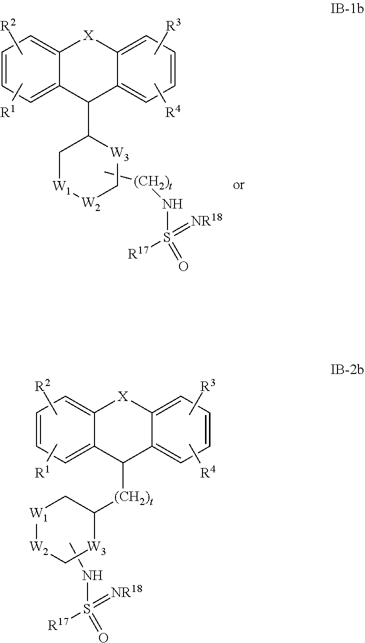

wherein:

all of W₁, W₂ and W₃ are —CH₂—; or

one of W₁, W₂ and W₃ is —O—, —NR^{14′}—, —C(O)—, —S—, —S(O)— or —S(O)₂ and the other two are —CH₂—; or
one of W₁, W₂ and W₃ is —O—, —NR^{14′}—, —C(O)—, —S—, —S(O)— or —S(O)₂ and the other two are —CH₂— and —CH(OH)—; or
one of W₁, W₂ and W₃ is —CH(OH)— and the other two are —CH₂, and

X, R^{D}, R¹, R², R³, R⁴, R⁶, R⁷, R⁸, R⁹, R¹⁰, R^{14′}, R¹⁷, R¹⁸, and t are as defined for compound of formula I.

In an embodiment of compound of formula (IB-1) or (IB-2), B is a six-membered ring

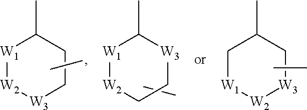

wherein
W₁, W₂ and W₃ are as defined for compound of formula of (IB-1b) or (IB-2b) above.

In an embodiment, the compound is of formula (IB-1b) or (IB-2b), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl, and X, R^{D}, R¹, R², R³, R⁴, R⁶, R⁷, R^{14′}, R⁸, R⁹, R¹⁰, R¹⁸ and t are as defined for compound of formula (I), and W₁, W₂, W₃ are as defined for compound of formula (IB-1b) or (IB-2b) above.

In an embodiment, the compound is of formula (IB-1b) or (IB-2b), wherein R¹, R², R³, and R⁴ are independently selected from the group consisting of: H, halo, (C₁-C₆)alkyl, (C₁-C₆)haloalkyl and —CN;

and X, R^{D}, R⁸, R⁹, R¹⁰, R^{14′}, R¹⁸ and t are as defined for compound of formula (I), and R¹⁷, W₁, W₂, W₃ are as defined for compound of formula (IB-1b) or (IB-2b) above.

In an embodiment, the compound is of formula (IB-1b) or (IB-2b), wherein

R¹, R², R³, R⁴, R¹⁷, W₁, W₂ and W₃ are as defined for compound of formula (IB-1b) or (IB-2b) above,
and X, R^{D}, R⁶, and R¹⁸ are as defined for compound of formula (I),
t is 0 or 1,
R^{14′}, when present, is hydrogen or optionally substituted (C₁-C₆)alkyl, (C₃-C₇) cycloalkyl, aryl, or heteroaryl; —SO₂R⁸; —SO₂NR⁸R⁹; —C(═O) R¹⁰; —C(═O)OR¹⁰; or —C(═O)NR⁸R⁹; wherein said substituents on the (C₁-C₆)alkyl, (C₃-C₇) cycloalkyl, aryl, or heteroaryl are selected from the group consisting of hydroxy, halogen, (C₁-C₆)alkyl, R⁸ and R⁹ are independently selected in each instance from hydrogen, (C₁-C₆)alkyl, aryl, and arylalkyl, wherein said aryl or the aryl of the arylalkyl is optionally substituted with hydroxy, halogen, cyano, nitro, amino, (C₁-C₆)alkylamino, (C₁-C₆)dialkylamino, (C₁-C₆)alkyl, (C₁-C₆)haloalkyl, (C₁-C₆)haloalkoxy, or (C₁-C₆)alkoxy;
R¹⁰ is selected from hydrogen, optionally substituted (C₁-C₆)alkyl, or optionally substituted aryl, wherein said optional substituents are selected from the group consisting of (C₁-C₆)alkyl, OR⁶, NH₂, NHMe, N(Me)₂, and heterocycle.

In an embodiment, the compound is of formula (IB-1b) or (IB-2b), wherein X is absent,

t is 0,
R¹, R², R³, R⁴, R¹⁷, W₁, W₂, W₃, R^{14′}, R⁸, R⁹ and R¹⁰ are as defined for compound of formula (IB-1b) or (IB-2b) above,
and R⁶ and R¹⁸ are as defined for compound of formula (I).
In an embodiment, the compound is of formula (IB-1b) or (IB-2b), wherein X is direct bond,
t is 0,
R¹, R², R³, R⁴, R¹⁷, W₁, W₂, W₃, R^{14′}, R⁸, R⁹ and R¹⁰ are as defined for compound of formula (IB-1b) or (IB-2b) above,
and R⁶ and R¹⁸ are as defined for compound of formula (I).

In an embodiment, the compound is of formula (IB-1b) or (IB-2b), wherein X is —S—,

t is 0,
R¹, R², R³, R⁴, R¹⁷, W₁, W₂, W₃, R^{14′}, R⁸, R⁹ and R¹⁰ are as defined for compound of formula (IB-1b) or (IB-2b) above,
and R⁶ and R¹⁸ are as defined for compound of formula (I).

In an embodiment, the compound is of formula (IB-1b) or (IB-2b), wherein X is —(CH₂CH₂)—,

t is 0,
R¹, R², R³, R⁴, R¹⁷, W₁, W₂, W₃, R^{14′}, R⁸, R⁹ and R¹⁰ are as defined for compound of formula (IB-1b) or (IB-2b) above,
and R⁶ and R¹⁸ are as defined for compound of formula (I).

In an embodiment, the compound is of formula (IB-1b) or (IB-2b), wherein X is —CH═CH—;

t is 0,
R¹, R², R³, R⁴, R¹⁷, W₁, W₂, W₃, R^{14′}, R⁸, R⁹ and R¹⁰ are as defined for compound of formula (IB-1b) or (IB-2b) above,
and R⁶ and R¹⁸ are as defined for compound of formula (I).

In an embodiment, the compound is of formula (IB-1b) or (IB-2b), wherein X is —O—,

t is 0,
R¹, R², R³, R⁴, R¹⁷, W₁, W₂, W₃, R^{14′}, R⁸, R⁹ and R¹⁰ are as defined for compound of formula (IB-1b) or (IB-2b) above,
and R⁶ and R¹⁸ are as defined for compound of formula (I).

In an embodiment, the compound is of formula (IB-1b) or (IB-2b), wherein X is —CH₂O—,

t is 0,
R¹, R², R³, R⁴, R¹⁷, W₁, W₂, W₃, R^{14′}, R⁸, R⁹ and R¹⁰ are as defined for compound of formula (IB-1b) or (IB-2b) above,
and R⁶ and R¹⁸ are as defined for compound of formula (I).

In an embodiment, the compound is of formula (IB-1b) or (IB-2b), wherein X is —OCH₂—,

t is 0,
R¹, R², R³, R⁴, R¹⁷, W₁, W₂, W₃, R^{14′}, R⁸, R⁹ and R¹⁰ are as defined for compound of formula (IB-1b) or (IB-2b) above,
and R⁶ and R¹⁸ are as defined for compound of formula (I).

In an embodiment, the compound is of formula (IB-1b) or (IB-2b), wherein X is —C(═O)NR^{D}—, R^{D} is hydrogen or (C₁-C₆)alkyl;

t is 0,
R¹, R², R³, R⁴, R¹⁷, W₁, W₂, W₃, R^{14′}, R⁸, R⁹ and R¹⁰ are as defined for compound of formula (IB-1b) or (IB-2b) above, and R⁶ and R¹⁸ are as defined for compound of formula (I).

In an embodiment, the compound is of formula (IB), wherein p and q are both zero, R¹⁵ is H and R¹⁶ is selected from H and OH. In one embodiment, the compound is of formula (IB-3) or (IB-3′):

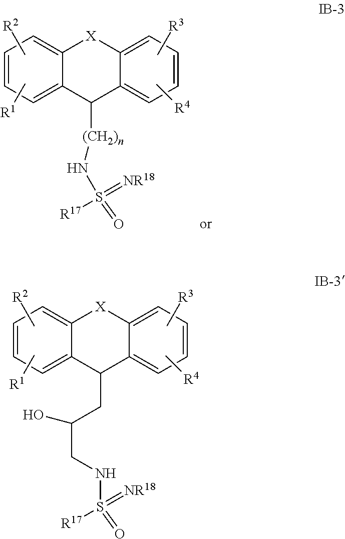

wherein
n is 2, 3 or 4,
R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇)cycloalkyl, (C₁-C₆)alkenyl, (C₁-C₆)alkynyl or aryl, R¹, R², R³, and R⁴ are independently selected from the group consisting of: H, halo, (C₁-C₆)alkyl, (C₁-C₆)haloalkyl and —CN;
and X is as defined for compound of formula (I).

In an embodiment, the compound is of formula (IB), wherein p is zero, q is 1, and wherein two of R¹⁵ and/or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, and X, R^{D}, R¹, R², R³, R⁴, R⁶, R⁷, R¹⁷, R¹⁸, Q, R^{14′}, R⁸, R⁹, R¹⁰ and n are as defined for compound of formula (I).

In an embodiment of the compound of formula I, T is a benzene ring, U is a benzene ring, and Y is —C(R¹⁴)(R⁵)—, R¹⁴ is hydrogen and R⁵ is

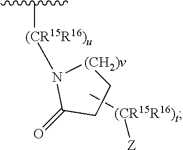

wherein X, R^{D}, R¹, R², R³, R⁴, R⁶, R⁷, R¹⁵, R¹⁶, Z, R¹⁷ and R¹⁸, u, v and t are as defined for compound of formula (I).

In an embodiment, R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl, and X, R^{D}, R¹, R², R³, R⁴, R⁶, R⁷, R¹⁵, R¹⁶, Z, and R¹⁸, u, v and t are as defined for compound of formula (I).

In an embodiment, R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, (C₁-C₆)haloalkylthio, —CHC(═O)O(C₁-C₆)alkyl, and X, R^{D}, R⁶, R⁷, R¹⁵, R¹⁶, Z, R¹⁷ and R¹⁸, u, v and t are as defined for compound of formula (I).

In an embodiment of the compound of formula (I), T is a benzene ring, U is a benzene ring, and Y is —C(R¹⁴)(R⁵)—, wherein

R¹⁴ is hydrogen,
R⁵ is —(CR¹⁵R¹⁶)ₚ-Q_{q}-(CR¹⁵R¹⁶)ₙ₋ₚ—Z,
p is 0, q is 1,
Q is —NR^{14′}—, wherein R^{14′} taken together with R¹⁵ or R¹⁶ form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;
and X, R^{D}, R¹, R², R³, R⁴, R⁶, R⁷, R¹⁵, R¹⁶, R¹⁷ and R¹⁸, and n are as defined for compound of formula (I).

In an embodiment of the compound of formula (I), R^{14′} taken together with R¹⁵ or R¹⁶ form a six membered non-aromatic heterocycle wherein said six membered heterocycle is optionally substituted with OH.

In an embodiment of the compound of formula I, T is a benzene ring, U is a benzene ring, and Y is —C(═R^{5a})—, i.e.

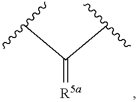

and R¹⁴ is hydrogen. In one embodiment, the compound is of formula (IC)

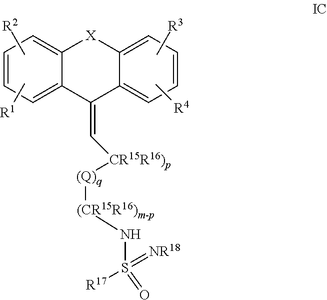

wherein X, R^{D}, R¹, R², R³, R⁴, R⁶, R⁷, R¹⁵, R¹⁶, R¹⁷ and R¹⁸, Q, m, p, and q are as defined for compound of formula I.

In one embodiment, the compound is of formula (IC), wherein X, R^{D}, R¹, R², R³, R⁴, R⁶, R⁷, R¹⁵, R¹⁶, R¹⁷ and R¹⁸, Q, m, p, and q are as defined for the compound of formula (I),

provided that when q is 1 and Q is —C(O)—, m-p is not 0.

In an embodiment, the compound is of formula (IC), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,

and X, R^{D}, R¹, R², R³, R⁴, R⁶, R⁷, R¹⁵, R¹⁶ and R¹⁸, Q, R^{14′}, R⁸, R⁹, R¹⁰, m, p, and q are as defined for compound of formula (I).

In an embodiment, the compound is of formula (IC), wherein

R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, (C₁-C₆)haloalkylthio, —CHC(═O)O(C₁-C₆)alkyl,

and R¹⁷ is as defined for compound of formula (IC),

and X, R^{D}, R¹⁵, R¹⁶ and R¹⁸, Q, R^{14′}, R⁸, R⁹, R¹⁰, n, p, and q are as defined for compound of formula (I).

In an embodiment, the compound is of formula (IC), wherein
m is 2 or 3, p is 0 or 1,
q is 0,

and R¹⁷, R¹, R², R³, R⁴, R⁶ and R⁷, are as defined for compound of formula (IC),

and X, R^{D}, R¹⁵, R¹⁶ and R¹⁸ are as defined for compound of formula (I).

In an embodiment of compound of Formula IC, R¹⁵ and R¹⁶ in each occurrence are selected independently from the group consisting of from H, OH, cyano, amino, (C₁-C₃)alkylamino, (C₁-C₃)dialkylamino, (C₁-C₃)alkyl, (C₁-C₃)haloalkyl, (C₁-C₃)haloalkoxy, and (C₁-C₃)alkoxy, or, taken together, two of R¹⁵ and R¹⁶ form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₃)alkylamino, (C₁-C₃)dialkylamino, (C₁-C₃)alkyl, (C₁-C₃)haloalkyl, (C₁-C₃)haloalkoxy, and (C₁-C₃)alkoxy.

In an embodiment of compound of Formula (IC), two of R¹⁵ and/or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy.

In an embodiment, two R¹⁵ and/or R¹⁶ groups from different —(CR¹⁵R¹⁶)— groups form a direct bond or an alkylene —(CH₂)_{n′}—, wherein n′ is 1, 2, 3, 4 or 5, thus forming a three to seven membered non-aromatic carbocycle or heterocycle B, wherein H atoms of alkylene are optionally replaced with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy.

In an embodiment, said compound is of formula (IC-1)

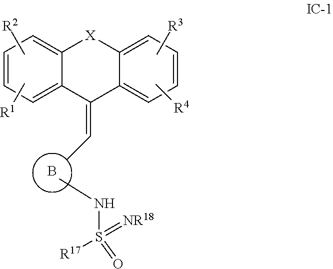

wherein
B is as defined for compound of formula (IC) above,
and X, R^{D}, R¹, R², R³, R⁴, R⁶, R⁷, R¹⁷ and R¹⁸ are as defined for compound of formula (I).

In an embodiment, the compound is of formula (IC-1), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,

and X, B, R^{D}, R¹, R², R³, R⁴, R⁶, R⁷, and R¹⁸ are as defined for compound of formula (I),
B is as defined for compound of formula (IC-1) above.

In an embodiment, the compound is of formula (IC-1), wherein R¹, R², R³, and R⁴ are independently selected from the group consisting of: H, halo, (C₁-C₆)alkyl, (C₁-C₆)haloalkyl and —CN; and X, R^{D} and R¹⁸ are as defined for compound of formula I, B and R¹⁷ are as defined for compound of formula (IC-1) above.

In an embodiment, the compound is of formula (IC-1), wherein B is three to seven membered non-aromatic carbocycle or heterocycle, optionally substituted with one or two substituents selected independently from the group consisting of OH and (C₁-C₆)alkyl;

and X, R^{D}, and R¹⁸ are as defined for compound of formula (I), and R¹⁷, R¹, R², R³, and R⁴ are as defined for compound of formula (IC-1) above.

In an embodiment of the compound of formula (I), T is a benzene ring, U is a benzene ring, and Y is —C(═R^{5a})— and —R¹⁴ and one R¹⁵ or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy. In an embodiment, said compound is of formula (IC-2):

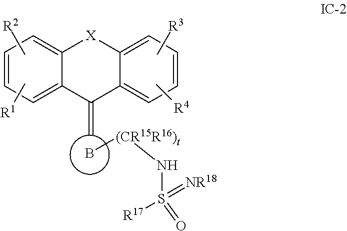

wherein

B is as defined for compound of formula (I) above, and X, t, R^{D}, R¹, R², R³, R⁴, R⁶, R⁷, R¹⁵, R¹⁶, R¹⁷ and R¹⁸ are as defined for compound of formula (I).

In an embodiment, the compound is of formula (IC-2), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,

and X, t, R^{D}, R¹, R², R³, R⁴, R⁶, R⁷, and R¹⁸ are as defined for compound of formula (I),
B is as defined for compound of formula (IC-2) above.

In an embodiment, the compound is of formula (IC-2), wherein R¹, R², R³, and R⁴ are independently selected from the group consisting of: H, halo, (C₁-C₆)alkyl, (C₁-C₆)haloalkyl and —CN;

and X, R^{D}, R¹⁸ and t are as defined for compound of formula (I), B and R¹⁷ are as defined for compound of formula (IC-2) above.

In an embodiment, the compound is of formula (IC-2), wherein t is 0;

and X, R^{D}, R¹⁸ and t are as defined for compound of formula (I), B, R¹, R², R³, and R⁴ and R¹⁷ are as defined for compound of formula (IC-2) above.

In an embodiment of compound of formula (IC-1) or (IC-2), B is a five-membered ring as set forth in formula:

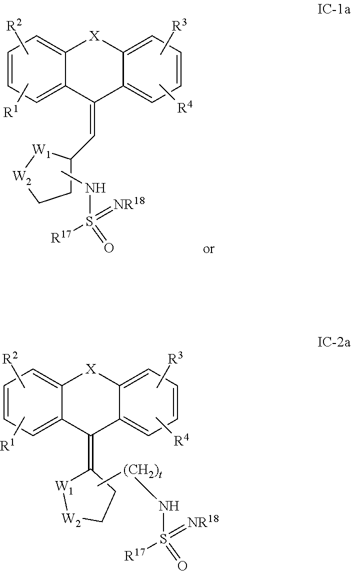

wherein:

W₁ and W₂ are both —CH₂—; or
one of W₁ and W₂ is selected from a group consisting of —O—, —NR^{14′}—, —C(O)—, —S—, —S(O)— or —S(O)₂ and the other is —CH₂—; or

one of W₁ and W₂ is —CH(OH)— and the other is —CH₂—;
and wherein
X, R^{D}, R¹, R², R³, R⁴, R⁶, R⁷, R⁸, R⁹, R¹⁰, R^{14′}, R¹⁷ and R¹⁸, and t are as defined for compound of formula (I).

In an embodiment of compound of formula (IC-1) or (IC-2), B is a five-membered ring or

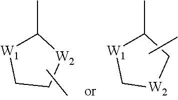

wherein
W₁ and W₂ are as defined for compound of formula of (IC-1a) or (IC-2a) above.

In an embodiment, the compound is of formula (IC-1a) or (IC-2a), wherein

R¹, R², R³, and R⁴ are independently selected from the group consisting of: H, halo, (C₁-C₆)alkyl, (C₁-C₆)haloalkyl and —CN;

R¹⁷ from phenyl, pyridinyl, thiazolyl, furanyl, thiophenyl, pyrrolyl, thienyl, each optionally substituted with one or two substituents selected independently from the group consisting of OH, halogen, amino, (C₁-C₆)alkylthio, (C₁-C₆)alkyl, (C₁-C₆)haloalkyl, (C₁-C₆)haloalkoxy, (C₁-C₆)alkoxy, (C₁-C₆) hydroxyalkyl, (C₁-C₆)haloalkylthio and N₃;

t is 0 or 1;

X, R^{D}, R⁸, R⁹, R¹⁰, R^{14′}, R⁸ are as defined for compound of formula (i).

In an embodiment, the compound is of formula IC-1a or IC-2a, wherein

X is —CH₂CH₂—,

t is 0;

R¹, R², R³, R⁴ and R¹⁷ are as defined for compound of formula IC-1a or IC-2a above,

R¹⁸ are as defined for compound of formula I.

In an embodiment of compound of formula (IC-1) or (IC-2), B is a six-membered ring as set forth in formula:

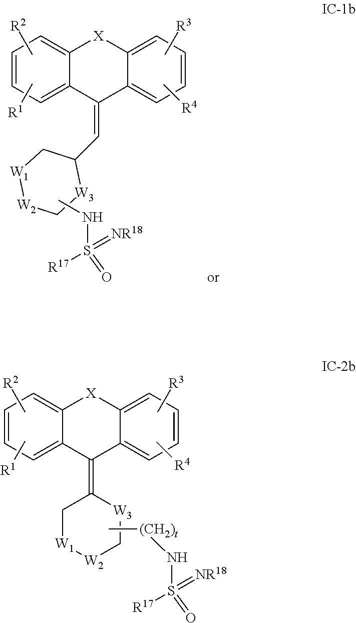

wherein:
all of W₁, W₂ and W₃ are —CH₂—; or
one of W₁, W₂ and W₃ is —O—, —NR^{14′}—, —C(O)—, —S—, —S(O)— or —S(O)₂ and the other two are —CH₂—; or
one of W₁, W₂ and W₃ is —O—, —NR^{14′}—, —C(O)—, —S—, —S(O)— or —S(O)₂ and the other two are —CH₂— and —CH(OH)—; or
   one of W₁, W₂ and W₃ is —CH(OH)— and the other two are —CH₂, and wherein

X, R^{D}, R¹, R², R³, R⁴, R⁶, R⁷, R⁸, R⁹, R¹⁰, R^{14′}, R¹⁷ and R¹⁸, and t are as defined for compound of formula (I).

In an embodiment of compound of formula (IC-1) or (IC-2), B is a six-membered ring

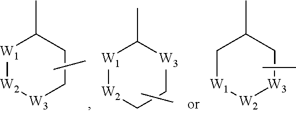

wherein
W₁, W₂ and W₃ are as defined for compound of formula of (IC-1b) or (IC-2b) above.

In an embodiment, the compound is of formula (IC-1b) or (IC-2b), wherein R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl, and X, R^{D}, R¹, R², R³, R⁴, R⁶, R⁷, R⁸, R⁹, R¹⁰, R^{14′} and R¹⁸ and t are as defined for compound of formula (I), and W₁, W₂, W₃ are as defined for compound of formula IC-1b or IC-2b above.

In an embodiment, the compound is of formula (IC-1b) or (IC-2b), wherein R¹, R², R³, and R⁴ are independently selected from the group consisting of: H, halo, (C₁-C₆)alkyl, (C₁-C₆)haloalkyl and —CN;

and X, R^{D}, R⁸, R⁹, R¹⁰, R^{14′}, R¹⁸ and t are as defined for compound of formula (I), and R¹⁷, W₁, W₂, W₃ are as defined for compound of formula (IC-1b) or (IC-2b) above.

In an embodiment, the compound is of formula (IC-1b) or (IC-2b), wherein

R¹, R², R³, R⁴, R¹⁷, W₁, W₂ and W₃ are as defined for compound of formula (IC-1b) or (IC-2b) above,
and X, R^{D}, R⁶, and R¹⁸ are as defined for compound of formula (I),
t is 0 or 1,
R^{14′}, when present, is optionally substituted (C₁-C₆)alkyl, (C₃-C₇) cycloalkyl, aryl, or heteroaryl; —SO₂R⁸; —SO₂NR⁸R⁹; —C(═O) R¹⁰; —C(═O)OR¹⁰; or —C(═O)NR⁸R⁹; wherein said substituents on the (C₁-C₆)alkyl, (C₃-C₇)cycloalkyl, aryl, or heteroaryl are selected from the group consisting of hydroxy, halogen, (C₁-C₆)alkyl, R⁸ and R⁹ are independently selected in each instance from hydrogen, (C₁-C₆)alkyl, aryl, and arylalkyl, wherein said aryl or the aryl of the arylalkyl is optionally substituted with hydroxy, halogen, cyano, nitro, amino, (C₁-C₆)alkylamino, (C₁-C₆)dialkylamino, (C₁-C₆)alkyl, (C₁-C₆)haloalkyl, (C₁-C₆)haloalkoxy, or (C₁-C₆)alkoxy;
R¹⁰ is selected from hydrogen, optionally substituted (C₁-C₆)alkyl, or optionally substituted aryl, wherein said optional substituents are selected from the group consisting of (C₁-C₆)alkyl, OR⁶, NH₂, NHMe, N(Me)₂, and heterocycle.

In an embodiment of the compound of formula (I), T is a benzene ring, U is a benzene ring, Y is —C(R^{A}R^{B}), and R^{A} and R^{B} together with the C atom which they are attached form a three to six membered aliphatic carbocycle or heterocycle A which is substituted with Z, i.e.

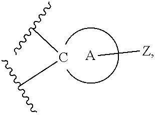

and said carbocycle or heterocycle A which is substituted with Z is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy. In an embodiment, the compound is of formula (ID)

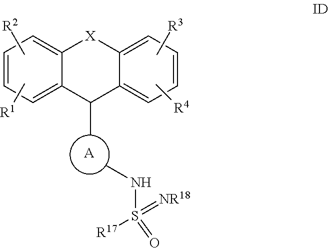

wherein
A is as defined for compound of formula (I) above,
X, R^{D}, R¹, R², R³, R⁴, R⁶, R, R¹⁷ and R¹⁸ are as defined for compound of formula (I).

In an embodiment of the compound of formula (I), T and U are heteroaromatic rings each independently selected from the group consisting of a benzene ring, furan, imidazole, isothiazole, isoxazole, oxadiazole, oxazole, pyran, pyrazine, pyrazole, pyridazine, pyridine, pyrimidine, pyrrole, thiadiazole, thiazine, thiazole, thiophene, triazine, and triazole, and X, R^{D}, Y, R^{A} and R^{B}, R¹, R², R³, R⁴, R⁶, R⁷, R⁸, R⁹, R¹⁰, R^{14′}, R¹⁴, R¹⁵, R¹⁶, R¹⁷ and R¹⁸, m, n, p, q, t, u and v are as defined for compound of formula (I).

In an embodiment of the compound of formula (I), T and U are heteroaromatic rings each independently selected from the group consisting of a benzene ring, furan, imidazole, isothiazole, isoxazole, oxadiazole, oxazole, pyran, pyrazine, pyrazole, pyridazine, pyridine, pyrimidine, pyrrole, thiadiazole, thiazine, thiazole, thiophene, triazine, and triazole,

and R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl, and X, R^{D}, Y, R^{A} and R^{B}, R¹, R², R³, R⁴, R⁶, R⁷, R, R⁹, R¹⁰, R^{4′}, R¹⁴, R¹⁵, R¹⁶ and R¹⁸, Q, m, n, p, q, t, u and v are as defined for compound of formula (I).

In an embodiment, T and U are heteroaromatic rings each independently selected from a pyridine, pyrimidine, pyridazine, thiophene, thiazole, oxazole, imidazole, pyrrole, and furan, R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,

and X, R^{D}, Y, R^{A} and R^{B}, R¹, R², R³, R⁴, R⁶, R⁷, R⁸, R⁹, R¹⁰, R^{14′}, R¹⁴, R¹⁵, R¹⁶ and R¹⁸, Q, m, n, p, q, t, u and v are as defined for compound of formula I.

In an embodiment, one of T and U is a benzene ring, and the other of T and U is selected from pyridine, pyrimidine, and thiophene,

and X, R^{D}, Y, R^{A} and R^{B}, R¹, R², R³, R⁴, R⁶, R⁷, R⁸, R⁹, R¹⁰, R^{14′}, R¹⁴, R¹⁵, R¹⁶, R¹⁷ and R¹⁸, Q, m, n, p, q, t, u and v are as defined for compound of formula (I).

In an embodiment, one of T and U is a benzene ring, and the other of T and U is selected from pyridine, pyrimidine, and thiophene,

R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,
and X, R^{D}, Y, R^{A} and R^{B}, R¹, R², R³, R⁴, R⁶, R⁷, R⁸, R⁹, R¹⁰, R^{14′}, R¹⁴, R¹⁵, R¹⁶ and R¹⁸, Q, m, n, p, q, t, u and v are as defined for compound of formula (I).

In an embodiment, T and U are each independently selected from a benzene ring and pyridine,

and X, R^{D}, Y, R^{A} and R^{B}, R¹, R², R³, R⁴, R⁶, R⁷, R⁸, R⁹, R¹⁰, R^{14′}, R¹⁴, R¹⁵, R¹⁶, R¹⁷ and R¹⁸, Q, m, n, p, q, t, u and v are as defined for compound of formula (I).

In an embodiment, T and U are each independently selected from a benzene ring and pyridine,

R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆) acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O) (C₁-C₆)alkyl, —C(═O) H, (C₁-C₆) hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇) cycloalkyl, (C₁-C₆)alkenyl, (C₁-C₆)alkynyl or aryl,
and X, R^{D}, Y, R^{A} and R^{B}, R¹, R², R³, R⁴, R⁶, R⁷, R⁸, R⁹, R¹⁰, R^{14′}, R¹⁴, R¹⁵, R¹⁶, R¹⁷ and R¹⁸, Q, m, n, p, q, t, u and v are as defined for compound of formula (I).
In an embodiment of the compound of formula (I) selected from:

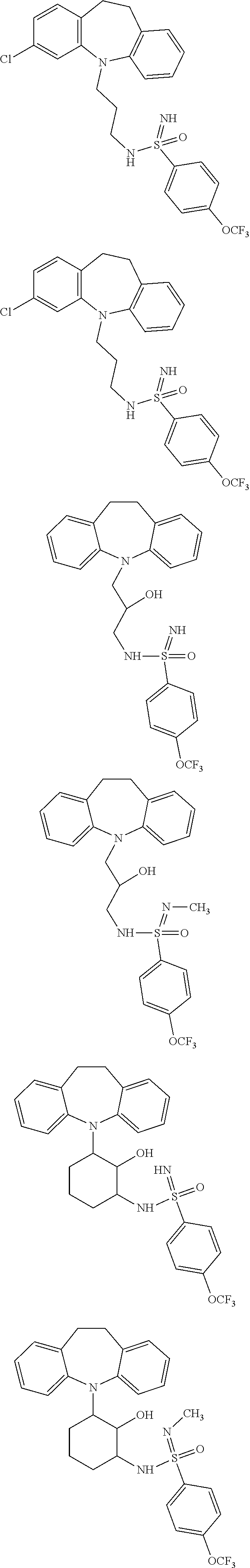

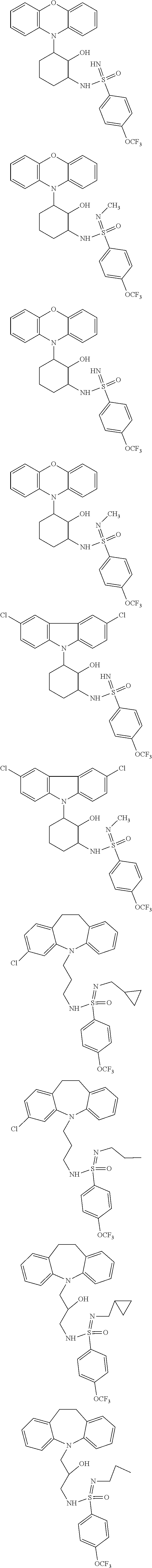

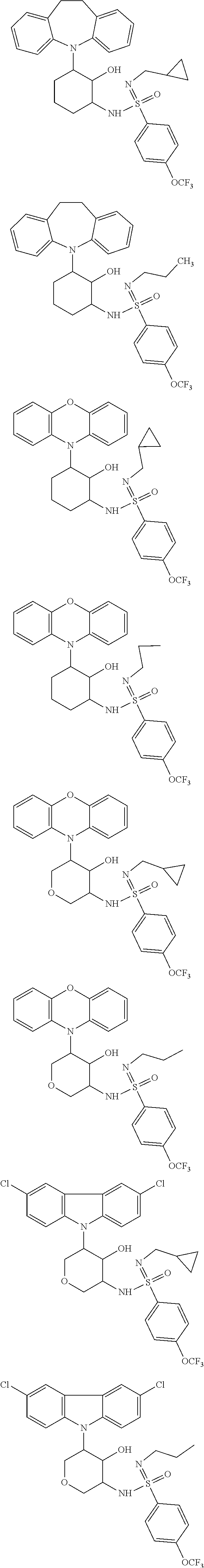

or an enantiomer, a diastereomer, a tautomer or a pharmaceutically acceptable salt thereof.

The enantiomers and diastereomers of the above compounds are shown in Table 1:

TABLE 1

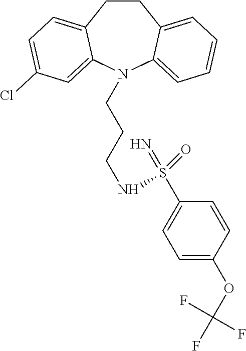 [SMILES: N=[S@](=O)(NCCCN1c2ccccc2CCc2ccc(Cl)cc21)c1ccc(OC(F)(F)F)cc1]

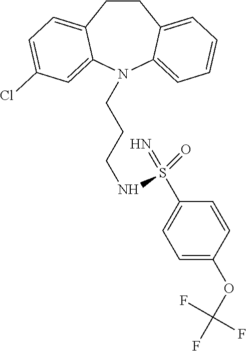 [SMILES: N=[S@@](=O)(NCCCN1c2ccccc2CCc2ccc(Cl)cc21)c1ccc(OC(F)(F)F)cc1]

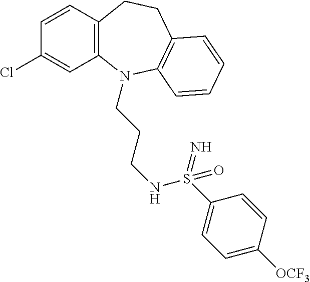 [SMILES: N=S(=O)(NCCCN1c2ccccc2CCc2ccc(Cl)cc21)c1ccc(OC(F)(F)F)cc1]

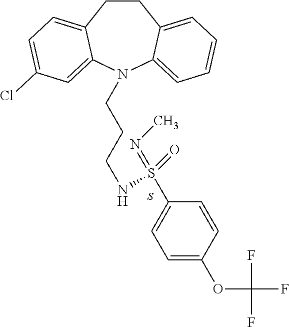

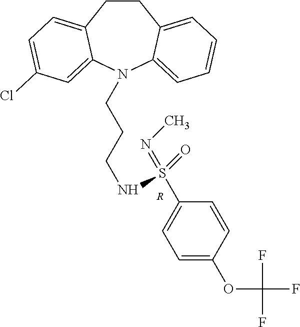

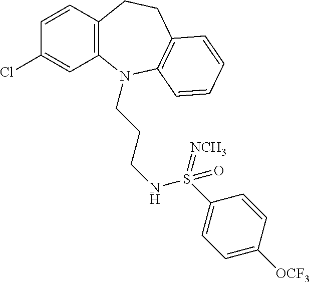

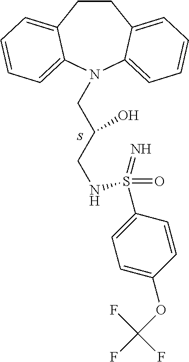

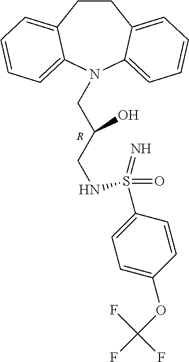

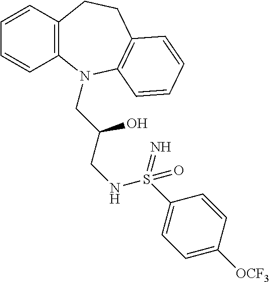 [SMILES: N=S(=O)(NC[C@H](O)CN1c2ccccc2CCc2ccccc21)c1ccc(OC(F)(F)F)cc1]

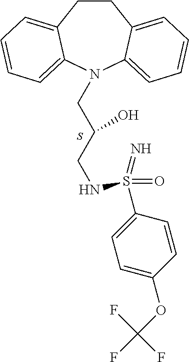

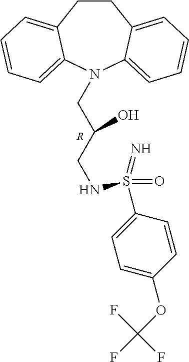

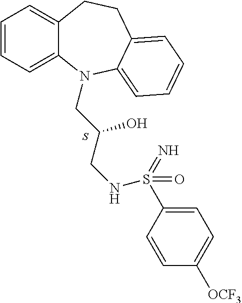

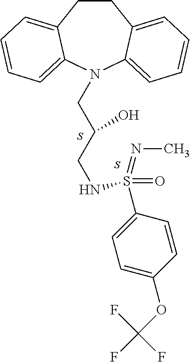

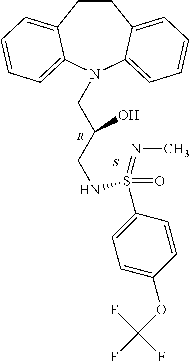

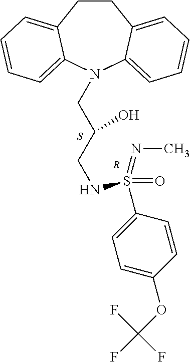

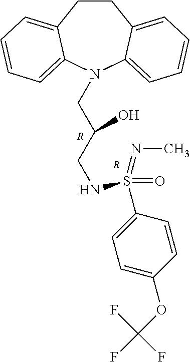

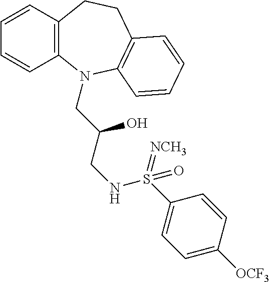

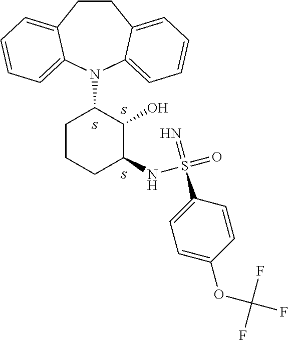

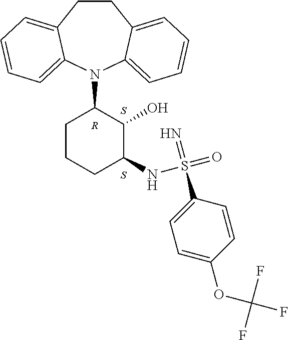

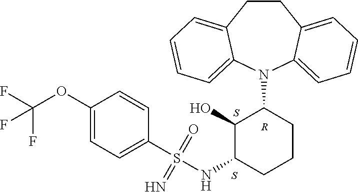

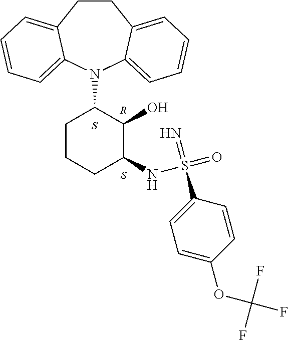

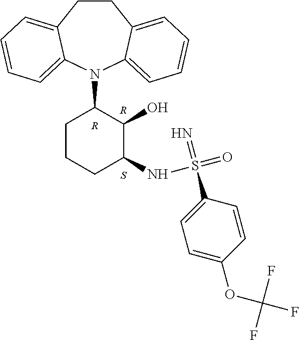

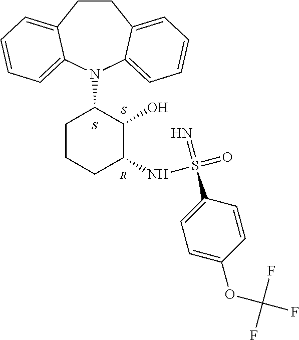

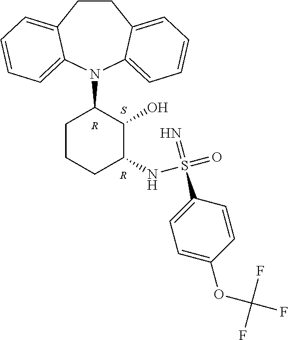

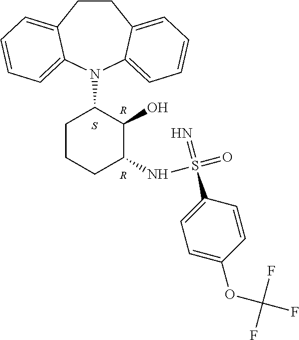

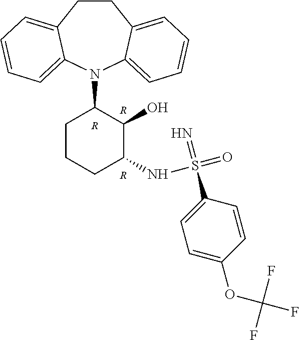

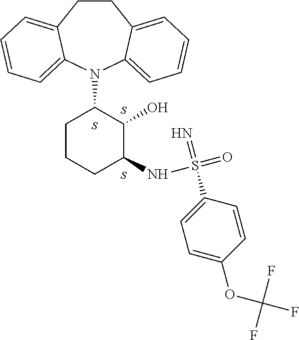

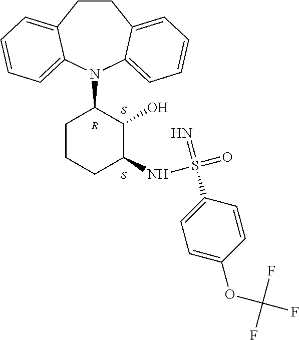

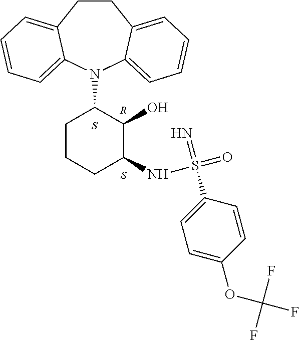

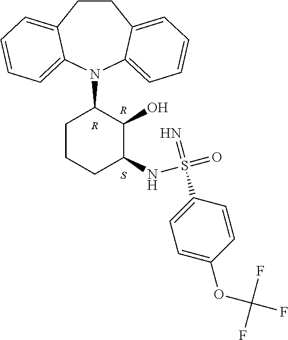

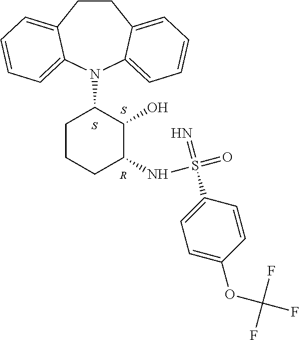

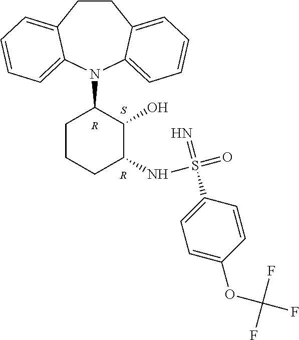

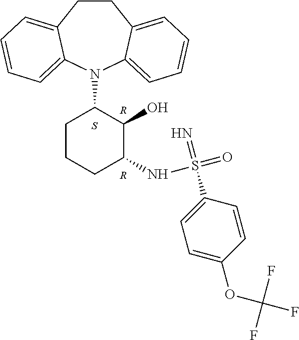

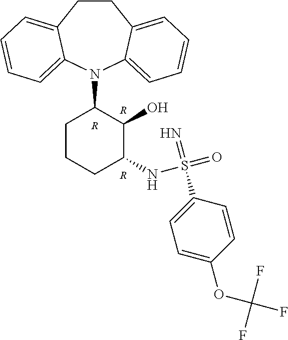

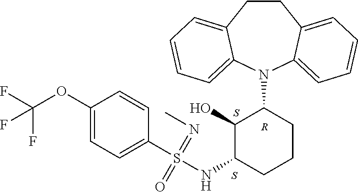

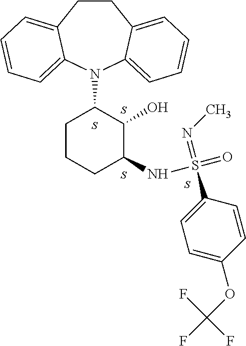

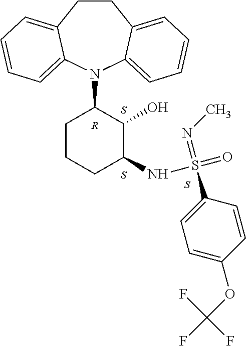

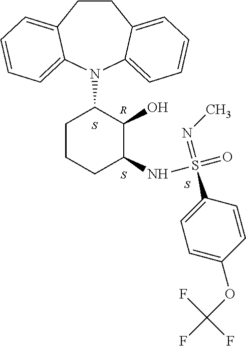

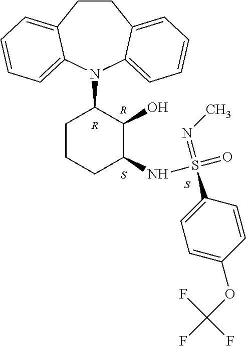

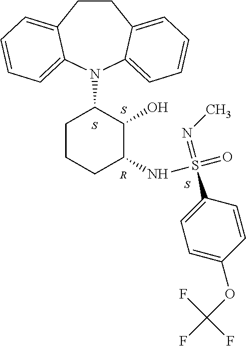

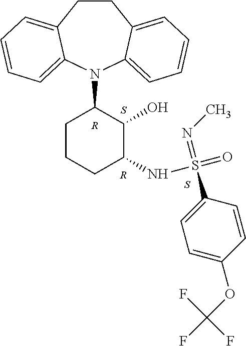

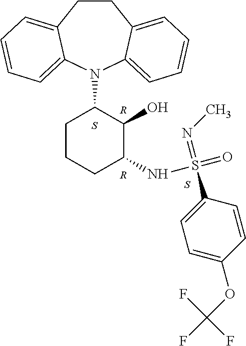

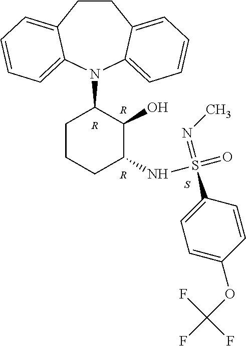

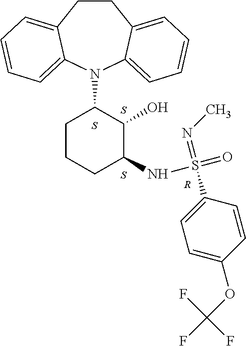

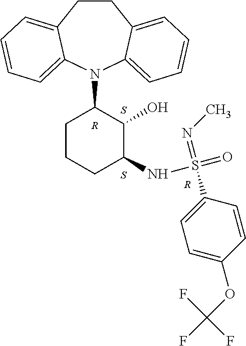

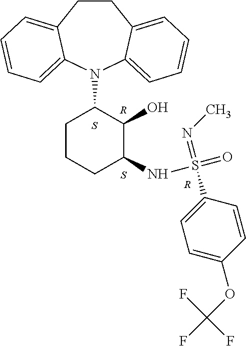

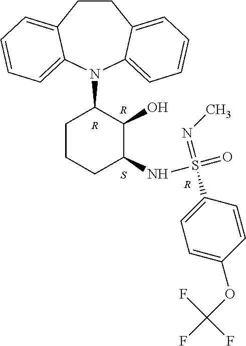

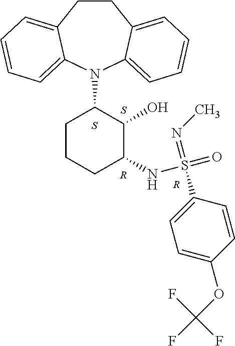

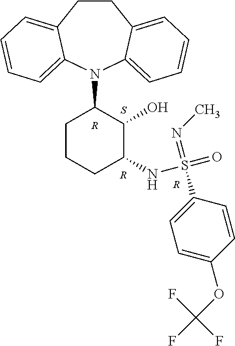

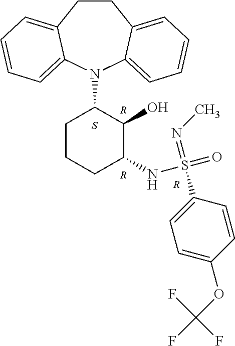

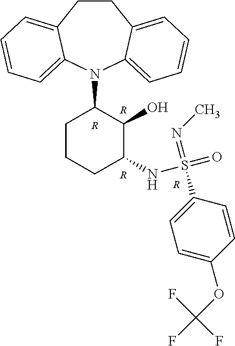

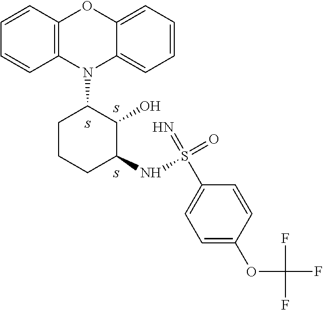

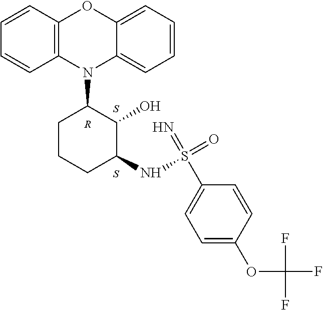

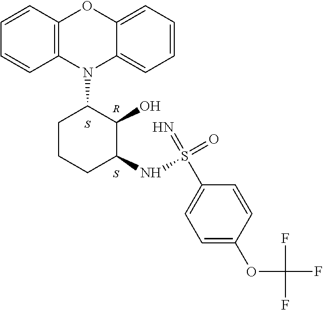

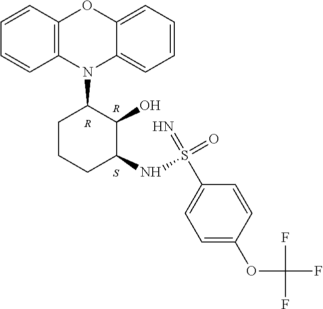

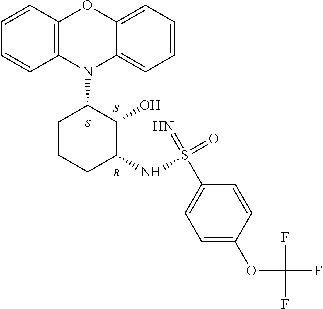

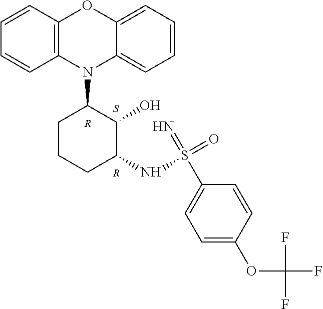

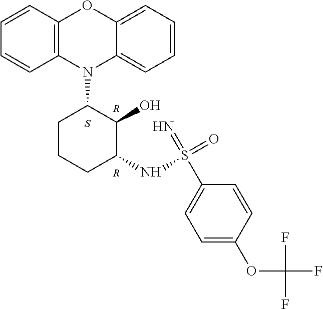

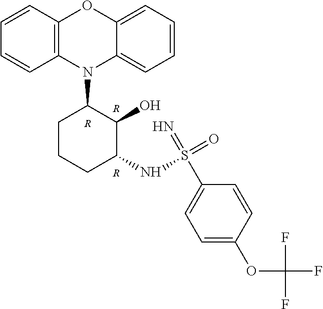

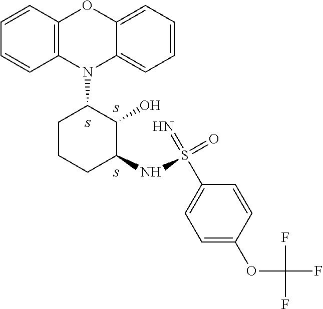

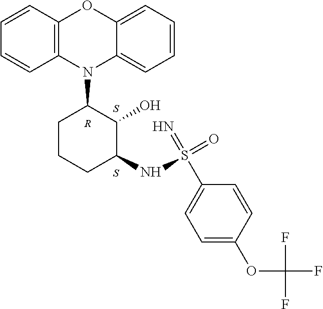

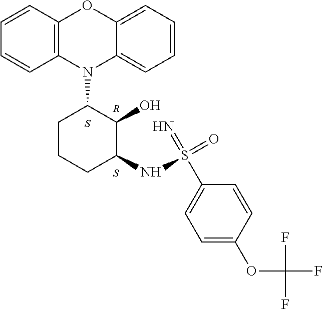

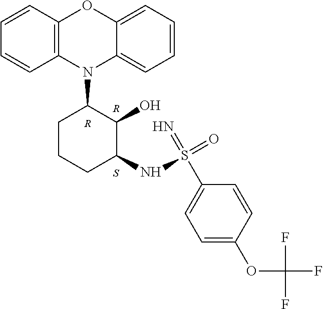

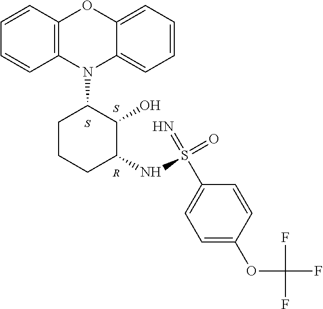

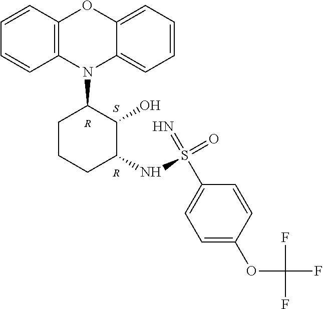

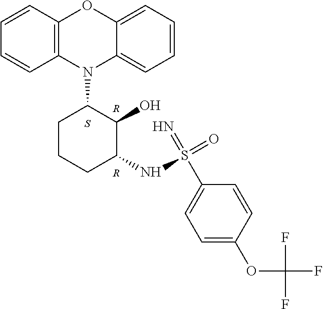

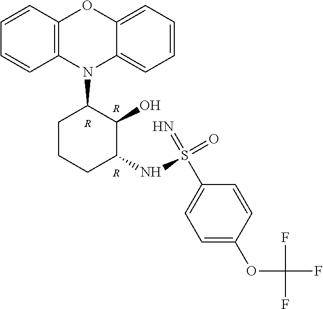

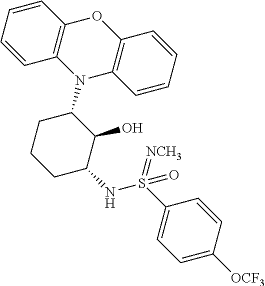

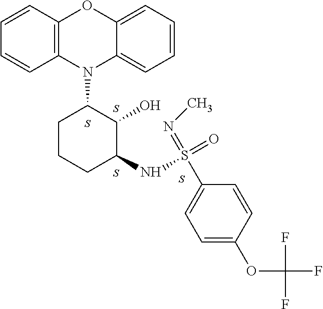

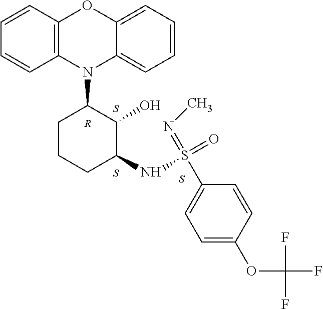

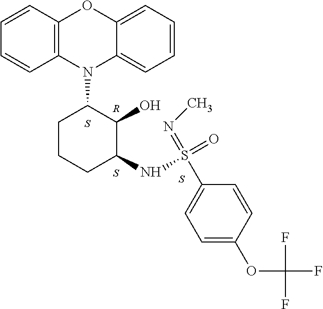

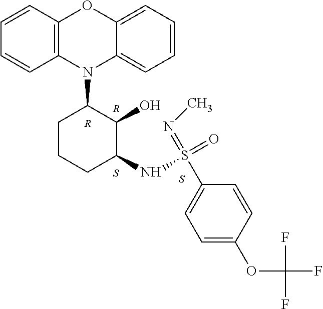

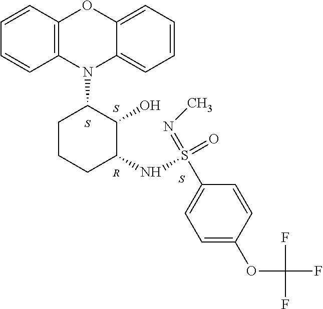

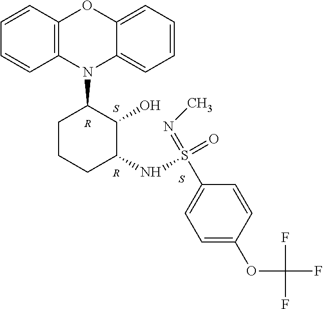

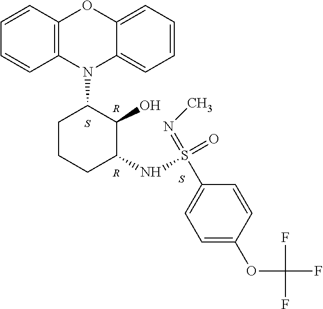

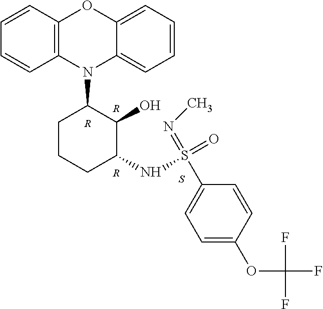

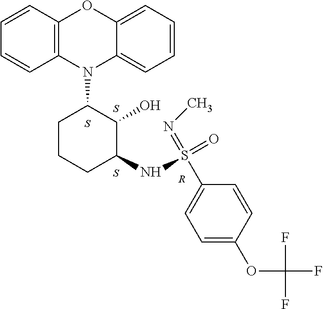

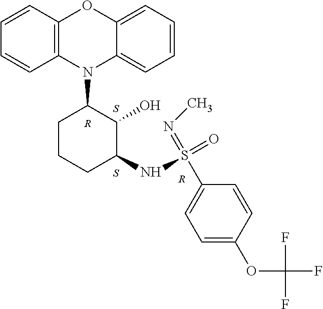

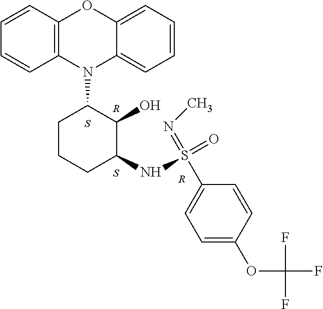

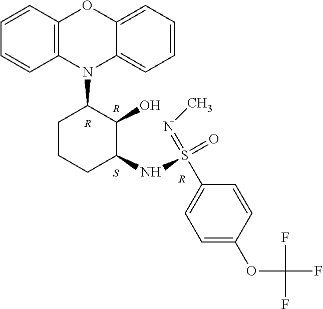

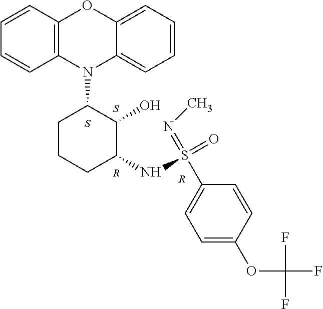

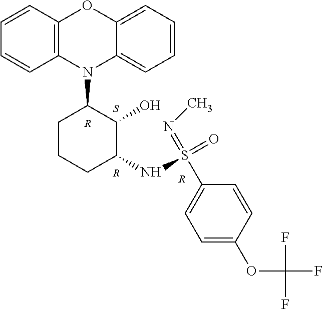

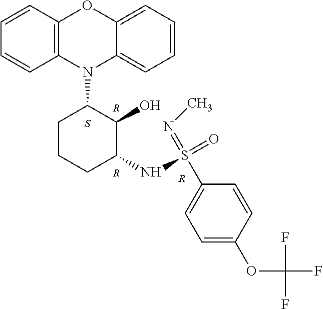

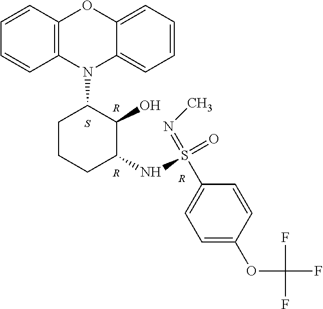

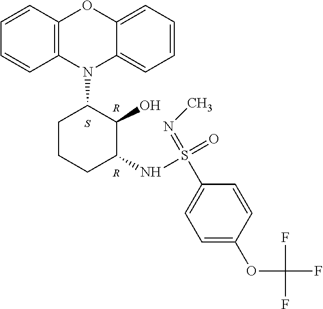

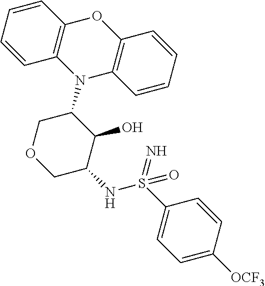 [SMILES: N=S(=O)(N[C@@H]1COC[C@H](N2c3ccccc3Oc3ccccc32)[C@H]1O)c1ccc(OC(F)(F)F)cc1]

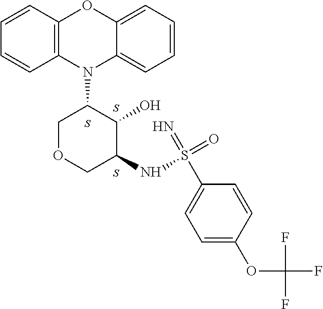

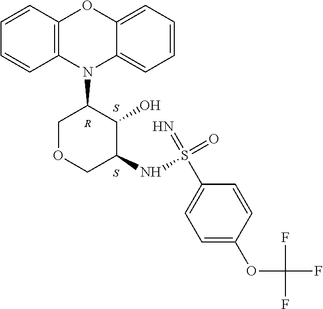

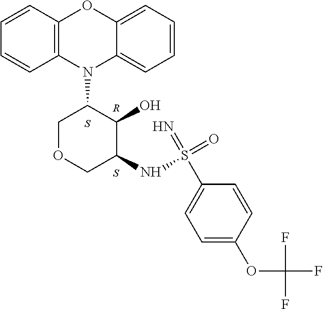

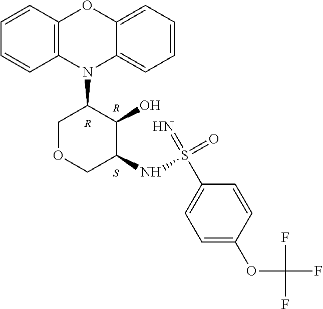

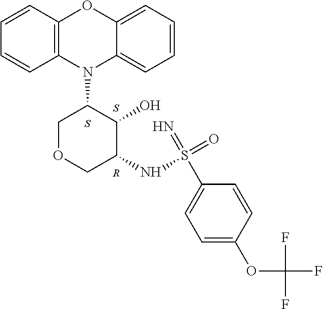

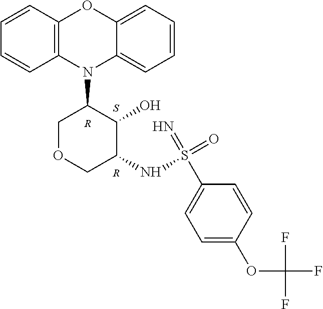

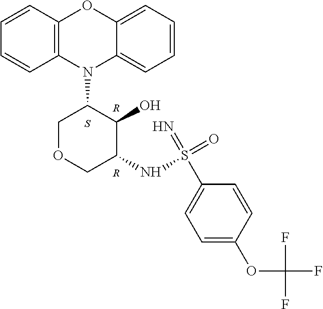

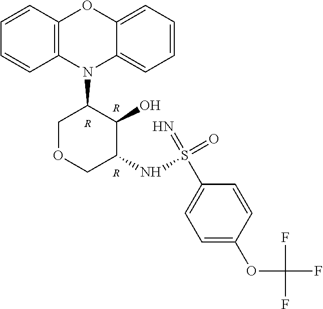

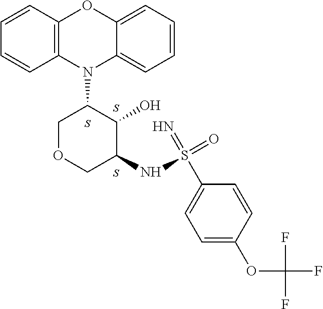

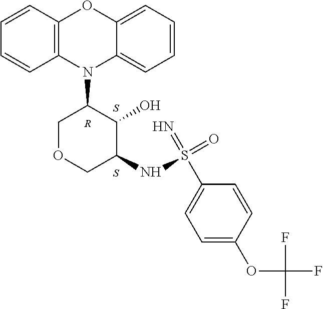

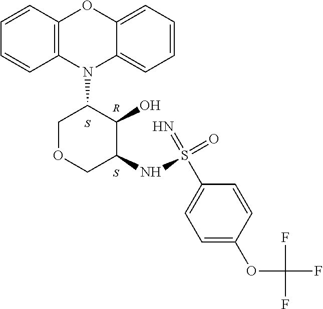

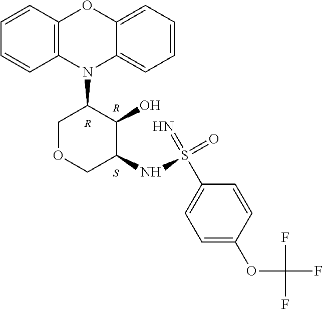

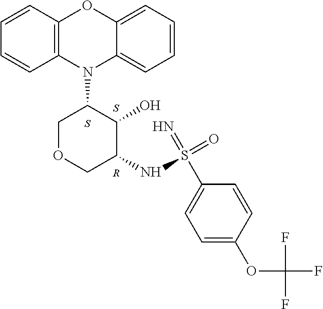

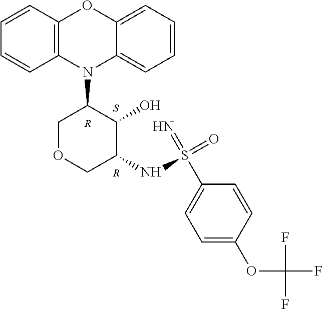

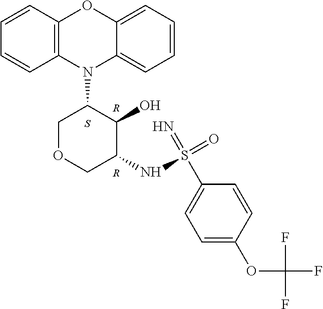

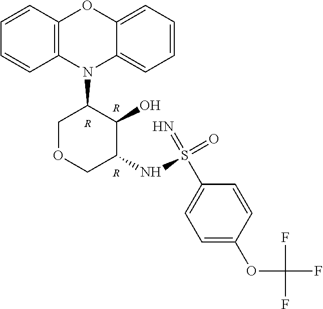

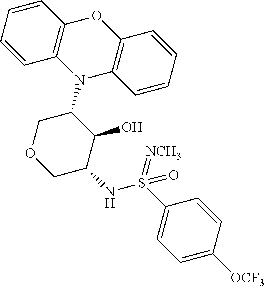

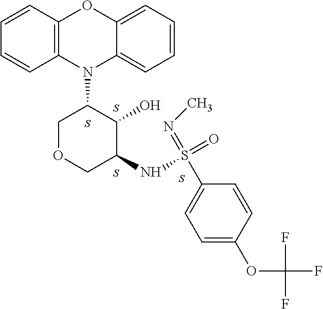

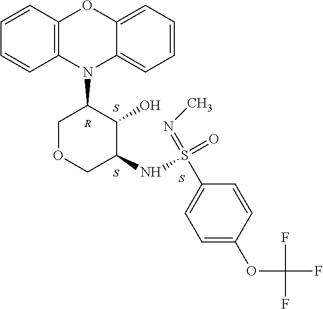

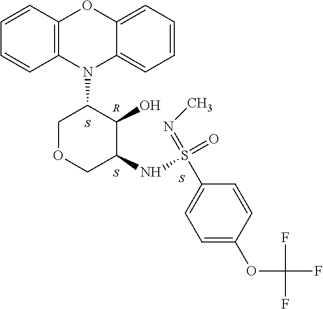

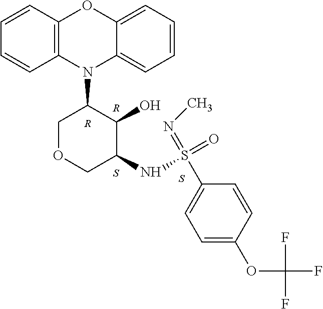

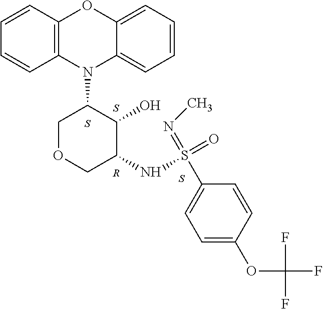

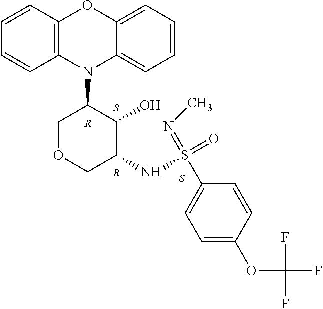

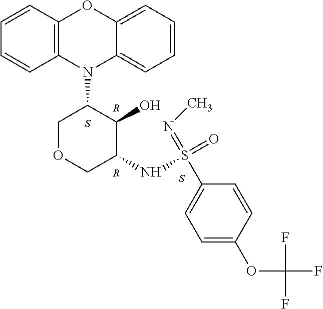

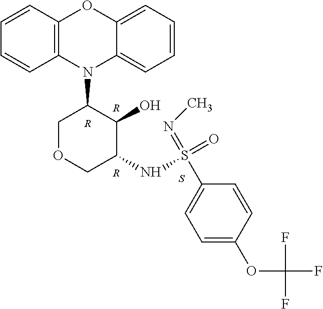

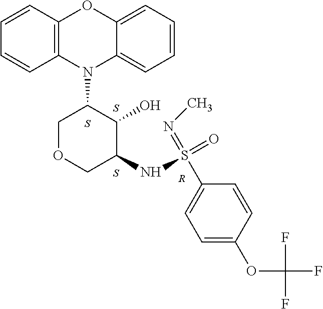

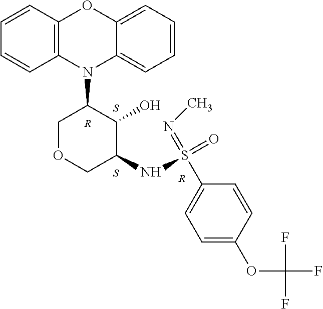

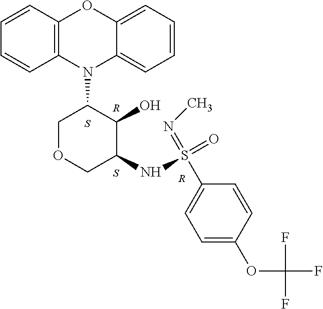

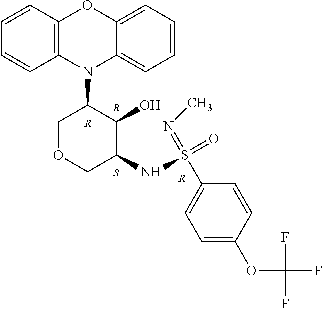

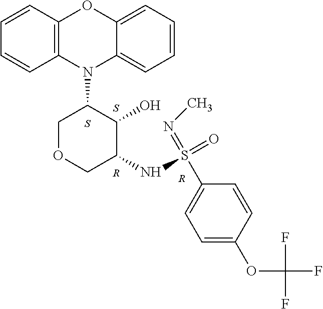

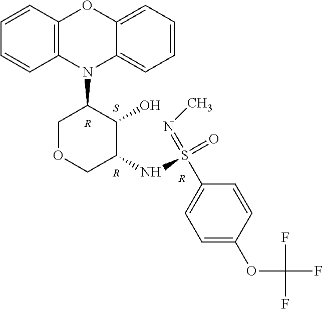

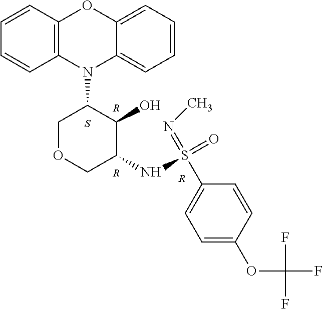

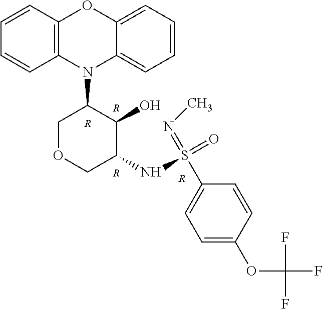

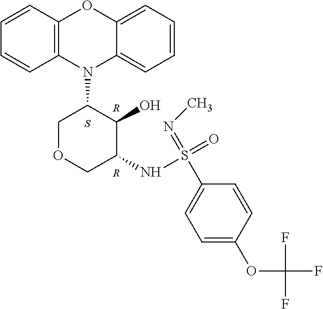

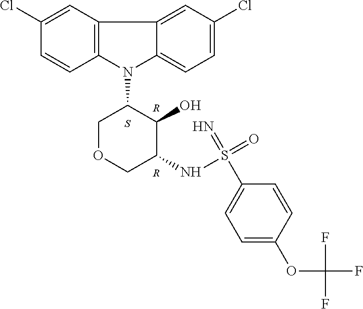

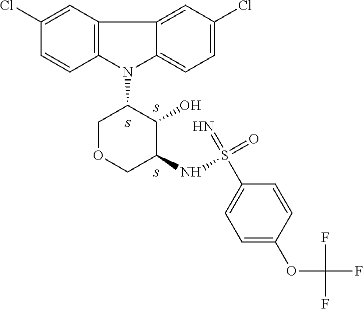

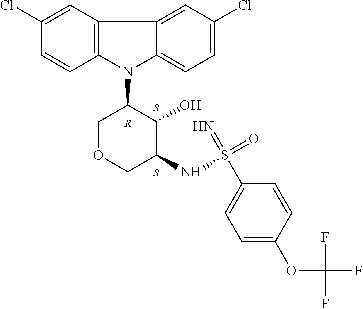

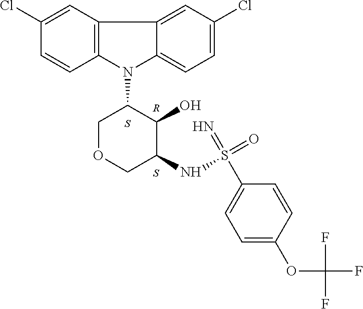

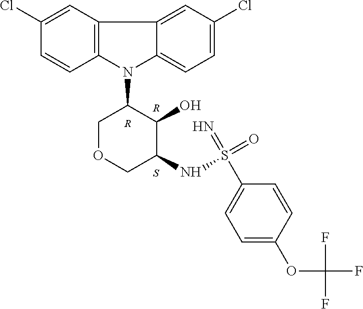

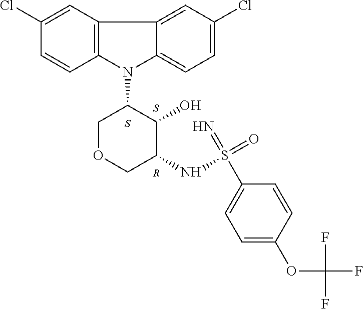

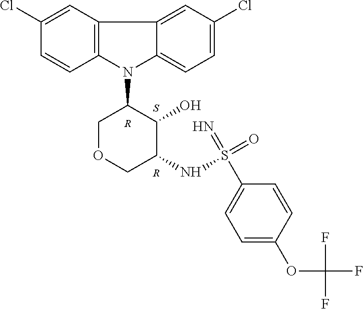

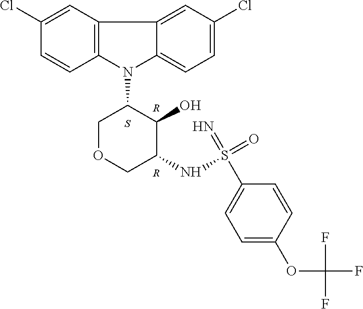

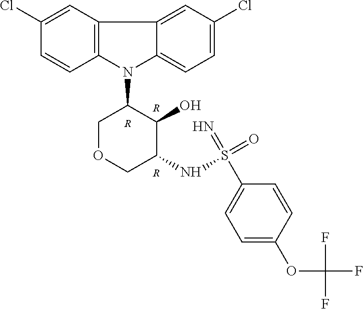

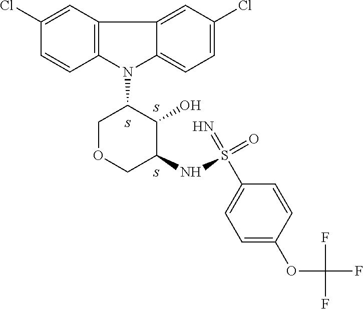

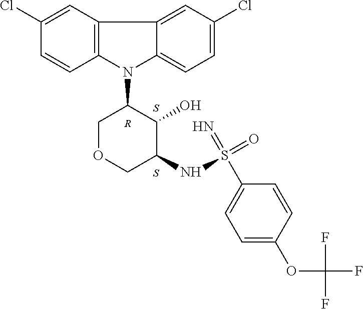

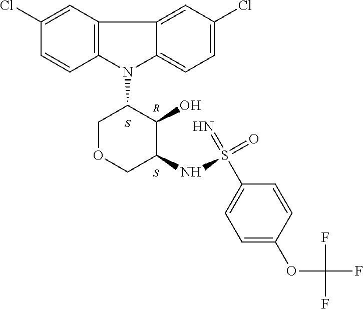

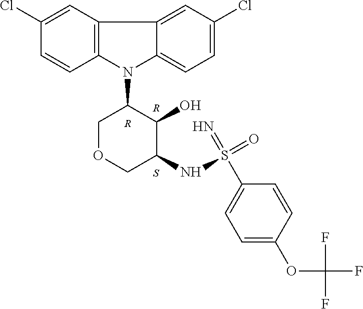

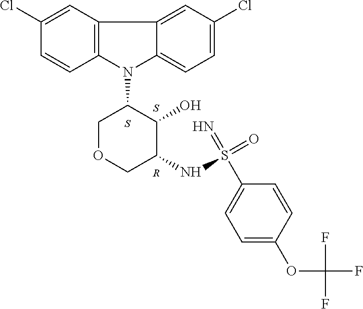

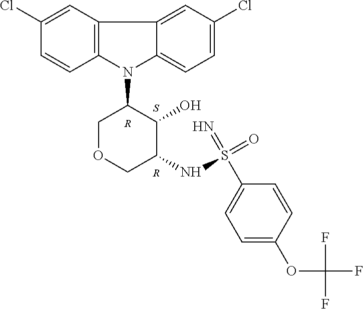

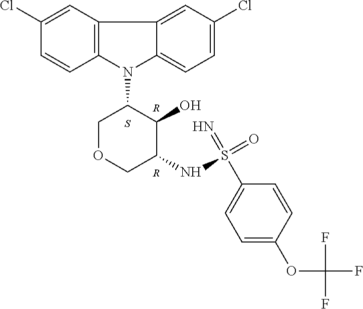

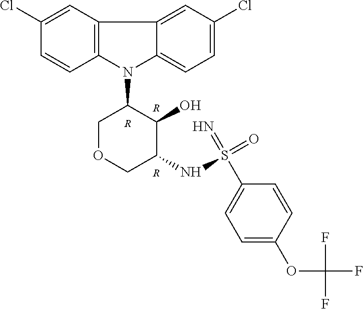

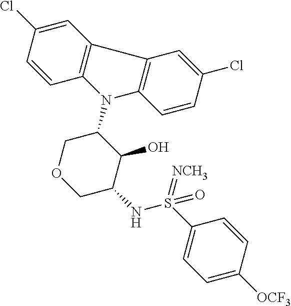

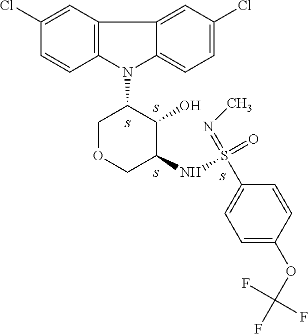

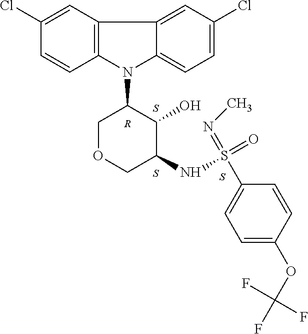

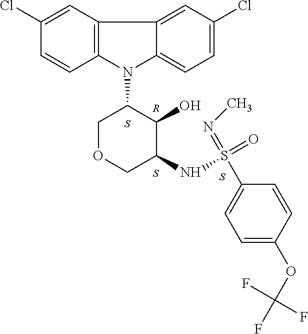

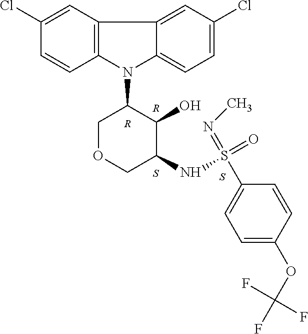

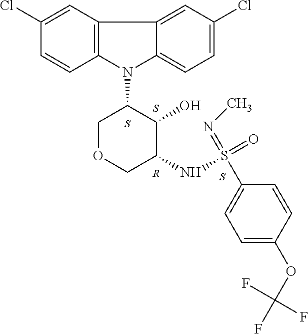

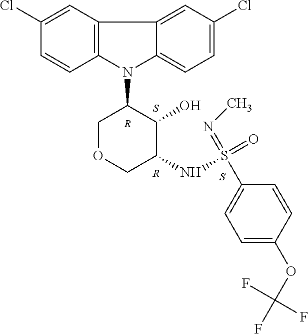

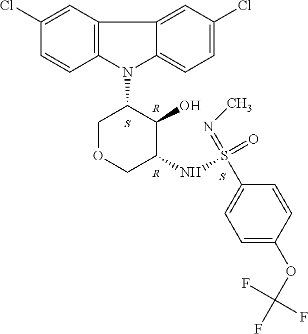

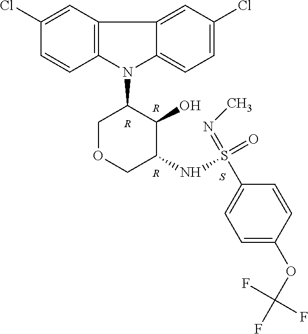

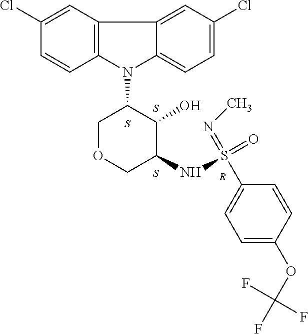

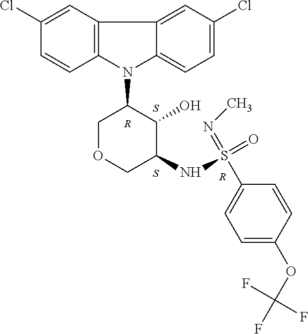

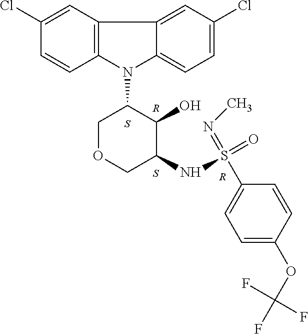

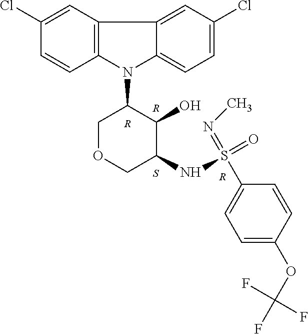

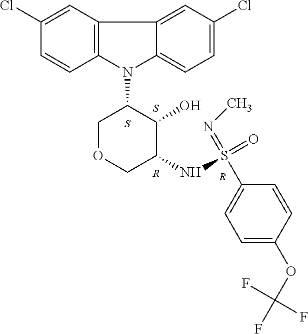

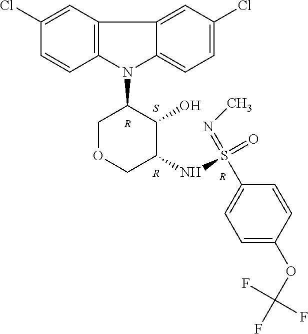

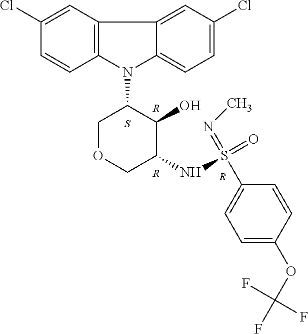

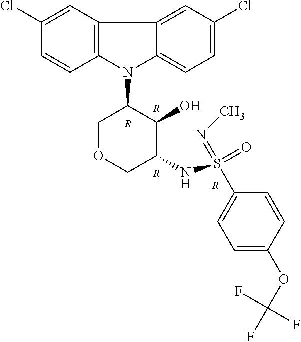

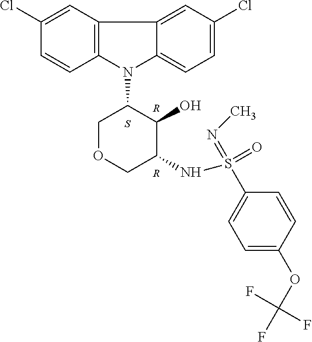

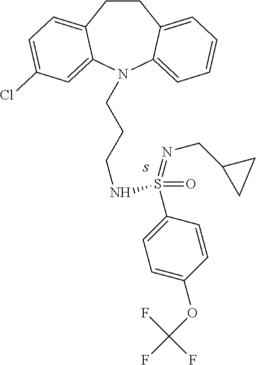

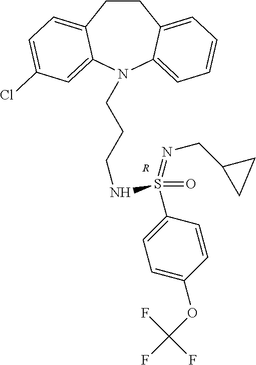

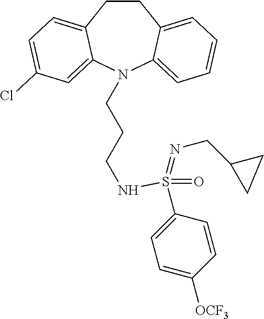 [SMILES: O=S(=NCC1CC1)(NCCCN1c2ccccc2CCc2ccc(Cl)cc21)c1ccc(OC(F)(F)F)cc1]

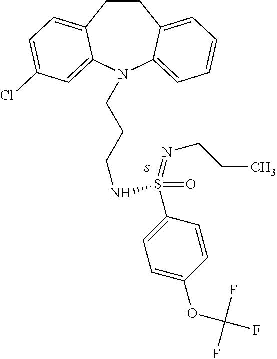

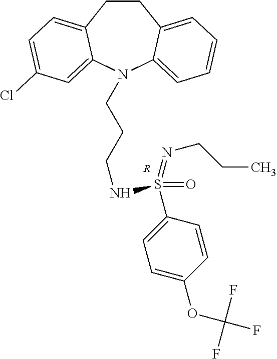

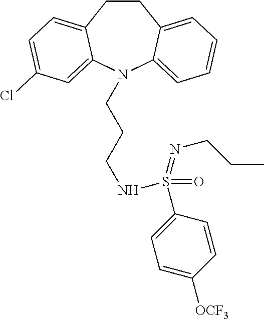 [SMILES: CCCN=S(=O)(NCCCN1c2ccccc2CCc2ccc(Cl)cc21)c1ccc(OC(F)(F)F)cc1]

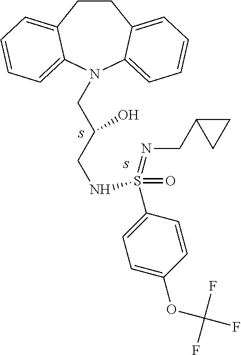

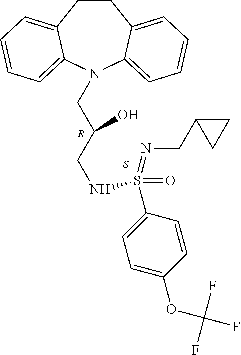

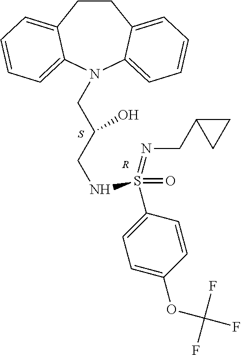

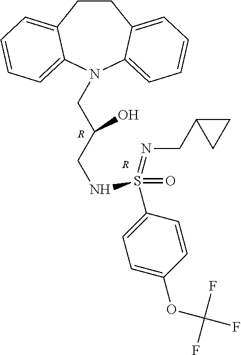

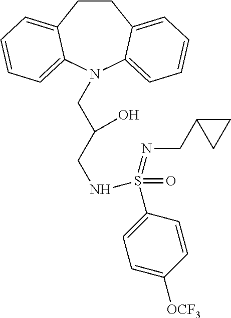 [SMILES: O=S(=NCC1CC1)(NCC(O)CN1c2ccccc2CCc2ccccc21)c1ccc(OC(F)(F)F)cc1]

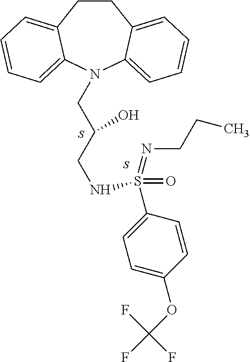

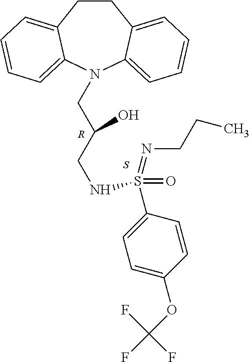

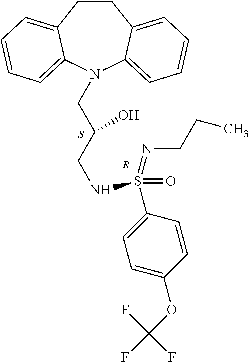

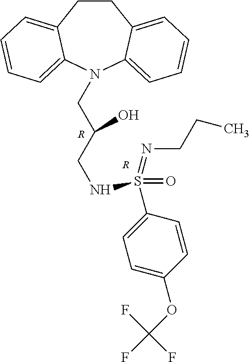

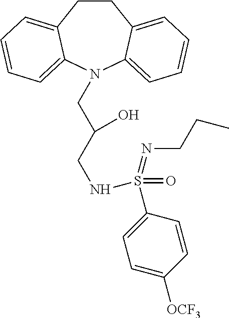 [SMILES: CCCN=S(=O)(NCC(O)CN1c2ccccc2CCc2ccccc21)c1ccc(OC(F)(F)F)cc1]

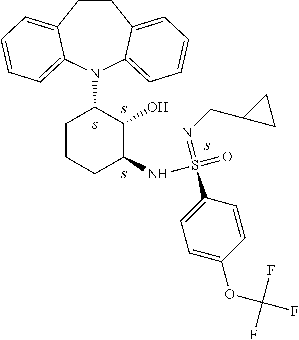

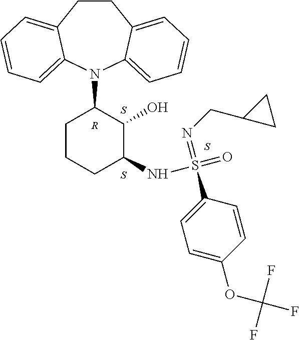

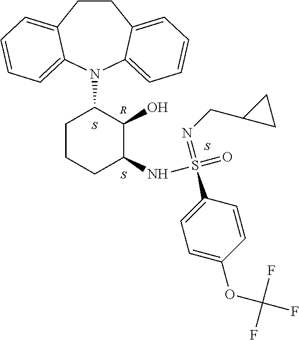

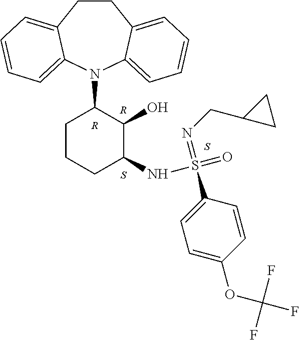

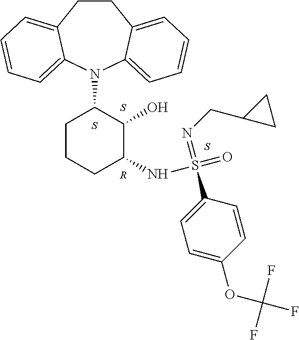

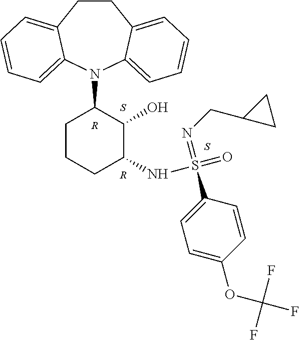

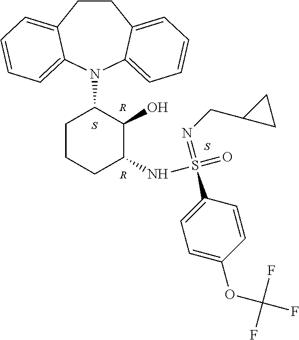

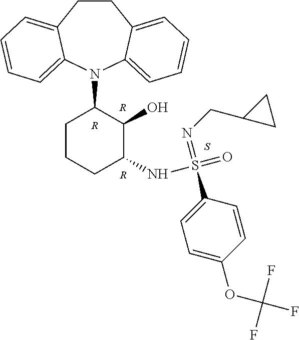

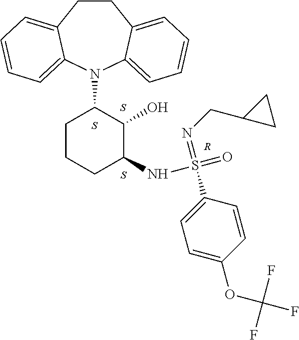

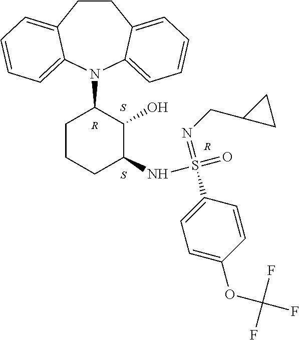

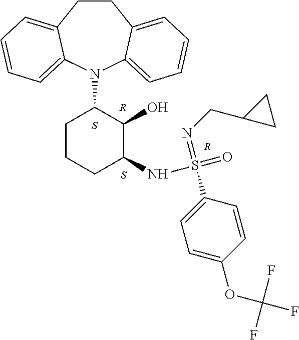

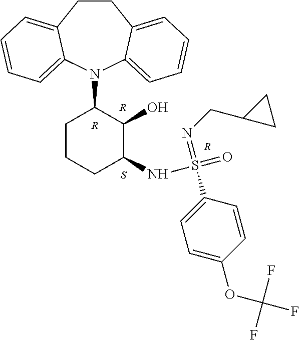

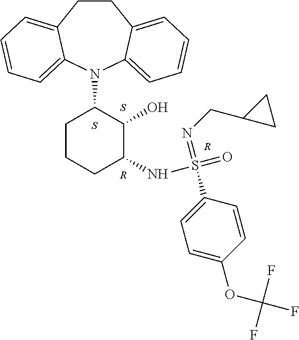

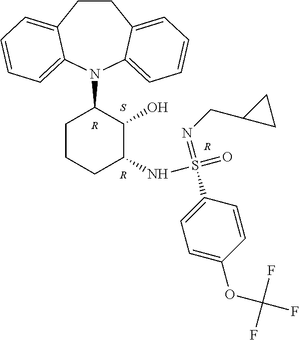

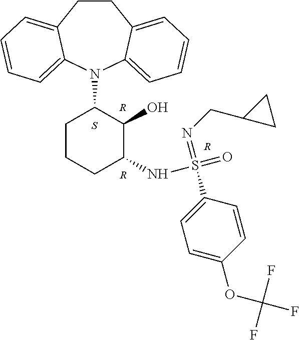

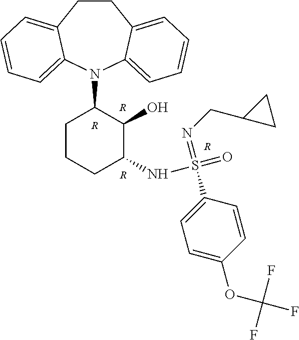

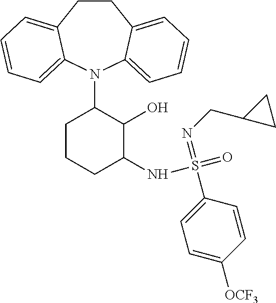 [SMILES: O=S(=NCC1CC1)(NC1CCCC(N2c3ccccc3CCc3ccccc32)C1O)c1ccc(OC(F)(F)F)cc1]

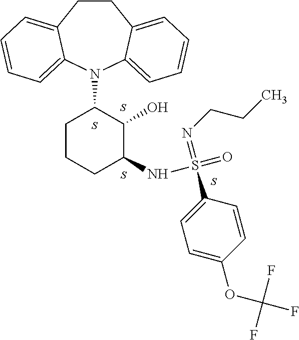

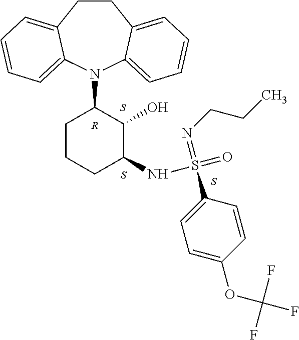

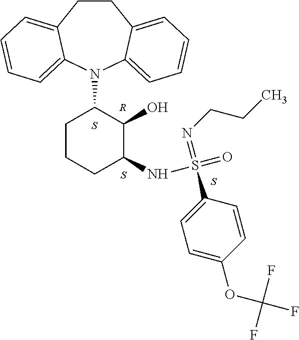

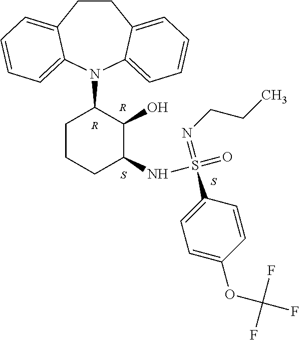

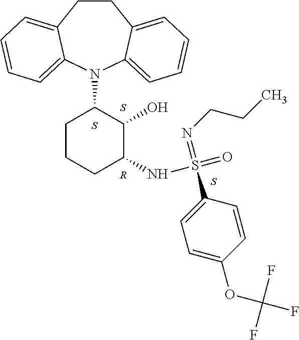

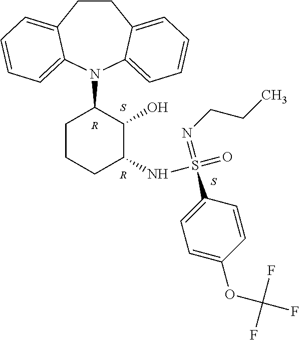

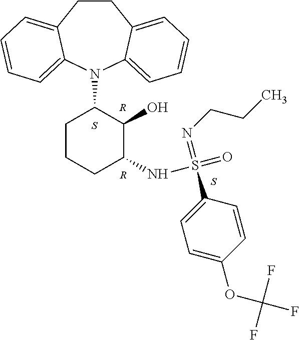

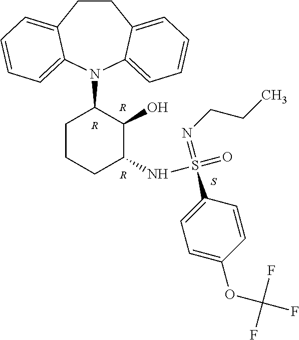

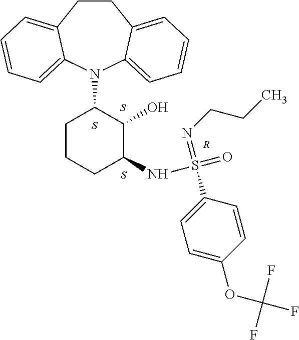

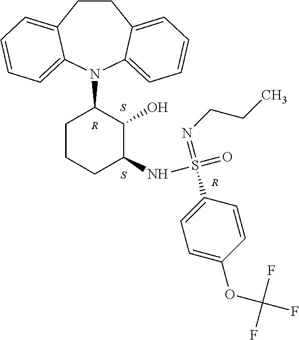

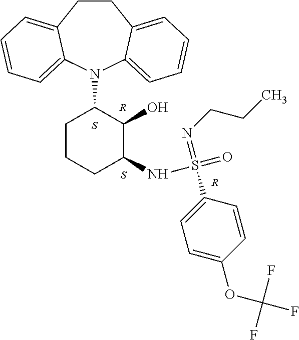

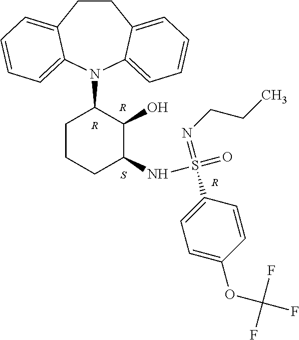

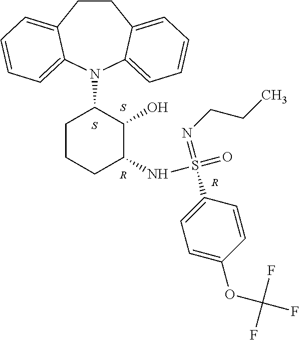

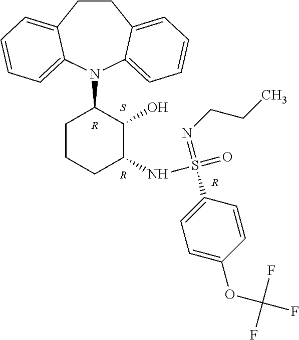

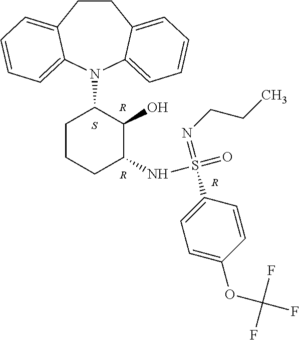

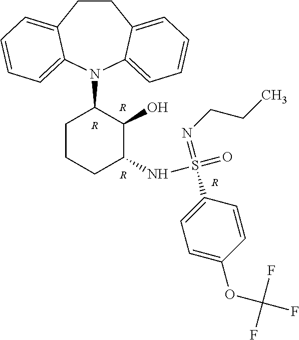

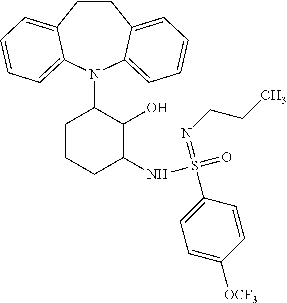 [SMILES: CCCN=S(=O)(NC1CCCC(N2c3ccccc3CCc3ccccc32)C1O)c1ccc(OC(F)(F)F)cc1]

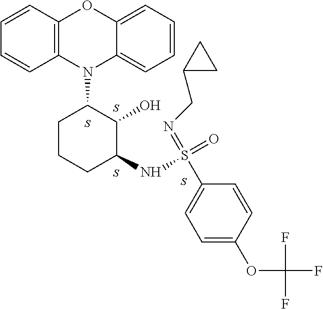

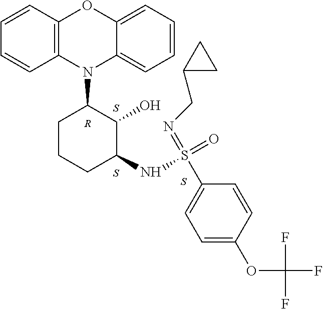

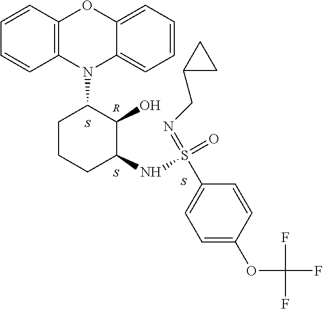

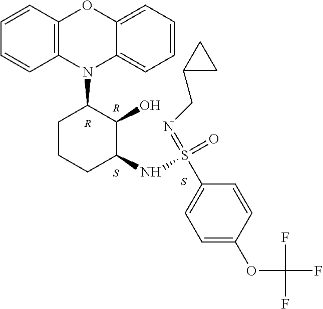

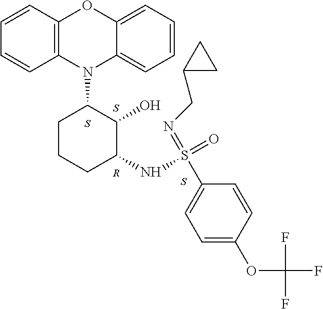

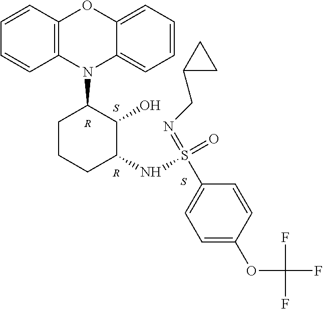

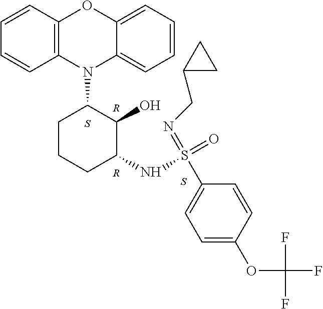

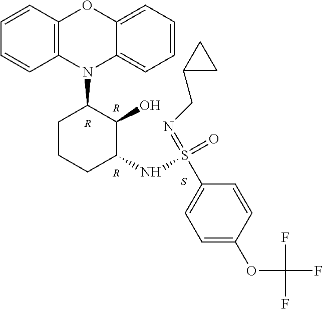

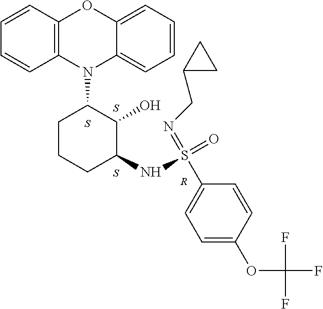

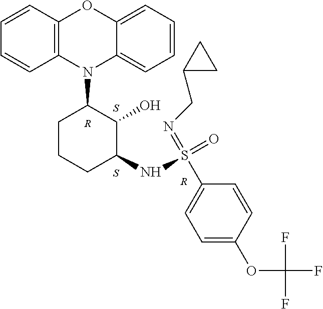

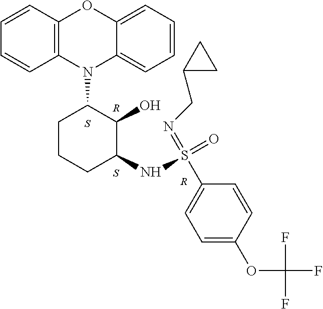

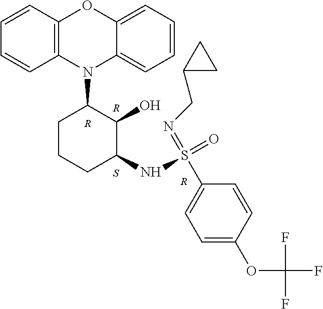

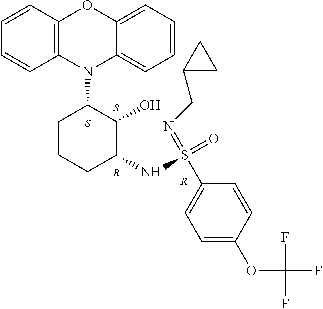

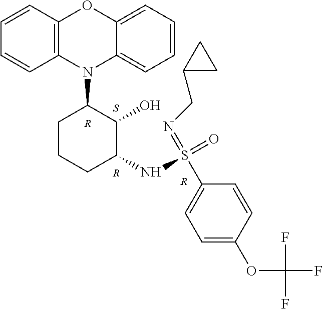

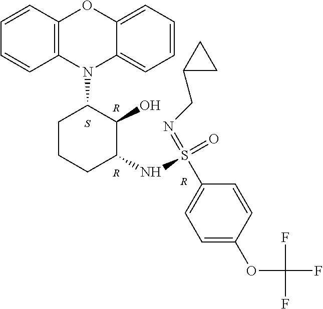

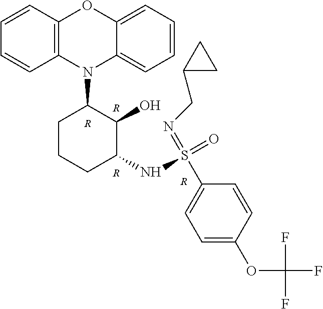

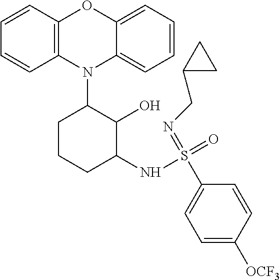 [SMILES: O=S(=NCC1CC1)(NC1CCCC(N2c3ccccc3Oc3ccccc32)C1O)c1ccc(OC(F)(F)F)cc1]

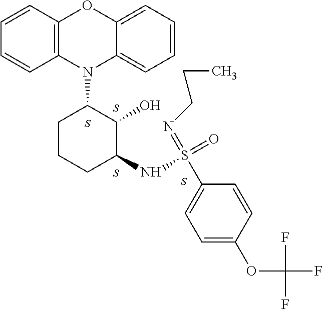

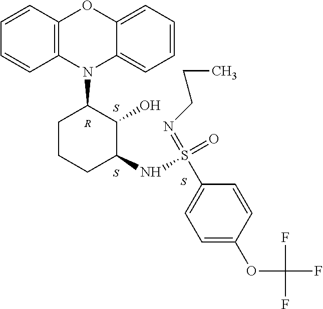

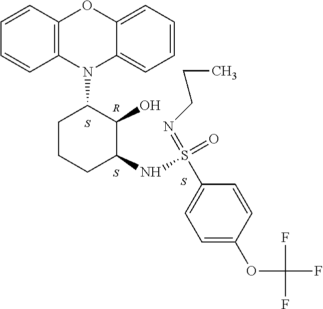

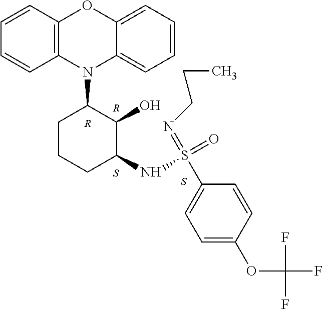

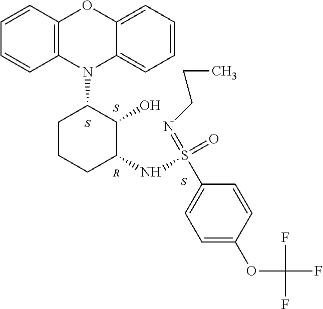

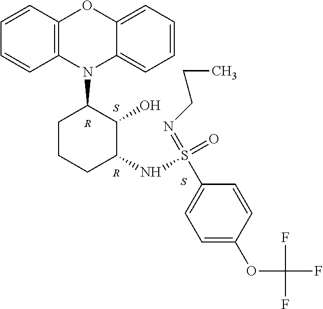

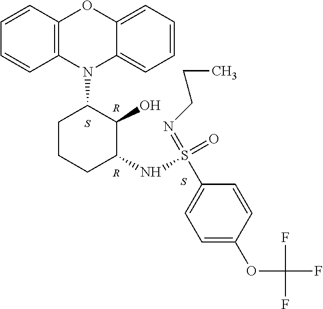

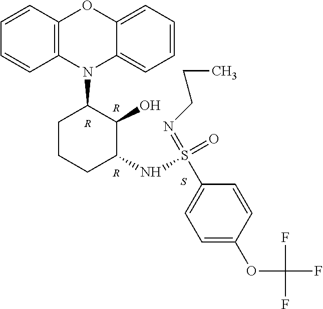

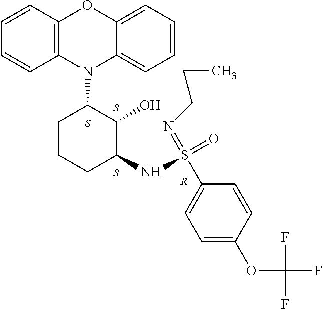

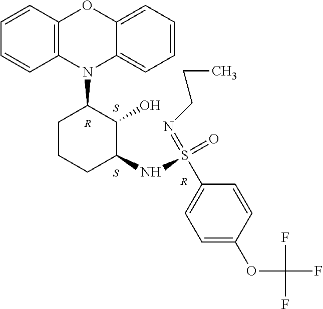

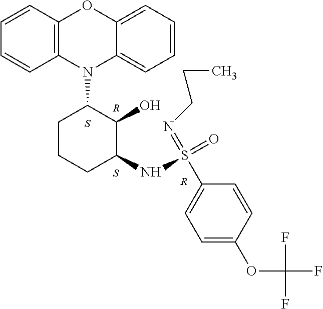

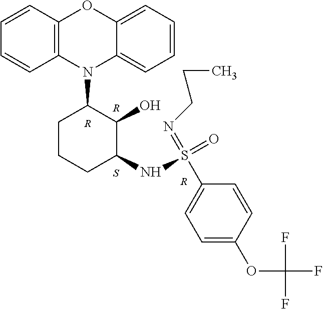

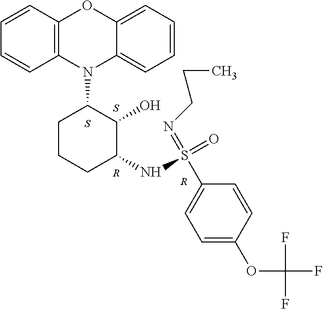

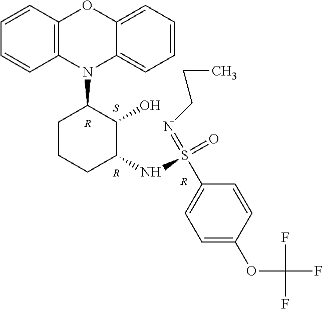

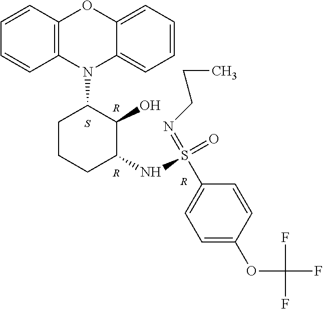

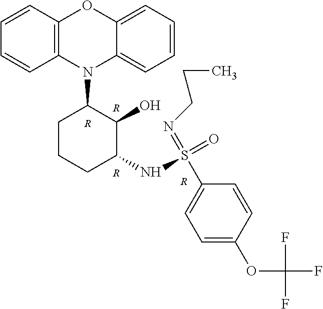

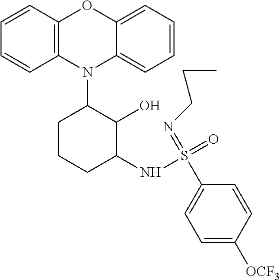 [SMILES: CCCN=S(=O)(NC1CCCC(N2c3ccccc3Oc3ccccc32)C1O)c1ccc(OC(F)(F)F)cc1]

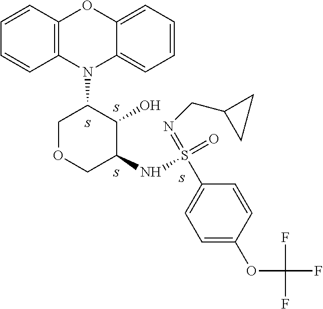

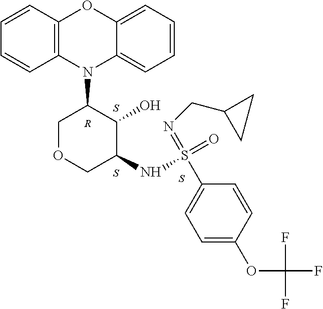

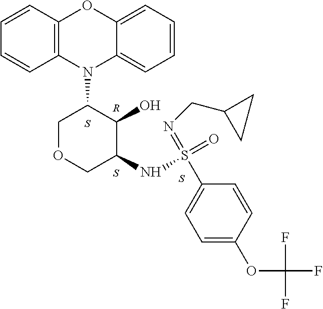

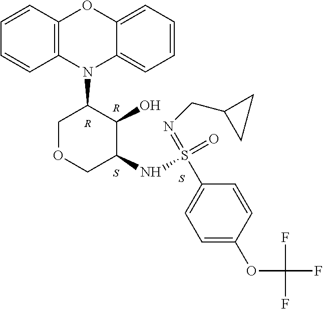

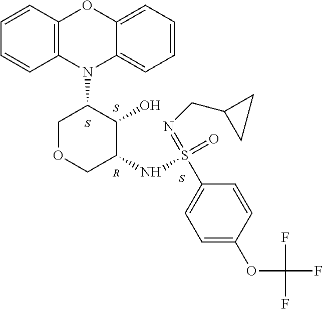

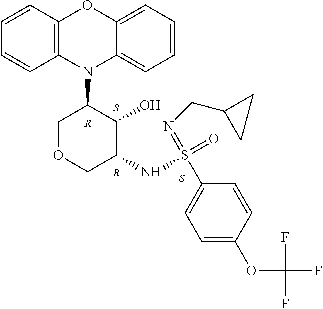

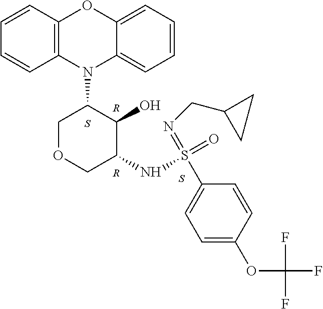

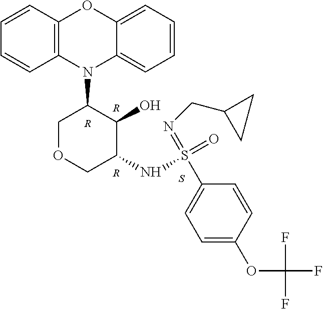

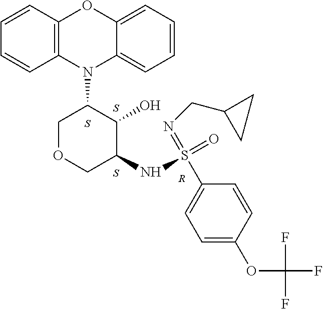

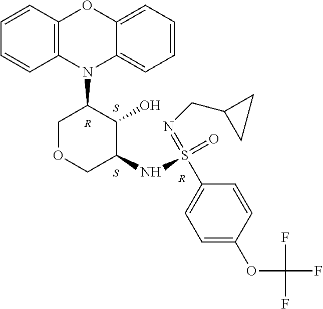

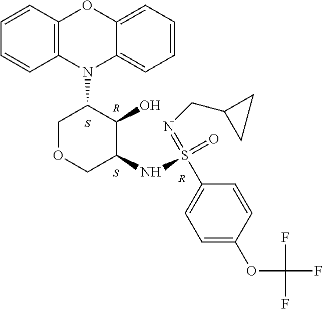

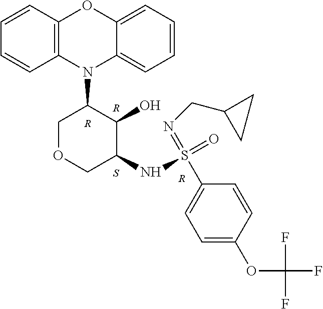

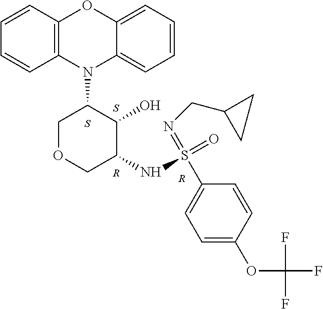

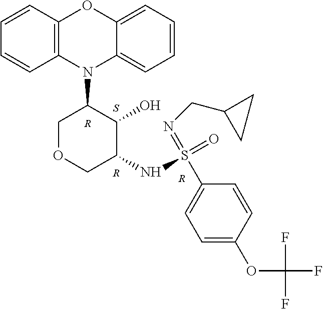

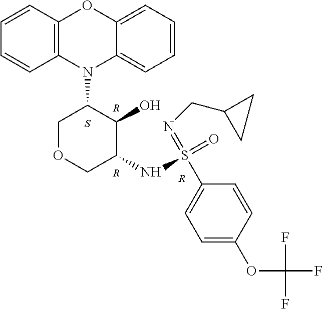

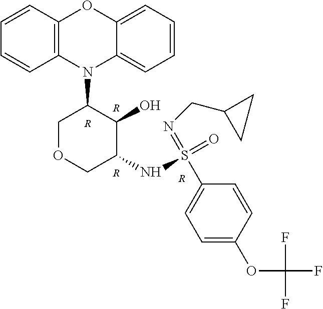

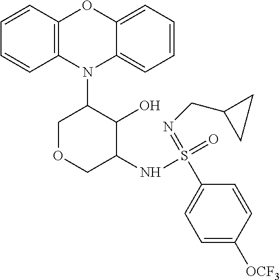 [SMILES: O=S(=NCC1CC1)(NC1COCC(N2c3ccccc3Oc3ccccc32)C1O)c1ccc(OC(F)(F)F)cc1]

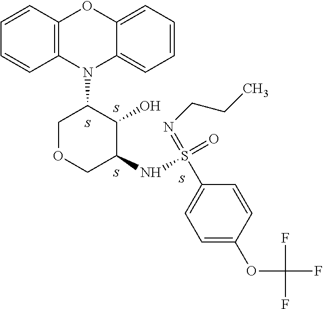

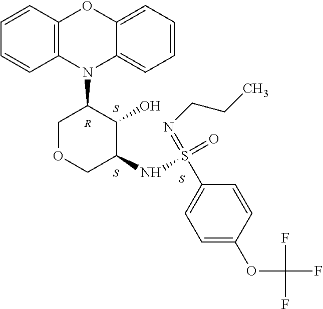

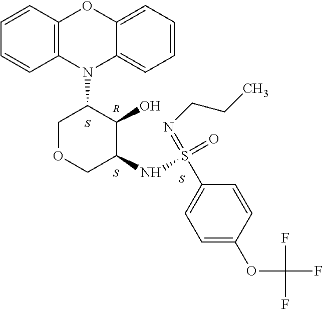

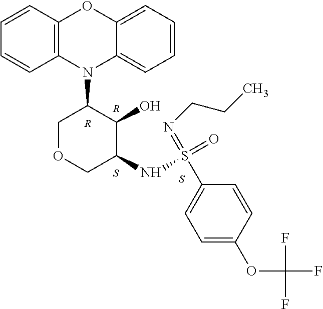

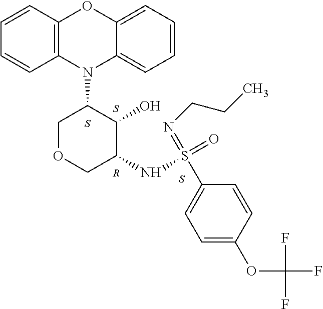

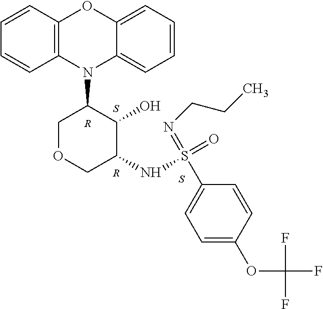

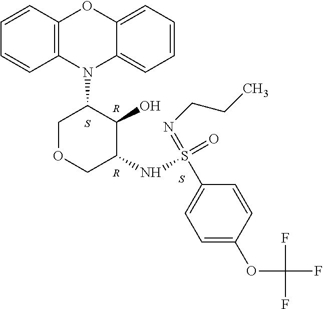

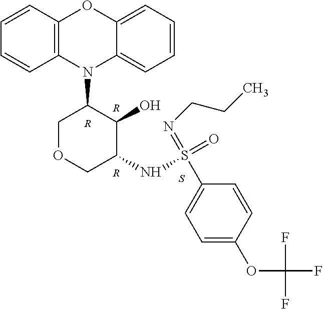

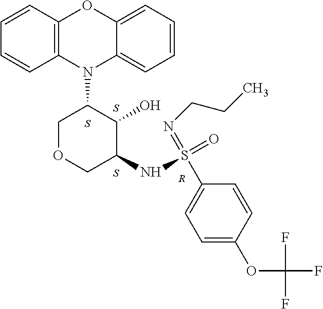

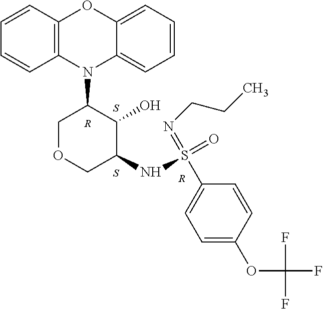

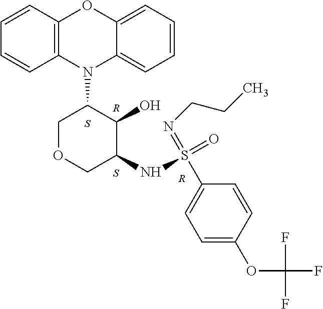

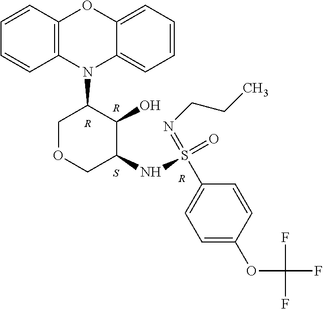

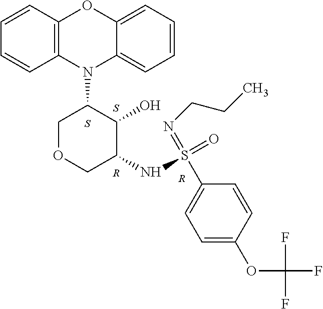

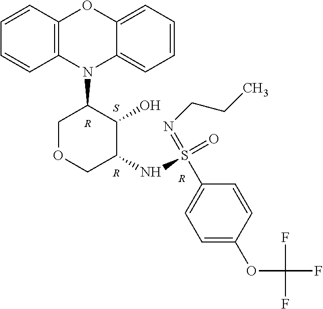

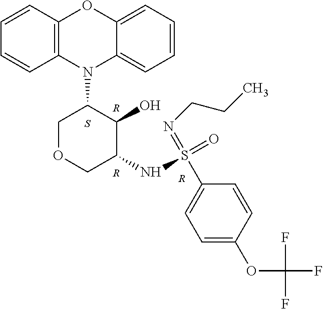

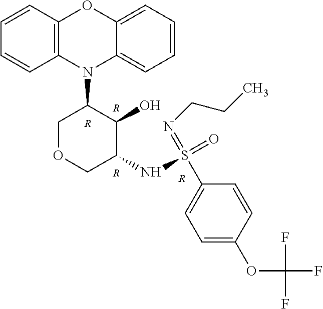

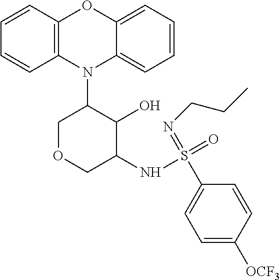 [SMILES: CCCN=S(=O)(NC1COCC(N2c3ccccc3Oc3ccccc32)C1O)c1ccc(OC(F)(F)F)cc1]

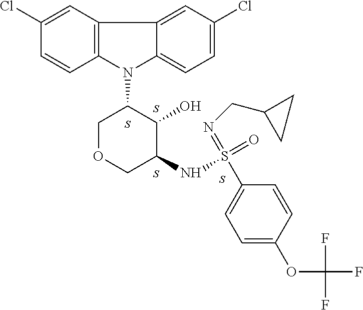

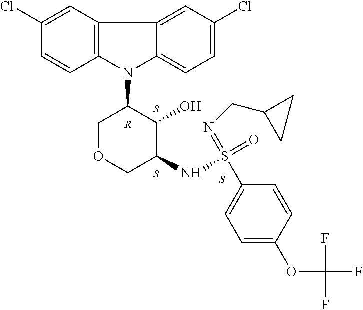

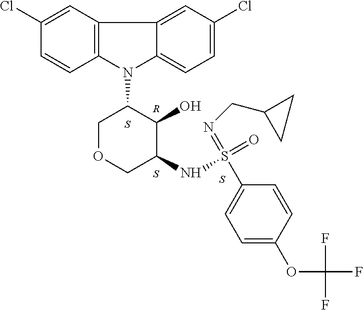

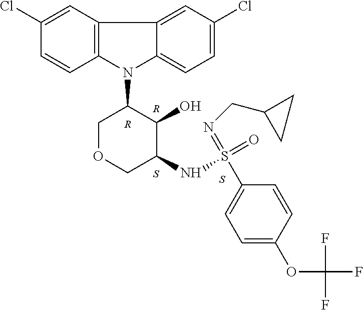

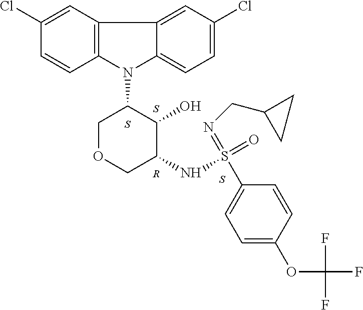

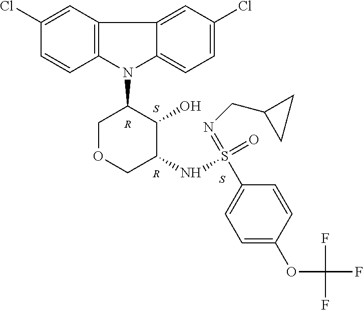

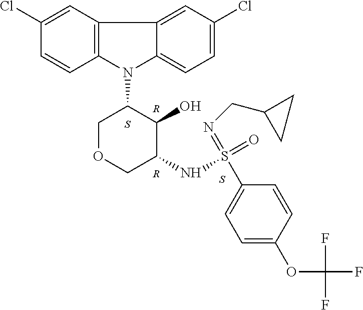

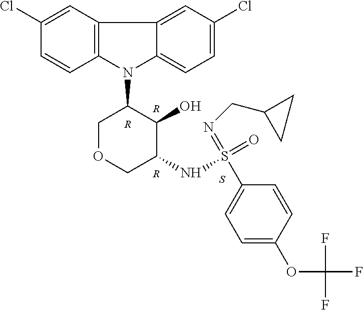

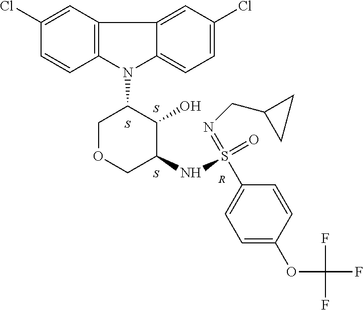

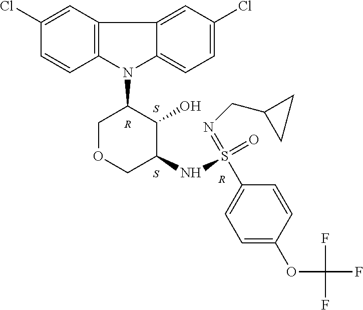

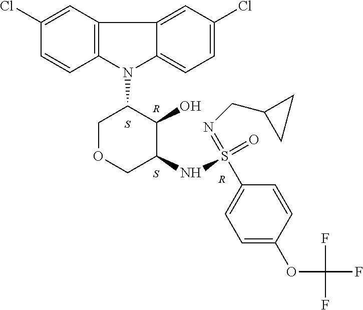

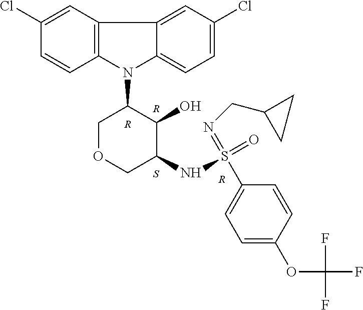

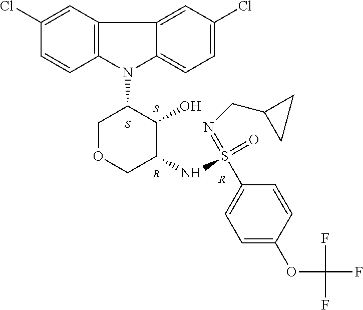

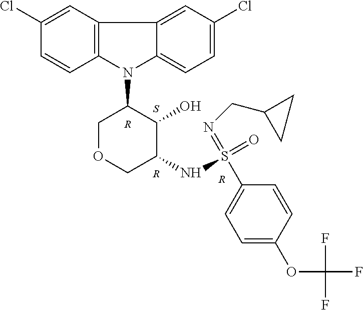

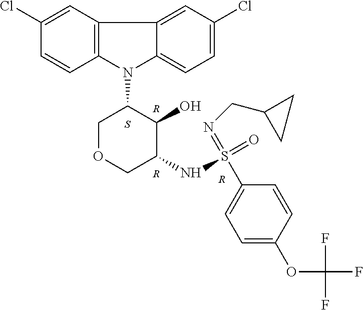

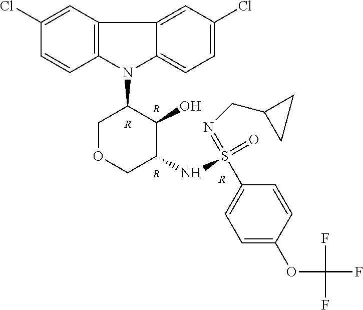

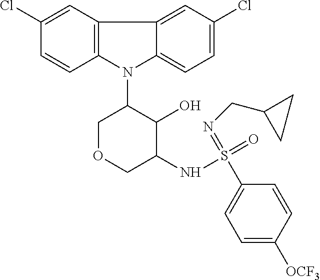 [SMILES: O=S(=NCC1CC1)(NC1COCC(n2c3ccc(Cl)cc3c3cc(Cl)ccc32)C1O)c1ccc(OC(F)(F)F)cc1]

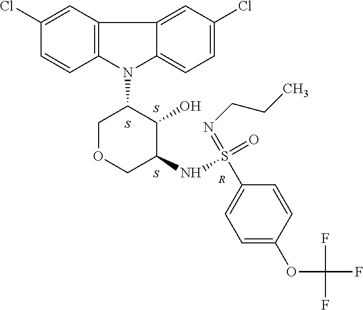

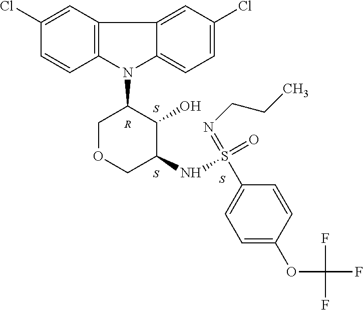

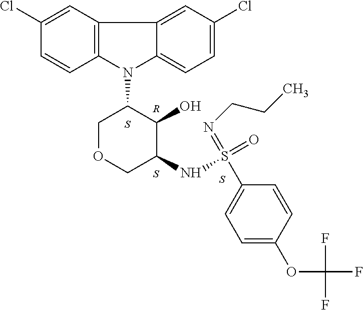

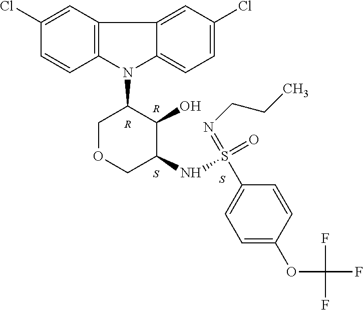

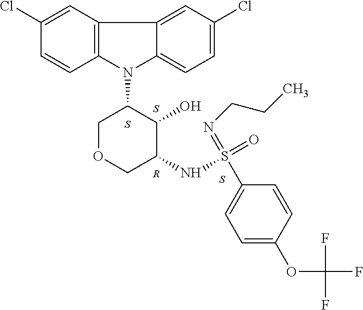

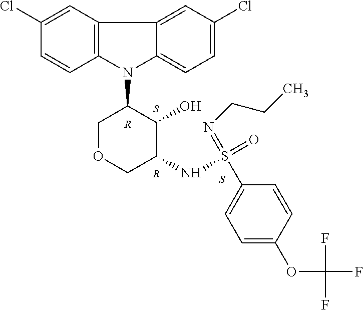

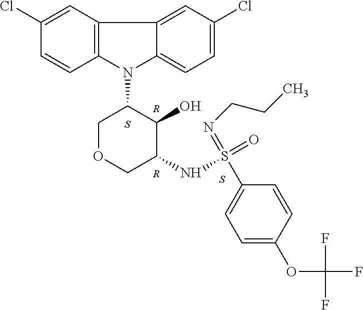

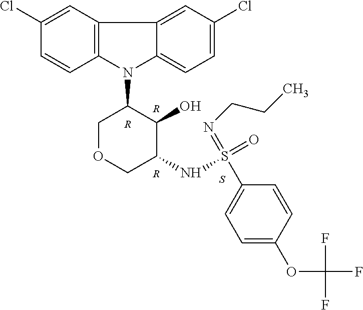

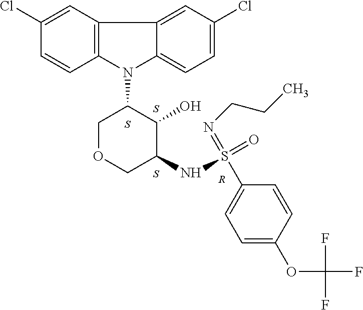

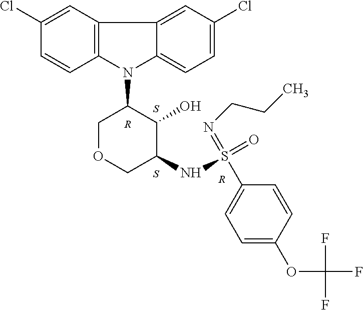

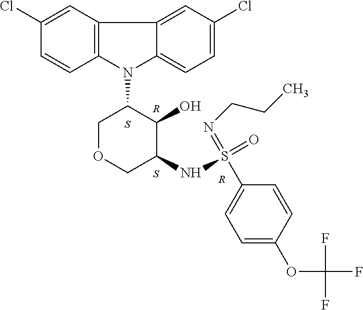

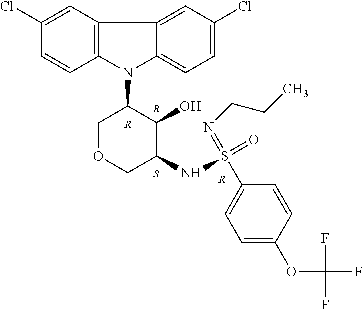

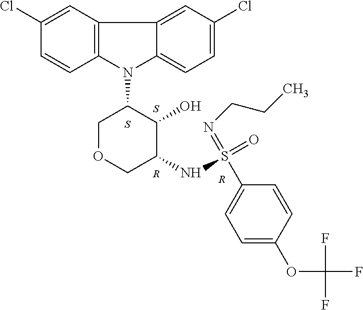

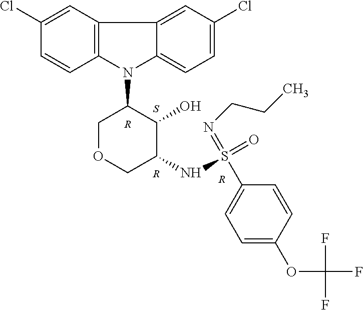

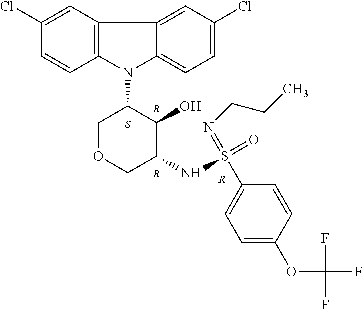

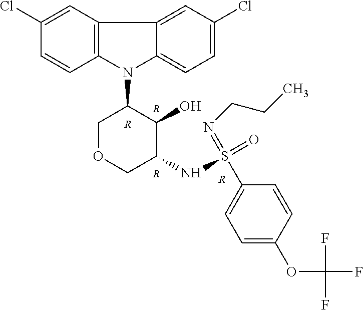

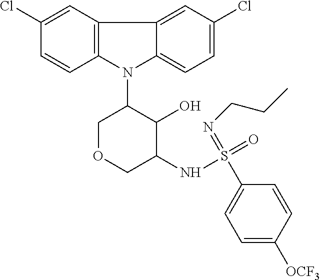 [SMILES: CCCN=S(=O)(NC1COCC(n2c3ccc(Cl)cc3c3cc(Cl)ccc32)C1O)c1ccc(OC(F)(F)F)cc1]

In certain embodiments, the compound is selected from the group consisting of the compounds of Table 2.

In certain embodiments, any of the compounds of Table 2 may be substituted on the nitrogen of the sulfonimidamide similarly as those is Table 1. In certain embodiments, in any of the compounds of Table 2, the imine-nitrogen of the sulfonimidamide (the substituent corresponding to R¹⁸ in compounds of formula (I)) is substituted with H, methyl, propyl, or methylcyclopropyl.

TABLE 2

| | |
|---|---|
| 1 | 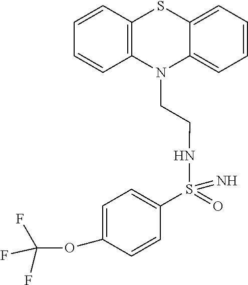 [SMILES: N=S(=O)(NCCN1c2ccccc2Sc2ccccc21)c1ccc(OC(F)(F)F)cc1] |
| 2 | 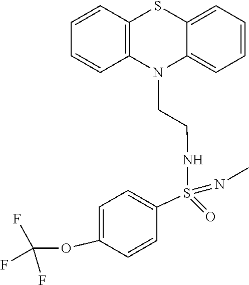 [SMILES: CN=S(=O)(NCCN1c2ccccc2Sc2ccccc21)c1ccc(OC(F)(F)F)cc1] |
| 3 | 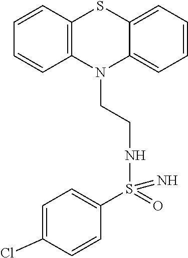 [SMILES: N=S(=O)(NCCN1c2ccccc2Sc2ccccc21)c1ccc(Cl)cc1] |
| 4 | 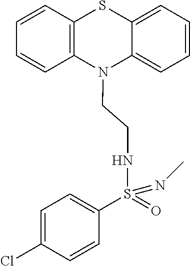 [SMILES: CN=S(=O)(NCCN1c2ccccc2Sc2ccccc21)c1ccc(Cl)cc1] |
| 5 | 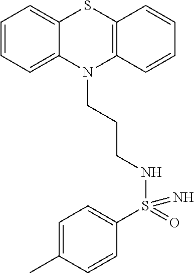 [SMILES: Cc1ccc(S(=N)(=O)NCCCN2c3ccccc3Sc3ccccc32)cc1] |
| 6 | 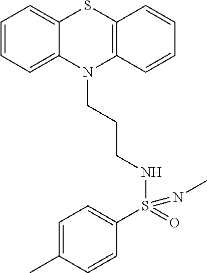 [SMILES: CN=S(=O)(NCCCN1c2ccccc2Sc2ccccc21)c1ccc(C)cc1] |
| 7 | 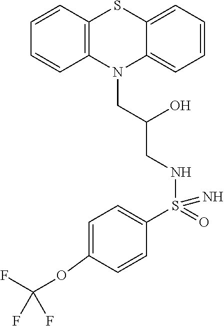 [SMILES: N=S(=O)(NCC(O)CN1c2ccccc2Sc2ccccc21)c1ccc(OC(F)(F)F)cc1] |
| 8 | 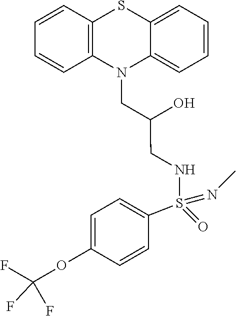 [SMILES: CN=S(=O)(NCC(O)CN1c2ccccc2Sc2ccccc21)c1ccc(OC(F)(F)F)cc1] |
| 9 | 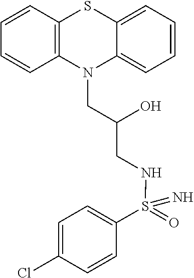 [SMILES: N=S(=O)(NCC(O)CN1c2ccccc2Sc2ccccc21)c1ccc(Cl)cc1] |
| 10 | 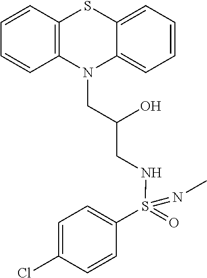 [SMILES: CN=S(=O)(NCC(O)CN1c2ccccc2Sc2ccccc21)c1ccc(Cl)cc1] |
| 11 | 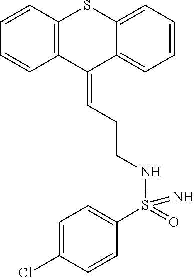 [SMILES: N=S(=O)(NCCC=C1c2ccccc2Sc2ccccc21)c1ccc(Cl)cc1] |
| 12 | 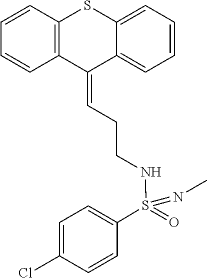 [SMILES: CN=S(=O)(NCCC=C1c2ccccc2Sc2ccccc21)c1ccc(Cl)cc1] |
| 13 | 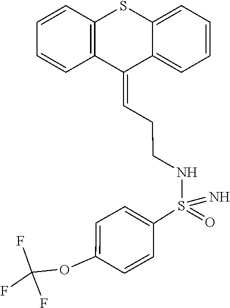 [SMILES: N=S(=O)(NCCC=C1c2ccccc2Sc2ccccc21)c1ccc(OC(F)(F)F)cc1] |
| 14 | 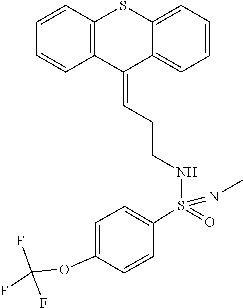 [SMILES: CN=S(=O)(NCCC=C1c2ccccc2Sc2ccccc21)c1ccc(OC(F)(F)F)cc1] |
| 15 | 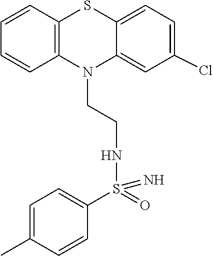 [SMILES: Cc1ccc(S(=N)(=O)NCCN2c3ccccc3Sc3ccc(Cl)cc32)cc1] |
| 16 | 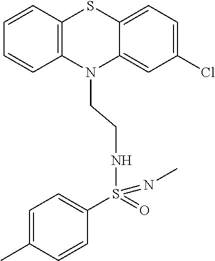 [SMILES: CN=S(=O)(NCCN1c2ccccc2Sc2ccc(Cl)cc21)c1ccc(C)cc1] |
| 17 | 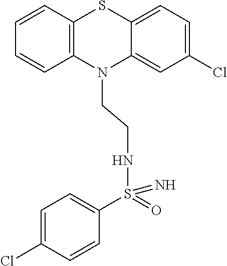 [SMILES: N=S(=O)(NCCN1c2ccccc2Sc2ccc(Cl)cc21)c1ccc(Cl)cc1] |
| 18 | 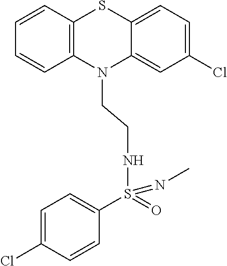 [SMILES: CN=S(=O)(NCCN1c2ccccc2Sc2ccc(Cl)cc21)c1ccc(Cl)cc1] |
| 19 | 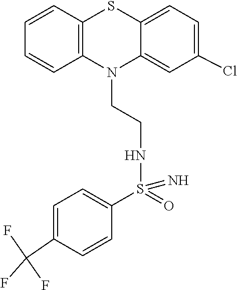 [SMILES: N=S(=O)(NCCN1c2ccccc2Sc2ccc(Cl)cc21)c1ccc(C(F)(F)F)cc1] |
| 20 | 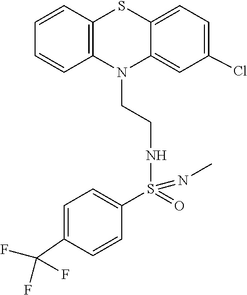 [SMILES: CN=S(=O)(NCCN1c2ccccc2Sc2ccc(Cl)cc21)c1ccc(C(F)(F)F)cc1] |
| 21 | 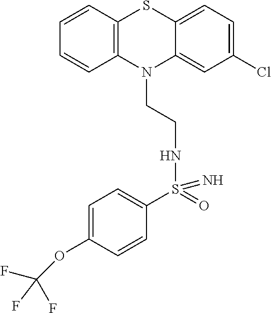 [SMILES: N=S(=O)(NCCN1c2ccccc2Sc2ccc(Cl)cc21)c1ccc(OC(F)(F)F)cc1] |
| 22 | 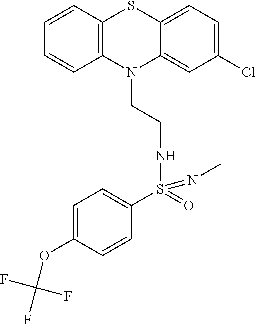 [SMILES: CN=S(=O)(NCCN1c2ccccc2Sc2ccc(Cl)cc21)c1ccc(OC(F)(F)F)cc1] |
| 23 | 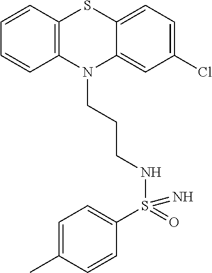 [SMILES: Cc1ccc(S(=N)(=O)NCCCN2c3ccccc3Sc3ccc(Cl)cc32)cc1] |
| 24 | 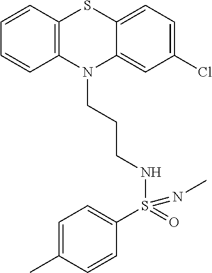 [SMILES: CN=S(=O)(NCCCN1c2ccccc2Sc2ccc(Cl)cc21)c1ccc(C)cc1] |
| 25 | 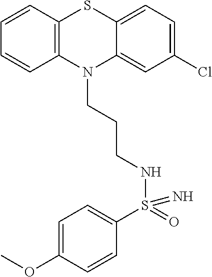 [SMILES: COc1ccc(S(=N)(=O)NCCCN2c3ccccc3Sc3ccc(Cl)cc32)cc1] |
| 26 | 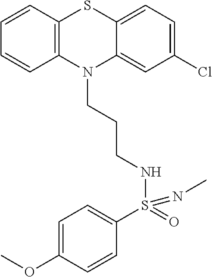 [SMILES: CN=S(=O)(NCCCN1c2ccccc2Sc2ccc(Cl)cc21)c1ccc(OC)cc1] |
| 27 | 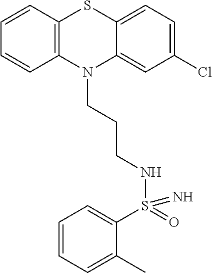 [SMILES: Cc1ccccc1S(=N)(=O)NCCCN1c2ccccc2Sc2ccc(Cl)cc21] |
| 28 | 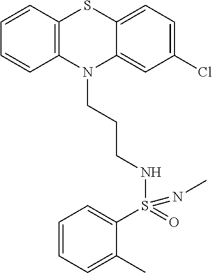 [SMILES: CN=S(=O)(NCCCN1c2ccccc2Sc2ccc(Cl)cc21)c1ccccc1C] |
| 29 | 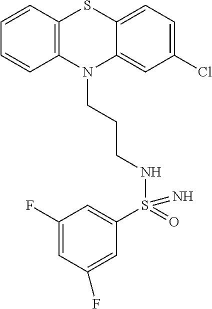 [SMILES: N=S(=O)(NCCCN1c2ccccc2Sc2ccc(Cl)cc21)c1cc(F)cc(F)c1] |
| 30 | 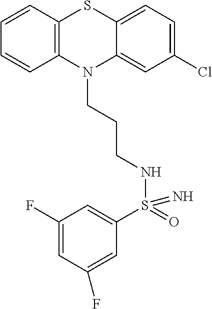 [SMILES: N=S(=O)(NCCCN1c2ccccc2Sc2ccc(Cl)cc21)c1cc(F)cc(F)c1] |
| 31 | 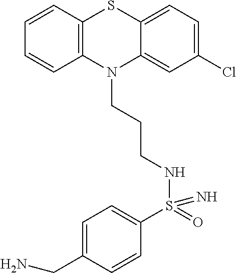 [SMILES: N=S(=O)(NCCCN1c2ccccc2Sc2ccc(Cl)cc21)c1ccc(CN)cc1] |
| 32 | 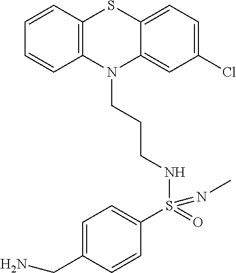 [SMILES: CN=S(=O)(NCCCN1c2ccccc2Sc2ccc(Cl)cc21)c1ccc(CN)cc1] |
| 33 | 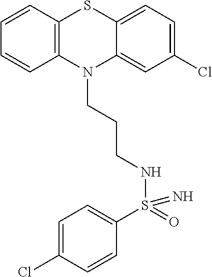 [SMILES: N=S(=O)(NCCCN1c2ccccc2Sc2ccc(Cl)cc21)c1ccc(Cl)cc1] |
| 34 | 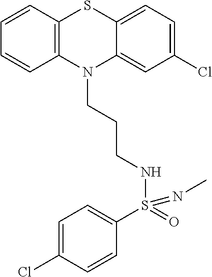 [SMILES: CN=S(=O)(NCCCN1c2ccccc2Sc2ccc(Cl)cc21)c1ccc(Cl)cc1] |
| 35 | 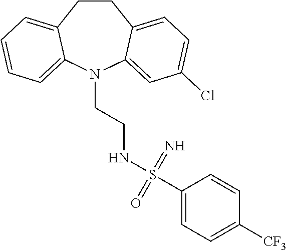 [SMILES: N=S(=O)(NCCN1c2ccccc2CCc2ccc(Cl)cc21)c1ccc(C(F)(F)F)cc1] |
| 36 | 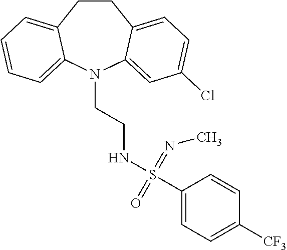 [SMILES: CN=S(=O)(NCCN1c2ccccc2CCc2ccc(Cl)cc21)c1ccc(C(F)(F)F)cc1] |
| 37 | 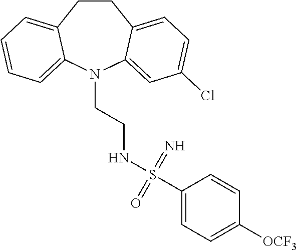 [SMILES: N=S(=O)(NCCN1c2ccccc2CCc2ccc(Cl)cc21)c1ccc(OC(F)(F)F)cc1] |
| 38 | 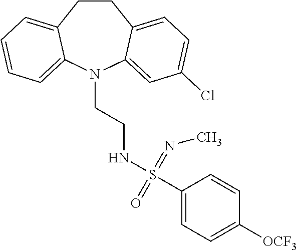 [SMILES: CN=S(=O)(NCCN1c2ccccc2CCc2ccc(Cl)cc21)c1ccc(OC(F)(F)F)cc1] |
| 39 | 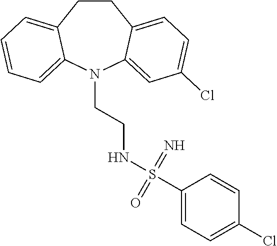 [SMILES: N=S(=O)(NCCN1c2ccccc2CCc2ccc(Cl)cc21)c1ccc(Cl)cc1] |
| 40 | 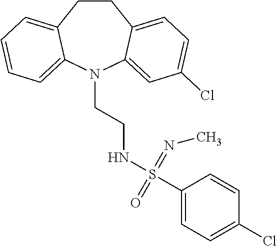 [SMILES: CN=S(=O)(NCCN1c2ccccc2CCc2ccc(Cl)cc21)c1ccc(Cl)cc1] |
| 41 | 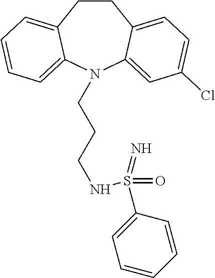 [SMILES: N=S(=O)(NCCCN1c2ccccc2CCc2ccc(Cl)cc21)c1ccccc1] |
| 42 | 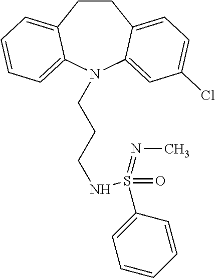 [SMILES: CN=S(=O)(NCCCN1c2ccccc2CCc2ccc(Cl)cc21)c1ccccc1] |
| 43 | 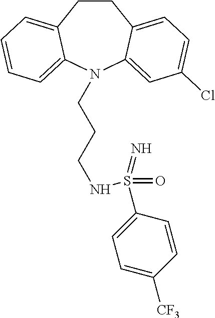 [SMILES: N=S(=O)(NCCCN1c2ccccc2CCc2ccc(Cl)cc21)c1ccc(C(F)(F)F)cc1] |
| 44 | 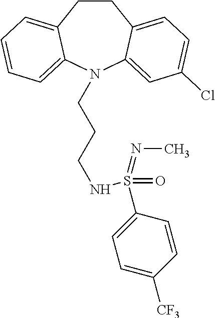 [SMILES: CN=S(=O)(NCCCN1c2ccccc2CCc2ccc(Cl)cc21)c1ccc(C(F)(F)F)cc1] |
| 45 | 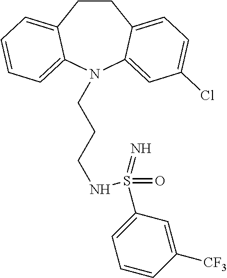 [SMILES: N=S(=O)(NCCCN1c2ccccc2CCc2ccc(Cl)cc21)c1cccc(C(F)(F)F)c1] |
| 46 | 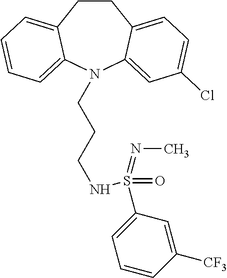 [SMILES: CN=S(=O)(NCCCN1c2ccccc2CCc2ccc(Cl)cc21)c1cccc(C(F)(F)F)c1] |
| 47 | 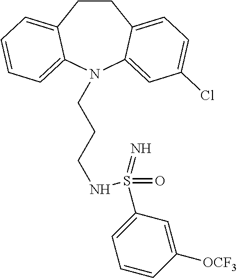 [SMILES: N=S(=O)(NCCCN1c2ccccc2CCc2ccc(Cl)cc21)c1cccc(OC(F)(F)F)c1] |
| 48 | 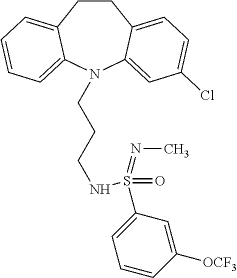 [SMILES: CN=S(=O)(NCCCN1c2ccccc2CCc2ccc(Cl)cc21)c1cccc(OC(F)(F)F)c1] |
| 49 | 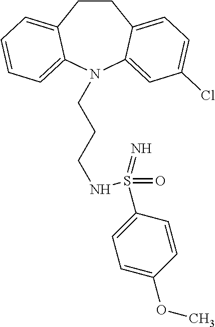 [SMILES: COc1ccc(S(=N)(=O)NCCCN2c3ccccc3CCc3ccc(Cl)cc32)cc1] |
| 50 | 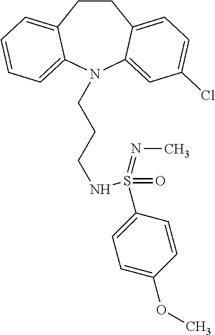 [SMILES: CN=S(=O)(NCCCN1c2ccccc2CCc2ccc(Cl)cc21)c1ccc(OC)cc1] |
| 51 | 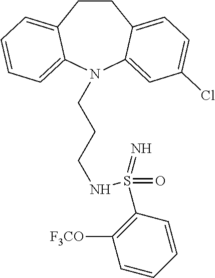 [SMILES: N=S(=O)(NCCCN1c2ccccc2CCc2ccc(Cl)cc21)c1ccccc1OC(F)(F)F] |
| 52 | 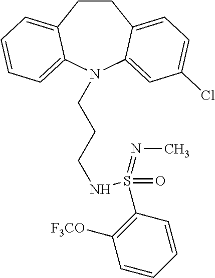 [SMILES: CN=S(=O)(NCCCN1c2ccccc2CCc2ccc(Cl)cc21)c1ccccc1OC(F)(F)F] |
| 53 | 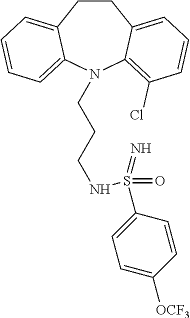 [SMILES: N=S(=O)(NCCCN1c2ccccc2CCc2cccc(Cl)c21)c1ccc(OC(F)(F)F)cc1] |
| 54 | 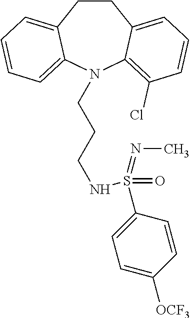 [SMILES: CN=S(=O)(NCCCN1c2ccccc2CCc2cccc(Cl)c21)c1ccc(OC(F)(F)F)cc1] |
| 55 | 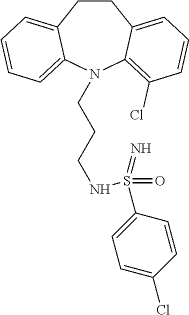 [SMILES: N=S(=O)(NCCCN1c2ccccc2CCc2cccc(Cl)c21)c1ccc(Cl)cc1] |
| 56 | 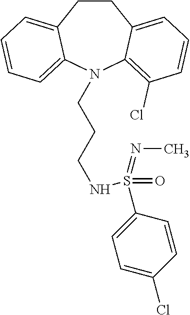 [SMILES: CN=S(=O)(NCCCN1c2ccccc2CCc2cccc(Cl)c21)c1ccc(Cl)cc1] |
| 57 | 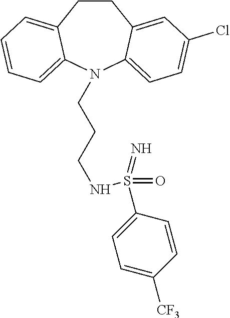 [SMILES: N=S(=O)(NCCCN1c2ccccc2CCc2cc(Cl)ccc21)c1ccc(C(F)(F)F)cc1] |
| 58 | 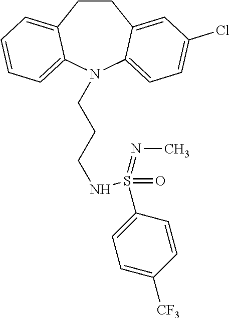 [SMILES: CN=S(=O)(NCCCN1c2ccccc2CCc2cc(Cl)ccc21)c1ccc(C(F)(F)F)cc1] |
| 59 | 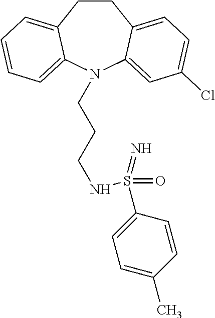 [SMILES: Cc1ccc(S(=N)(=O)NCCCN2c3ccccc3CCc3ccc(Cl)cc32)cc1] |
| 60 | 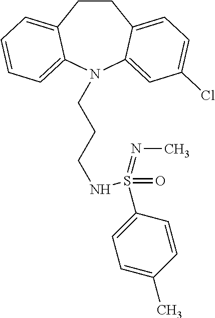 [SMILES: CN=S(=O)(NCCCN1c2ccccc2CCc2ccc(Cl)cc21)c1ccc(C)cc1] |
| 61 | 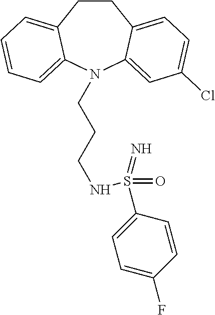 [SMILES: N=S(=O)(NCCCN1c2ccccc2CCc2ccc(Cl)cc21)c1ccc(F)cc1] |
| 62 | 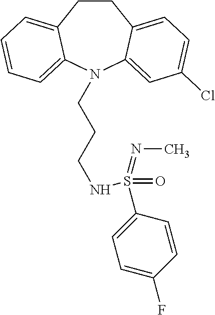 [SMILES: CN=S(=O)(NCCCN1c2ccccc2CCc2ccc(Cl)cc21)c1ccc(F)cc1] |
| 63 | 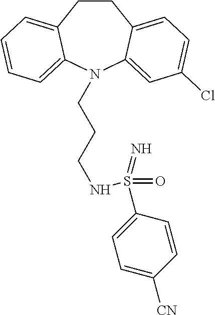 [SMILES: N#Cc1ccc(S(=N)(=O)NCCCN2c3ccccc3CCc3ccc(Cl)cc32)cc1] |
| 64 | 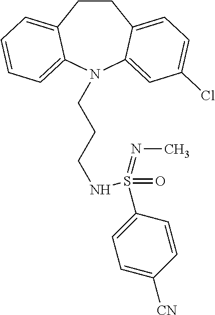 [SMILES: CN=S(=O)(NCCCN1c2ccccc2CCc2ccc(Cl)cc21)c1ccc(C#N)cc1] |
| 65 | 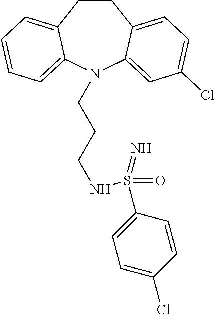 [SMILES: N=S(=O)(NCCCN1c2ccccc2CCc2ccc(Cl)cc21)c1ccc(Cl)cc1] |
| 66 | 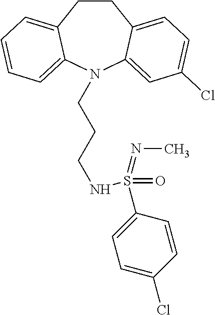 [SMILES: CN=S(=O)(NCCCN1c2ccccc2CCc2ccc(Cl)cc21)c1ccc(Cl)cc1] |
| 67 | 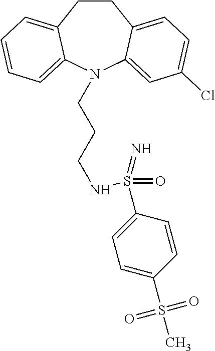 [SMILES: CS(=O)(=O)c1ccc(S(=N)(=O)NCCCN2c3ccccc3CCc3ccc(Cl)cc32)cc1] |
| 68 | 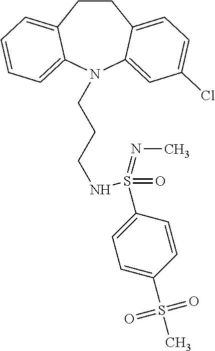 [SMILES: CN=S(=O)(NCCCN1c2ccccc2CCc2ccc(Cl)cc21)c1ccc(S(C)(=O)=O)cc1] |
| 69 | 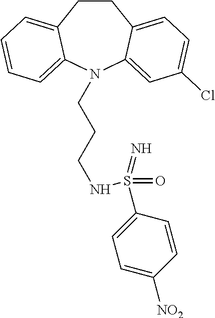 [SMILES: N=S(=O)(NCCCN1c2ccccc2CCc2ccc(Cl)cc21)c1ccc([N+](=O)[O-])cc1] |
| 70 | 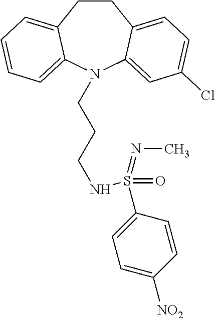 [SMILES: CN=S(=O)(NCCCN1c2ccccc2CCc2ccc(Cl)cc21)c1ccc([N+](=O)[O-])cc1] |
| 71 | 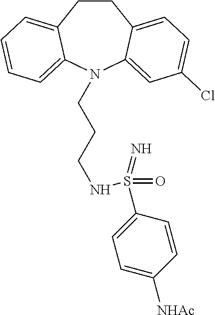 [SMILES: CC(=O)Nc1ccc(S(=N)(=O)NCCCN2c3ccccc3CCc3ccc(Cl)cc32)cc1] |
| 72 | 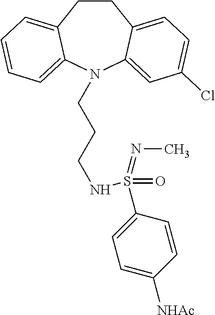 [SMILES: CN=S(=O)(NCCCN1c2ccccc2CCc2ccc(Cl)cc21)c1ccc(NC(C)=O)cc1] |
| 73 | 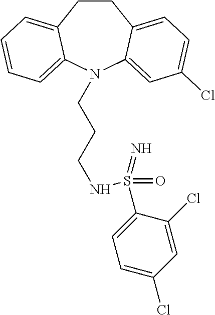 [SMILES: N=S(=O)(NCCCN1c2ccccc2CCc2ccc(Cl)cc21)c1ccc(Cl)cc1Cl] |
| 74 | 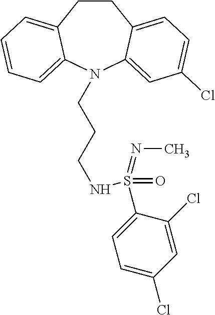 [SMILES: CN=S(=O)(NCCCN1c2ccccc2CCc2ccc(Cl)cc21)c1ccc(Cl)cc1Cl] |
| 75 | 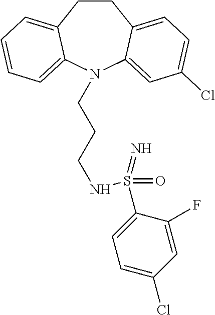 [SMILES: N=S(=O)(NCCCN1c2ccccc2CCc2ccc(Cl)cc21)c1ccc(Cl)cc1F] |
| 76 | 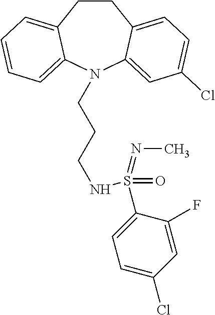 [SMILES: CN=S(=O)(NCCCN1c2ccccc2CCc2ccc(Cl)cc21)c1ccc(Cl)cc1F] |
| 77 | 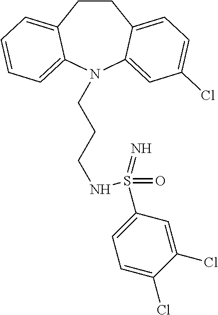 [SMILES: N=S(=O)(NCCCN1c2ccccc2CCc2ccc(Cl)cc21)c1ccc(Cl)c(Cl)c1] |
| 78 | 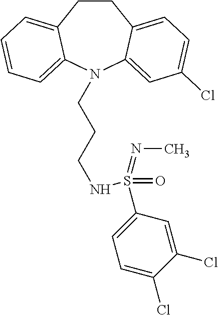 [SMILES: CN=S(=O)(NCCCN1c2ccccc2CCc2ccc(Cl)cc21)c1ccc(Cl)c(Cl)c1] |
| 79 | 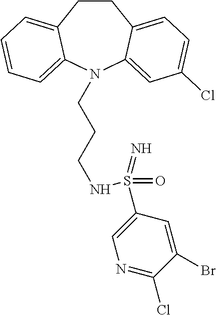 [SMILES: N=S(=O)(NCCCN1c2ccccc2CCc2ccc(Cl)cc21)c1cnc(Cl)c(Br)c1] |
| 80 | 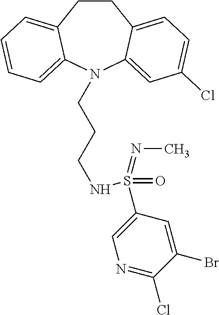 [SMILES: CN=S(=O)(NCCCN1c2ccccc2CCc2ccc(Cl)cc21)c1cnc(Cl)c(Br)c1] |
| 81 | 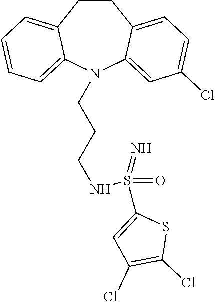 [SMILES: N=S(=O)(NCCCN1c2ccccc2CCc2ccc(Cl)cc21)c1cc(Cl)c(Cl)s1] |
| 82 | 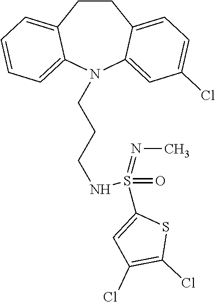 [SMILES: CN=S(=O)(NCCCN1c2ccccc2CCc2ccc(Cl)cc21)c1cc(Cl)c(Cl)s1] |
| 83 | 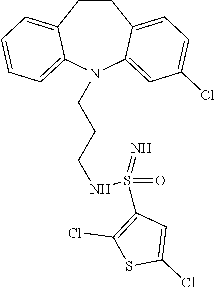 [SMILES: N=S(=O)(NCCCN1c2ccccc2CCc2ccc(Cl)cc21)c1cc(Cl)sc1Cl] |
| 84 | 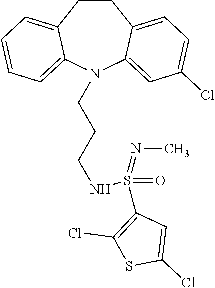 [SMILES: CN=S(=O)(NCCCN1c2ccccc2CCc2ccc(Cl)cc21)c1cc(Cl)sc1Cl] |
| 85 | 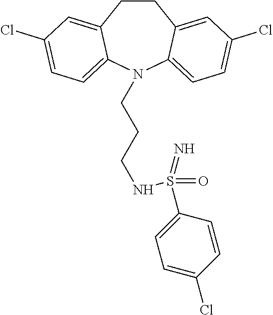 [SMILES: N=S(=O)(NCCCN1c2ccc(Cl)cc2CCc2cc(Cl)ccc21)c1ccc(Cl)cc1] |
| 86 | 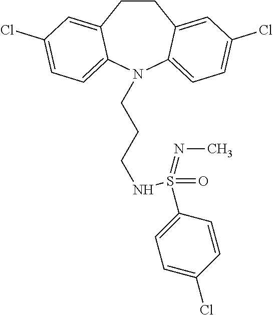 [SMILES: CN=S(=O)(NCCCN1c2ccc(Cl)cc2CCc2cc(Cl)ccc21)c1ccc(Cl)cc1] |
| 87 | 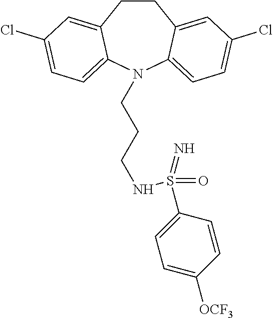 [SMILES: N=S(=O)(NCCCN1c2ccc(Cl)cc2CCc2cc(Cl)ccc21)c1ccc(OC(F)(F)F)cc1] |
| 88 | 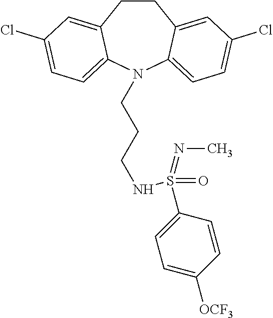 [SMILES: CN=S(=O)(NCCCN1c2ccc(Cl)cc2CCc2cc(Cl)ccc21)c1ccc(OC(F)(F)F)cc1] |
| 89 | 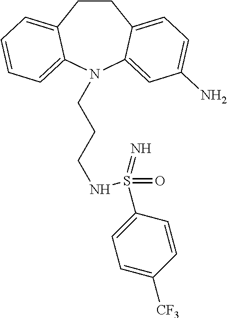 [SMILES: N=S(=O)(NCCCN1c2ccccc2CCc2ccc(N)cc21)c1ccc(C(F)(F)F)cc1] |
| 90 | 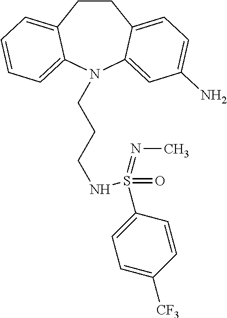 [SMILES: CN=S(=O)(NCCCN1c2ccccc2CCc2ccc(N)cc21)c1ccc(C(F)(F)F)cc1] |
| 91 | 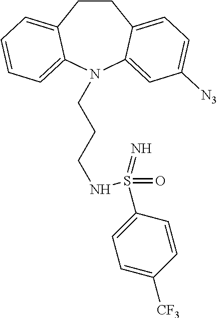 [SMILES: [N-]=[N+]=Nc1ccc2c(c1)N(CCCNS(=N)(=O)c1ccc(C(F)(F)F)cc1)c1ccccc1CC2] |
| 92 | 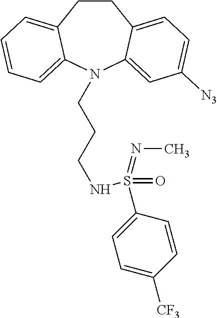 [SMILES: CN=S(=O)(NCCCN1c2ccccc2CCc2ccc(N=[N+]=[N-])cc21)c1ccc(C(F)(F)F)cc1] |
| 93 | 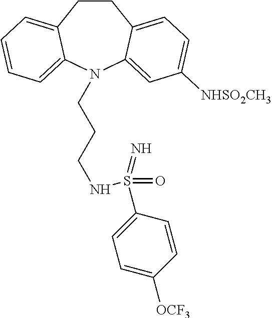 |
| 94 | 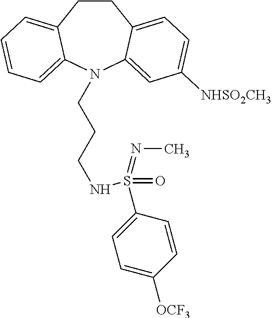 |
| 95 | 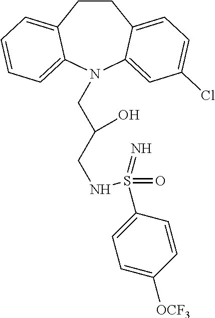 [SMILES: N=S(=O)(NCC(O)CN1c2ccccc2CCc2ccc(Cl)cc21)c1ccc(OC(F)(F)F)cc1] |
| 96 | 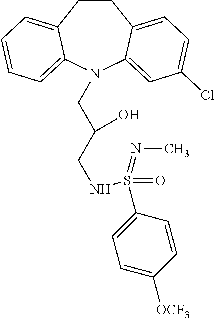 [SMILES: CN=S(=O)(NCC(O)CN1c2ccccc2CCc2ccc(Cl)cc21)c1ccc(OC(F)(F)F)cc1] |
| 97 | 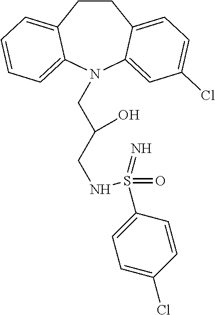 [SMILES: N=S(=O)(NCC(O)CN1c2ccccc2CCc2ccc(Cl)cc21)c1ccc(Cl)cc1] |
| 98 | 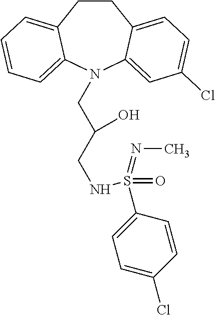 [SMILES: CN=S(=O)(NCC(O)CN1c2ccccc2CCc2ccc(Cl)cc21)c1ccc(Cl)cc1] |
| 99 | 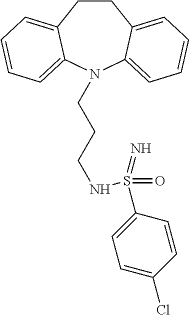 [SMILES: N=S(=O)(NCCCN1c2ccccc2CCc2ccccc21)c1ccc(Cl)cc1] |
| 100 | 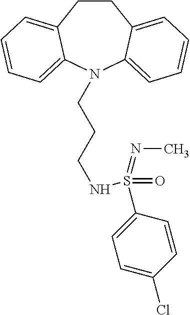 [SMILES: CN=S(=O)(NCCCN1c2ccccc2CCc2ccccc21)c1ccc(Cl)cc1] |
| 101 | 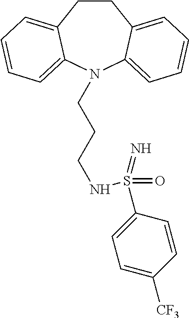 [SMILES: N=S(=O)(NCCCN1c2ccccc2CCc2ccccc21)c1ccc(C(F)(F)F)cc1] |
| 102 | 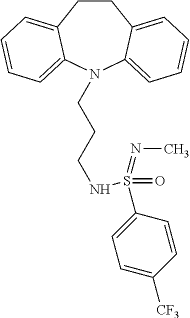 [SMILES: CN=S(=O)(NCCCN1c2ccccc2CCc2ccccc21)c1ccc(C(F)(F)F)cc1] |
| 103 | 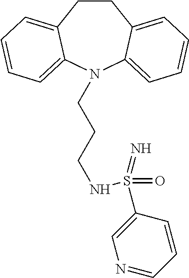 [SMILES: N=S(=O)(NCCCN1c2ccccc2CCc2ccccc21)c1cccnc1] |
| 104 | 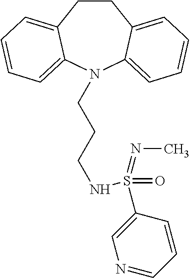 [SMILES: CN=S(=O)(NCCCN1c2ccccc2CCc2ccccc21)c1cccnc1] |
| 105 | 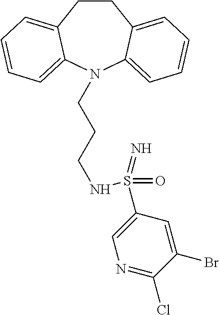 [SMILES: N=S(=O)(NCCCN1c2ccccc2CCc2ccccc21)c1cnc(Cl)c(Br)c1] |
| 106 | 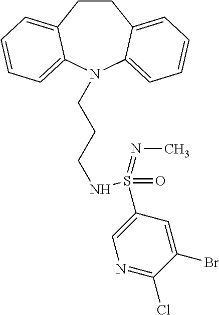 [SMILES: CN=S(=O)(NCCCN1c2ccccc2CCc2ccccc21)c1cnc(Cl)c(Br)c1] |
| 107 | 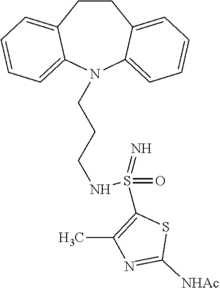 [SMILES: CC(=O)Nc1nc(C)c(S(=N)(=O)NCCCN2c3ccccc3CCc3ccccc32)s1] |
| 108 | 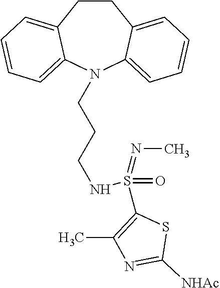 [SMILES: CN=S(=O)(NCCCN1c2ccccc2CCc2ccccc21)c1sc(NC(C)=O)nc1C] |
| 109 | 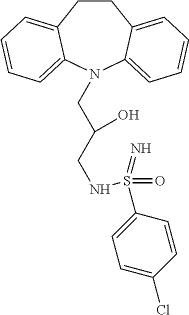 [SMILES: N=S(=O)(NCC(O)CN1c2ccccc2CCc2ccccc21)c1ccc(Cl)cc1] |
| 110 | 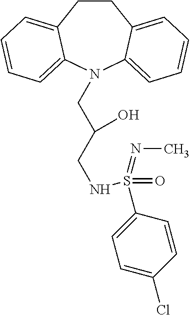 [SMILES: CN=S(=O)(NCC(O)CN1c2ccccc2CCc2ccccc21)c1ccc(Cl)cc1] |
| 111 | 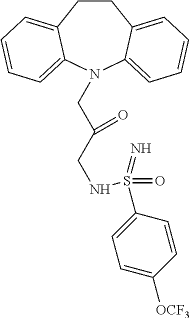 [SMILES: N=S(=O)(NCC(=O)CN1c2ccccc2CCc2ccccc21)c1ccc(OC(F)(F)F)cc1] |
| 112 | 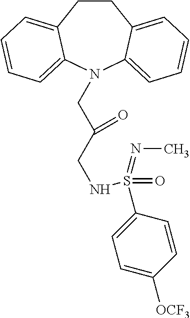 [SMILES: CN=S(=O)(NCC(=O)CN1c2ccccc2CCc2ccccc21)c1ccc(OC(F)(F)F)cc1] |
| 113 | 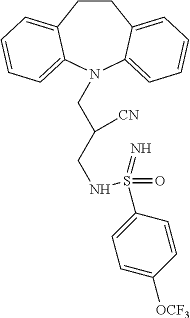 [SMILES: N#CC(CNS(=N)(=O)c1ccc(OC(F)(F)F)cc1)CN1c2ccccc2CCc2ccccc21] |
| 114 | 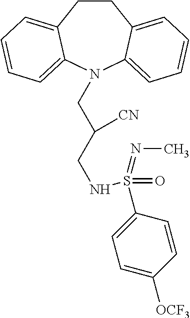 [SMILES: CN=S(=O)(NCC(C#N)CN1c2ccccc2CCc2ccccc21)c1ccc(OC(F)(F)F)cc1] |
| 115 | 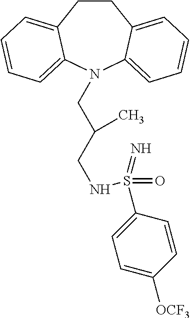 [SMILES: CC(CNS(=N)(=O)c1ccc(OC(F)(F)F)cc1)CN1c2ccccc2CCc2ccccc21] |
| 116 | 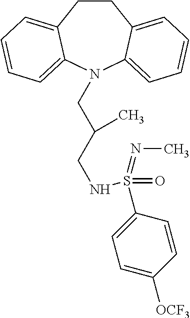 [SMILES: CN=S(=O)(NCC(C)CN1c2ccccc2CCc2ccccc21)c1ccc(OC(F)(F)F)cc1] |
| 117 | 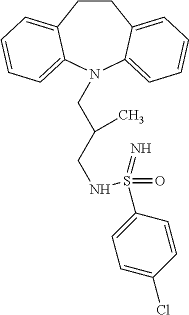 [SMILES: CC(CNS(=N)(=O)c1ccc(Cl)cc1)CN1c2ccccc2CCc2ccccc21] |
| 118 | 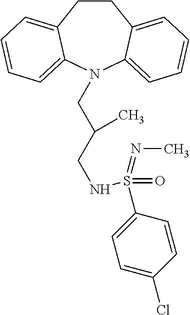 [SMILES: CN=S(=O)(NCC(C)CN1c2ccccc2CCc2ccccc21)c1ccc(Cl)cc1] |
| 119 | 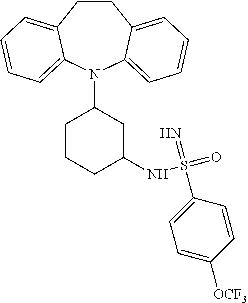 [SMILES: N=S(=O)(NC1CCCC(N2c3ccccc3CCc3ccccc32)C1)c1ccc(OC(F)(F)F)cc1] |
| 120 | 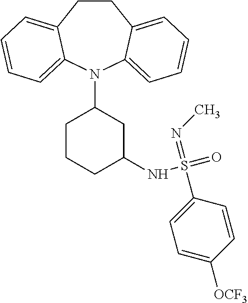 [SMILES: CN=S(=O)(NC1CCCC(N2c3ccccc3CCc3ccccc32)C1)c1ccc(OC(F)(F)F)cc1] |
| 121 | 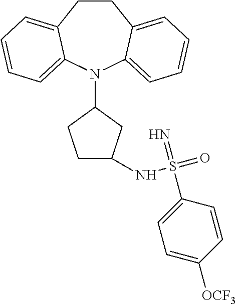 [SMILES: N=S(=O)(NC1CCC(N2c3ccccc3CCc3ccccc32)C1)c1ccc(OC(F)(F)F)cc1] |
| 122 | 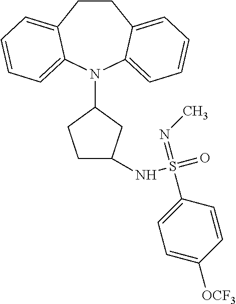 [SMILES: CN=S(=O)(NC1CCC(N2c3ccccc3CCc3ccccc32)C1)c1ccc(OC(F)(F)F)cc1] |
| 123 | 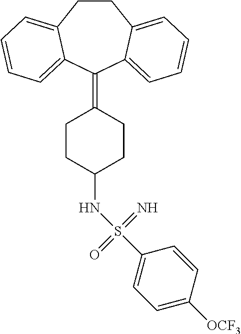 [SMILES: N=S(=O)(NC1CCC(=C2c3ccccc3CCc3ccccc32)CC1)c1ccc(OC(F)(F)F)cc1] |
| 124 | 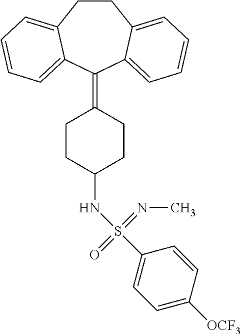 [SMILES: CN=S(=O)(NC1CCC(=C2c3ccccc3CCc3ccccc32)CC1)c1ccc(OC(F)(F)F)cc1] |
| 125 | 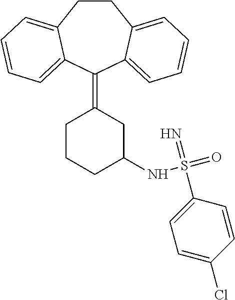 [SMILES: N=S(=O)(NC1CCCC(=C2c3ccccc3CCc3ccccc32)C1)c1ccc(Cl)cc1] |
| 126 | 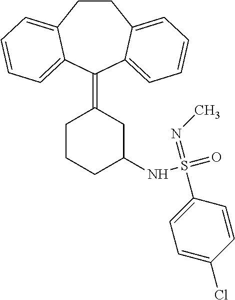 [SMILES: CN=S(=O)(NC1CCCC(=C2c3ccccc3CCc3ccccc32)C1)c1ccc(Cl)cc1] |
| 127 | 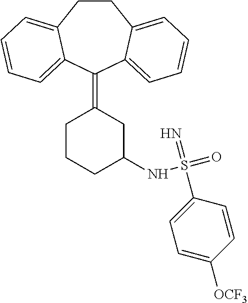 [SMILES: N=S(=O)(NC1CCCC(=C2c3ccccc3CCc3ccccc32)C1)c1ccc(OC(F)(F)F)cc1] |
| 128 | 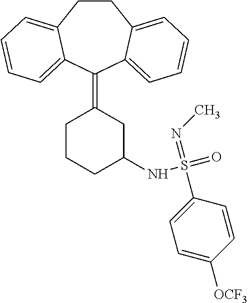 [SMILES: CN=S(=O)(NC1CCCC(=C2c3ccccc3CCc3ccccc32)C1)c1ccc(OC(F)(F)F)cc1] |
| 129 | 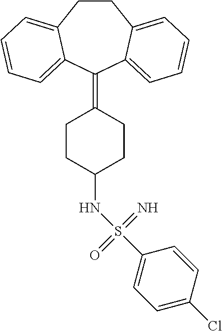 [SMILES: N=S(=O)(NC1CCC(=C2c3ccccc3CCc3ccccc32)CC1)c1ccc(Cl)cc1] |
| 130 | 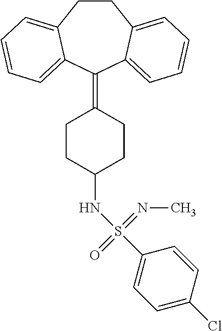 [SMILES: CN=S(=O)(NC1CCC(=C2c3ccccc3CCc3ccccc32)CC1)c1ccc(Cl)cc1] |
| 131 | 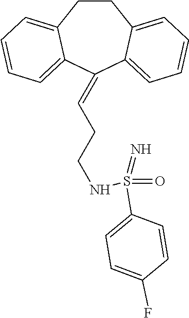 [SMILES: N=S(=O)(NCCC=C1c2ccccc2CCc2ccccc21)c1ccc(F)cc1] |
| 132 | 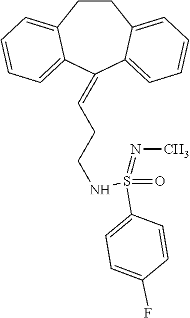 [SMILES: CN=S(=O)(NCCC=C1c2ccccc2CCc2ccccc21)c1ccc(F)cc1] |
| 133 | 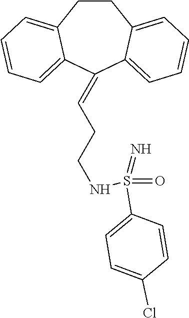 [SMILES: N=S(=O)(NCCC=C1c2ccccc2CCc2ccccc21)c1ccc(Cl)cc1] |
| 134 | 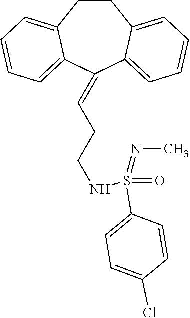 [SMILES: CN=S(=O)(NCCC=C1c2ccccc2CCc2ccccc21)c1ccc(Cl)cc1] |
| 135 | 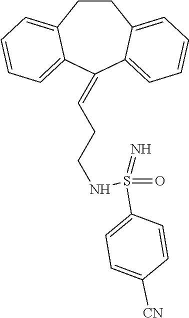 [SMILES: N#Cc1ccc(S(=N)(=O)NCCC=C2c3ccccc3CCc3ccccc32)cc1] |
| 136 | 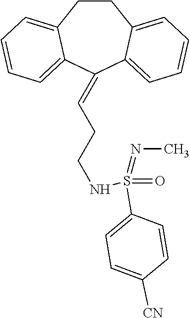 [SMILES: CN=S(=O)(NCCC=C1c2ccccc2CCc2ccccc21)c1ccc(C#N)cc1] |
| 137 | 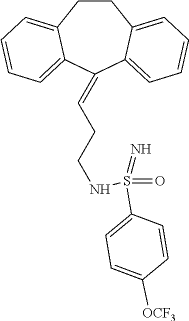 [SMILES: N=S(=O)(NCCC=C1c2ccccc2CCc2ccccc21)c1ccc(OC(F)(F)F)cc1] |
| 138 | 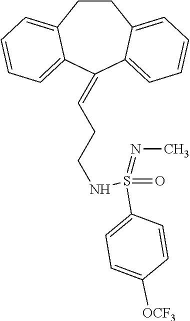 [SMILES: CN=S(=O)(NCCC=C1c2ccccc2CCc2ccccc21)c1ccc(OC(F)(F)F)cc1] |
| 139 | 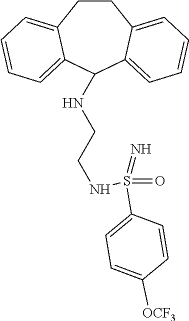 [SMILES: N=S(=O)(NCCNC1c2ccccc2CCc2ccccc21)c1ccc(OC(F)(F)F)cc1] |
| 140 | 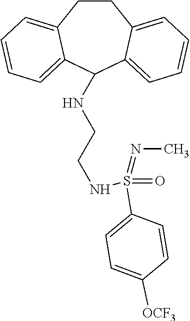 [SMILES: CN=S(=O)(NCCNC1c2ccccc2CCc2ccccc21)c1ccc(OC(F)(F)F)cc1] |
| 141 | 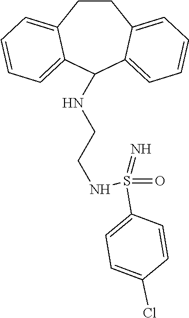 [SMILES: N=S(=O)(NCCNC1c2ccccc2CCc2ccccc21)c1ccc(Cl)cc1] |
| 142 | 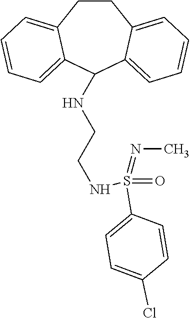 [SMILES: CN=S(=O)(NCCNC1c2ccccc2CCc2ccccc21)c1ccc(Cl)cc1] |
| 143 | 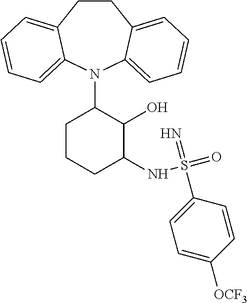 [SMILES: N=S(=O)(NC1CCCC(N2c3ccccc3CCc3ccccc32)C1O)c1ccc(OC(F)(F)F)cc1] |
| 144 | 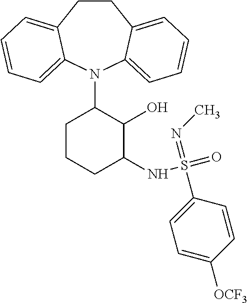 [SMILES: CN=S(=O)(NC1CCCC(N2c3ccccc3CCc3ccccc32)C1O)c1ccc(OC(F)(F)F)cc1] |
| 145 | 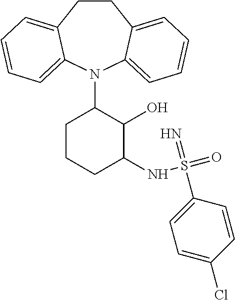 [SMILES: N=S(=O)(NC1CCCC(N2c3ccccc3CCc3ccccc32)C1O)c1ccc(Cl)cc1] |
| 146 | 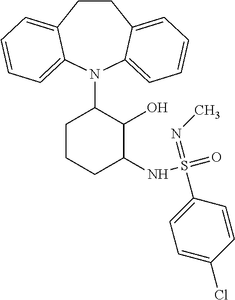 [SMILES: CN=S(=O)(NC1CCCC(N2c3ccccc3CCc3ccccc32)C1O)c1ccc(Cl)cc1] |
| 147 | 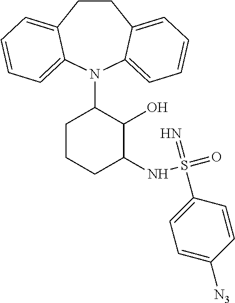 [SMILES: [N-]=[N+]=Nc1ccc(S(=N)(=O)NC2CCCC(N3c4ccccc4CCc4ccccc43)C2O)cc1] |
| 148 | 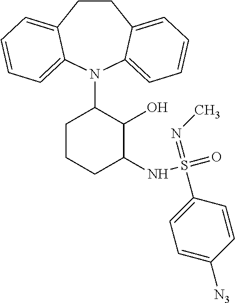 [SMILES: CN=S(=O)(NC1CCCC(N2c3ccccc3CCc3ccccc32)C1O)c1ccc(N=[N+]=[N-])cc1] |
| 149 | 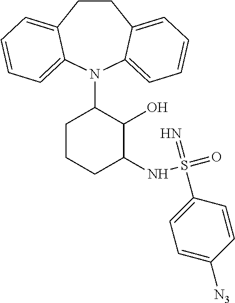 [SMILES: [N-]=[N+]=Nc1ccc(S(=N)(=O)NC2CCCC(N3c4ccccc4CCc4ccccc43)C2O)cc1] |
| 150 | 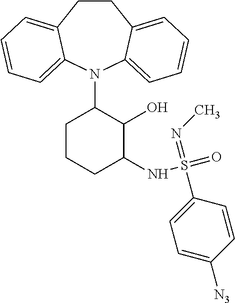 [SMILES: CN=S(=O)(NC1CCCC(N2c3ccccc3CCc3ccccc32)C1O)c1ccc(N=[N+]=[N-])cc1] |
| 151 | 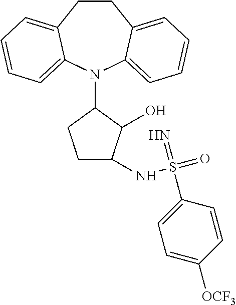 [SMILES: N=S(=O)(NC1CCC(N2c3ccccc3CCc3ccccc32)C1O)c1ccc(OC(F)(F)F)cc1] |
| 152 | 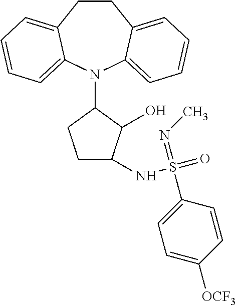 [SMILES: CN=S(=O)(NC1CCC(N2c3ccccc3CCc3ccccc32)C1O)c1ccc(OC(F)(F)F)cc1] |
| 153 | 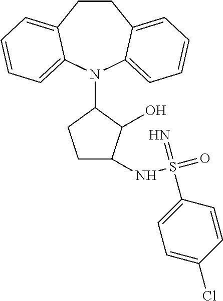 [SMILES: N=S(=O)(NC1CCC(N2c3ccccc3CCc3ccccc32)C1O)c1ccc(Cl)cc1] |
| 154 | 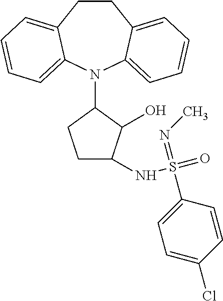 [SMILES: CN=S(=O)(NC1CCC(N2c3ccccc3CCc3ccccc32)C1O)c1ccc(Cl)cc1] |
| 155 | 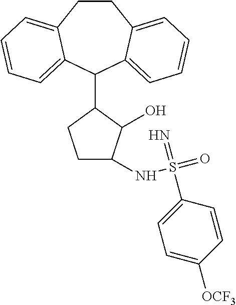 [SMILES: N=S(=O)(NC1CCC(C2c3ccccc3CCc3ccccc32)C1O)c1ccc(OC(F)(F)F)cc1] |
| 156 | 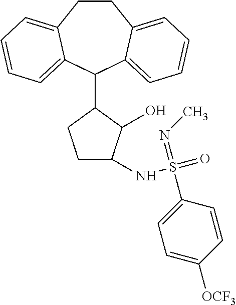 [SMILES: CN=S(=O)(NC1CCC(C2c3ccccc3CCc3ccccc32)C1O)c1ccc(OC(F)(F)F)cc1] |
| 157 | 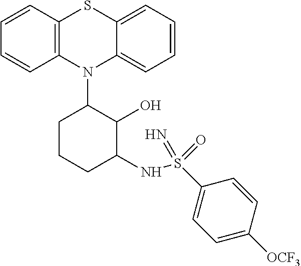 [SMILES: N=S(=O)(NC1CCCC(N2c3ccccc3Sc3ccccc32)C1O)c1ccc(OC(F)(F)F)cc1] |
| 158 | 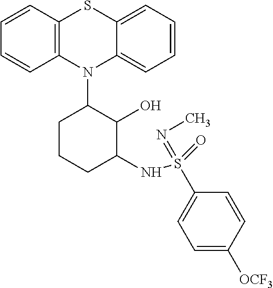 [SMILES: CN=S(=O)(NC1CCCC(N2c3ccccc3Sc3ccccc32)C1O)c1ccc(OC(F)(F)F)cc1] |
| 159 | 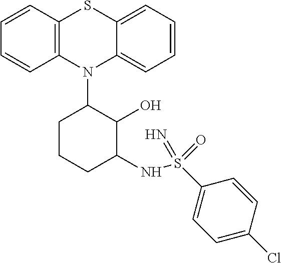 [SMILES: N=S(=O)(NC1CCCC(N2c3ccccc3Sc3ccccc32)C1O)c1ccc(Cl)cc1] |
| 160 | 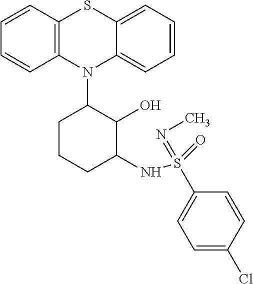 [SMILES: CN=S(=O)(NC1CCCC(N2c3ccccc3Sc3ccccc32)C1O)c1ccc(Cl)cc1] |
| 161 | 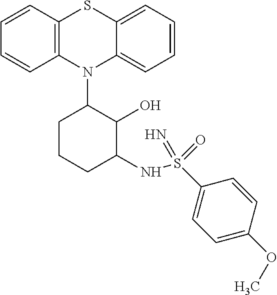 [SMILES: COc1ccc(S(=N)(=O)NC2CCCC(N3c4ccccc4Sc4ccccc43)C2O)cc1] |
| 162 | 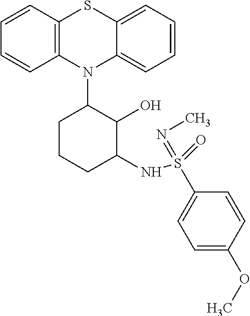 [SMILES: CN=S(=O)(NC1CCCC(N2c3ccccc3Sc3ccccc32)C1O)c1ccc(OC)cc1] |
| 163 | 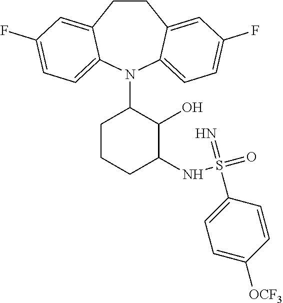 [SMILES: N=S(=O)(NC1CCCC(N2c3ccc(F)cc3CCc3cc(F)ccc32)C1O)c1ccc(OC(F)(F)F)cc1] |
| 164 | 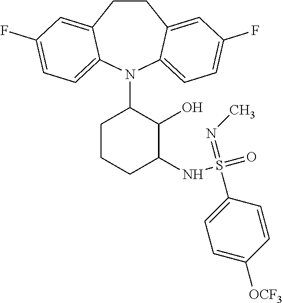 [SMILES: CN=S(=O)(NC1CCCC(N2c3ccc(F)cc3CCc3cc(F)ccc32)C1O)c1ccc(OC(F)(F)F)cc1] |
| 165 | 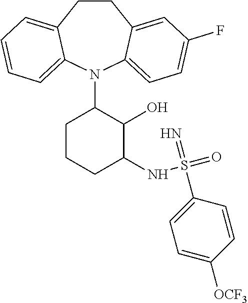 [SMILES: N=S(=O)(NC1CCCC(N2c3ccccc3CCc3cc(F)ccc32)C1O)c1ccc(OC(F)(F)F)cc1] |
| 166 | 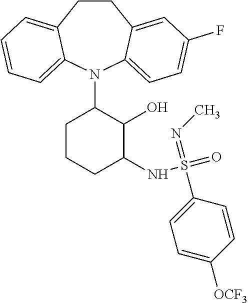 [SMILES: CN=S(=O)(NC1CCCC(N2c3ccccc3CCc3cc(F)ccc32)C1O)c1ccc(OC(F)(F)F)cc1] |
| 167 | 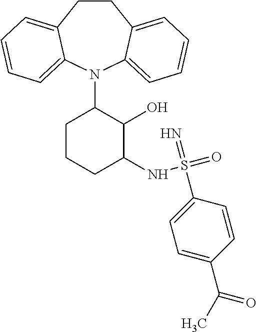 [SMILES: CC(=O)c1ccc(S(=N)(=O)NC2CCCC(N3c4ccccc4CCc4ccccc43)C2O)cc1] |
| 168 | 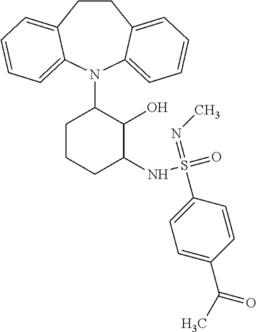 [SMILES: CN=S(=O)(NC1CCCC(N2c3ccccc3CCc3ccccc32)C1O)c1ccc(C(C)=O)cc1] |
| 169 | 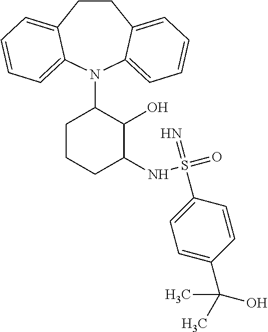 [SMILES: CC(C)(O)c1ccc(S(=N)(=O)NC2CCCC(N3c4ccccc4CCc4ccccc43)C2O)cc1] |
| 170 | 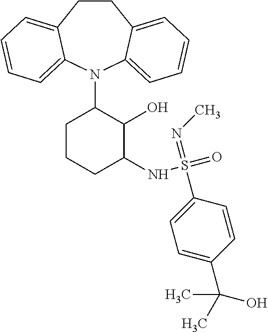 [SMILES: CN=S(=O)(NC1CCCC(N2c3ccccc3CCc3ccccc32)C1O)c1ccc(C(C)(C)O)cc1] |
| 171 | 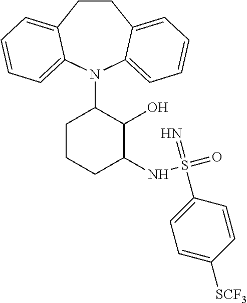 |
| 172 | 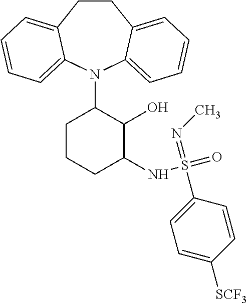 |
| 173 | 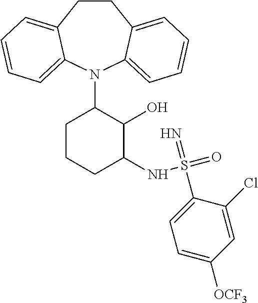 [SMILES: N=S(=O)(NC1CCCC(N2c3ccccc3CCc3ccccc32)C1O)c1ccc(OC(F)(F)F)cc1Cl] |
| 174 | 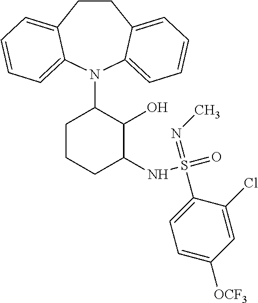 [SMILES: CN=S(=O)(NC1CCCC(N2c3ccccc3CCc3ccccc32)C1O)c1ccc(OC(F)(F)F)cc1Cl] |
| 175 | 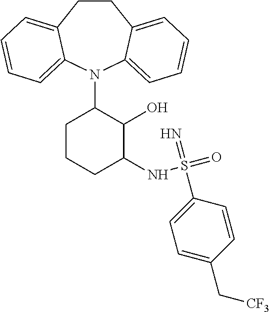 [SMILES: N=S(=O)(NC1CCCC(N2c3ccccc3CCc3ccccc32)C1O)c1ccc(CC(F)(F)F)cc1] |
| 176 | 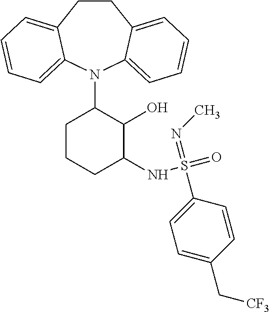 [SMILES: CN=S(=O)(NC1CCCC(N2c3ccccc3CCc3ccccc32)C1O)c1ccc(CC(F)(F)F)cc1] |
| 177 | 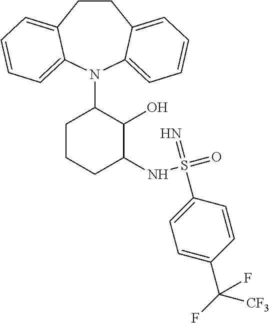 [SMILES: N=S(=O)(NC1CCCC(N2c3ccccc3CCc3ccccc32)C1O)c1ccc(C(F)(F)C(F)(F)F)cc1] |
| 178 | 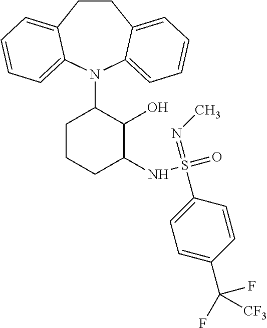 [SMILES: CN=S(=O)(NC1CCCC(N2c3ccccc3CCc3ccccc32)C1O)c1ccc(C(F)(F)C(F)(F)F)cc1] |
| 179 | 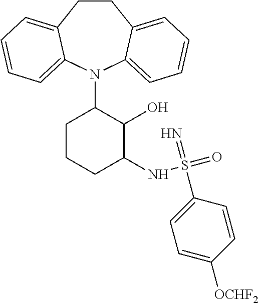 [SMILES: N=S(=O)(NC1CCCC(N2c3ccccc3CCc3ccccc32)C1O)c1ccc(OC(F)F)cc1] |
| 180 | 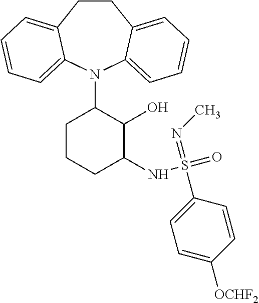 [SMILES: CN=S(=O)(NC1CCCC(N2c3ccccc3CCc3ccccc32)C1O)c1ccc(OC(F)F)cc1] |
| 181 | 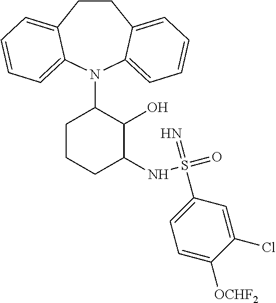 [SMILES: N=S(=O)(NC1CCCC(N2c3ccccc3CCc3ccccc32)C1O)c1ccc(OC(F)F)c(Cl)c1] |
| 182 | 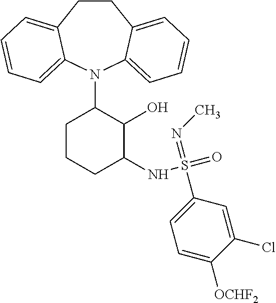 [SMILES: CN=S(=O)(NC1CCCC(N2c3ccccc3CCc3ccccc32)C1O)c1ccc(OC(F)F)c(Cl)c1] |
| 183 | 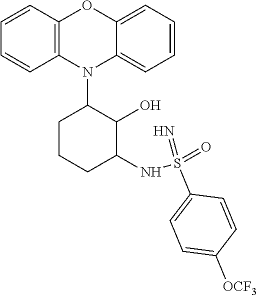 [SMILES: N=S(=O)(NC1CCCC(N2c3ccccc3Oc3ccccc32)C1O)c1ccc(OC(F)(F)F)cc1] |
| 184 | 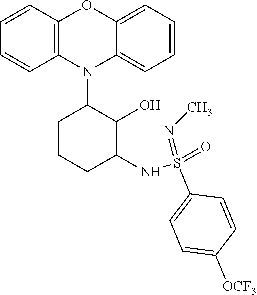 [SMILES: CN=S(=O)(NC1CCCC(N2c3ccccc3Oc3ccccc32)C1O)c1ccc(OC(F)(F)F)cc1] |
| 185 | 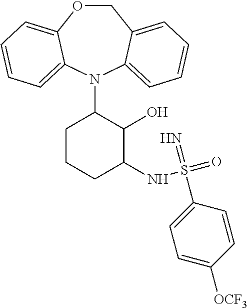 [SMILES: N=S(=O)(NC1CCCC(N2c3ccccc3COc3ccccc32)C1O)c1ccc(OC(F)(F)F)cc1] |
| 186 | 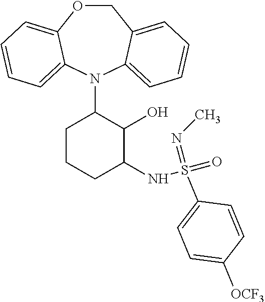 [SMILES: CN=S(=O)(NC1CCCC(N2c3ccccc3COc3ccccc32)C1O)c1ccc(OC(F)(F)F)cc1] |
| 187 | 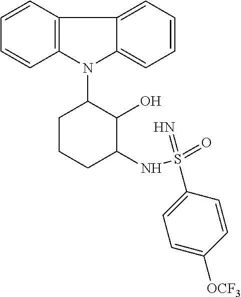 [SMILES: N=S(=O)(NC1CCCC(n2c3ccccc3c3ccccc32)C1O)c1ccc(OC(F)(F)F)cc1] |
| 188 | 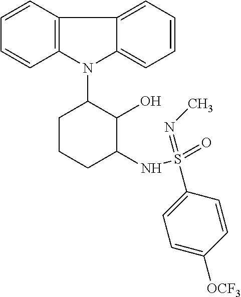 [SMILES: CN=S(=O)(NC1CCCC(n2c3ccccc3c3ccccc32)C1O)c1ccc(OC(F)(F)F)cc1] |
| 189 | 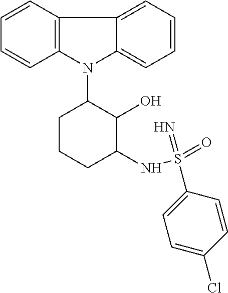 [SMILES: N=S(=O)(NC1CCCC(n2c3ccccc3c3ccccc32)C1O)c1ccc(Cl)cc1] |
| 190 | 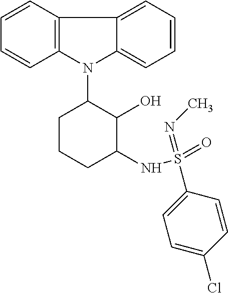 [SMILES: CN=S(=O)(NC1CCCC(n2c3ccccc3c3ccccc32)C1O)c1ccc(Cl)cc1] |
| 191 | 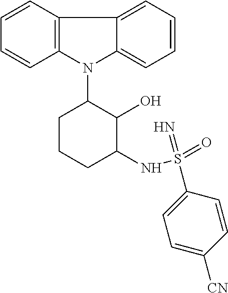 [SMILES: N#Cc1ccc(S(=N)(=O)NC2CCCC(n3c4ccccc4c4ccccc43)C2O)cc1] |
| 192 | 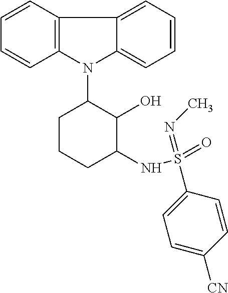 [SMILES: CN=S(=O)(NC1CCCC(n2c3ccccc3c3ccccc32)C1O)c1ccc(C#N)cc1] |
| 193 | 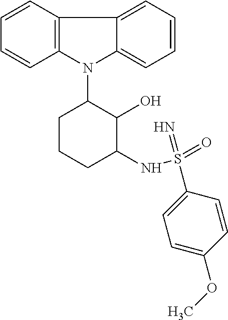 [SMILES: COc1ccc(S(=N)(=O)NC2CCCC(n3c4ccccc4c4ccccc43)C2O)cc1] |
| 194 | 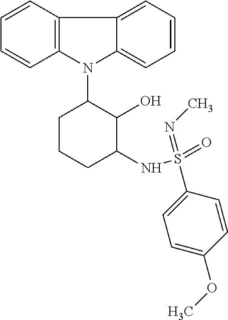 [SMILES: CN=S(=O)(NC1CCCC(n2c3ccccc3c3ccccc32)C1O)c1ccc(OC)cc1] |
| 195 | 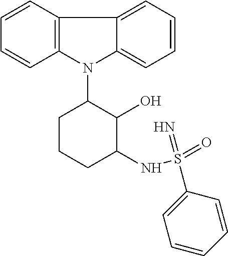 [SMILES: N=S(=O)(NC1CCCC(n2c3ccccc3c3ccccc32)C1O)c1ccccc1] |
| 196 | 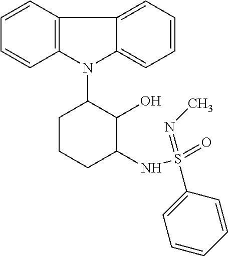 [SMILES: CN=S(=O)(NC1CCCC(n2c3ccccc3c3ccccc32)C1O)c1ccccc1] |
| 197 | 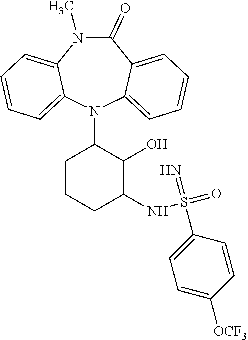 [SMILES: CN1C(=O)c2ccccc2N(C2CCCC(NS(=N)(=O)c3ccc(OC(F)(F)F)cc3)C2O)c2ccccc21] |
| 198 | 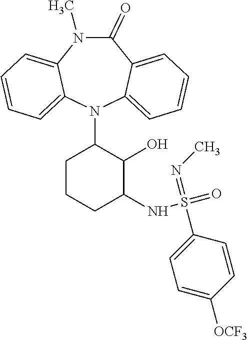 [SMILES: CN=S(=O)(NC1CCCC(N2c3ccccc3C(=O)N(C)c3ccccc32)C1O)c1ccc(OC(F)(F)F)cc1] |
| 199 | 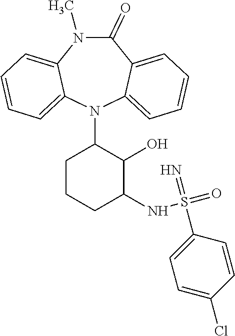 [SMILES: CN1C(=O)c2ccccc2N(C2CCCC(NS(=N)(=O)c3ccc(Cl)cc3)C2O)c2ccccc21] |
| 200 | 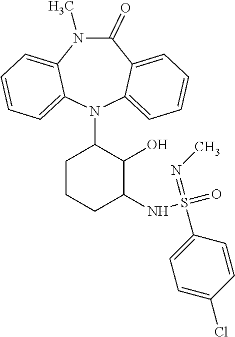 [SMILES: CN=S(=O)(NC1CCCC(N2c3ccccc3C(=O)N(C)c3ccccc32)C1O)c1ccc(Cl)cc1] |
| 201 | 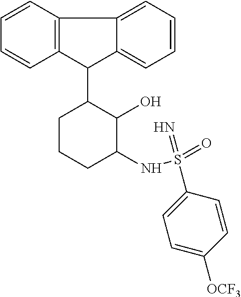 [SMILES: N=S(=O)(NC1CCCC(C2c3ccccc3-c3ccccc32)C1O)c1ccc(OC(F)(F)F)cc1] |
| 202 | 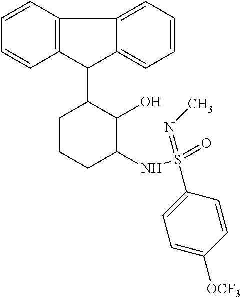 [SMILES: CN=S(=O)(NC1CCCC(C2c3ccccc3-c3ccccc32)C1O)c1ccc(OC(F)(F)F)cc1] |
| 203 | 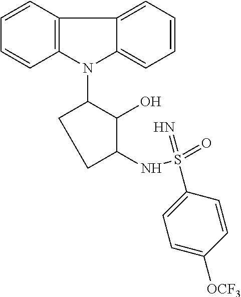 [SMILES: N=S(=O)(NC1CCC(n2c3ccccc3c3ccccc32)C1O)c1ccc(OC(F)(F)F)cc1] |
| 204 | 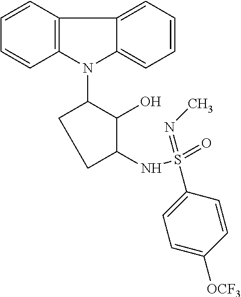 [SMILES: CN=S(=O)(NC1CCC(n2c3ccccc3c3ccccc32)C1O)c1ccc(OC(F)(F)F)cc1] |
| 205 | 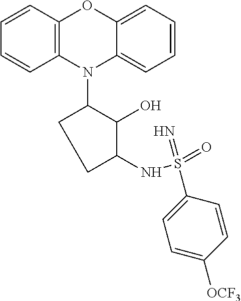 [SMILES: N=S(=O)(NC1CCC(N2c3ccccc3Oc3ccccc32)C1O)c1ccc(OC(F)(F)F)cc1] |
| 206 | 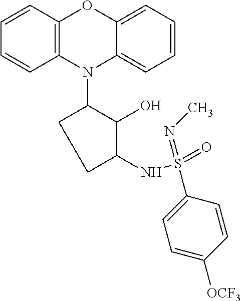 [SMILES: CN=S(=O)(NC1CCC(N2c3ccccc3Oc3ccccc32)C1O)c1ccc(OC(F)(F)F)cc1] |
| 207 | 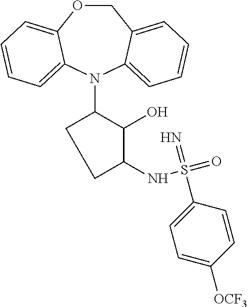 [SMILES: N=S(=O)(NC1CCC(N2c3ccccc3COc3ccccc32)C1O)c1ccc(OC(F)(F)F)cc1] |
| 208 | 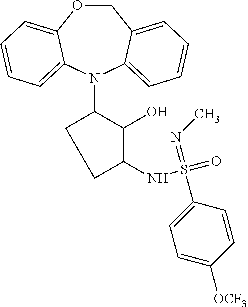 [SMILES: CN=S(=O)(NC1CCC(N2c3ccccc3COc3ccccc32)C1O)c1ccc(OC(F)(F)F)cc1] |
| 209 | 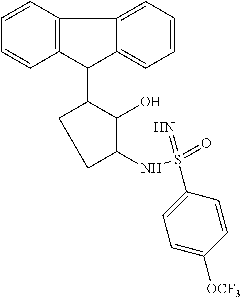 [SMILES: N=S(=O)(NC1CCC(C2c3ccccc3-c3ccccc32)C1O)c1ccc(OC(F)(F)F)cc1] |
| 210 | 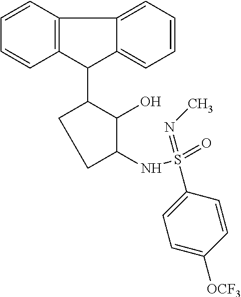 [SMILES: CN=S(=O)(NC1CCC(C2c3ccccc3-c3ccccc32)C1O)c1ccc(OC(F)(F)F)cc1] |
| 211 | 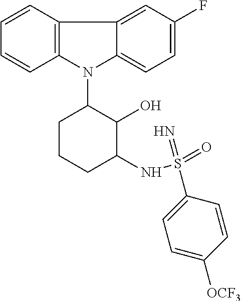 [SMILES: N=S(=O)(NC1CCCC(n2c3ccccc3c3cc(F)ccc32)C1O)c1ccc(OC(F)(F)F)cc1] |
| 212 | 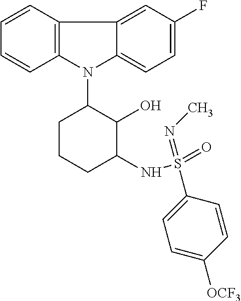 [SMILES: CN=S(=O)(NC1CCCC(n2c3ccccc3c3cc(F)ccc32)C1O)c1ccc(OC(F)(F)F)cc1] |
| 213 | 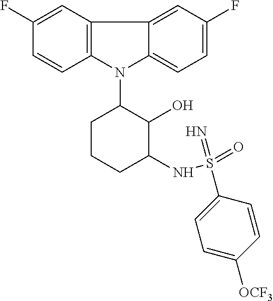 [SMILES: N=S(=O)(NC1CCCC(n2c3ccc(F)cc3c3cc(F)ccc32)C1O)c1ccc(OC(F)(F)F)cc1] |
| 214 | 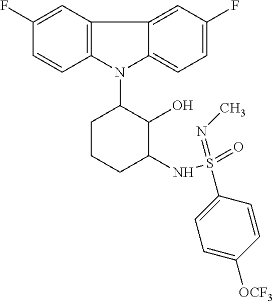 [SMILES: CN=S(=O)(NC1CCCC(n2c3ccc(F)cc3c3cc(F)ccc32)C1O)c1ccc(OC(F)(F)F)cc1] |
| 215 | 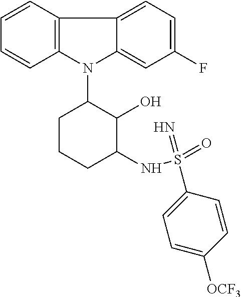 [SMILES: N=S(=O)(NC1CCCC(n2c3ccccc3c3ccc(F)cc32)C1O)c1ccc(OC(F)(F)F)cc1] |
| 216 | 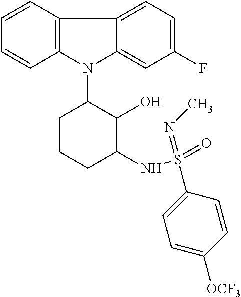 [SMILES: CN=S(=O)(NC1CCCC(n2c3ccccc3c3ccc(F)cc32)C1O)c1ccc(OC(F)(F)F)cc1] |
| 217 | 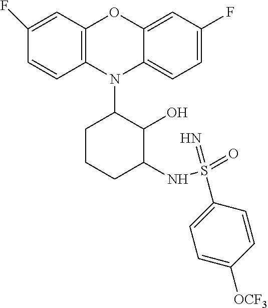 [SMILES: N=S(=O)(NC1CCCC(N2c3ccc(F)cc3Oc3cc(F)ccc32)C1O)c1ccc(OC(F)(F)F)cc1] |
| 218 | 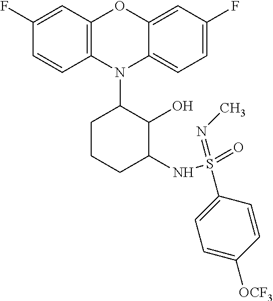 [SMILES: CN=S(=O)(NC1CCCC(N2c3ccc(F)cc3Oc3cc(F)ccc32)C1O)c1ccc(OC(F)(F)F)cc1] |
| 219 | 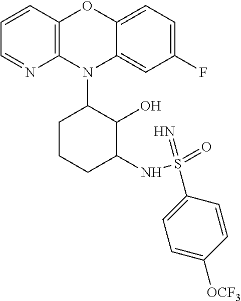 [SMILES: N=S(=O)(NC1CCCC(N2c3cc(F)ccc3Oc3cccnc32)C1O)c1ccc(OC(F)(F)F)cc1] |
| 220 | 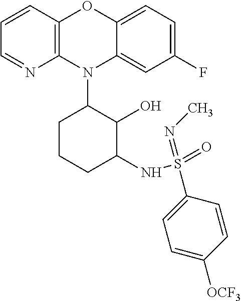 [SMILES: CN=S(=O)(NC1CCCC(N2c3cc(F)ccc3Oc3cccnc32)C1O)c1ccc(OC(F)(F)F)cc1] |
| 221 | 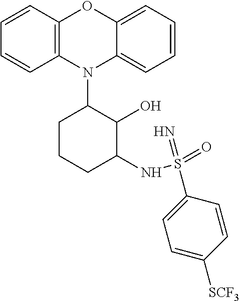 |
| 222 | 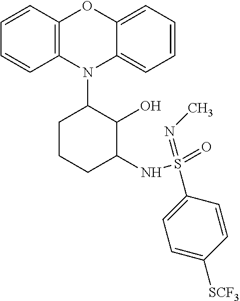 |
| 223 | 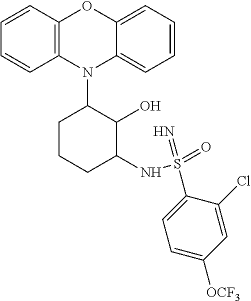 [SMILES: N=S(=O)(NC1CCCC(N2c3ccccc3Oc3ccccc32)C1O)c1ccc(OC(F)(F)F)cc1Cl] |
| 224 | 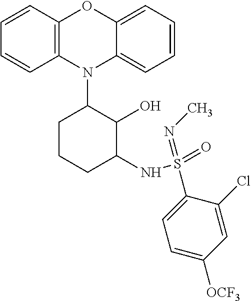 [SMILES: CN=S(=O)(NC1CCCC(N2c3ccccc3Oc3ccccc32)C1O)c1ccc(OC(F)(F)F)cc1Cl] |
| 225 | 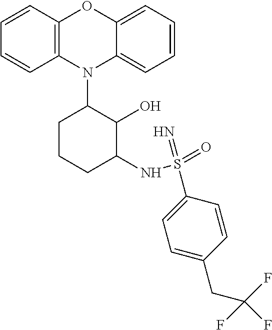 [SMILES: N=S(=O)(NC1CCCC(N2c3ccccc3Oc3ccccc32)C1O)c1ccc(CC(F)(F)F)cc1] |
| 226 | 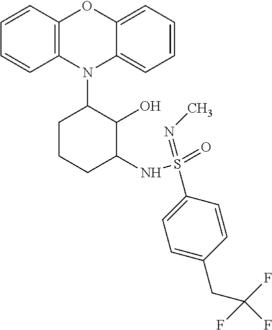 [SMILES: CN=S(=O)(NC1CCCC(N2c3ccccc3Oc3ccccc32)C1O)c1ccc(CC(F)(F)F)cc1] |
| 227 | 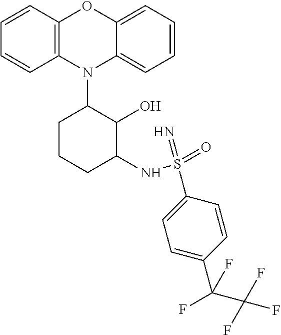 [SMILES: N=S(=O)(NC1CCCC(N2c3ccccc3Oc3ccccc32)C1O)c1ccc(C(F)(F)C(F)(F)F)cc1] |
| 228 | 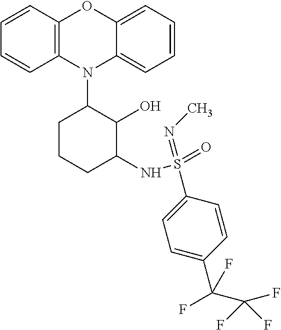 [SMILES: CN=S(=O)(NC1CCCC(N2c3ccccc3Oc3ccccc32)C1O)c1ccc(C(F)(F)C(F)(F)F)cc1] |
| 229 | 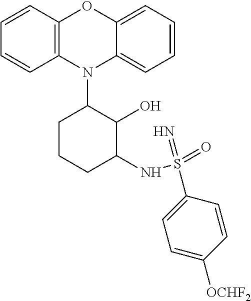 [SMILES: N=S(=O)(NC1CCCC(N2c3ccccc3Oc3ccccc32)C1O)c1ccc(OC(F)F)cc1] |
| 230 | 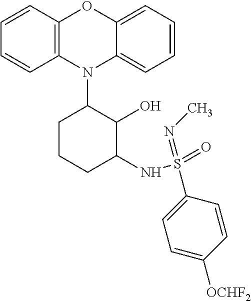 [SMILES: CN=S(=O)(NC1CCCC(N2c3ccccc3Oc3ccccc32)C1O)c1ccc(OC(F)F)cc1] |
| 231 | 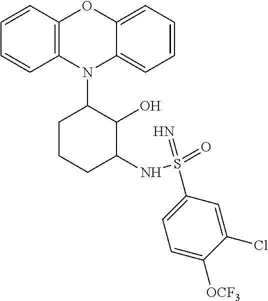 [SMILES: N=S(=O)(NC1CCCC(N2c3ccccc3Oc3ccccc32)C1O)c1ccc(OC(F)(F)F)c(Cl)c1] |
| 232 | 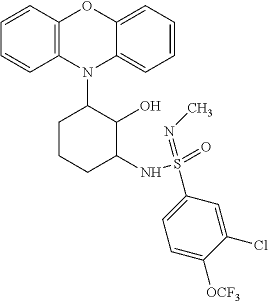 [SMILES: CN=S(=O)(NC1CCCC(N2c3ccccc3Oc3ccccc32)C1O)c1ccc(OC(F)(F)F)c(Cl)c1] |
| 233 | 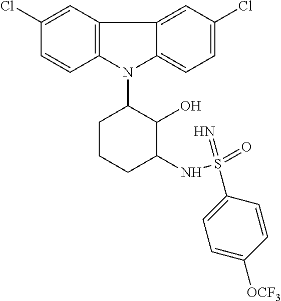 [SMILES: N=S(=O)(NC1CCCC(n2c3ccc(Cl)cc3c3cc(Cl)ccc32)C1O)c1ccc(OC(F)(F)F)cc1] |
| 234 | 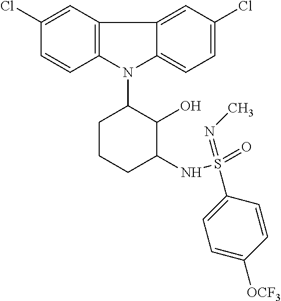 [SMILES: CN=S(=O)(NC1CCCC(n2c3ccc(Cl)cc3c3cc(Cl)ccc32)C1O)c1ccc(OC(F)(F)F)cc1] |
| 235 | 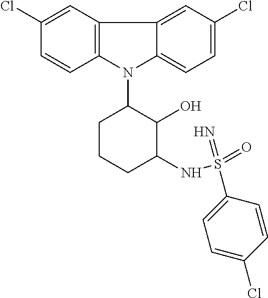 [SMILES: N=S(=O)(NC1CCCC(n2c3ccc(Cl)cc3c3cc(Cl)ccc32)C1O)c1ccc(Cl)cc1] |
| 236 | 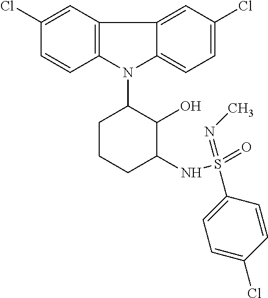 [SMILES: CN=S(=O)(NC1CCCC(n2c3ccc(Cl)cc3c3cc(Cl)ccc32)C1O)c1ccc(Cl)cc1] |
| 237 | 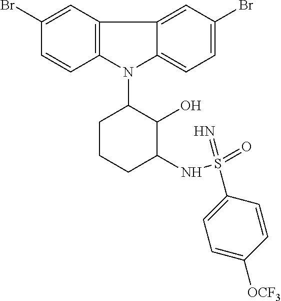 [SMILES: N=S(=O)(NC1CCCC(n2c3ccc(Br)cc3c3cc(Br)ccc32)C1O)c1ccc(OC(F)(F)F)cc1] |
| 238 | 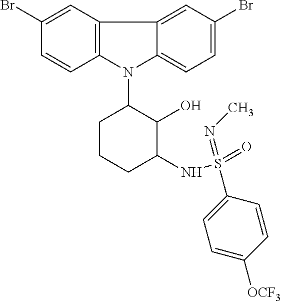 [SMILES: CN=S(=O)(NC1CCCC(n2c3ccc(Br)cc3c3cc(Br)ccc32)C1O)c1ccc(OC(F)(F)F)cc1] |
| 239 | 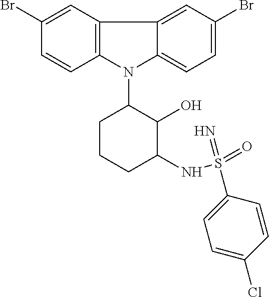 [SMILES: N=S(=O)(NC1CCCC(n2c3ccc(Br)cc3c3cc(Br)ccc32)C1O)c1ccc(Cl)cc1] |
| 240 | 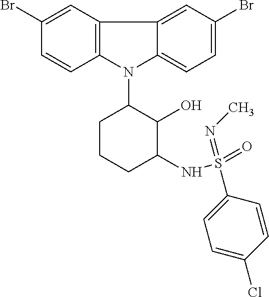 [SMILES: CN=S(=O)(NC1CCCC(n2c3ccc(Br)cc3c3cc(Br)ccc32)C1O)c1ccc(Cl)cc1] |
| 241 | 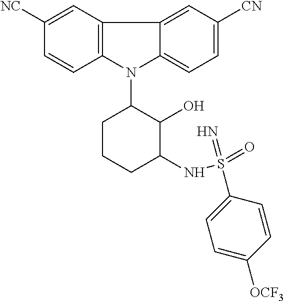 [SMILES: N#Cc1ccc2c(c1)c1cc(C#N)ccc1n2C1CCCC(NS(=N)(=O)c2ccc(OC(F)(F)F)cc2)C1O] |
| 242 | 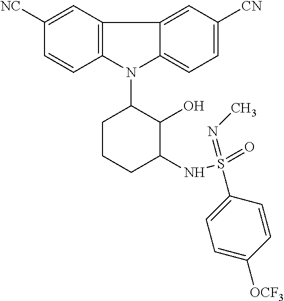 [SMILES: CN=S(=O)(NC1CCCC(n2c3ccc(C#N)cc3c3cc(C#N)ccc32)C1O)c1ccc(OC(F)(F)F)cc1] |
| 243 | 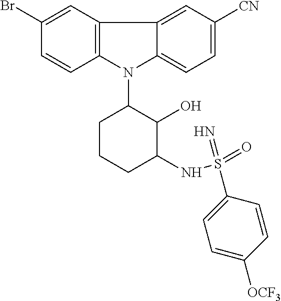 [SMILES: N#Cc1ccc2c(c1)c1cc(Br)ccc1n2C1CCCC(NS(=N)(=O)c2ccc(OC(F)(F)F)cc2)C1O] |
| 244 | 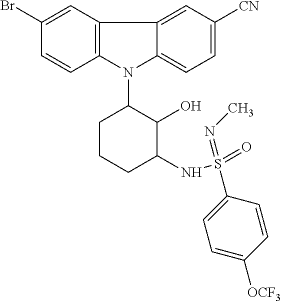 [SMILES: CN=S(=O)(NC1CCCC(n2c3ccc(Br)cc3c3cc(C#N)ccc32)C1O)c1ccc(OC(F)(F)F)cc1] |
| 245 | 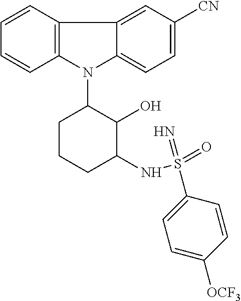 [SMILES: N#Cc1ccc2c(c1)c1ccccc1n2C1CCCC(NS(=N)(=O)c2ccc(OC(F)(F)F)cc2)C1O] |
| 246 | 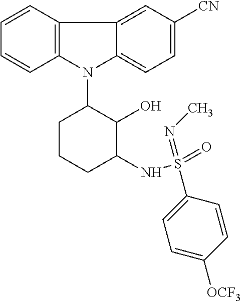 [SMILES: CN=S(=O)(NC1CCCC(n2c3ccccc3c3cc(C#N)ccc32)C1O)c1ccc(OC(F)(F)F)cc1] |
| 247 | 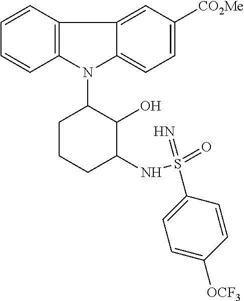 [SMILES: COC(=O)c1ccc2c(c1)c1ccccc1n2C1CCCC(NS(=N)(=O)c2ccc(OC(F)(F)F)cc2)C1O] |
| 248 | 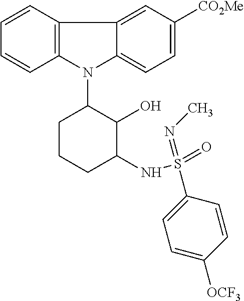 [SMILES: CN=S(=O)(NC1CCCC(n2c3ccccc3c3cc(C(=O)OC)ccc32)C1O)c1ccc(OC(F)(F)F)cc1] |
| 249 | 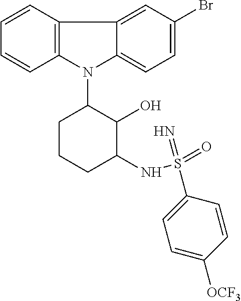 [SMILES: N=S(=O)(NC1CCCC(n2c3ccccc3c3cc(Br)ccc32)C1O)c1ccc(OC(F)(F)F)cc1] |
| 250 | 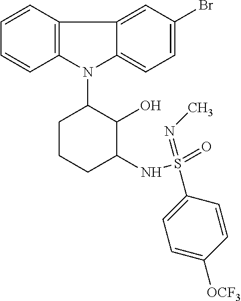 [SMILES: CN=S(=O)(NC1CCCC(n2c3ccccc3c3cc(Br)ccc32)C1O)c1ccc(OC(F)(F)F)cc1] |
| 251 | 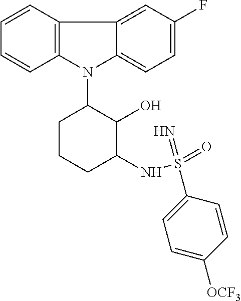 [SMILES: N=S(=O)(NC1CCCC(n2c3ccccc3c3cc(F)ccc32)C1O)c1ccc(OC(F)(F)F)cc1] |
| 252 | 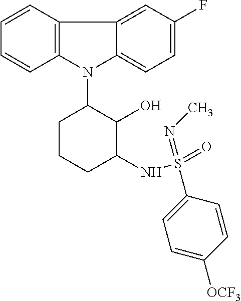 [SMILES: CN=S(=O)(NC1CCCC(n2c3ccccc3c3cc(F)ccc32)C1O)c1ccc(OC(F)(F)F)cc1] |
| 253 | 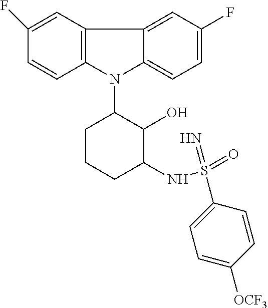 [SMILES: N=S(=O)(NC1CCCC(n2c3ccc(F)cc3c3cc(F)ccc32)C1O)c1ccc(OC(F)(F)F)cc1] |
| 254 | 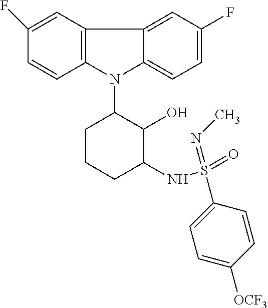 [SMILES: CN=S(=O)(NC1CCCC(n2c3ccc(F)cc3c3cc(F)ccc32)C1O)c1ccc(OC(F)(F)F)cc1] |
| 255 | 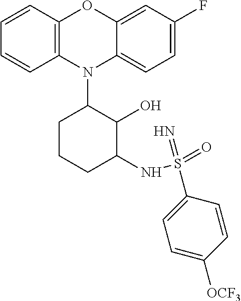 [SMILES: N=S(=O)(NC1CCCC(N2c3ccccc3Oc3cc(F)ccc32)C1O)c1ccc(OC(F)(F)F)cc1] |
| 256 | 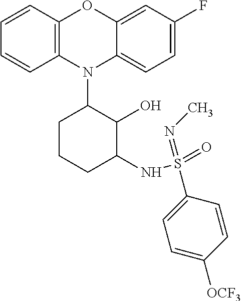 [SMILES: CN=S(=O)(NC1CCCC(N2c3ccccc3Oc3cc(F)ccc32)C1O)c1ccc(OC(F)(F)F)cc1] |
| 257 | 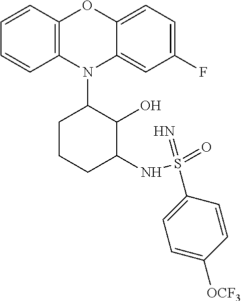 [SMILES: N=S(=O)(NC1CCCC(N2c3ccccc3Oc3ccc(F)cc32)C1O)c1ccc(OC(F)(F)F)cc1] |
| 258 | 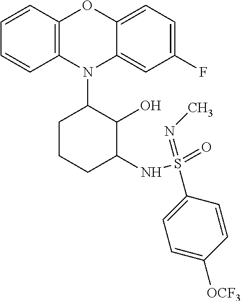 [SMILES: CN=S(=O)(NC1CCCC(N2c3ccccc3Oc3ccc(F)cc32)C1O)c1ccc(OC(F)(F)F)cc1] |
| 259 | 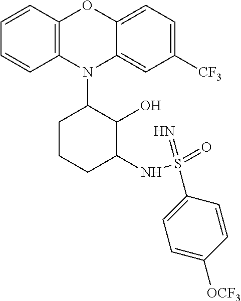 [SMILES: N=S(=O)(NC1CCCC(N2c3ccccc3Oc3ccc(C(F)(F)F)cc32)C1O)c1ccc(OC(F)(F)F)cc1] |
| 260 | 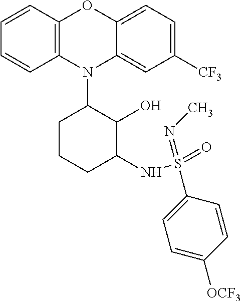 [SMILES: CN=S(=O)(NC1CCCC(N2c3ccccc3Oc3ccc(C(F)(F)F)cc32)C1O)c1ccc(OC(F)(F)F)cc1] |
| 261 | 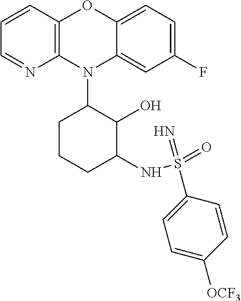 [SMILES: N=S(=O)(NC1CCCC(N2c3cc(F)ccc3Oc3cccnc32)C1O)c1ccc(OC(F)(F)F)cc1] |
| 262 | 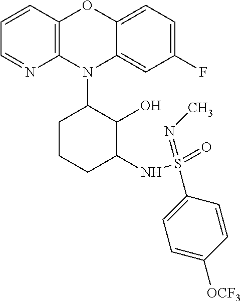 [SMILES: CN=S(=O)(NC1CCCC(N2c3cc(F)ccc3Oc3cccnc32)C1O)c1ccc(OC(F)(F)F)cc1] |
| 263 | 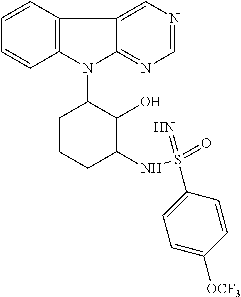 [SMILES: N=S(=O)(NC1CCCC(n2c3ccccc3c3cncnc32)C1O)c1ccc(OC(F)(F)F)cc1] |
| 264 | 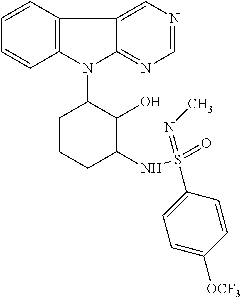 [SMILES: CN=S(=O)(NC1CCCC(n2c3ccccc3c3cncnc32)C1O)c1ccc(OC(F)(F)F)cc1] |
| 265 | 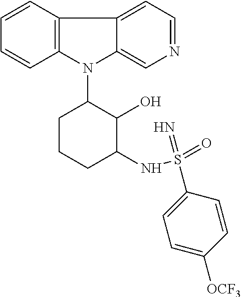 [SMILES: N=S(=O)(NC1CCCC(n2c3ccccc3c3ccncc32)C1O)c1ccc(OC(F)(F)F)cc1] |
| 266 | 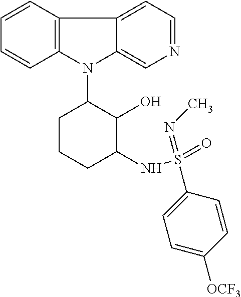 [SMILES: CN=S(=O)(NC1CCCC(n2c3ccccc3c3ccncc32)C1O)c1ccc(OC(F)(F)F)cc1] |
| 267 | 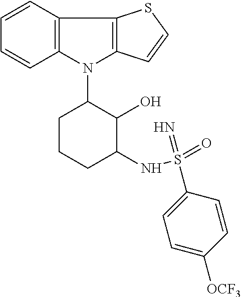 [SMILES: N=S(=O)(NC1CCCC(n2c3ccccc3c3sccc32)C1O)c1ccc(OC(F)(F)F)cc1] |
| 268 | 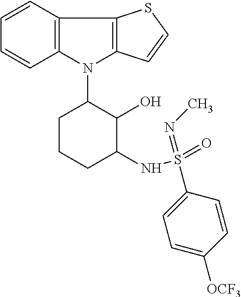 [SMILES: CN=S(=O)(NC1CCCC(n2c3ccccc3c3sccc32)C1O)c1ccc(OC(F)(F)F)cc1] |
| 269 | 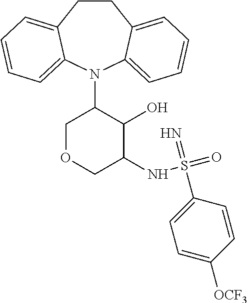 [SMILES: N=S(=O)(NC1COCC(N2c3ccccc3CCc3ccccc32)C1O)c1ccc(OC(F)(F)F)cc1] |
| 270 | 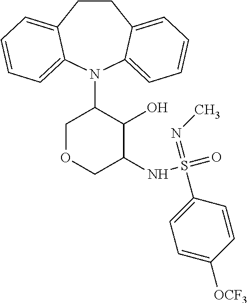 [SMILES: CN=S(=O)(NC1COCC(N2c3ccccc3CCc3ccccc32)C1O)c1ccc(OC(F)(F)F)cc1] |
| 271 | 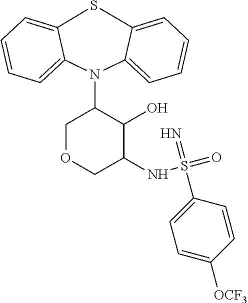 [SMILES: N=S(=O)(NC1COCC(N2c3ccccc3Sc3ccccc32)C1O)c1ccc(OC(F)(F)F)cc1] |
| 272 | 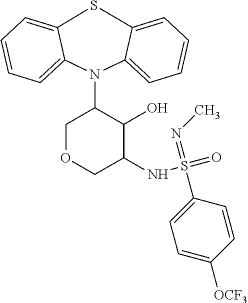 [SMILES: CN=S(=O)(NC1COCC(N2c3ccccc3Sc3ccccc32)C1O)c1ccc(OC(F)(F)F)cc1] |
| 273 | 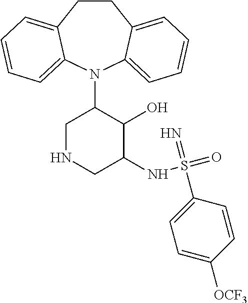 [SMILES: N=S(=O)(NC1CNCC(N2c3ccccc3CCc3ccccc32)C1O)c1ccc(OC(F)(F)F)cc1] |
| 274 | 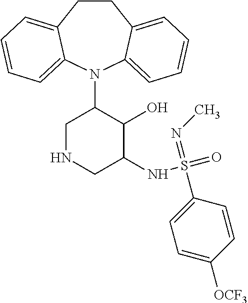 [SMILES: CN=S(=O)(NC1CNCC(N2c3ccccc3CCc3ccccc32)C1O)c1ccc(OC(F)(F)F)cc1] |
| 275 | 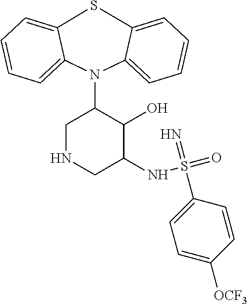 [SMILES: N=S(=O)(NC1CNCC(N2c3ccccc3Sc3ccccc32)C1O)c1ccc(OC(F)(F)F)cc1] |
| 276 | 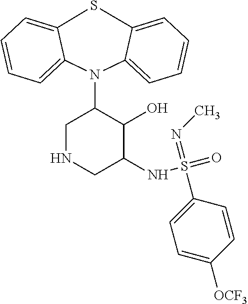 [SMILES: CN=S(=O)(NC1CNCC(N2c3ccccc3Sc3ccccc32)C1O)c1ccc(OC(F)(F)F)cc1] |
| 277 | 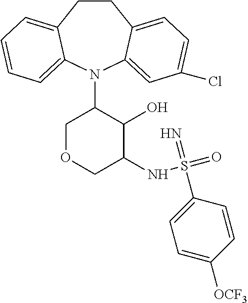 [SMILES: N=S(=O)(NC1COCC(N2c3ccccc3CCc3ccc(Cl)cc32)C1O)c1ccc(OC(F)(F)F)cc1] |
| 278 | 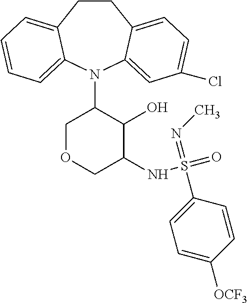 [SMILES: CN=S(=O)(NC1COCC(N2c3ccccc3CCc3ccc(Cl)cc32)C1O)c1ccc(OC(F)(F)F)cc1] |
| 279 | 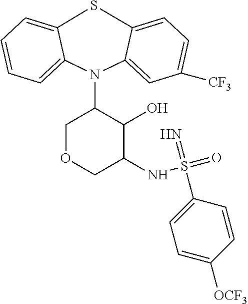 [SMILES: N=S(=O)(NC1COCC(N2c3ccccc3Sc3ccc(C(F)(F)F)cc32)C1O)c1ccc(OC(F)(F)F)cc1] |
| 280 | 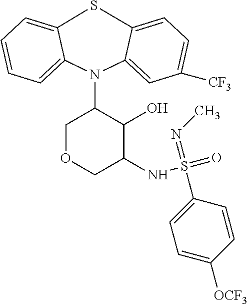 [SMILES: CN=S(=O)(NC1COCC(N2c3ccccc3Sc3ccc(C(F)(F)F)cc32)C1O)c1ccc(OC(F)(F)F)cc1] |
| 281 | 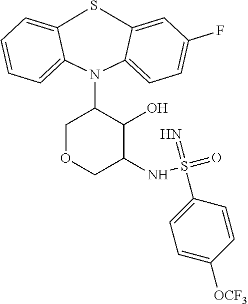 [SMILES: N=S(=O)(NC1COCC(N2c3ccccc3Sc3cc(F)ccc32)C1O)c1ccc(OC(F)(F)F)cc1] |
| 282 | 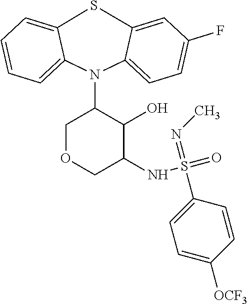 [SMILES: CN=S(=O)(NC1COCC(N2c3ccccc3Sc3cc(F)ccc32)C1O)c1ccc(OC(F)(F)F)cc1] |
| 283 | 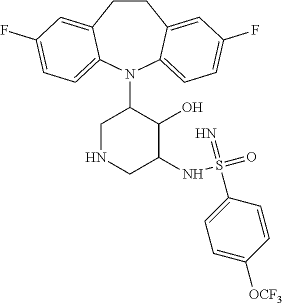 [SMILES: N=S(=O)(NC1CNCC(N2c3ccc(F)cc3CCc3cc(F)ccc32)C1O)c1ccc(OC(F)(F)F)cc1] |
| 284 | 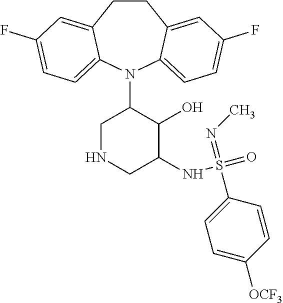 [SMILES: CN=S(=O)(NC1CNCC(N2c3ccc(F)cc3CCc3cc(F)ccc32)C1O)c1ccc(OC(F)(F)F)cc1] |
| 285 | 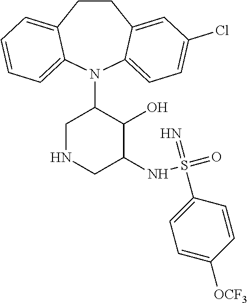 [SMILES: N=S(=O)(NC1CNCC(N2c3ccccc3CCc3cc(Cl)ccc32)C1O)c1ccc(OC(F)(F)F)cc1] |
| 286 | 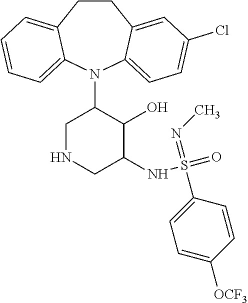 [SMILES: CN=S(=O)(NC1CNCC(N2c3ccccc3CCc3cc(Cl)ccc32)C1O)c1ccc(OC(F)(F)F)cc1] |
| 287 | 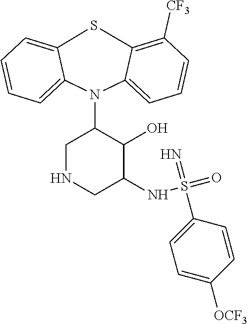 [SMILES: N=S(=O)(NC1CNCC(N2c3ccccc3Sc3c2cccc3C(F)(F)F)C1O)c1ccc(OC(F)(F)F)cc1] |
| 288 | 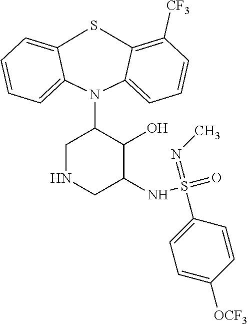 [SMILES: CN=S(=O)(NC1CNCC(N2c3ccccc3Sc3c2cccc3C(F)(F)F)C1O)c1ccc(OC(F)(F)F)cc1] |
| 289 | 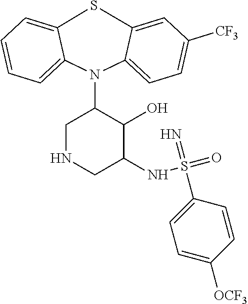 [SMILES: N=S(=O)(NC1CNCC(N2c3ccccc3Sc3cc(C(F)(F)F)ccc32)C1O)c1ccc(OC(F)(F)F)cc1] |
| 290 | 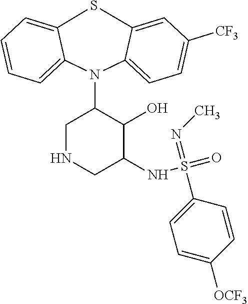 [SMILES: CN=S(=O)(NC1CNCC(N2c3ccccc3Sc3cc(C(F)(F)F)ccc32)C1O)c1ccc(OC(F)(F)F)cc1] |
| 291 | 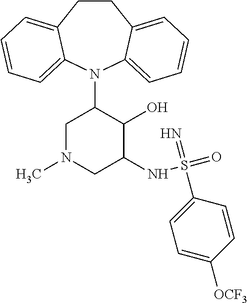 [SMILES: CN1CC(NS(=N)(=O)c2ccc(OC(F)(F)F)cc2)C(O)C(N2c3ccccc3CCc3ccccc32)C1] |
| 292 | 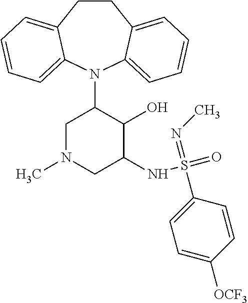 [SMILES: CN=S(=O)(NC1CN(C)CC(N2c3ccccc3CCc3ccccc32)C1O)c1ccc(OC(F)(F)F)cc1] |
| 293 | 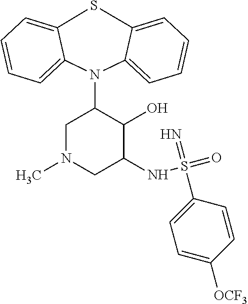 [SMILES: CN1CC(NS(=N)(=O)c2ccc(OC(F)(F)F)cc2)C(O)C(N2c3ccccc3Sc3ccccc32)C1] |
| 294 | 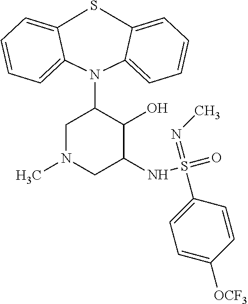 [SMILES: CN=S(=O)(NC1CN(C)CC(N2c3ccccc3Sc3ccccc32)C1O)c1ccc(OC(F)(F)F)cc1] |
| 295 | 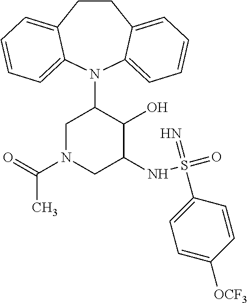 [SMILES: CC(=O)N1CC(NS(=N)(=O)c2ccc(OC(F)(F)F)cc2)C(O)C(N2c3ccccc3CCc3ccccc32)C1] |
| 296 | 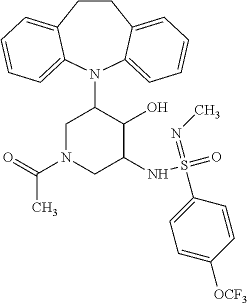 [SMILES: CN=S(=O)(NC1CN(C(C)=O)CC(N2c3ccccc3CCc3ccccc32)C1O)c1ccc(OC(F)(F)F)cc1] |
| 297 | 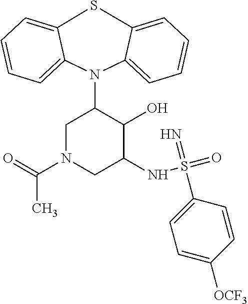 [SMILES: CC(=O)N1CC(NS(=N)(=O)c2ccc(OC(F)(F)F)cc2)C(O)C(N2c3ccccc3Sc3ccccc32)C1] |
| 298 | 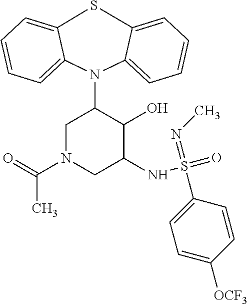 [SMILES: CN=S(=O)(NC1CN(C(C)=O)CC(N2c3ccccc3Sc3ccccc32)C1O)c1ccc(OC(F)(F)F)cc1] |
| 299 | 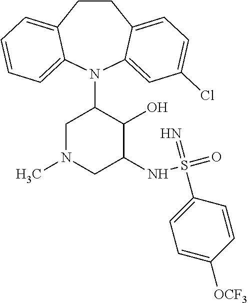 [SMILES: CN1CC(NS(=N)(=O)c2ccc(OC(F)(F)F)cc2)C(O)C(N2c3ccccc3CCc3ccc(Cl)cc32)C1] |
| 300 | 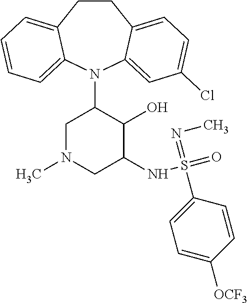 [SMILES: CN=S(=O)(NC1CN(C)CC(N2c3ccccc3CCc3ccc(Cl)cc32)C1O)c1ccc(OC(F)(F)F)cc1] |
| 301 | 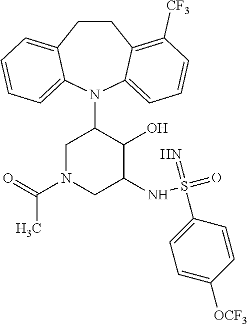 [SMILES: CC(=O)N1CC(NS(=N)(=O)c2ccc(OC(F)(F)F)cc2)C(O)C(N2c3ccccc3CCc3c2cccc3C(F)(F)F)C1] |
| 301 | 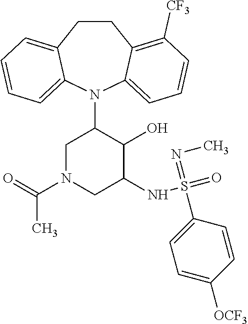 [SMILES: CN=S(=O)(NC1CN(C(C)=O)CC(N2c3ccccc3CCc3c2cccc3C(F)(F)F)C1O)c1ccc(OC(F)(F)F)cc1] |
| 303 | 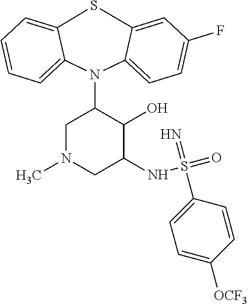 [SMILES: CN1CC(NS(=N)(=O)c2ccc(OC(F)(F)F)cc2)C(O)C(N2c3ccccc3Sc3cc(F)ccc32)C1] |
| 304 | 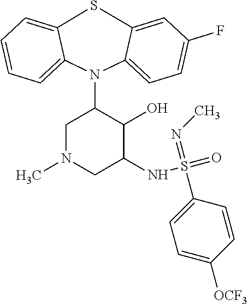 [SMILES: CN=S(=O)(NC1CN(C)CC(N2c3ccccc3Sc3cc(F)ccc32)C1O)c1ccc(OC(F)(F)F)cc1] |
| 305 | 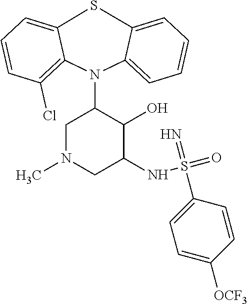 [SMILES: CN1CC(NS(=N)(=O)c2ccc(OC(F)(F)F)cc2)C(O)C(N2c3ccccc3Sc3cccc(Cl)c32)C1] |
| 306 | 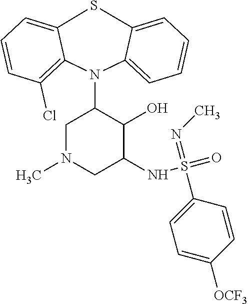 [SMILES: CN=S(=O)(NC1CN(C)CC(N2c3ccccc3Sc3cccc(Cl)c32)C1O)c1ccc(OC(F)(F)F)cc1] |
| 307 | 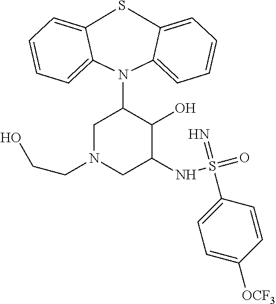 [SMILES: N=S(=O)(NC1CN(CCO)CC(N2c3ccccc3Sc3ccccc32)C1O)c1ccc(OC(F)(F)F)cc1] |
| 308 | 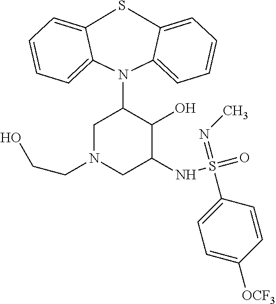 [SMILES: CN=S(=O)(NC1CN(CCO)CC(N2c3ccccc3Sc3ccccc32)C1O)c1ccc(OC(F)(F)F)cc1] |
| 309 | 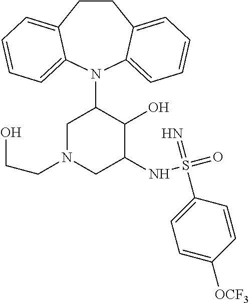 [SMILES: N=S(=O)(NC1CN(CCO)CC(N2c3ccccc3CCc3ccccc32)C1O)c1ccc(OC(F)(F)F)cc1] |
| 310 | 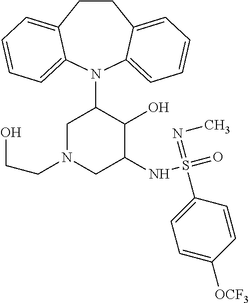 [SMILES: CN=S(=O)(NC1CN(CCO)CC(N2c3ccccc3CCc3ccccc32)C1O)c1ccc(OC(F)(F)F)cc1] |
| 311 | 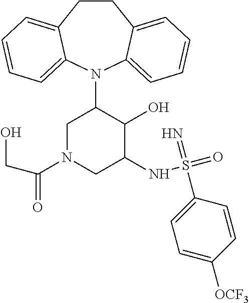 [SMILES: N=S(=O)(NC1CN(C(=O)CO)CC(N2c3ccccc3CCc3ccccc32)C1O)c1ccc(OC(F)(F)F)cc1] |
| 312 | 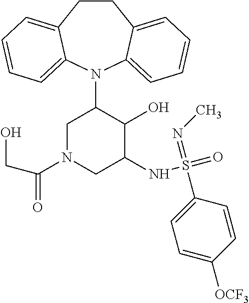 [SMILES: CN=S(=O)(NC1CN(C(=O)CO)CC(N2c3ccccc3CCc3ccccc32)C1O)c1ccc(OC(F)(F)F)cc1] |
| 313 | 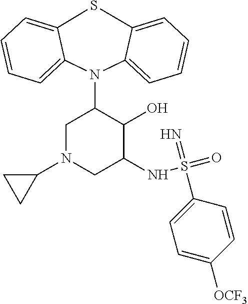 [SMILES: N=S(=O)(NC1CN(C2CC2)CC(N2c3ccccc3Sc3ccccc32)C1O)c1ccc(OC(F)(F)F)cc1] |
| 314 | 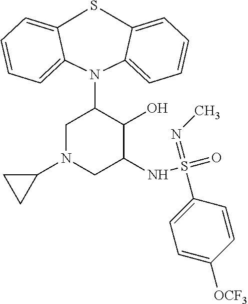 [SMILES: CN=S(=O)(NC1CN(C2CC2)CC(N2c3ccccc3Sc3ccccc32)C1O)c1ccc(OC(F)(F)F)cc1] |
| 315 | 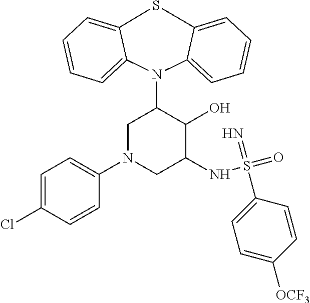 [SMILES: N=S(=O)(NC1CN(c2ccc(Cl)cc2)CC(N2c3ccccc3Sc3ccccc32)C1O)c1ccc(OC(F)(F)F)cc1] |
| 316 | 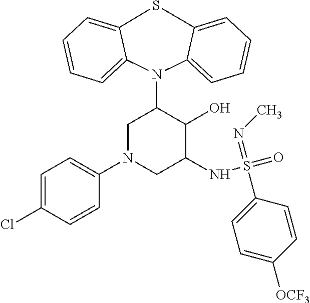 [SMILES: CN=S(=O)(NC1CN(c2ccc(Cl)cc2)CC(N2c3ccccc3Sc3ccccc32)C1O)c1ccc(OC(F)(F)F)cc1] |
| 317 | 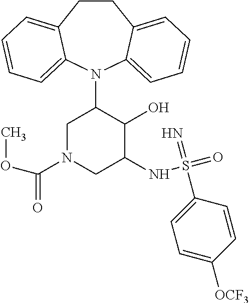 [SMILES: COC(=O)N1CC(NS(=N)(=O)c2ccc(OC(F)(F)F)cc2)C(O)C(N2c3ccccc3CCc3ccccc32)C1] |
| 318 | 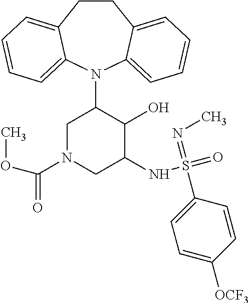 [SMILES: CN=S(=O)(NC1CN(C(=O)OC)CC(N2c3ccccc3CCc3ccccc32)C1O)c1ccc(OC(F)(F)F)cc1] |
| 319 | 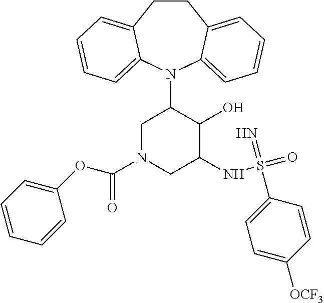 [SMILES: N=S(=O)(NC1CN(C(=O)Oc2ccccc2)CC(N2c3ccccc3CCc3ccccc32)C1O)c1ccc(OC(F)(F)F)cc1] |
| 320 | 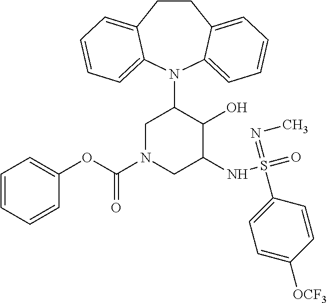 [SMILES: CN=S(=O)(NC1CN(C(=O)Oc2ccccc2)CC(N2c3ccccc3CCc3ccccc32)C1O)c1ccc(OC(F)(F)F)cc1] |
| 321 | 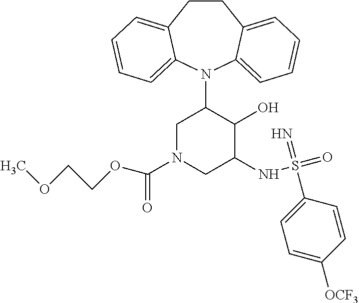 [SMILES: COCCOC(=O)N1CC(NS(=N)(=O)c2ccc(OC(F)(F)F)cc2)C(O)C(N2c3ccccc3CCc3ccccc32)C1] |
| 322 | 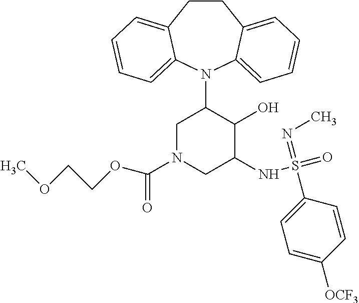 [SMILES: CN=S(=O)(NC1CN(C(=O)OCCOC)CC(N2c3ccccc3CCc3ccccc32)C1O)c1ccc(OC(F)(F)F)cc1] |
| 323 | 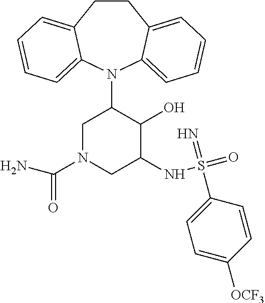 [SMILES: N=S(=O)(NC1CN(C(N)=O)CC(N2c3ccccc3CCc3ccccc32)C1O)c1ccc(OC(F)(F)F)cc1] |
| 324 | 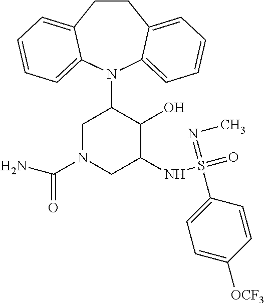 [SMILES: CN=S(=O)(NC1CN(C(N)=O)CC(N2c3ccccc3CCc3ccccc32)C1O)c1ccc(OC(F)(F)F)cc1] |
| 325 | 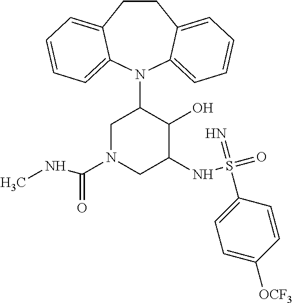 [SMILES: CNC(=O)N1CC(NS(=N)(=O)c2ccc(OC(F)(F)F)cc2)C(O)C(N2c3ccccc3CCc3ccccc32)C1] |
| 326 | 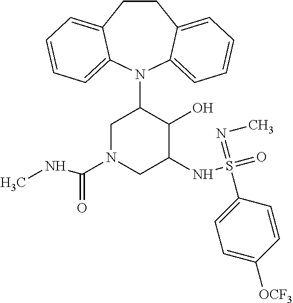 [SMILES: CN=S(=O)(NC1CN(C(=O)NC)CC(N2c3ccccc3CCc3ccccc32)C1O)c1ccc(OC(F)(F)F)cc1] |
| 327 | 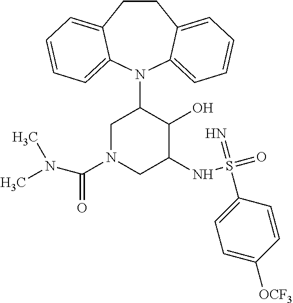 [SMILES: CN(C)C(=O)N1CC(NS(=N)(=O)c2ccc(OC(F)(F)F)cc2)C(O)C(N2c3ccccc3CCc3ccccc32)C1] |
| 328 | 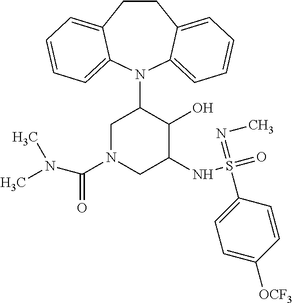 [SMILES: CN=S(=O)(NC1CN(C(=O)N(C)C)CC(N2c3ccccc3CCc3ccccc32)C1O)c1ccc(OC(F)(F)F)cc1] |
| 329 | 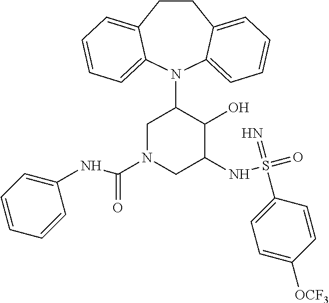 [SMILES: N=S(=O)(NC1CN(C(=O)Nc2ccccc2)CC(N2c3ccccc3CCc3ccccc32)C1O)c1ccc(OC(F)(F)F)cc1] |
| 330 | 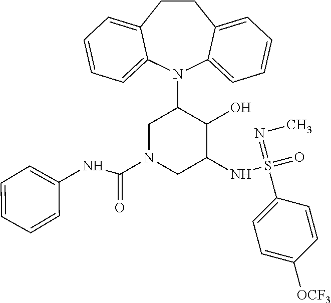 [SMILES: CN=S(=O)(NC1CN(C(=O)Nc2ccccc2)CC(N2c3ccccc3CCc3ccccc32)C1O)c1ccc(OC(F)(F)F)cc1] |
| 331 | 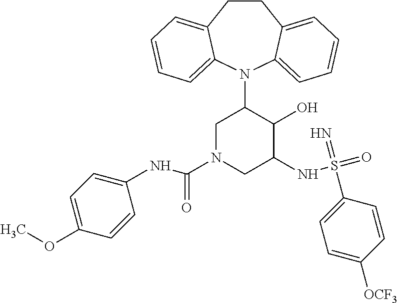 [SMILES: COc1ccc(NC(=O)N2CC(NS(=N)(=O)c3ccc(OC(F)(F)F)cc3)C(O)C(N3c4ccccc4CCc4ccccc43)C2)cc1] |
| 332 | 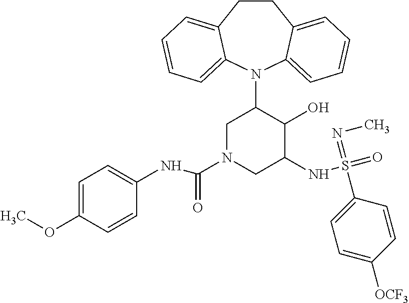 [SMILES: CN=S(=O)(NC1CN(C(=O)Nc2ccc(OC)cc2)CC(N2c3ccccc3CCc3ccccc32)C1O)c1ccc(OC(F)(F)F)cc1] |
| 333 | 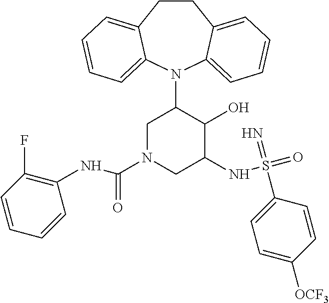 [SMILES: N=S(=O)(NC1CN(C(=O)Nc2ccccc2F)CC(N2c3ccccc3CCc3ccccc32)C1O)c1ccc(OC(F)(F)F)cc1] |
| 334 | 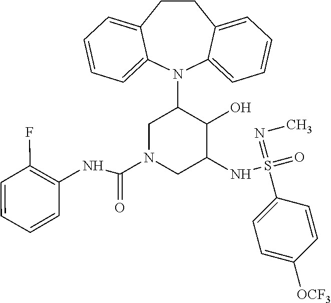 [SMILES: CN=S(=O)(NC1CN(C(=O)Nc2ccccc2F)CC(N2c3ccccc3CCc3ccccc32)C1O)c1ccc(OC(F)(F)F)cc1] |
| 335 | 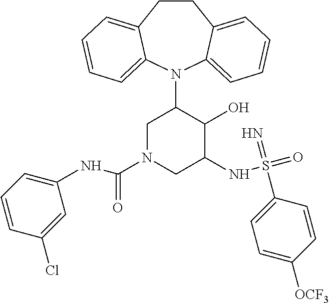 [SMILES: N=S(=O)(NC1CN(C(=O)Nc2cccc(Cl)c2)CC(N2c3ccccc3CCc3ccccc32)C1O)c1ccc(OC(F)(F)F)cc1] |
| 336 | 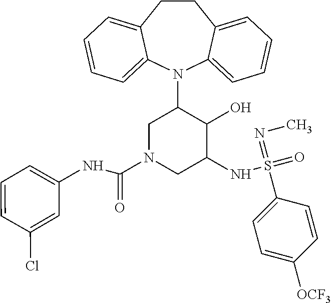 [SMILES: CN=S(=O)(NC1CN(C(=O)Nc2cccc(Cl)c2)CC(N2c3ccccc3CCc3ccccc32)C1O)c1ccc(OC(F)(F)F)cc1] |
| 337 | 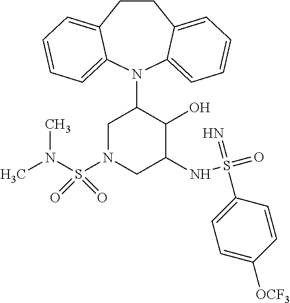 [SMILES: CN(C)S(=O)(=O)N1CC(NS(=N)(=O)c2ccc(OC(F)(F)F)cc2)C(O)C(N2c3ccccc3CCc3ccccc32)C1] |
| 338 | 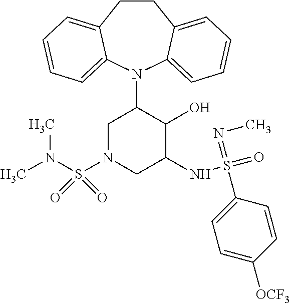 [SMILES: CN=S(=O)(NC1CN(S(=O)(=O)N(C)C)CC(N2c3ccccc3CCc3ccccc32)C1O)c1ccc(OC(F)(F)F)cc1] |
| 339 | 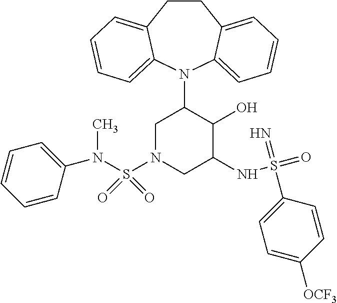 [SMILES: CN(c1ccccc1)S(=O)(=O)N1CC(NS(=N)(=O)c2ccc(OC(F)(F)F)cc2)C(O)C(N2c3ccccc3CCc3ccccc32)C1] |
| 340 | 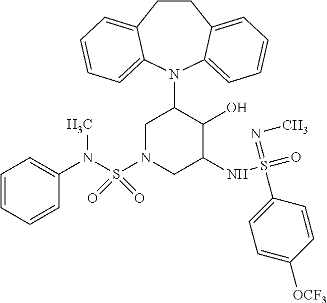 [SMILES: CN=S(=O)(NC1CN(S(=O)(=O)N(C)c2ccccc2)CC(N2c3ccccc3CCc3ccccc32)C1O)c1ccc(OC(F)(F)F)cc1] |
| 341 | 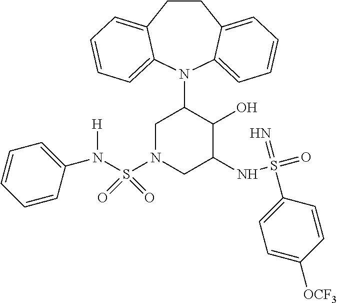 [SMILES: N=S(=O)(NC1CN(S(=O)(=O)Nc2ccccc2)CC(N2c3ccccc3CCc3ccccc32)C1O)c1ccc(OC(F)(F)F)cc1] |
| 342 | 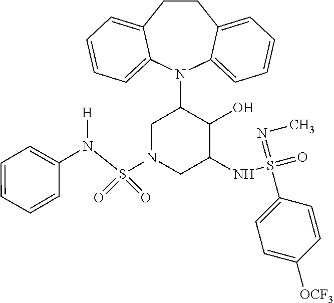 [SMILES: CN=S(=O)(NC1CN(S(=O)(=O)Nc2ccccc2)CC(N2c3ccccc3CCc3ccccc32)C1O)c1ccc(OC(F)(F)F)cc1] |
| 343 | 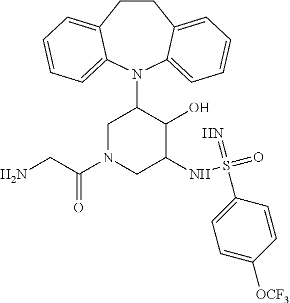 [SMILES: N=S(=O)(NC1CN(C(=O)CN)CC(N2c3ccccc3CCc3ccccc32)C1O)c1ccc(OC(F)(F)F)cc1] |
| 344 | 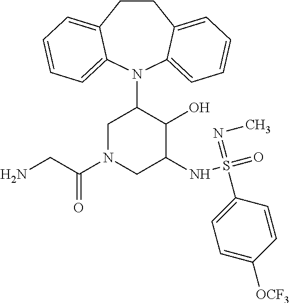 [SMILES: CN=S(=O)(NC1CN(C(=O)CN)CC(N2c3ccccc3CCc3ccccc32)C1O)c1ccc(OC(F)(F)F)cc1] |
| 345 | 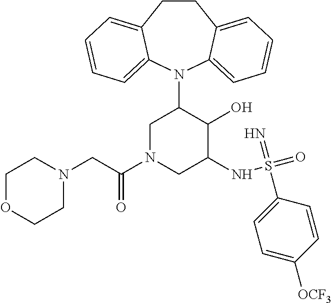 [SMILES: N=S(=O)(NC1CN(C(=O)CN2CCOCC2)CC(N2c3ccccc3CCc3ccccc32)C1O)c1ccc(OC(F)(F)F)cc1] |
| 346 | 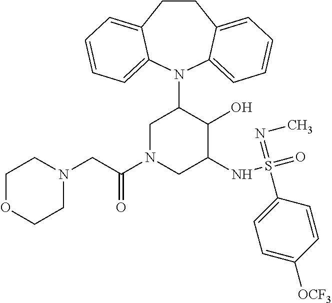 [SMILES: CN=S(=O)(NC1CN(C(=O)CN2CCOCC2)CC(N2c3ccccc3CCc3ccccc32)C1O)c1ccc(OC(F)(F)F)cc1] |
| 347 | 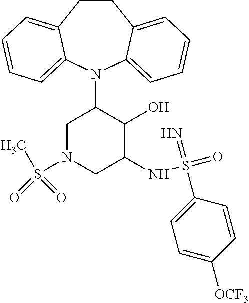 [SMILES: CS(=O)(=O)N1CC(NS(=N)(=O)c2ccc(OC(F)(F)F)cc2)C(O)C(N2c3ccccc3CCc3ccccc32)C1] |
| 348 | 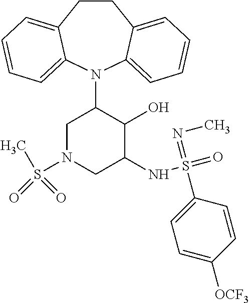 [SMILES: CN=S(=O)(NC1CN(S(C)(=O)=O)CC(N2c3ccccc3CCc3ccccc32)C1O)c1ccc(OC(F)(F)F)cc1] |
| 349 | 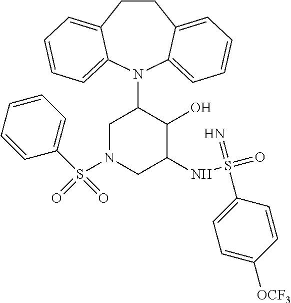 [SMILES: N=S(=O)(NC1CN(S(=O)(=O)c2ccccc2)CC(N2c3ccccc3CCc3ccccc32)C1O)c1ccc(OC(F)(F)F)cc1] |
| 350 | 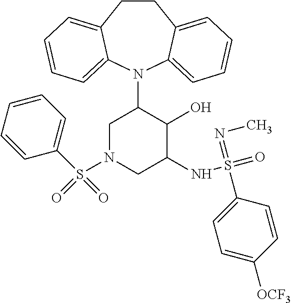 [SMILES: CN=S(=O)(NC1CN(S(=O)(=O)c2ccccc2)CC(N2c3ccccc3CCc3ccccc32)C1O)c1ccc(OC(F)(F)F)cc1] |
| 351 | 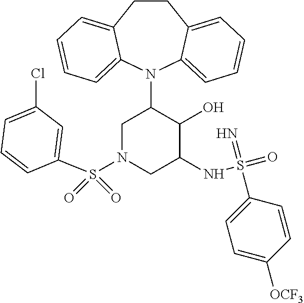 [SMILES: N=S(=O)(NC1CN(S(=O)(=O)c2cccc(Cl)c2)CC(N2c3ccccc3CCc3ccccc32)C1O)c1ccc(OC(F)(F)F)cc1] |
| 352 | 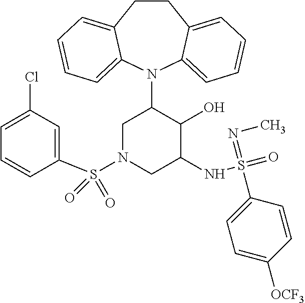 [SMILES: CN=S(=O)(NC1CN(S(=O)(=O)c2cccc(Cl)c2)CC(N2c3ccccc3CCc3ccccc32)C1O)c1ccc(OC(F)(F)F)cc1] |
| 353 | 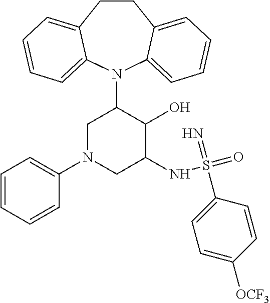 [SMILES: N=S(=O)(NC1CN(c2ccccc2)CC(N2c3ccccc3CCc3ccccc32)C1O)c1ccc(OC(F)(F)F)cc1] |
| 354 | 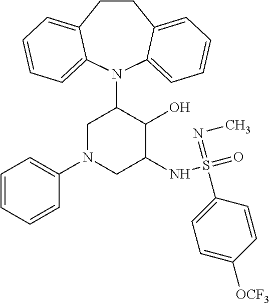 [SMILES: CN=S(=O)(NC1CN(c2ccccc2)CC(N2c3ccccc3CCc3ccccc32)C1O)c1ccc(OC(F)(F)F)cc1] |
| 355 | 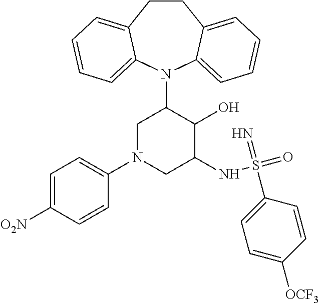 [SMILES: N=S(=O)(NC1CN(c2ccc([N+](=O)[O-])cc2)CC(N2c3ccccc3CCc3ccccc32)C1O)c1ccc(OC(F)(F)F)cc1] |
| 356 | 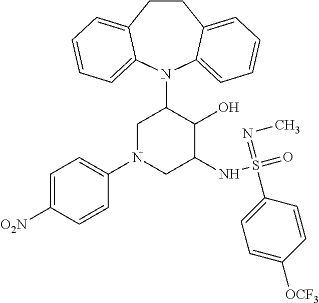 [SMILES: CN=S(=O)(NC1CN(c2ccc([N+](=O)[O-])cc2)CC(N2c3ccccc3CCc3ccccc32)C1O)c1ccc(OC(F)(F)F)cc1] |
| 357 | 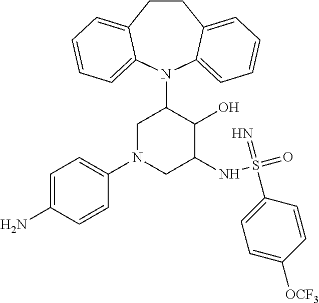 [SMILES: N=S(=O)(NC1CN(c2ccc(N)cc2)CC(N2c3ccccc3CCc3ccccc32)C1O)c1ccc(OC(F)(F)F)cc1] |
| 358 | 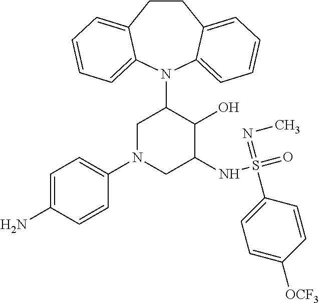 [SMILES: CN=S(=O)(NC1CN(c2ccc(N)cc2)CC(N2c3ccccc3CCc3ccccc32)C1O)c1ccc(OC(F)(F)F)cc1] |
| 359 | 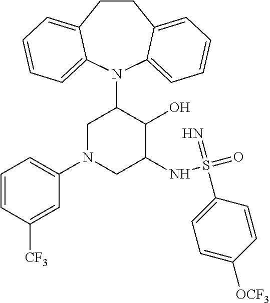 [SMILES: N=S(=O)(NC1CN(c2cccc(C(F)(F)F)c2)CC(N2c3ccccc3CCc3ccccc32)C1O)c1ccc(OC(F)(F)F)cc1] |
| 360 | 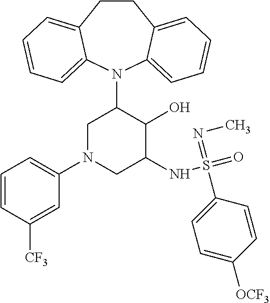 [SMILES: CN=S(=O)(NC1CN(c2cccc(C(F)(F)F)c2)CC(N2c3ccccc3CCc3ccccc32)C1O)c1ccc(OC(F)(F)F)cc1] |
| 361 | 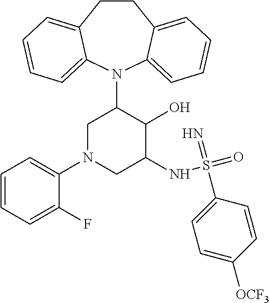 [SMILES: N=S(=O)(NC1CN(c2ccccc2F)CC(N2c3ccccc3CCc3ccccc32)C1O)c1ccc(OC(F)(F)F)cc1] |
| 362 | 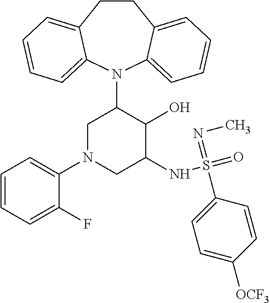 [SMILES: CN=S(=O)(NC1CN(c2ccccc2F)CC(N2c3ccccc3CCc3ccccc32)C1O)c1ccc(OC(F)(F)F)cc1] |
| 363 | 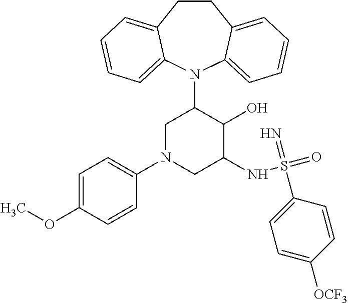 [SMILES: COc1ccc(N2CC(NS(=N)(=O)c3ccc(OC(F)(F)F)cc3)C(O)C(N3c4ccccc4CCc4ccccc43)C2)cc1] |
| 364 | 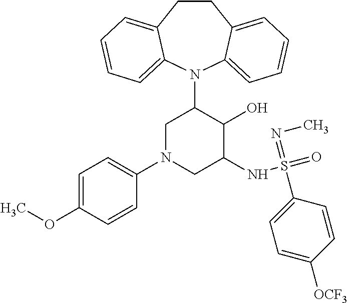 [SMILES: CN=S(=O)(NC1CN(c2ccc(OC)cc2)CC(N2c3ccccc3CCc3ccccc32)C1O)c1ccc(OC(F)(F)F)cc1] |
| 365 | 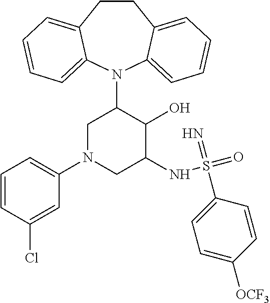 [SMILES: N=S(=O)(NC1CN(c2cccc(Cl)c2)CC(N2c3ccccc3CCc3ccccc32)C1O)c1ccc(OC(F)(F)F)cc1] |
| 366 | 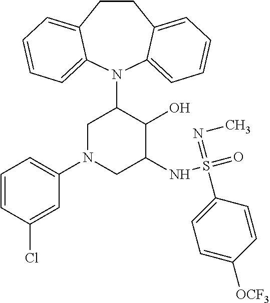 [SMILES: CN=S(=O)(NC1CN(c2cccc(Cl)c2)CC(N2c3ccccc3CCc3ccccc32)C1O)c1ccc(OC(F)(F)F)cc1] |
| 367 | 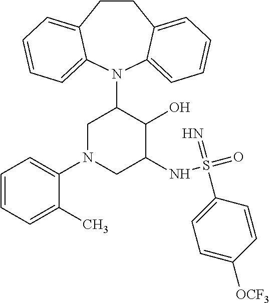 [SMILES: Cc1ccccc1N1CC(NS(=N)(=O)c2ccc(OC(F)(F)F)cc2)C(O)C(N2c3ccccc3CCc3ccccc32)C1] |
| 368 | 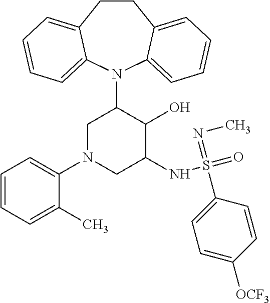 [SMILES: CN=S(=O)(NC1CN(c2ccccc2C)CC(N2c3ccccc3CCc3ccccc32)C1O)c1ccc(OC(F)(F)F)cc1] |
| 369 | 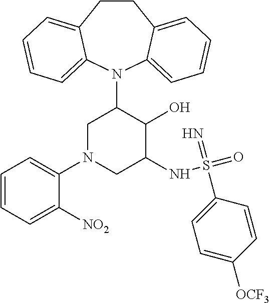 [SMILES: N=S(=O)(NC1CN(c2ccccc2[N+](=O)[O-])CC(N2c3ccccc3CCc3ccccc32)C1O)c1ccc(OC(F)(F)F)cc1] |
| 370 | 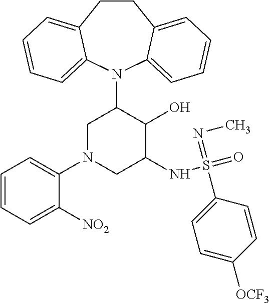 [SMILES: CN=S(=O)(NC1CN(c2ccccc2[N+](=O)[O-])CC(N2c3ccccc3CCc3ccccc32)C1O)c1ccc(OC(F)(F)F)cc1] |
| 371 | 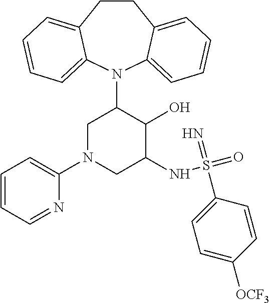 [SMILES: N=S(=O)(NC1CN(c2ccccn2)CC(N2c3ccccc3CCc3ccccc32)C1O)c1ccc(OC(F)(F)F)cc1] |
| 372 | 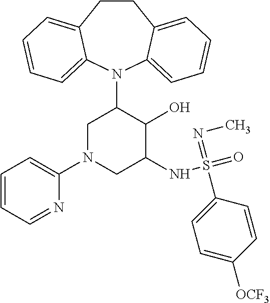 [SMILES: CN=S(=O)(NC1CN(c2ccccn2)CC(N2c3ccccc3CCc3ccccc32)C1O)c1ccc(OC(F)(F)F)cc1] |
| 373 | 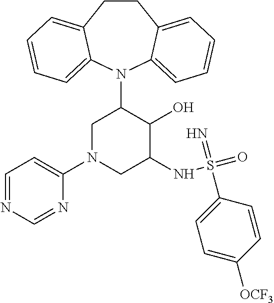 [SMILES: N=S(=O)(NC1CN(c2ccncn2)CC(N2c3ccccc3CCc3ccccc32)C1O)c1ccc(OC(F)(F)F)cc1] |
| 374 | 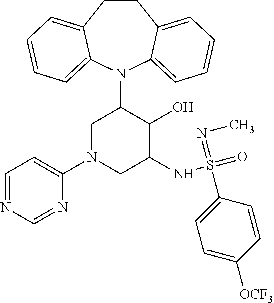 [SMILES: CN=S(=O)(NC1CN(c2ccncn2)CC(N2c3ccccc3CCc3ccccc32)C1O)c1ccc(OC(F)(F)F)cc1] |
| 375 | 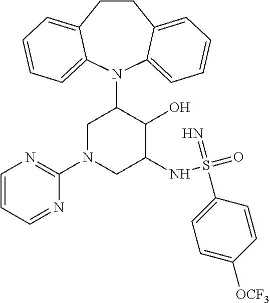 [SMILES: N=S(=O)(NC1CN(c2ncccn2)CC(N2c3ccccc3CCc3ccccc32)C1O)c1ccc(OC(F)(F)F)cc1] |
| 376 | 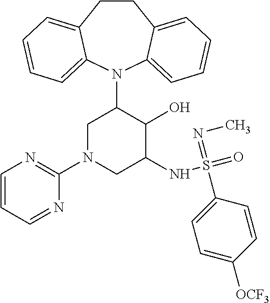 [SMILES: CN=S(=O)(NC1CN(c2ncccn2)CC(N2c3ccccc3CCc3ccccc32)C1O)c1ccc(OC(F)(F)F)cc1] |
| 377 | 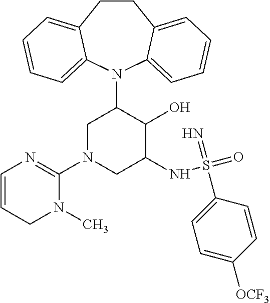 [SMILES: CN1CC=CN=C1N1CC(NS(=N)(=O)c2ccc(OC(F)(F)F)cc2)C(O)C(N2c3ccccc3CCc3ccccc32)C1] |
| 378 | 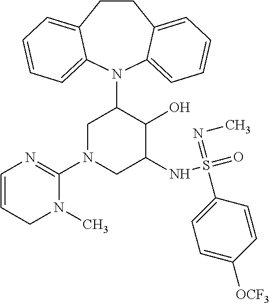 [SMILES: CN=S(=O)(NC1CN(C2=NC=CCN2C)CC(N2c3ccccc3CCc3ccccc32)C1O)c1ccc(OC(F)(F)F)cc1] |
| 379 | 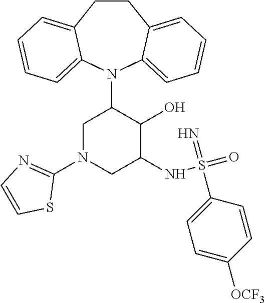 [SMILES: N=S(=O)(NC1CN(c2nccs2)CC(N2c3ccccc3CCc3ccccc32)C1O)c1ccc(OC(F)(F)F)cc1] |
| 380 | 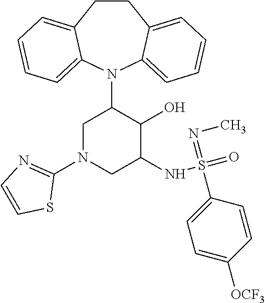 [SMILES: CN=S(=O)(NC1CN(c2nccs2)CC(N2c3ccccc3CCc3ccccc32)C1O)c1ccc(OC(F)(F)F)cc1] |
| 381 | 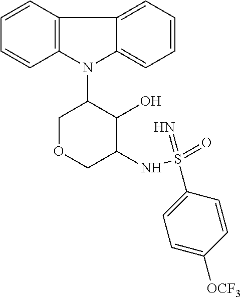 [SMILES: N=S(=O)(NC1COCC(n2c3ccccc3c3ccccc32)C1O)c1ccc(OC(F)(F)F)cc1] |
| 382 | 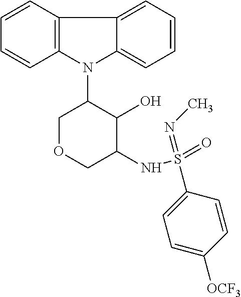 [SMILES: CN=S(=O)(NC1COCC(n2c3ccccc3c3ccccc32)C1O)c1ccc(OC(F)(F)F)cc1] |
| 383 | 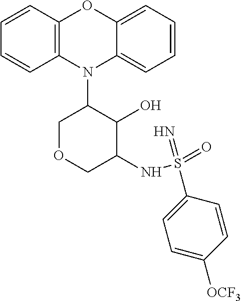 [SMILES: N=S(=O)(NC1COCC(N2c3ccccc3Oc3ccccc32)C1O)c1ccc(OC(F)(F)F)cc1] |
| 384 | 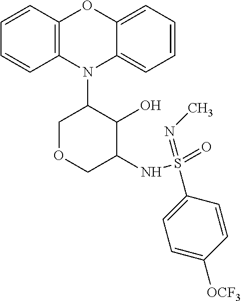 [SMILES: CN=S(=O)(NC1COCC(N2c3ccccc3Oc3ccccc32)C1O)c1ccc(OC(F)(F)F)cc1] |
| 385 | 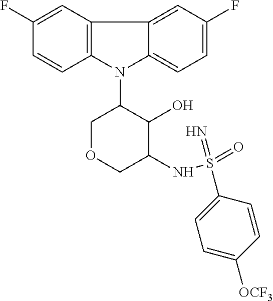 [SMILES: N=S(=O)(NC1COCC(n2c3ccc(F)cc3c3cc(F)ccc32)C1O)c1ccc(OC(F)(F)F)cc1] |
| 386 | 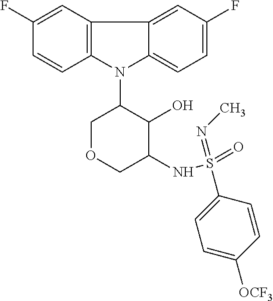 [SMILES: CN=S(=O)(NC1COCC(n2c3ccc(F)cc3c3cc(F)ccc32)C1O)c1ccc(OC(F)(F)F)cc1] |
| 387 | 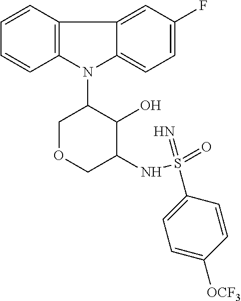 [SMILES: N=S(=O)(NC1COCC(n2c3ccccc3c3cc(F)ccc32)C1O)c1ccc(OC(F)(F)F)cc1] |
| 388 | 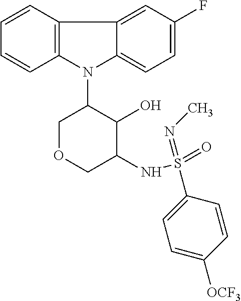 [SMILES: CN=S(=O)(NC1COCC(n2c3ccccc3c3cc(F)ccc32)C1O)c1ccc(OC(F)(F)F)cc1] |
| 389 | 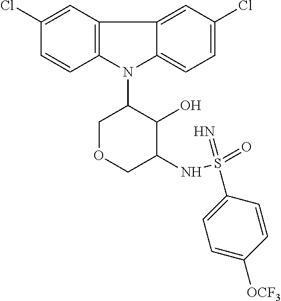 [SMILES: N=S(=O)(NC1COCC(n2c3ccc(Cl)cc3c3cc(Cl)ccc32)C1O)c1ccc(OC(F)(F)F)cc1] |
| 390 | 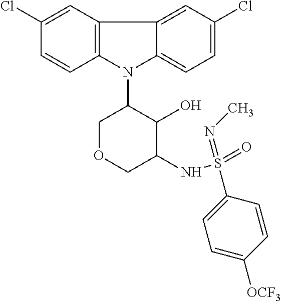 [SMILES: CN=S(=O)(NC1COCC(n2c3ccc(Cl)cc3c3cc(Cl)ccc32)C1O)c1ccc(OC(F)(F)F)cc1] |
| 391 | 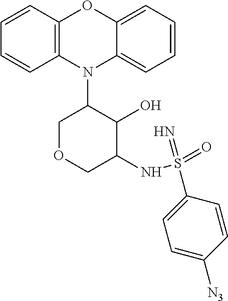 [SMILES: [N-]=[N+]=Nc1ccc(S(=N)(=O)NC2COCC(N3c4ccccc4Oc4ccccc43)C2O)cc1] |
| 392 | 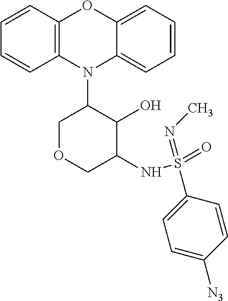 [SMILES: CN=S(=O)(NC1COCC(N2c3ccccc3Oc3ccccc32)C1O)c1ccc(N=[N+]=[N-])cc1] |
| 393 | 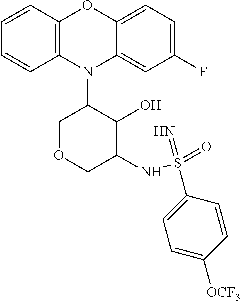 [SMILES: N=S(=O)(NC1COCC(N2c3ccccc3Oc3ccc(F)cc32)C1O)c1ccc(OC(F)(F)F)cc1] |
| 394 | 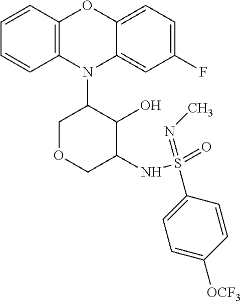 [SMILES: CN=S(=O)(NC1COCC(N2c3ccccc3Oc3ccc(F)cc32)C1O)c1ccc(OC(F)(F)F)cc1] |
| 395 | 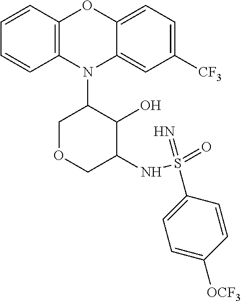 [SMILES: N=S(=O)(NC1COCC(N2c3ccccc3Oc3ccc(C(F)(F)F)cc32)C1O)c1ccc(OC(F)(F)F)cc1] |
| 396 | 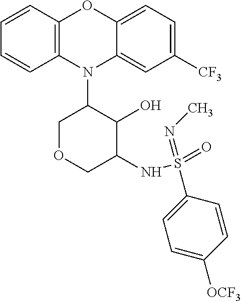 [SMILES: CN=S(=O)(NC1COCC(N2c3ccccc3Oc3ccc(C(F)(F)F)cc32)C1O)c1ccc(OC(F)(F)F)cc1] |
| 397 | 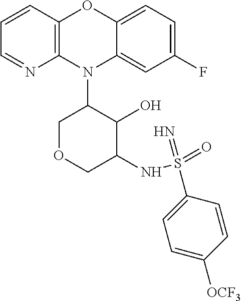 [SMILES: N=S(=O)(NC1COCC(N2c3cc(F)ccc3Oc3cccnc32)C1O)c1ccc(OC(F)(F)F)cc1] |
| 398 | 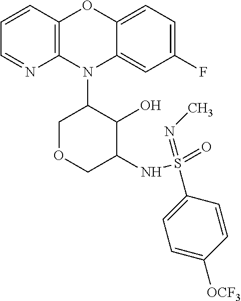 [SMILES: CN=S(=O)(NC1COCC(N2c3cc(F)ccc3Oc3cccnc32)C1O)c1ccc(OC(F)(F)F)cc1] |
| 399 | 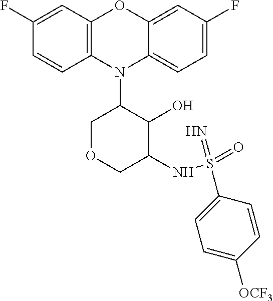 [SMILES: N=S(=O)(NC1COCC(N2c3ccc(F)cc3Oc3cc(F)ccc32)C1O)c1ccc(OC(F)(F)F)cc1] |
| 400 | 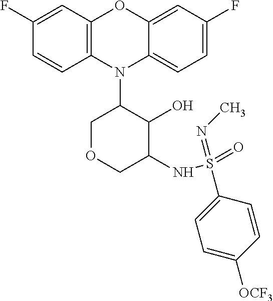 [SMILES: CN=S(=O)(NC1COCC(N2c3ccc(F)cc3Oc3cc(F)ccc32)C1O)c1ccc(OC(F)(F)F)cc1] |
| 401 | 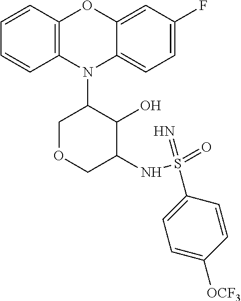 [SMILES: N=S(=O)(NC1COCC(N2c3ccccc3Oc3cc(F)ccc32)C1O)c1ccc(OC(F)(F)F)cc1] |
| 402 | 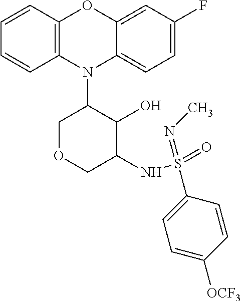 [SMILES: CN=S(=O)(NC1COCC(N2c3ccccc3Oc3cc(F)ccc32)C1O)c1ccc(OC(F)(F)F)cc1] |
| 403 | 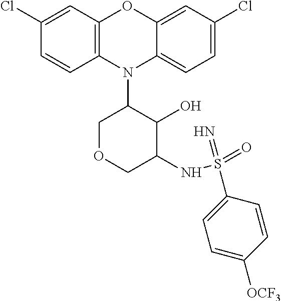 [SMILES: N=S(=O)(NC1COCC(N2c3ccc(Cl)cc3Oc3cc(Cl)ccc32)C1O)c1ccc(OC(F)(F)F)cc1] |
| 404 | 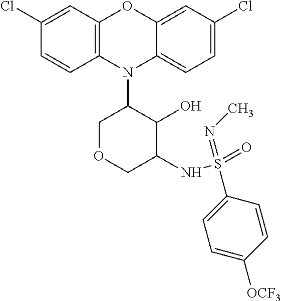 [SMILES: CN=S(=O)(NC1COCC(N2c3ccc(Cl)cc3Oc3cc(Cl)ccc32)C1O)c1ccc(OC(F)(F)F)cc1] |
| 405 | 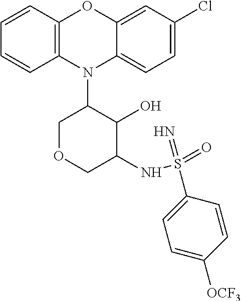 [SMILES: N=S(=O)(NC1COCC(N2c3ccccc3Oc3cc(Cl)ccc32)C1O)c1ccc(OC(F)(F)F)cc1] |
| 406 | 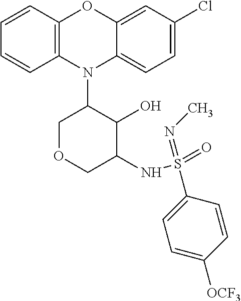 [SMILES: CN=S(=O)(NC1COCC(N2c3ccccc3Oc3cc(Cl)ccc32)C1O)c1ccc(OC(F)(F)F)cc1] |
| 407 | 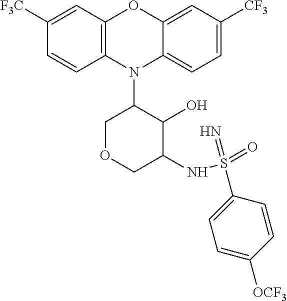 [SMILES: N=S(=O)(NC1COCC(N2c3ccc(C(F)(F)F)cc3Oc3cc(C(F)(F)F)ccc32)C1O)c1ccc(OC(F)(F)F)cc1] |
| 408 | 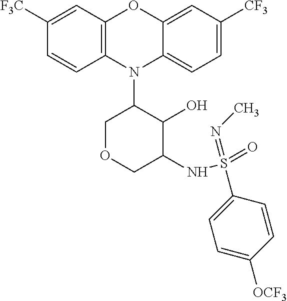 [SMILES: CN=S(=O)(NC1COCC(N2c3ccc(C(F)(F)F)cc3Oc3cc(C(F)(F)F)ccc32)C1O)c1ccc(OC(F)(F)F)cc1] |
| 409 | 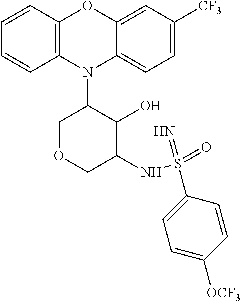 [SMILES: N=S(=O)(NC1COCC(N2c3ccccc3Oc3cc(C(F)(F)F)ccc32)C1O)c1ccc(OC(F)(F)F)cc1] |
| 410 | 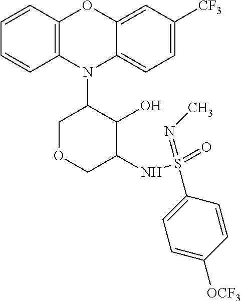 [SMILES: CN=S(=O)(NC1COCC(N2c3ccccc3Oc3cc(C(F)(F)F)ccc32)C1O)c1ccc(OC(F)(F)F)cc1] |
| 411 | 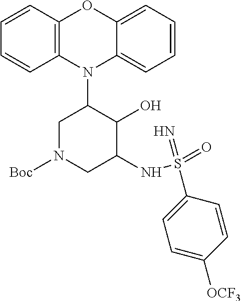 [SMILES: CC(C)(C)OC(=O)N1CC(NS(=N)(=O)c2ccc(OC(F)(F)F)cc2)C(O)C(N2c3ccccc3Oc3ccccc32)C1] |
| 412 | 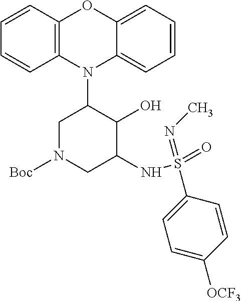 [SMILES: CN=S(=O)(NC1CN(C(=O)OC(C)(C)C)CC(N2c3ccccc3Oc3ccccc32)C1O)c1ccc(OC(F)(F)F)cc1] |
| 413 | 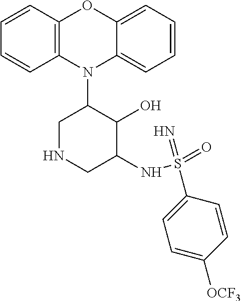 [SMILES: N=S(=O)(NC1CNCC(N2c3ccccc3Oc3ccccc32)C1O)c1ccc(OC(F)(F)F)cc1] |
| 414 | 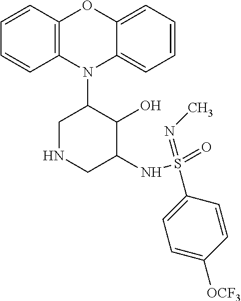 [SMILES: CN=S(=O)(NC1CNCC(N2c3ccccc3Oc3ccccc32)C1O)c1ccc(OC(F)(F)F)cc1] |
| 415 | 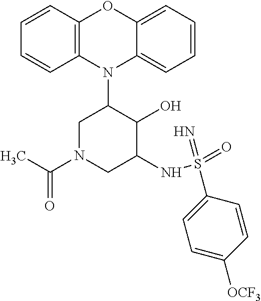 [SMILES: CC(=O)N1CC(NS(=N)(=O)c2ccc(OC(F)(F)F)cc2)C(O)C(N2c3ccccc3Oc3ccccc32)C1] |
| 416 | 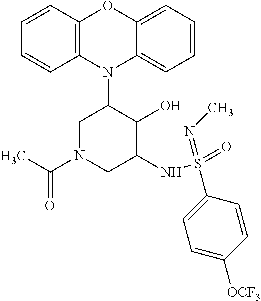 [SMILES: CN=S(=O)(NC1CN(C(C)=O)CC(N2c3ccccc3Oc3ccccc32)C1O)c1ccc(OC(F)(F)F)cc1] |
| 417 | 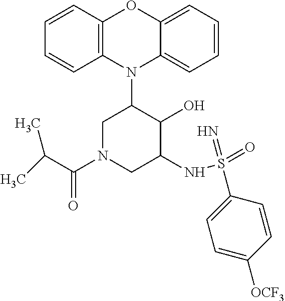 [SMILES: CC(C)C(=O)N1CC(NS(=N)(=O)c2ccc(OC(F)(F)F)cc2)C(O)C(N2c3ccccc3Oc3ccccc32)C1] |
| 418 | 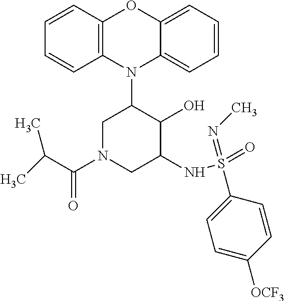 [SMILES: CN=S(=O)(NC1CN(C(=O)C(C)C)CC(N2c3ccccc3Oc3ccccc32)C1O)c1ccc(OC(F)(F)F)cc1] |
| 419 | 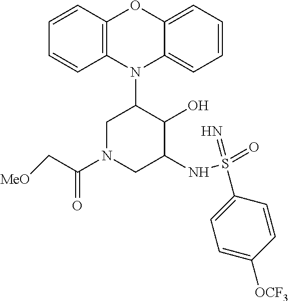 [SMILES: COCC(=O)N1CC(NS(=N)(=O)c2ccc(OC(F)(F)F)cc2)C(O)C(N2c3ccccc3Oc3ccccc32)C1] |
| 420 | 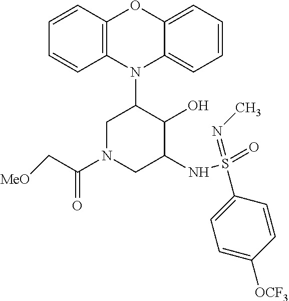 [SMILES: CN=S(=O)(NC1CN(C(=O)COC)CC(N2c3ccccc3Oc3ccccc32)C1O)c1ccc(OC(F)(F)F)cc1] |
| 421 | 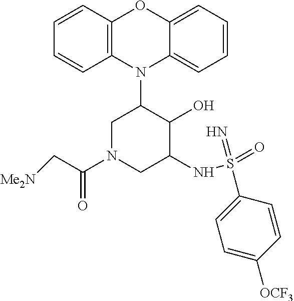 [SMILES: CN(C)CC(=O)N1CC(NS(=N)(=O)c2ccc(OC(F)(F)F)cc2)C(O)C(N2c3ccccc3Oc3ccccc32)C1] |
| 422 | 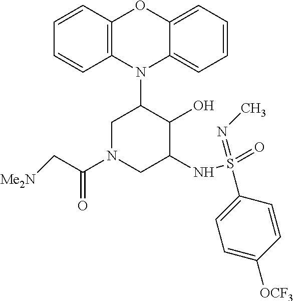 [SMILES: CN=S(=O)(NC1CN(C(=O)CN(C)C)CC(N2c3ccccc3Oc3ccccc32)C1O)c1ccc(OC(F)(F)F)cc1] |
| 423 | 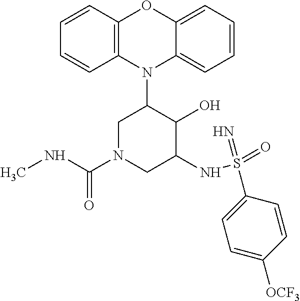 [SMILES: CNC(=O)N1CC(NS(=N)(=O)c2ccc(OC(F)(F)F)cc2)C(O)C(N2c3ccccc3Oc3ccccc32)C1] |
| 424 | 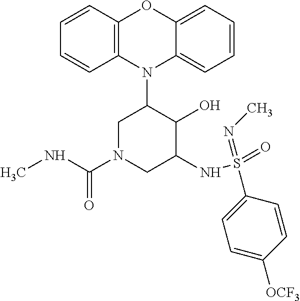 [SMILES: CN=S(=O)(NC1CN(C(=O)NC)CC(N2c3ccccc3Oc3ccccc32)C1O)c1ccc(OC(F)(F)F)cc1] |
| 425 | 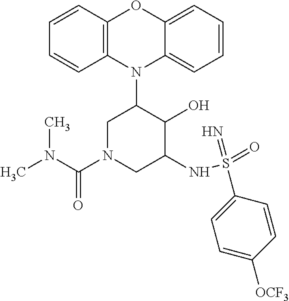 [SMILES: CN(C)C(=O)N1CC(NS(=N)(=O)c2ccc(OC(F)(F)F)cc2)C(O)C(N2c3ccccc3Oc3ccccc32)C1] |
| 426 | 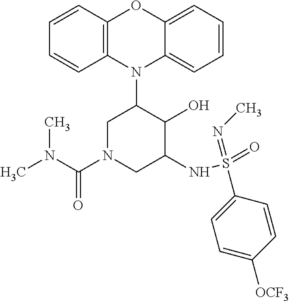 [SMILES: CN=S(=O)(NC1CN(C(=O)N(C)C)CC(N2c3ccccc3Oc3ccccc32)C1O)c1ccc(OC(F)(F)F)cc1] |
| 427 | 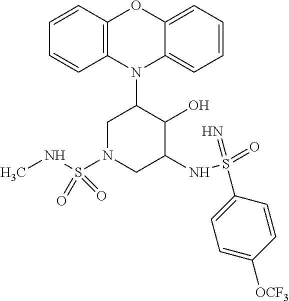 [SMILES: CNS(=O)(=O)N1CC(NS(=N)(=O)c2ccc(OC(F)(F)F)cc2)C(O)C(N2c3ccccc3Oc3ccccc32)C1] |
| 428 | 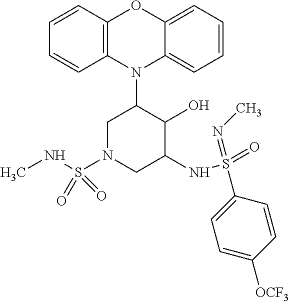 [SMILES: CN=S(=O)(NC1CN(S(=O)(=O)NC)CC(N2c3ccccc3Oc3ccccc32)C1O)c1ccc(OC(F)(F)F)cc1] |
| 429 | 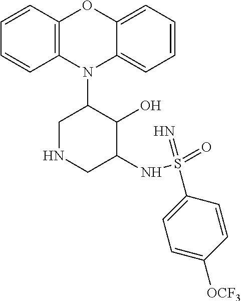 [SMILES: N=S(=O)(NC1CNCC(N2c3ccccc3Oc3ccccc32)C1O)c1ccc(OC(F)(F)F)cc1] |
| 430 | 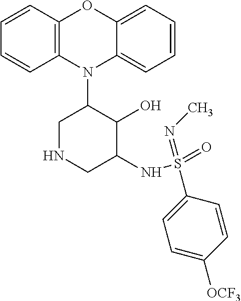 [SMILES: CN=S(=O)(NC1CNCC(N2c3ccccc3Oc3ccccc32)C1O)c1ccc(OC(F)(F)F)cc1] |
| 431 | 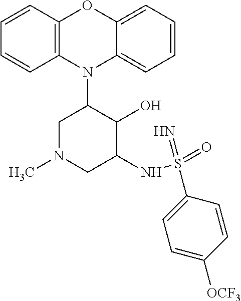 [SMILES: CN1CC(NS(=N)(=O)c2ccc(OC(F)(F)F)cc2)C(O)C(N2c3ccccc3Oc3ccccc32)C1] |
| 432 | 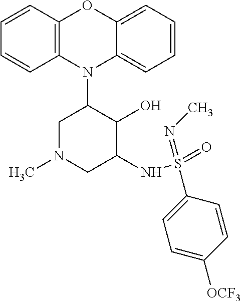 [SMILES: CN=S(=O)(NC1CN(C)CC(N2c3ccccc3Oc3ccccc32)C1O)c1ccc(OC(F)(F)F)cc1] |
| 433 | 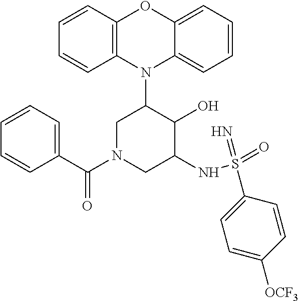 [SMILES: N=S(=O)(NC1CN(C(=O)c2ccccc2)CC(N2c3ccccc3Oc3ccccc32)C1O)c1ccc(OC(F)(F)F)cc1] |
| 434 | 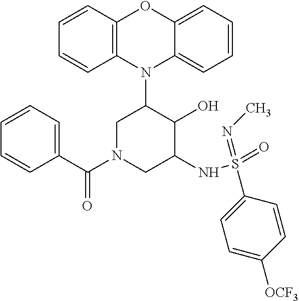 [SMILES: CN=S(=O)(NC1CN(C(=O)c2ccccc2)CC(N2c3ccccc3Oc3ccccc32)C1O)c1ccc(OC(F)(F)F)cc1] |
| 435 | 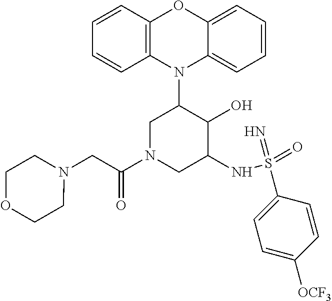 [SMILES: N=S(=O)(NC1CN(C(=O)CN2CCOCC2)CC(N2c3ccccc3Oc3ccccc32)C1O)c1ccc(OC(F)(F)F)cc1] |
| 436 | 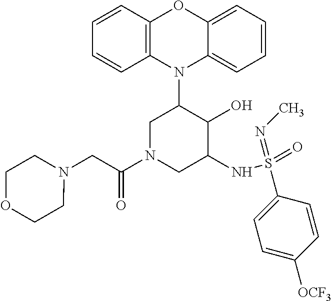 [SMILES: CN=S(=O)(NC1CN(C(=O)CN2CCOCC2)CC(N2c3ccccc3Oc3ccccc32)C1O)c1ccc(OC(F)(F)F)cc1] |
| 437 | 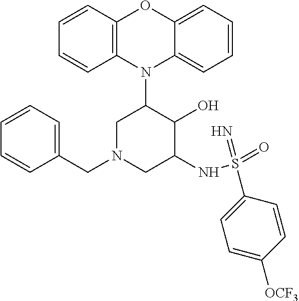 [SMILES: N=S(=O)(NC1CN(Cc2ccccc2)CC(N2c3ccccc3Oc3ccccc32)C1O)c1ccc(OC(F)(F)F)cc1] |
| 438 | 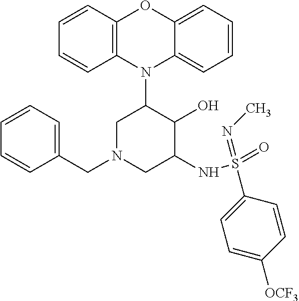 [SMILES: CN=S(=O)(NC1CN(Cc2ccccc2)CC(N2c3ccccc3Oc3ccccc32)C1O)c1ccc(OC(F)(F)F)cc1] |
| 439 | 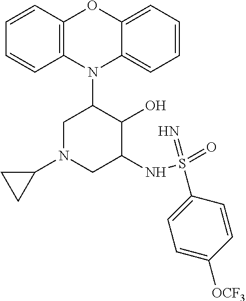 [SMILES: N=S(=O)(NC1CN(C2CC2)CC(N2c3ccccc3Oc3ccccc32)C1O)c1ccc(OC(F)(F)F)cc1] |
| 440 | 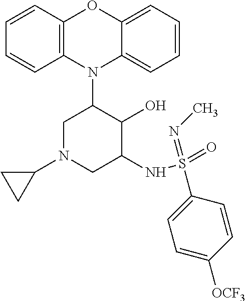 [SMILES: CN=S(=O)(NC1CN(C2CC2)CC(N2c3ccccc3Oc3ccccc32)C1O)c1ccc(OC(F)(F)F)cc1] |
| 441 | 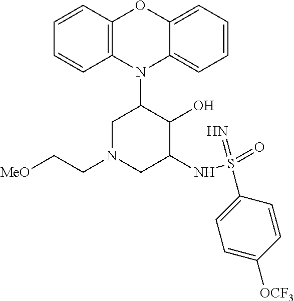 [SMILES: COCCN1CC(NS(=N)(=O)c2ccc(OC(F)(F)F)cc2)C(O)C(N2c3ccccc3Oc3ccccc32)C1] |
| 442 | 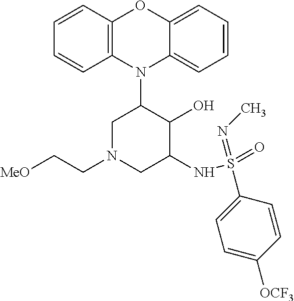 [SMILES: CN=S(=O)(NC1CN(CCOC)CC(N2c3ccccc3Oc3ccccc32)C1O)c1ccc(OC(F)(F)F)cc1] |
| 443 | 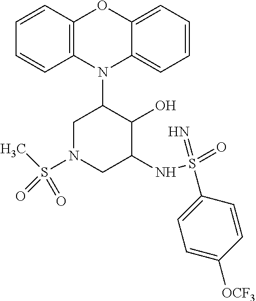 [SMILES: CS(=O)(=O)N1CC(NS(=N)(=O)c2ccc(OC(F)(F)F)cc2)C(O)C(N2c3ccccc3Oc3ccccc32)C1] |
| 444 | 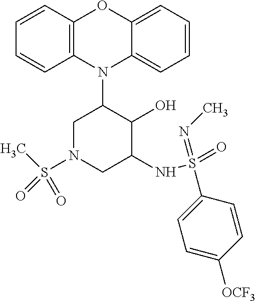 [SMILES: CN=S(=O)(NC1CN(S(C)(=O)=O)CC(N2c3ccccc3Oc3ccccc32)C1O)c1ccc(OC(F)(F)F)cc1] |
| 445 | 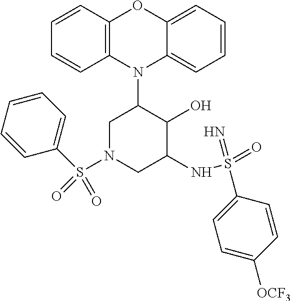 [SMILES: N=S(=O)(NC1CN(S(=O)(=O)c2ccccc2)CC(N2c3ccccc3Oc3ccccc32)C1O)c1ccc(OC(F)(F)F)cc1] |
| 446 | 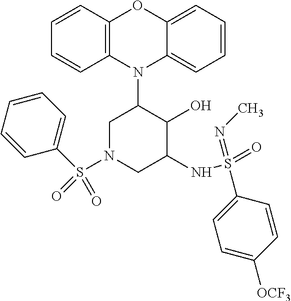 [SMILES: CN=S(=O)(NC1CN(S(=O)(=O)c2ccccc2)CC(N2c3ccccc3Oc3ccccc32)C1O)c1ccc(OC(F)(F)F)cc1] |
| 447 | 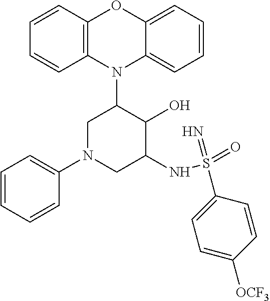 [SMILES: N=S(=O)(NC1CN(c2ccccc2)CC(N2c3ccccc3Oc3ccccc32)C1O)c1ccc(OC(F)(F)F)cc1] |
| 448 | 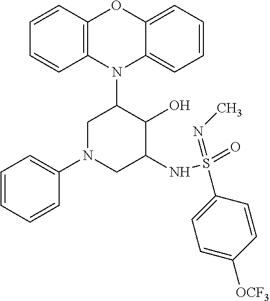 [SMILES: CN=S(=O)(NC1CN(c2ccccc2)CC(N2c3ccccc3Oc3ccccc32)C1O)c1ccc(OC(F)(F)F)cc1] |
| 449 | 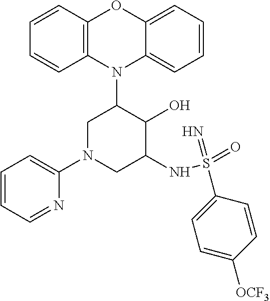 [SMILES: N=S(=O)(NC1CN(c2ccccn2)CC(N2c3ccccc3Oc3ccccc32)C1O)c1ccc(OC(F)(F)F)cc1] |
| 450 | 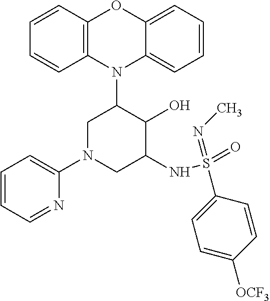 [SMILES: CN=S(=O)(NC1CN(c2ccccn2)CC(N2c3ccccc3Oc3ccccc32)C1O)c1ccc(OC(F)(F)F)cc1] |
| 451 | 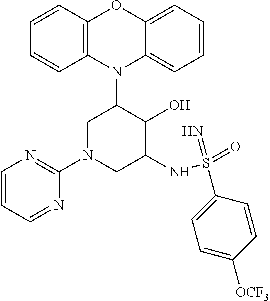 [SMILES: N=S(=O)(NC1CN(c2ncccn2)CC(N2c3ccccc3Oc3ccccc32)C1O)c1ccc(OC(F)(F)F)cc1] |
| 452 | 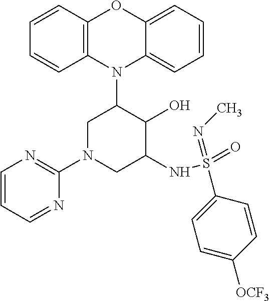 [SMILES: CN=S(=O)(NC1CN(c2ncccn2)CC(N2c3ccccc3Oc3ccccc32)C1O)c1ccc(OC(F)(F)F)cc1] |
| 453 | 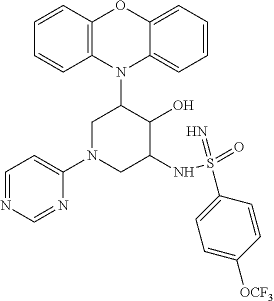 [SMILES: N=S(=O)(NC1CN(c2ccncn2)CC(N2c3ccccc3Oc3ccccc32)C1O)c1ccc(OC(F)(F)F)cc1] |
| 454 | 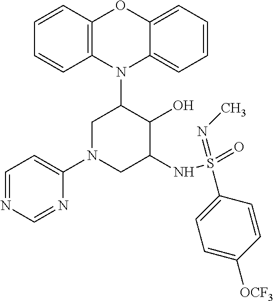 [SMILES: CN=S(=O)(NC1CN(c2ccncn2)CC(N2c3ccccc3Oc3ccccc32)C1O)c1ccc(OC(F)(F)F)cc1] |
| 455 | 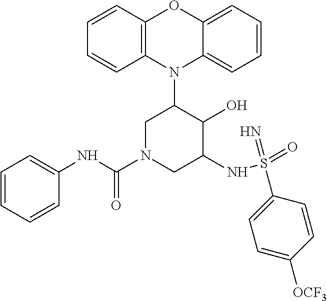 [SMILES: N=S(=O)(NC1CN(C(=O)Nc2ccccc2)CC(N2c3ccccc3Oc3ccccc32)C1O)c1ccc(OC(F)(F)F)cc1] |
| 456 | 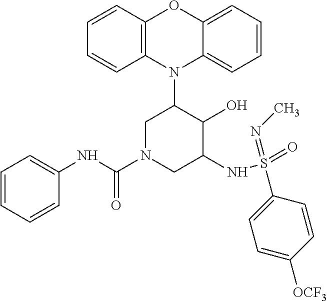 [SMILES: CN=S(=O)(NC1CN(C(=O)Nc2ccccc2)CC(N2c3ccccc3Oc3ccccc32)C1O)c1ccc(OC(F)(F)F)cc1] |
| 457 | 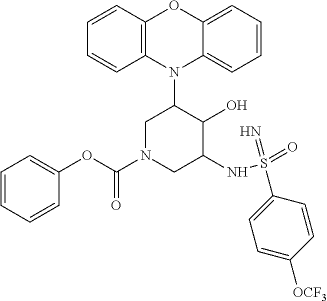 [SMILES: N=S(=O)(NC1CN(C(=O)Oc2ccccc2)CC(N2c3ccccc3Oc3ccccc32)C1O)c1ccc(OC(F)(F)F)cc1] |
| 458 | 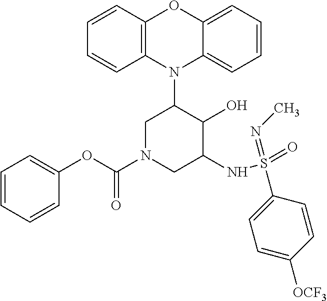 [SMILES: CN=S(=O)(NC1CN(C(=O)Oc2ccccc2)CC(N2c3ccccc3Oc3ccccc32)C1O)c1ccc(OC(F)(F)F)cc1] |
| 459 | 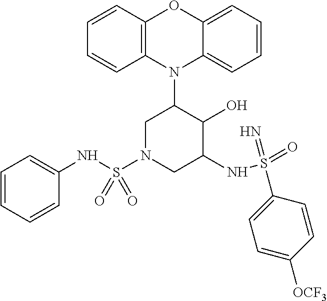 [SMILES: N=S(=O)(NC1CN(S(=O)(=O)Nc2ccccc2)CC(N2c3ccccc3Oc3ccccc32)C1O)c1ccc(OC(F)(F)F)cc1] |
| 460 | 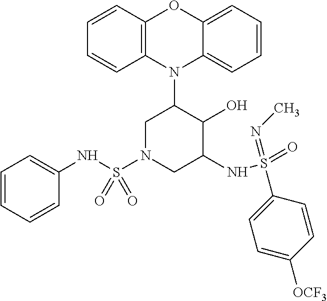 [SMILES: CN=S(=O)(NC1CN(S(=O)(=O)Nc2ccccc2)CC(N2c3ccccc3Oc3ccccc32)C1O)c1ccc(OC(F)(F)F)cc1] |
| 461 | 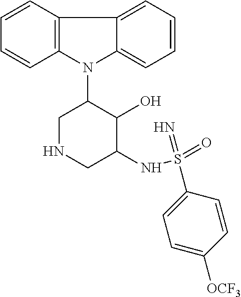 [SMILES: N=S(=O)(NC1CNCC(n2c3ccccc3c3ccccc32)C1O)c1ccc(OC(F)(F)F)cc1] |
| 462 | 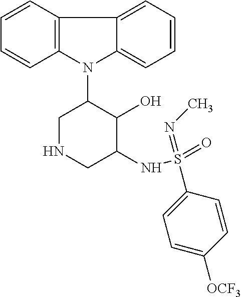 [SMILES: CN=S(=O)(NC1CNCC(n2c3ccccc3c3ccccc32)C1O)c1ccc(OC(F)(F)F)cc1] |
| 463 | 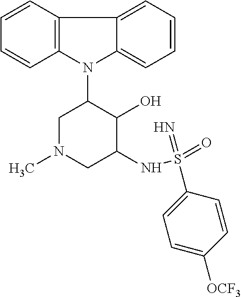 [SMILES: CN1CC(NS(=N)(=O)c2ccc(OC(F)(F)F)cc2)C(O)C(n2c3ccccc3c3ccccc32)C1] |
| 464 | 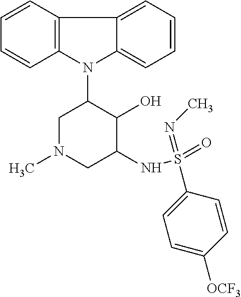 [SMILES: CN=S(=O)(NC1CN(C)CC(n2c3ccccc3c3ccccc32)C1O)c1ccc(OC(F)(F)F)cc1] |
| 465 | 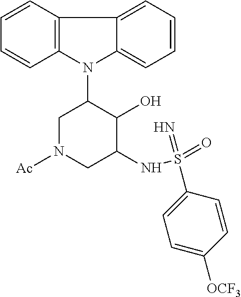 [SMILES: CC(=O)N1CC(NS(=N)(=O)c2ccc(OC(F)(F)F)cc2)C(O)C(n2c3ccccc3c3ccccc32)C1] |
| 466 | 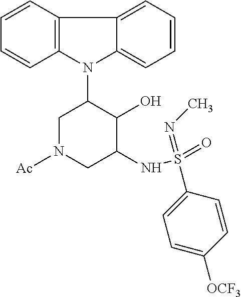 [SMILES: CN=S(=O)(NC1CN(C(C)=O)CC(n2c3ccccc3c3ccccc32)C1O)c1ccc(OC(F)(F)F)cc1] |
| 467 | 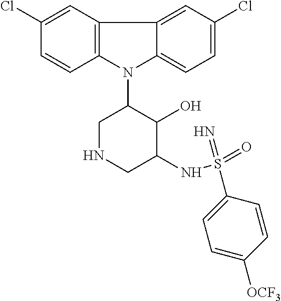 [SMILES: N=S(=O)(NC1CNCC(n2c3ccc(Cl)cc3c3cc(Cl)ccc32)C1O)c1ccc(OC(F)(F)F)cc1] |
| 468 | 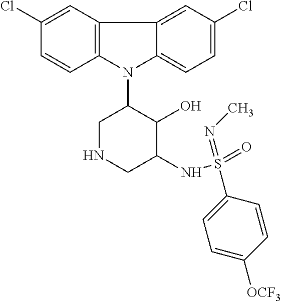 [SMILES: CN=S(=O)(NC1CNCC(n2c3ccc(Cl)cc3c3cc(Cl)ccc32)C1O)c1ccc(OC(F)(F)F)cc1] |
| 469 | 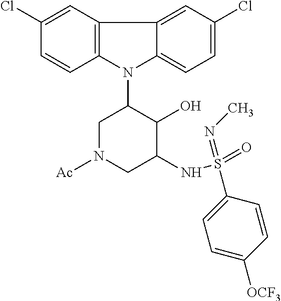 [SMILES: CN=S(=O)(NC1CN(C(C)=O)CC(n2c3ccc(Cl)cc3c3cc(Cl)ccc32)C1O)c1ccc(OC(F)(F)F)cc1] |
| 470 | 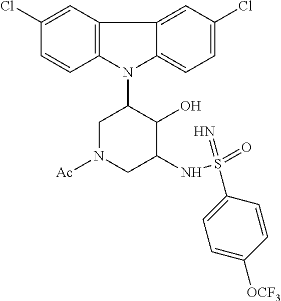 [SMILES: CC(=O)N1CC(NS(=N)(=O)c2ccc(OC(F)(F)F)cc2)C(O)C(n2c3ccc(Cl)cc3c3cc(Cl)ccc32)C1] |
| 471 | 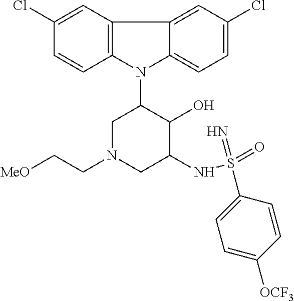 [SMILES: COCCN1CC(NS(=N)(=O)c2ccc(OC(F)(F)F)cc2)C(O)C(n2c3ccc(Cl)cc3c3cc(Cl)ccc32)C1] |
| 472 | 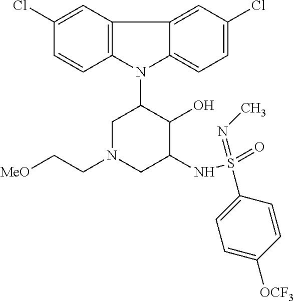 [SMILES: CN=S(=O)(NC1CN(CCOC)CC(n2c3ccc(Cl)cc3c3cc(Cl)ccc32)C1O)c1ccc(OC(F)(F)F)cc1] |
| 473 | 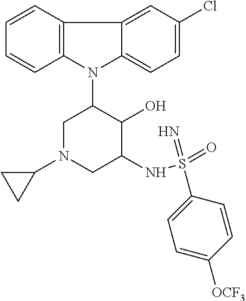 [SMILES: N=S(=O)(NC1CN(C2CC2)CC(n2c3ccccc3c3cc(Cl)ccc32)C1O)c1ccc(OC(F)(F)F)cc1] |
| 474 | 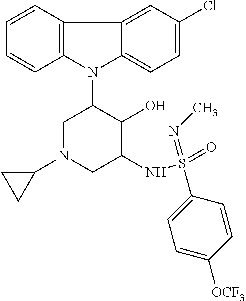 [SMILES: CN=S(=O)(NC1CN(C2CC2)CC(n2c3ccccc3c3cc(Cl)ccc32)C1O)c1ccc(OC(F)(F)F)cc1] |
| 475 | 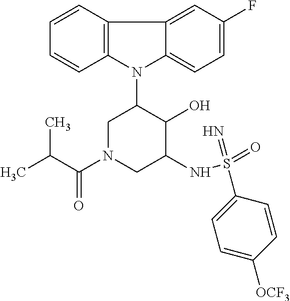 [SMILES: CC(C)C(=O)N1CC(NS(=N)(=O)c2ccc(OC(F)(F)F)cc2)C(O)C(n2c3ccccc3c3cc(F)ccc32)C1] |
| 476 | 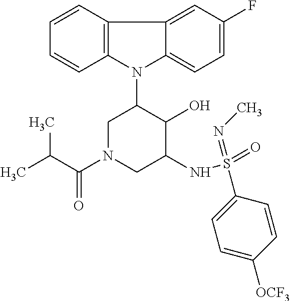 [SMILES: CN=S(=O)(NC1CN(C(=O)C(C)C)CC(n2c3ccccc3c3cc(F)ccc32)C1O)c1ccc(OC(F)(F)F)cc1] |
| 477 | 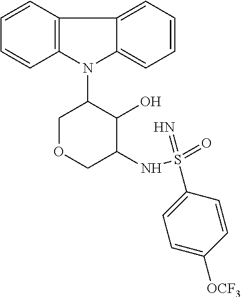 [SMILES: N=S(=O)(NC1COCC(n2c3ccccc3c3ccccc32)C1O)c1ccc(OC(F)(F)F)cc1] |
| 478 | 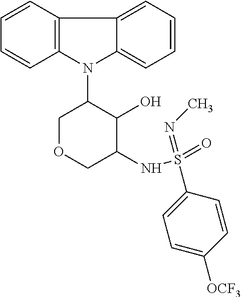 [SMILES: CN=S(=O)(NC1COCC(n2c3ccccc3c3ccccc32)C1O)c1ccc(OC(F)(F)F)cc1] |
| 479 | 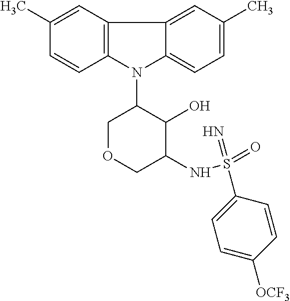 [SMILES: Cc1ccc2c(c1)c1cc(C)ccc1n2C1COCC(NS(=N)(=O)c2ccc(OC(F)(F)F)cc2)C1O] |
| 480 | 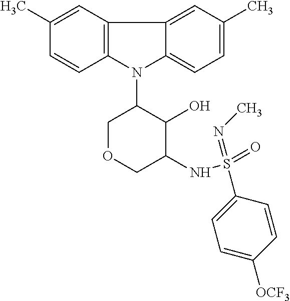 [SMILES: CN=S(=O)(NC1COCC(n2c3ccc(C)cc3c3cc(C)ccc32)C1O)c1ccc(OC(F)(F)F)cc1] |
| 481 | 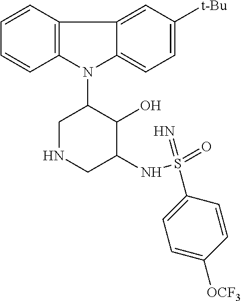 [SMILES: CC(C)(C)c1ccc2c(c1)c1ccccc1n2C1CNCC(NS(=N)(=O)c2ccc(OC(F)(F)F)cc2)C1O] |
| 482 | 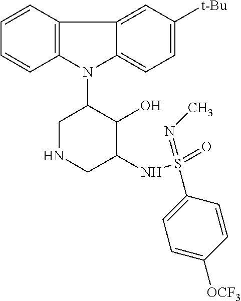 [SMILES: CN=S(=O)(NC1CNCC(n2c3ccccc3c3cc(C(C)(C)C)ccc32)C1O)c1ccc(OC(F)(F)F)cc1] |
| 483 | 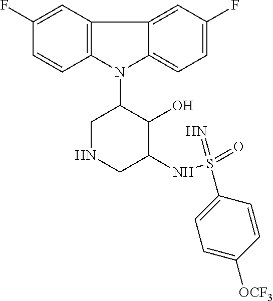 [SMILES: N=S(=O)(NC1CNCC(n2c3ccc(F)cc3c3cc(F)ccc32)C1O)c1ccc(OC(F)(F)F)cc1] |
| 484 | 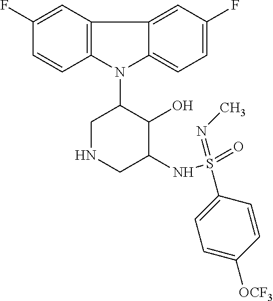 [SMILES: CN=S(=O)(NC1CNCC(n2c3ccc(F)cc3c3cc(F)ccc32)C1O)c1ccc(OC(F)(F)F)cc1] |
| 485 | 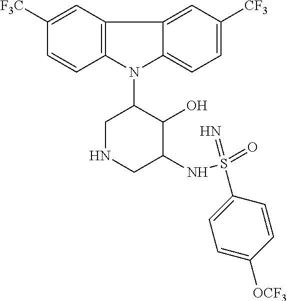 [SMILES: N=S(=O)(NC1CNCC(n2c3ccc(C(F)(F)F)cc3c3cc(C(F)(F)F)ccc32)C1O)c1ccc(OC(F)(F)F)cc1] |
| 486 | 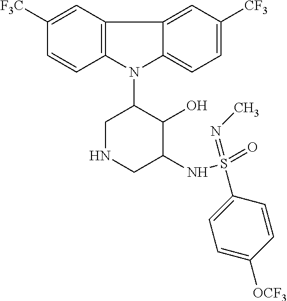 [SMILES: CN=S(=O)(NC1CNCC(n2c3ccc(C(F)(F)F)cc3c3cc(C(F)(F)F)ccc32)C1O)c1ccc(OC(F)(F)F)cc1] |
| 487 | 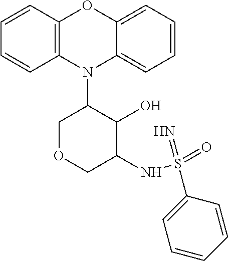 [SMILES: N=S(=O)(NC1COCC(N2c3ccccc3Oc3ccccc32)C1O)c1ccccc1] |
| 488 | 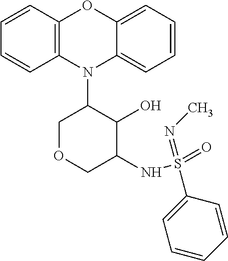 [SMILES: CN=S(=O)(NC1COCC(N2c3ccccc3Oc3ccccc32)C1O)c1ccccc1] |
| 489 | 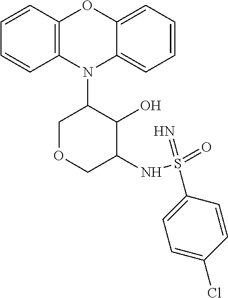 [SMILES: N=S(=O)(NC1COCC(N2c3ccccc3Oc3ccccc32)C1O)c1ccc(Cl)cc1] |
| 490 | 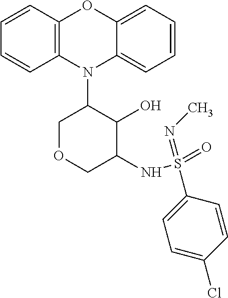 [SMILES: CN=S(=O)(NC1COCC(N2c3ccccc3Oc3ccccc32)C1O)c1ccc(Cl)cc1] |
| 491 | 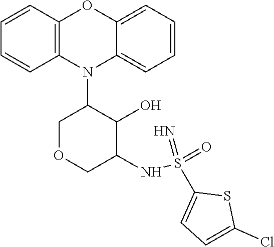 [SMILES: N=S(=O)(NC1COCC(N2c3ccccc3Oc3ccccc32)C1O)c1ccc(Cl)s1] |
| 492 | 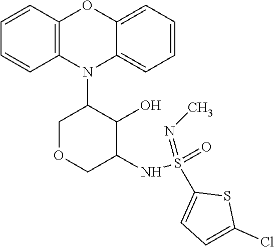 [SMILES: CN=S(=O)(NC1COCC(N2c3ccccc3Oc3ccccc32)C1O)c1ccc(Cl)s1] |
| 293 | 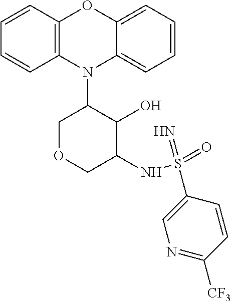 [SMILES: N=S(=O)(NC1COCC(N2c3ccccc3Oc3ccccc32)C1O)c1ccc(C(F)(F)F)nc1] |
| 494 | 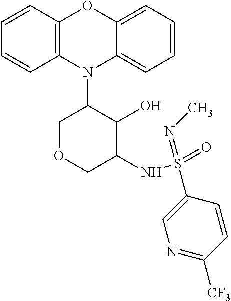 [SMILES: CN=S(=O)(NC1COCC(N2c3ccccc3Oc3ccccc32)C1O)c1ccc(C(F)(F)F)nc1] |
| 495 | 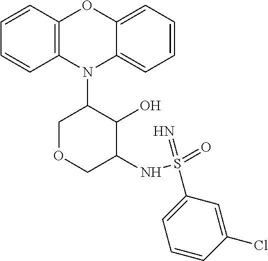 [SMILES: N=S(=O)(NC1COCC(N2c3ccccc3Oc3ccccc32)C1O)c1cccc(Cl)c1] |
| 496 | 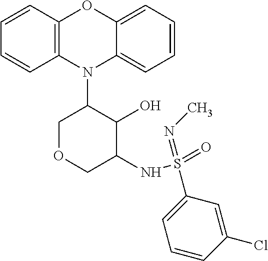 [SMILES: CN=S(=O)(NC1COCC(N2c3ccccc3Oc3ccccc32)C1O)c1cccc(Cl)c1] |
| 497 | 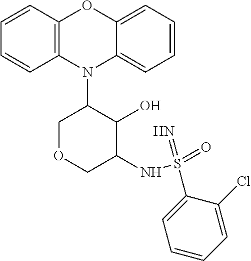 [SMILES: N=S(=O)(NC1COCC(N2c3ccccc3Oc3ccccc32)C1O)c1ccccc1Cl] |
| 498 | 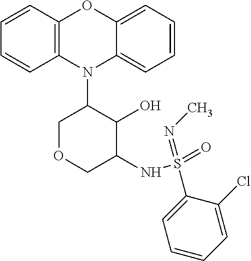 [SMILES: CN=S(=O)(NC1COCC(N2c3ccccc3Oc3ccccc32)C1O)c1ccccc1Cl] |
| 499 | 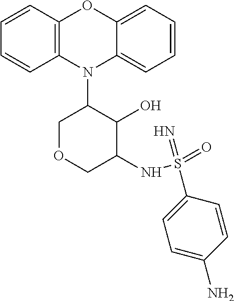 [SMILES: N=S(=O)(NC1COCC(N2c3ccccc3Oc3ccccc32)C1O)c1ccc(N)cc1] |
| 500 | 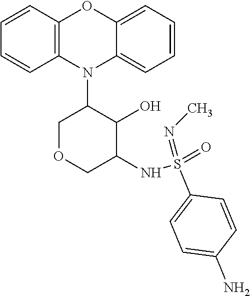 [SMILES: CN=S(=O)(NC1COCC(N2c3ccccc3Oc3ccccc32)C1O)c1ccc(N)cc1] |
| 501 | 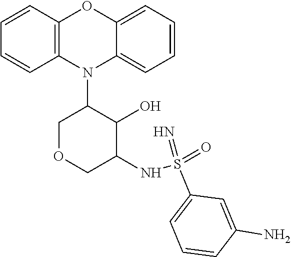 [SMILES: N=S(=O)(NC1COCC(N2c3ccccc3Oc3ccccc32)C1O)c1cccc(N)c1] |
| 502 | 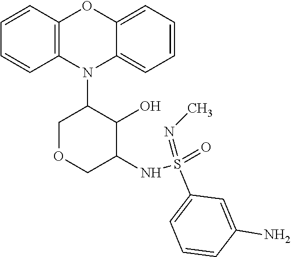 [SMILES: CN=S(=O)(NC1COCC(N2c3ccccc3Oc3ccccc32)C1O)c1cccc(N)c1] |
| 503 | 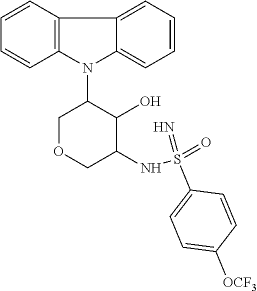 [SMILES: N=S(=O)(NC1COCC(n2c3ccccc3c3ccccc32)C1O)c1ccc(OC(F)(F)F)cc1] |
| 504 | 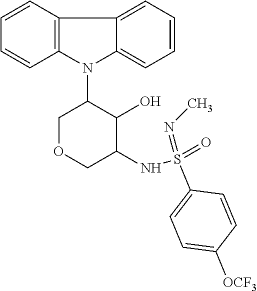 [SMILES: CN=S(=O)(NC1COCC(n2c3ccccc3c3ccccc32)C1O)c1ccc(OC(F)(F)F)cc1] |
| 505 | 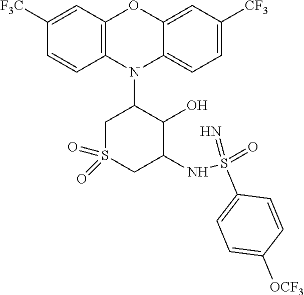 [SMILES: N=S(=O)(NC1CS(=O)(=O)CC(N2c3ccc(C(F)(F)F)cc3Oc3cc(C(F)(F)F)ccc32)C1O)c1ccc(OC(F)(F)F)cc1] |
| 506 | 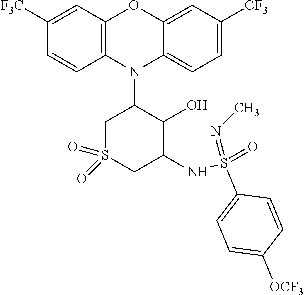 [SMILES: CN=S(=O)(NC1CS(=O)(=O)CC(N2c3ccc(C(F)(F)F)cc3Oc3cc(C(F)(F)F)ccc32)C1O)c1ccc(OC(F)(F)F)cc1] |
| 507 | 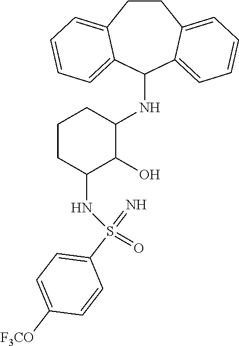 [SMILES: N=S(=O)(NC1CCCC(NC2c3ccccc3CCc3ccccc32)C1O)c1ccc(OC(F)(F)F)cc1] |
| 508 | 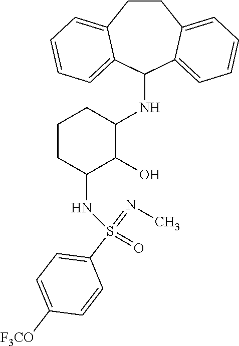 [SMILES: CN=S(=O)(NC1CCCC(NC2c3ccccc3CCc3ccccc32)C1O)c1ccc(OC(F)(F)F)cc1] |
| 509 | 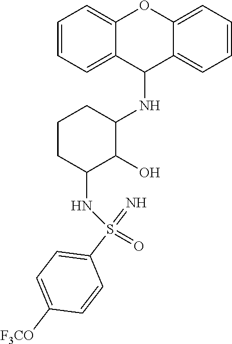 [SMILES: N=S(=O)(NC1CCCC(NC2c3ccccc3Oc3ccccc32)C1O)c1ccc(OC(F)(F)F)cc1] |
| 510 | 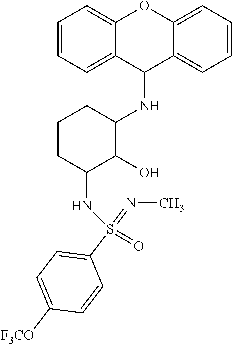 [SMILES: CN=S(=O)(NC1CCCC(NC2c3ccccc3Oc3ccccc32)C1O)c1ccc(OC(F)(F)F)cc1] |
| 511 | 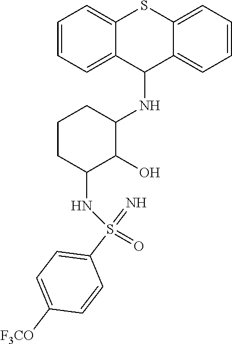 [SMILES: N=S(=O)(NC1CCCC(NC2c3ccccc3Sc3ccccc32)C1O)c1ccc(OC(F)(F)F)cc1] |
| 512 | 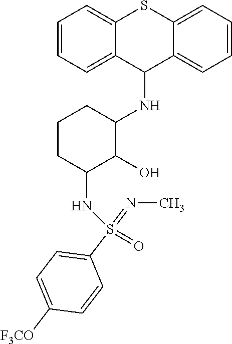 [SMILES: CN=S(=O)(NC1CCCC(NC2c3ccccc3Sc3ccccc32)C1O)c1ccc(OC(F)(F)F)cc1] |
| 513 | 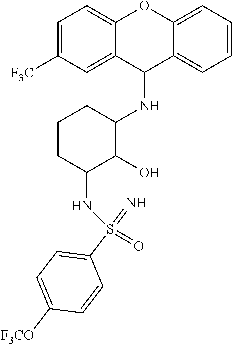 [SMILES: N=S(=O)(NC1CCCC(NC2c3ccccc3Oc3ccc(C(F)(F)F)cc32)C1O)c1ccc(OC(F)(F)F)cc1] |
| 514 | 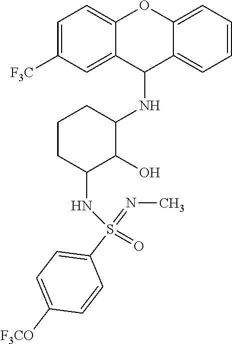 [SMILES: CN=S(=O)(NC1CCCC(NC2c3ccccc3Oc3ccc(C(F)(F)F)cc32)C1O)c1ccc(OC(F)(F)F)cc1] |
| 515 | 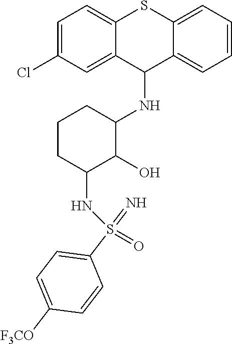 [SMILES: N=S(=O)(NC1CCCC(NC2c3ccccc3Sc3ccc(Cl)cc32)C1O)c1ccc(OC(F)(F)F)cc1] |
| 516 | 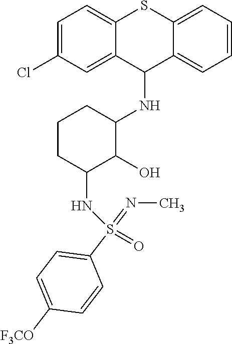 [SMILES: CN=S(=O)(NC1CCCC(NC2c3ccccc3Sc3ccc(Cl)cc32)C1O)c1ccc(OC(F)(F)F)cc1] |
| 517 | 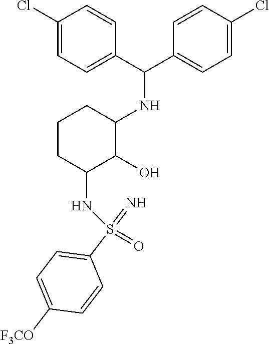 [SMILES: N=S(=O)(NC1CCCC(NC(c2ccc(Cl)cc2)c2ccc(Cl)cc2)C1O)c1ccc(OC(F)(F)F)cc1] |
| 518 | 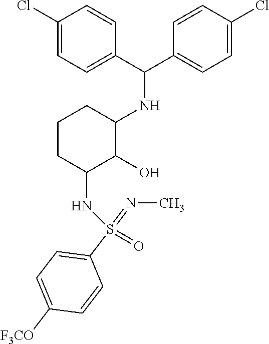 [SMILES: CN=S(=O)(NC1CCCC(NC(c2ccc(Cl)cc2)c2ccc(Cl)cc2)C1O)c1ccc(OC(F)(F)F)cc1] |
| 519 | 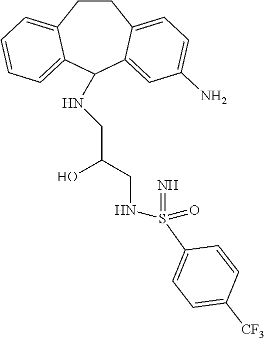 [SMILES: N=S(=O)(NCC(O)CNC1c2ccccc2CCc2ccc(N)cc21)c1ccc(C(F)(F)F)cc1] |
| 520 | 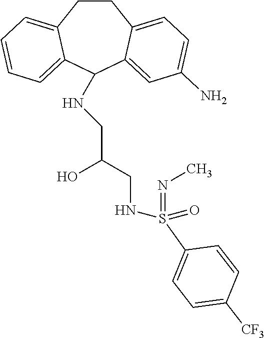 [SMILES: CN=S(=O)(NCC(O)CNC1c2ccccc2CCc2ccc(N)cc21)c1ccc(C(F)(F)F)cc1] |
| 521 | 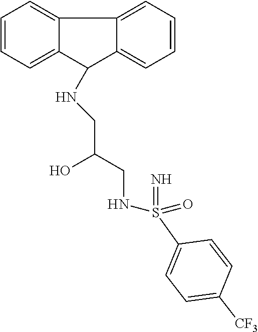 [SMILES: N=S(=O)(NCC(O)CNC1c2ccccc2-c2ccccc21)c1ccc(C(F)(F)F)cc1] |
| 522 | 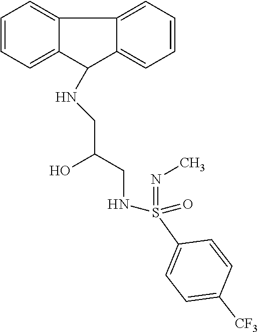 [SMILES: CN=S(=O)(NCC(O)CNC1c2ccccc2-c2ccccc21)c1ccc(C(F)(F)F)cc1] |
| 523 | 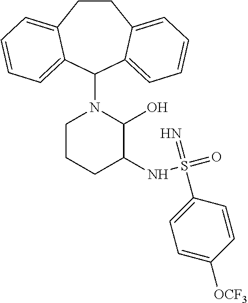 [SMILES: N=S(=O)(NC1CCCN(C2c3ccccc3CCc3ccccc32)C1O)c1ccc(OC(F)(F)F)cc1] |
| 524 | 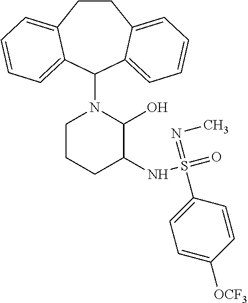 [SMILES: CN=S(=O)(NC1CCCN(C2c3ccccc3CCc3ccccc32)C1O)c1ccc(OC(F)(F)F)cc1] |
| 525 | 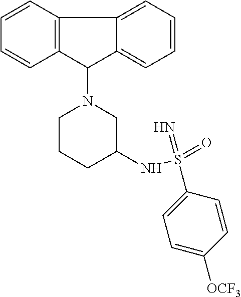 [SMILES: N=S(=O)(NC1CCCN(C2c3ccccc3-c3ccccc32)C1)c1ccc(OC(F)(F)F)cc1] |
| 526 | 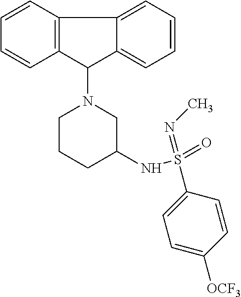 [SMILES: CN=S(=O)(NC1CCCN(C2c3ccccc3-c3ccccc32)C1)c1ccc(OC(F)(F)F)cc1] |
| 527 | 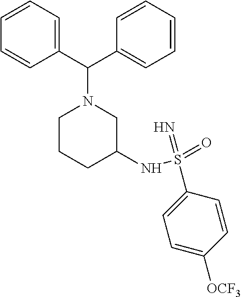 [SMILES: N=S(=O)(NC1CCCN(C(c2ccccc2)c2ccccc2)C1)c1ccc(OC(F)(F)F)cc1] |
| 528 | 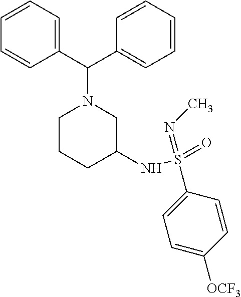 [SMILES: CN=S(=O)(NC1CCCN(C(c2ccccc2)c2ccccc2)C1)c1ccc(OC(F)(F)F)cc1] |
| 529 | 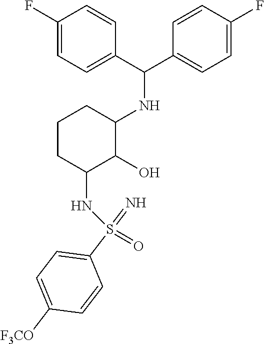 [SMILES: N=S(=O)(NC1CCCC(NC(c2ccc(F)cc2)c2ccc(F)cc2)C1O)c1ccc(OC(F)(F)F)cc1] |
| 530 | 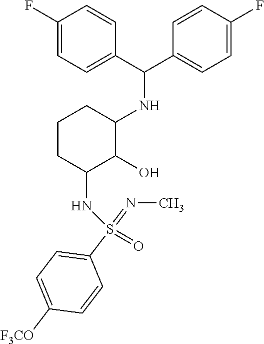 [SMILES: CN=S(=O)(NC1CCCC(NC(c2ccc(F)cc2)c2ccc(F)cc2)C1O)c1ccc(OC(F)(F)F)cc1] |
| 531 | 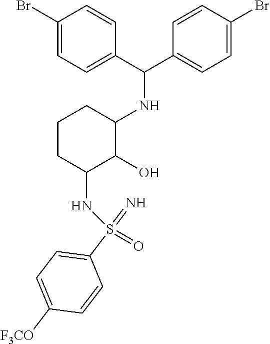 [SMILES: N=S(=O)(NC1CCCC(NC(c2ccc(Br)cc2)c2ccc(Br)cc2)C1O)c1ccc(OC(F)(F)F)cc1] |
| 532 | 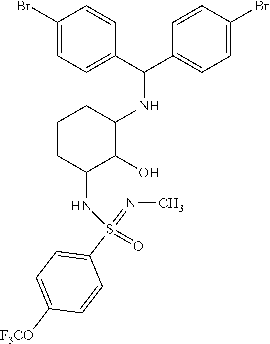 [SMILES: CN=S(=O)(NC1CCCC(NC(c2ccc(Br)cc2)c2ccc(Br)cc2)C1O)c1ccc(OC(F)(F)F)cc1] |
| 533 | 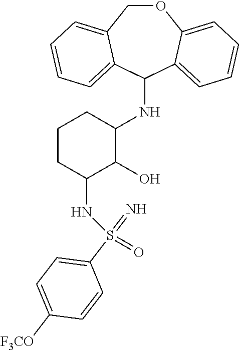 [SMILES: N=S(=O)(NC1CCCC(NC2c3ccccc3COc3ccccc32)C1O)c1ccc(OC(F)(F)F)cc1] |
| 534 | 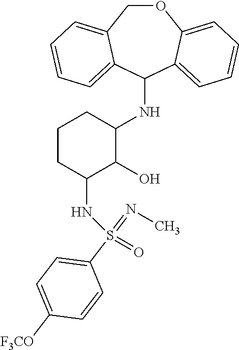 [SMILES: CN=S(=O)(NC1CCCC(NC2c3ccccc3COc3ccccc32)C1O)c1ccc(OC(F)(F)F)cc1] |
| 535 | 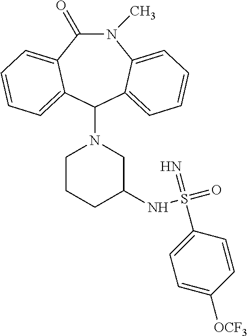 [SMILES: CN1C(=O)c2ccccc2C(N2CCCC(NS(=N)(=O)c3ccc(OC(F)(F)F)cc3)C2)c2ccccc21] |
| 536 | 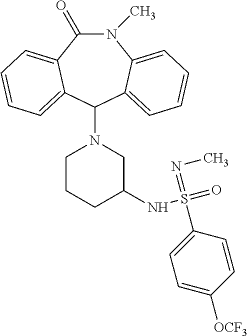 [SMILES: CN=S(=O)(NC1CCCN(C2c3ccccc3C(=O)N(C)c3ccccc32)C1)c1ccc(OC(F)(F)F)cc1] |
| 537 | 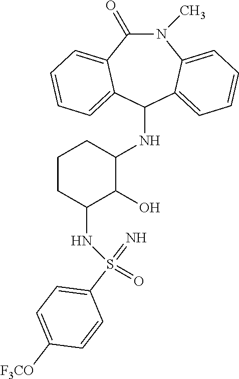 [SMILES: CN1C(=O)c2ccccc2C(NC2CCCC(NS(=N)(=O)c3ccc(OC(F)(F)F)cc3)C2O)c2ccccc21] |
| 538 | 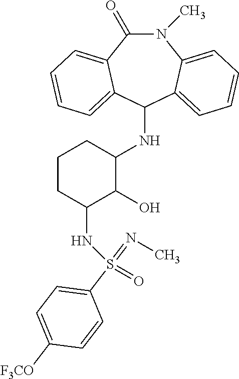 [SMILES: CN=S(=O)(NC1CCCC(NC2c3ccccc3C(=O)N(C)c3ccccc32)C1O)c1ccc(OC(F)(F)F)cc1] |
| 539 | 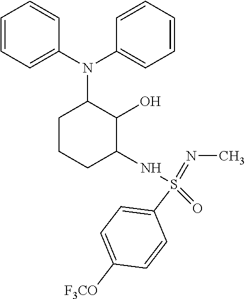 [SMILES: CN=S(=O)(NC1CCCC(N(c2ccccc2)c2ccccc2)C1O)c1ccc(OC(F)(F)F)cc1] |
| 540 | 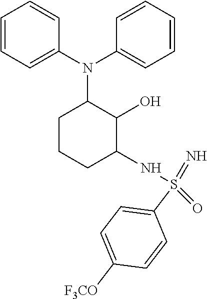 [SMILES: N=S(=O)(NC1CCCC(N(c2ccccc2)c2ccccc2)C1O)c1ccc(OC(F)(F)F)cc1] |
| 541 | 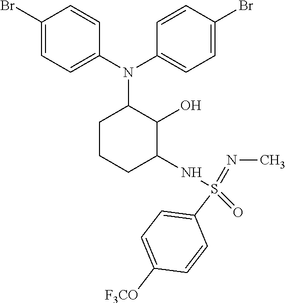 [SMILES: CN=S(=O)(NC1CCCC(N(c2ccc(Br)cc2)c2ccc(Br)cc2)C1O)c1ccc(OC(F)(F)F)cc1] |
| 542 | 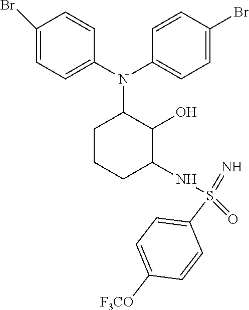 [SMILES: N=S(=O)(NC1CCCC(N(c2ccc(Br)cc2)c2ccc(Br)cc2)C1O)c1ccc(OC(F)(F)F)cc1] |
| 543 | 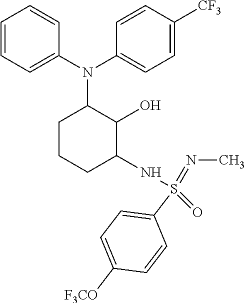 [SMILES: CN=S(=O)(NC1CCCC(N(c2ccccc2)c2ccc(C(F)(F)F)cc2)C1O)c1ccc(OC(F)(F)F)cc1] |
| 544 | 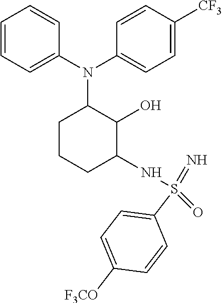 [SMILES: N=S(=O)(NC1CCCC(N(c2ccccc2)c2ccc(C(F)(F)F)cc2)C1O)c1ccc(OC(F)(F)F)cc1] |
| 545 | 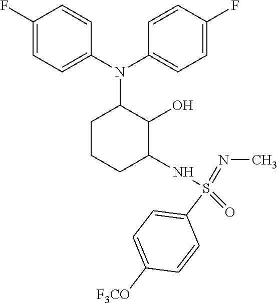 [SMILES: CN=S(=O)(NC1CCCC(N(c2ccc(F)cc2)c2ccc(F)cc2)C1O)c1ccc(OC(F)(F)F)cc1] |
| 546 | 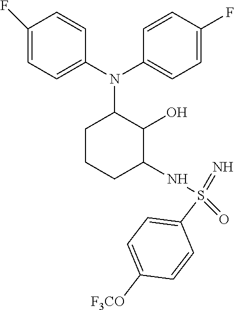 [SMILES: N=S(=O)(NC1CCCC(N(c2ccc(F)cc2)c2ccc(F)cc2)C1O)c1ccc(OC(F)(F)F)cc1] |
| 547 | 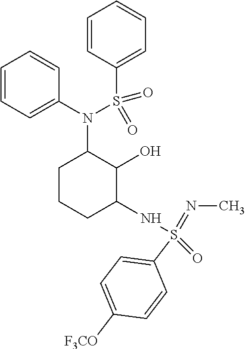 [SMILES: CN=S(=O)(NC1CCCC(N(c2ccccc2)S(=O)(=O)c2ccccc2)C1O)c1ccc(OC(F)(F)F)cc1] |
| 548 | 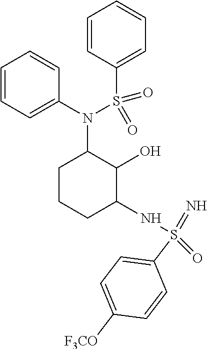 [SMILES: N=S(=O)(NC1CCCC(N(c2ccccc2)S(=O)(=O)c2ccccc2)C1O)c1ccc(OC(F)(F)F)cc1] |
| 549 | 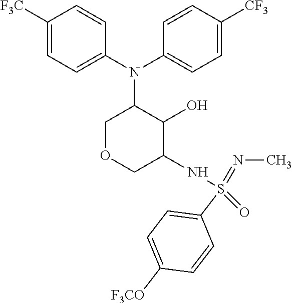 [SMILES: CN=S(=O)(NC1COCC(N(c2ccc(C(F)(F)F)cc2)c2ccc(C(F)(F)F)cc2)C1O)c1ccc(OC(F)(F)F)cc1] |
| 550 | 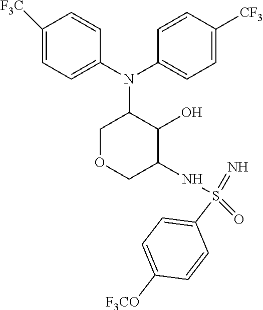 [SMILES: N=S(=O)(NC1COCC(N(c2ccc(C(F)(F)F)cc2)c2ccc(C(F)(F)F)cc2)C1O)c1ccc(OC(F)(F)F)cc1] |
| 551 | 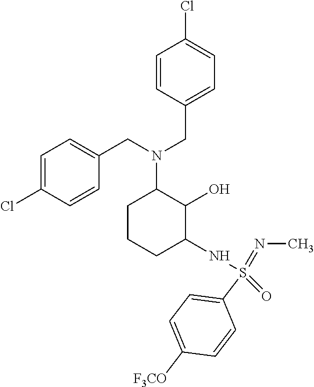 [SMILES: CN=S(=O)(NC1CCCC(N(Cc2ccc(Cl)cc2)Cc2ccc(Cl)cc2)C1O)c1ccc(OC(F)(F)F)cc1] |
| 552 | 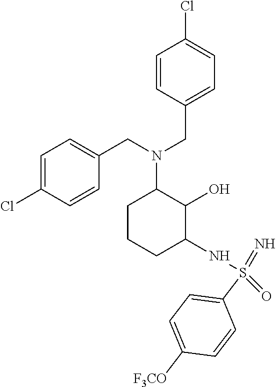 [SMILES: N=S(=O)(NC1CCCC(N(Cc2ccc(Cl)cc2)Cc2ccc(Cl)cc2)C1O)c1ccc(OC(F)(F)F)cc1] |
| 553 | 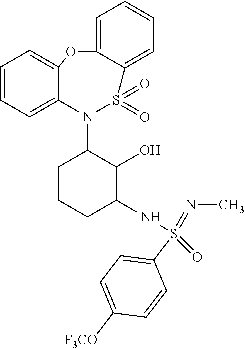 [SMILES: CN=S(=O)(NC1CCCC(N2c3ccccc3Oc3ccccc3S2(=O)=O)C1O)c1ccc(OC(F)(F)F)cc1] |
| 554 | 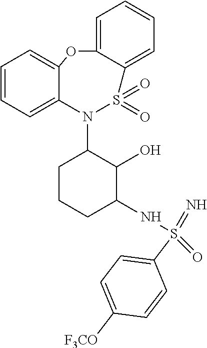 [SMILES: N=S(=O)(NC1CCCC(N2c3ccccc3Oc3ccccc3S2(=O)=O)C1O)c1ccc(OC(F)(F)F)cc1] |
| 555 | 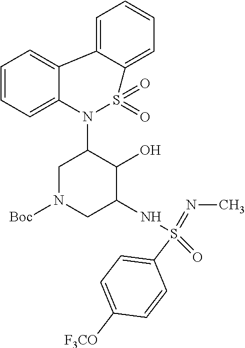 [SMILES: CN=S(=O)(NC1CN(C(=O)OC(C)(C)C)CC(N2c3ccccc3-c3ccccc3S2(=O)=O)C1O)c1ccc(OC(F)(F)F)cc1] |
| 556 | 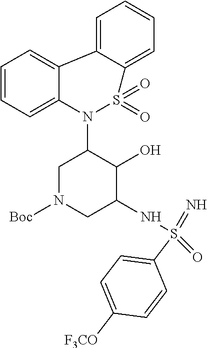 [SMILES: CC(C)(C)OC(=O)N1CC(NS(=N)(=O)c2ccc(OC(F)(F)F)cc2)C(O)C(N2c3ccccc3-c3ccccc3S2(=O)=O)C1] |
| 557 | 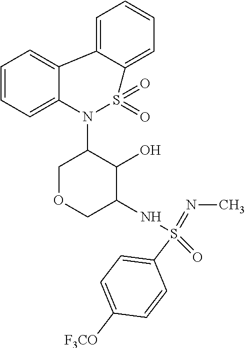 [SMILES: CN=S(=O)(NC1COCC(N2c3ccccc3-c3ccccc3S2(=O)=O)C1O)c1ccc(OC(F)(F)F)cc1] |
| 558 | 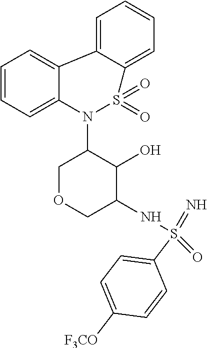 [SMILES: N=S(=O)(NC1COCC(N2c3ccccc3-c3ccccc3S2(=O)=O)C1O)c1ccc(OC(F)(F)F)cc1] |
| 559 | 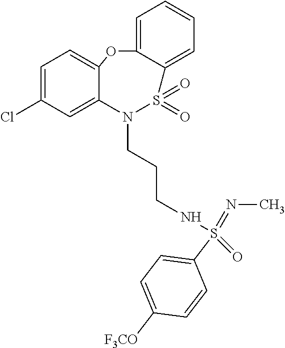 [SMILES: CN=S(=O)(NCCCN1c2cc(Cl)ccc2Oc2ccccc2S1(=O)=O)c1ccc(OC(F)(F)F)cc1] |
| 560 | 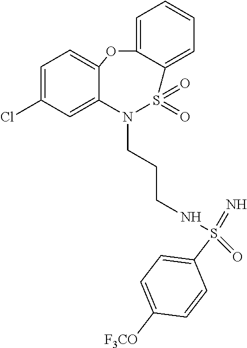 [SMILES: N=S(=O)(NCCCN1c2cc(Cl)ccc2Oc2ccccc2S1(=O)=O)c1ccc(OC(F)(F)F)cc1] |
| 561 | 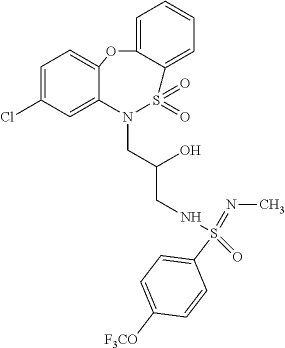 [SMILES: CN=S(=O)(NCC(O)CN1c2cc(Cl)ccc2Oc2ccccc2S1(=O)=O)c1ccc(OC(F)(F)F)cc1] |
| 562 | 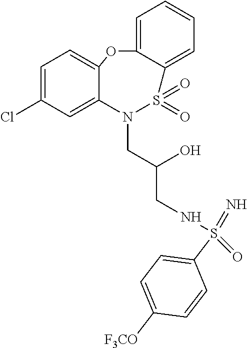 [SMILES: N=S(=O)(NCC(O)CN1c2cc(Cl)ccc2Oc2ccccc2S1(=O)=O)c1ccc(OC(F)(F)F)cc1] |
| 563 | 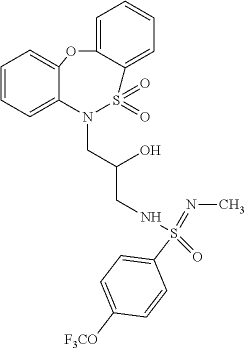 [SMILES: CN=S(=O)(NCC(O)CN1c2ccccc2Oc2ccccc2S1(=O)=O)c1ccc(OC(F)(F)F)cc1] |
| 564 | 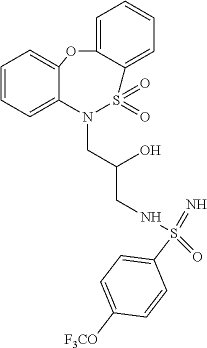 [SMILES: N=S(=O)(NCC(O)CN1c2ccccc2Oc2ccccc2S1(=O)=O)c1ccc(OC(F)(F)F)cc1] |
| 565 | 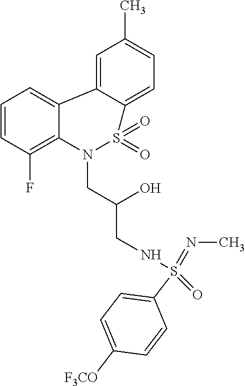 [SMILES: CN=S(=O)(NCC(O)CN1c2c(F)cccc2-c2cc(C)ccc2S1(=O)=O)c1ccc(OC(F)(F)F)cc1] |
| 566 | 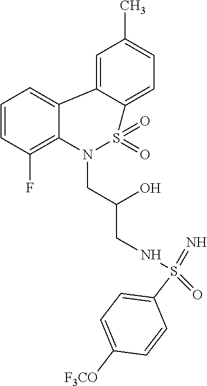 [SMILES: Cc1ccc2c(c1)-c1cccc(F)c1N(CC(O)CNS(=N)(=O)c1ccc(OC(F)(F)F)cc1)S2(=O)=O] |
| 567 | 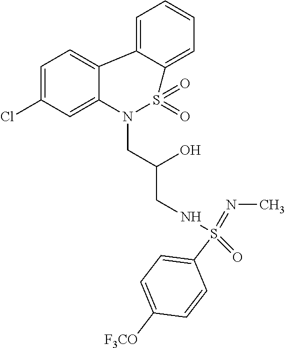 [SMILES: CN=S(=O)(NCC(O)CN1c2cc(Cl)ccc2-c2ccccc2S1(=O)=O)c1ccc(OC(F)(F)F)cc1] |
| 568 | 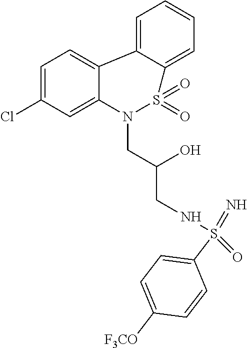 [SMILES: N=S(=O)(NCC(O)CN1c2cc(Cl)ccc2-c2ccccc2S1(=O)=O)c1ccc(OC(F)(F)F)cc1] |
| 569 | 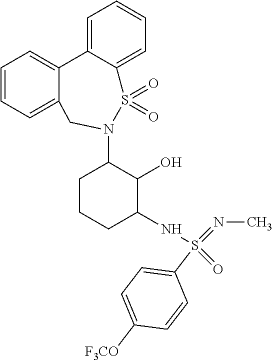 [SMILES: CN=S(=O)(NC1CCCC(N2Cc3ccccc3-c3ccccc3S2(=O)=O)C1O)c1ccc(OC(F)(F)F)cc1] |
| 570 | 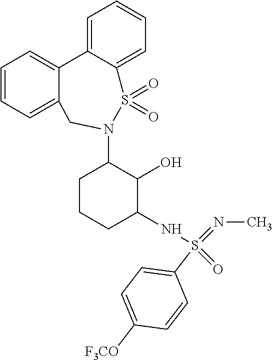 [SMILES: CN=S(=O)(NC1CCCC(N2Cc3ccccc3-c3ccccc3S2(=O)=O)C1O)c1ccc(OC(F)(F)F)cc1] |
| 571 | 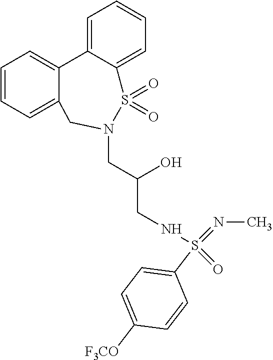 [SMILES: CN=S(=O)(NCC(O)CN1Cc2ccccc2-c2ccccc2S1(=O)=O)c1ccc(OC(F)(F)F)cc1] |
| 572 | 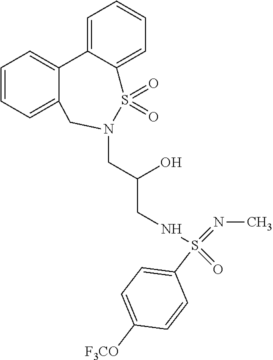 [SMILES: CN=S(=O)(NCC(O)CN1Cc2ccccc2-c2ccccc2S1(=O)=O)c1ccc(OC(F)(F)F)cc1] |
| 573 | 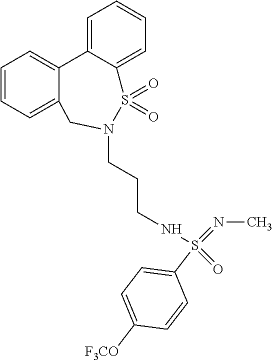 [SMILES: CN=S(=O)(NCCCN1Cc2ccccc2-c2ccccc2S1(=O)=O)c1ccc(OC(F)(F)F)cc1] |
| 574 | 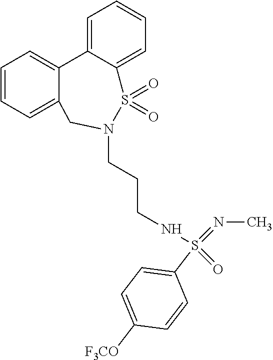 [SMILES: CN=S(=O)(NCCCN1Cc2ccccc2-c2ccccc2S1(=O)=O)c1ccc(OC(F)(F)F)cc1] |

or an enantiomer, a diastereomer, a tautomer or pharmaceutically acceptable salt thereof.

One skilled in the art can prepare compounds of formula (I) by using the following references: Bremerich M et al. Additions to N-Sulfinylamines as an Approach for the Metal-free Synthesis of Sulfonimidamides: O-Benzotriazolyl Sulfonimidates as Activated Intermediates. Angew Chem Int Ed Engl. 2019; 58:19014-20; Briggs E L et al. Synthesis of Sulfonimidamides from Sulfenamides via an Alkoxy-amino-λ(6)-sulfanenitrile Intermediate. Angew Chem Int Ed Engl. 20191; 58(40):14303-14310; Chen Y et al. Saccharin Aza Bioisosteres-Synthesis and Preclinical Property Comparisons. ACS Med Chem Lett. 2017; 8(6):672-677; Chinthakindi P K et al. Sulfonimidamides in Medicinal and Agricultural Chemistry. Angew Chem Int Ed Engl. 2017; 56(15):4100-4109; Davies T Q et al. One-Pot, Three-Component Sulfonimidamide Synthesis Exploiting the Sulfinylamine Reagent N-Sulfinyltritylamine, TrNSO. Angew Chem Int Ed Engl. 2017; 56(47):14937-14941; Gao B et al. SuFEx Chemistry of Thionyl Tetrafluoride (SOF(4)) with Organolithium Nucleophiles: Synthesis of Sulfonimidoyl Fluorides, Sulfoximines, Sulfonimidamides, and Sulfonimidates. Angew Chem Int Ed Engl. 2018; 57(7):1939-1943; Izzo F et al. A New, Practical One-Pot Synthesis of Unprotected Sulfonimidamides by Transfer of Electrophilic NH to Sulfinamides. Chemistry. 2017; 23(60):15189-15193; Izzo F et al. Exploration of Novel Chemical Space: Synthesis and in vitro Evaluation of N-Functionalized Tertiary Sulfonimidamides. Chemistry. 2018; 24(37):9295-9304; Mancheno O G and Bolm C. Synthesis of sulfonimidamides from sulfinamides by oxidation with N-chlorosuccinimide. Beilstein J Org Chem. 2007; 3:25; Nandi G C and Arvidsson P I. Sulfonimidamides: Synthesis and Applications in Preparative Organic Chemistry. Adv. Synth. Catal. 2018; 360:2976-3001; Sehgelmeble F et al. Sulfonimidamides as sulfonamides bioisosteres: rational evaluation through synthetic, in vitro, and in vivo studies with γ-secretase inhibitors. ChemMedChem. 2012; 7(3):396-9; Steinkamp A D et al. Synthesis of a Sulfonimidamide-Based Analog of Tasisulam and Its Biological Evaluation in the Melanoma Cell Lines SKMel23 and A375. Skin Pharmacol Physiol. 2016; 29(6):281-290; Wen J et al. Copper-Catalyzed S—C/S—N Bond Interconversions. Chemistry. 2016; 22(16):5547-50; Wimmer A and Konig B. N-Arylation of NHSulfoximines via Dual Nickel Photocatalysis. Org Lett. 2019; 21(8):2740-2744; Worch C et al. Synthesis of enantiopure sulfonimidamides and elucidation of their absolute configuration by comparison of measured and calculated CD spectra and X-ray crystal structure determination. Chemistry. 2010; 16(2):677-83; Yu H et al. Copper-Catalyzed Transsulfinamidation of Sulfinamides as a Key Step in the Preparation of Sulfonamides and Sulfonimidamides. Angew Chem Int Ed Engl. 2018; 57(47):15602-15605; Zasukha S V et al. Sulfonimidamides and Imidosulfuric Diamides: Compounds from an Underexplored Part of Biologically Relevant Chemical Space. Chemistry. 2019; 25(28):6928-6940.

Compounds according to the present invention consist of a tricyclic part and a sulfonimidamide part connected by a suitable linker. For example, compounds according to formula (I) can be prepared as shown in the following general procedure.

A compound of general formula 1, wherein Y, U, T, R¹, R², R³ and R⁴ are as defined for compound of formula (I) is alkylated with a haloalkyl to yield intermediates of general formula 2. Depending on the haloalkyl used, addition of a base such as NaH or NaNH₂ may be required. Suitable solvents for the alkylation reactions are e.g. toluene, DMF. Non-limiting examples of suitable linkers are —(CR¹⁵R¹⁶)ₚ-(Q)_{q}-(CR¹⁵R¹⁶)ₙ₋ₚ— as defined for compound of formula (I).

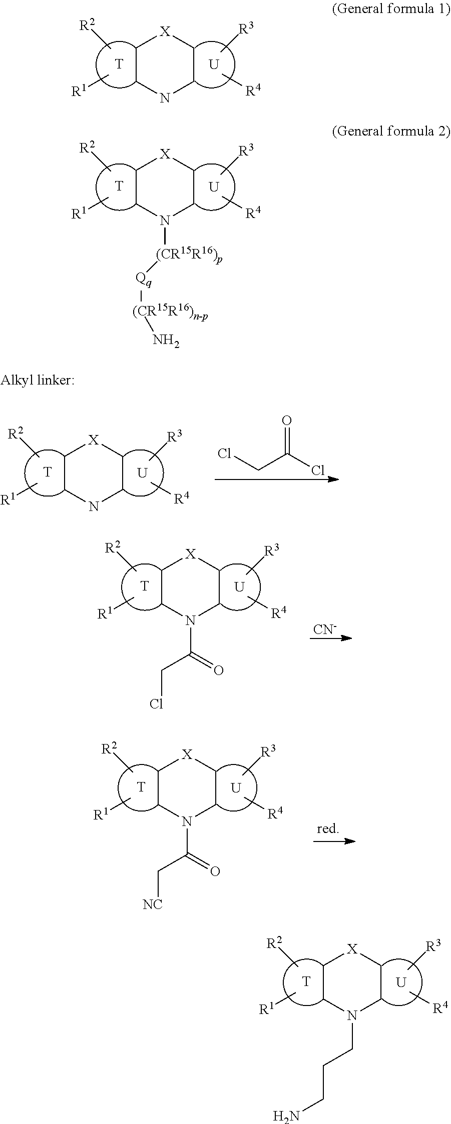

Following the alkylation of a compound of general formula 1 with a suitable carboxylic acid halide, the alkyl halide is converted to a nitrile which is reduced to an amine while simultaneously reducing the ketone to an alkyl to yield the intermediate 2 bearing an alkyl linker.

### Hydroxyalkyl Linker:

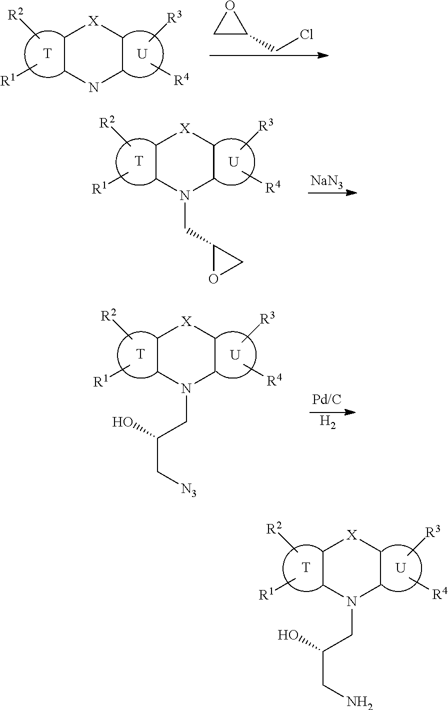

Following alkylation of a compound of general formula 1 with a halomethyl oxirane, the epoxide ring is opened under the influence of sodium azide followed by hydrogenation of the azide to yield the intermediate 2.

### Carbocyclic Linker:

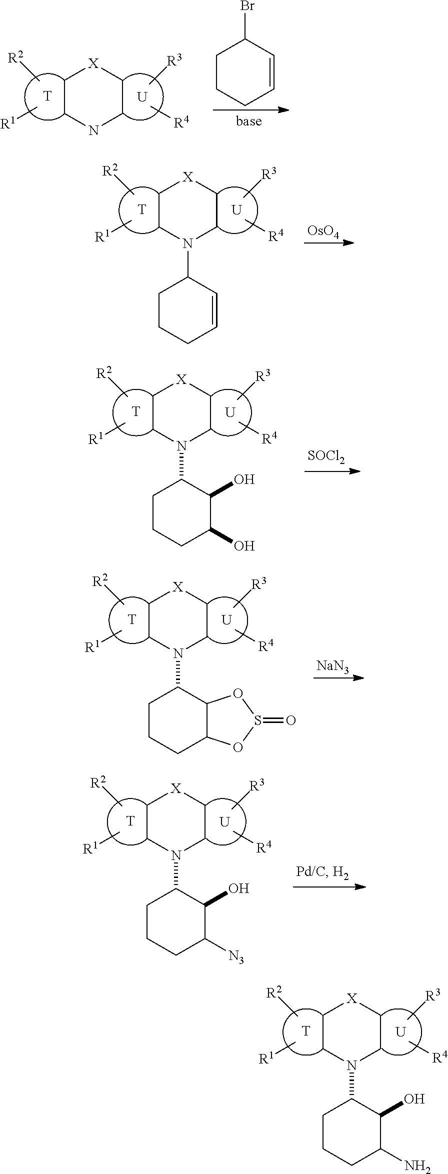

Following alkylation of a compound of general formula 1 with 3-halocyclohex-1-ene, the cycloalkene is oxidized using osmium tetroxide to yield a cis diol. The diol is converted into a cyclic sulfite ester using thionyl chloride followed by ring opening of the ester with sodium azide to form an azide intermediate. The azide moiety of the intermediate can be further hydrogenated to yield the hydroxyamine 2.

### Pyranyl Linker:

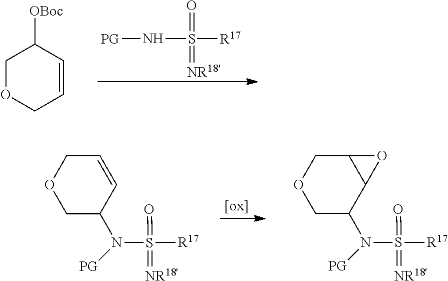

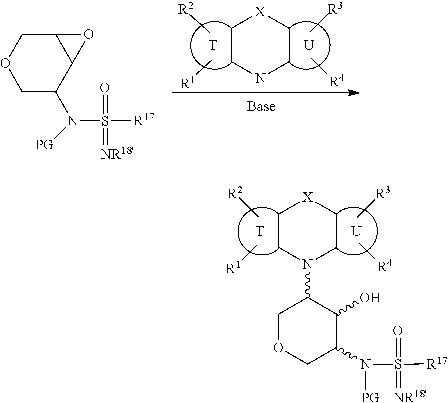

An N-protected sulfonimidamide can be coupled to a heterocyclic allylic carbonate (t-Boc-3,6-dihydro-2H-pyran-3-yl) in the presence of a chiral Palladium catalyst to give an enantiomerically enriched allyl sulfonimidamide. The allyl sulfonimidamide is epoxidized using a suitable oxidizing reagent (e.g. m-CPBA or OsO₄) followed by coupling of the formed epoxide to a compound of general formula (I) in the presence of a suitable base.

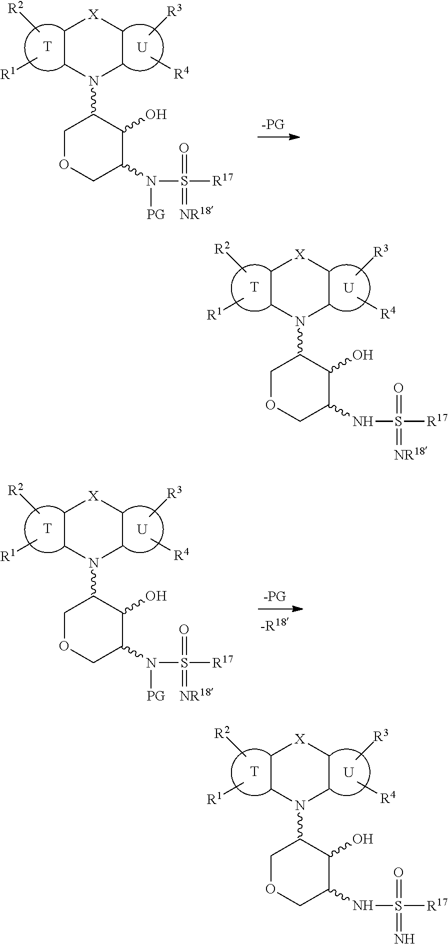

The protecting groups (PG and/or R^{18′}) may be selectively removed if needed to yield the desired product.

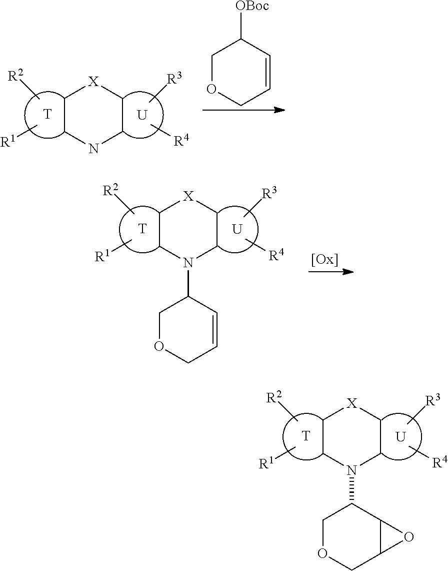

Alternatively, the heterocyclic allylic carbonate can be coupled to a compound of general formula (I) and further modified using similar reaction steps as in the case of the previously presented carbocyclic linker.

### Preparation of Compound of Formula (I):

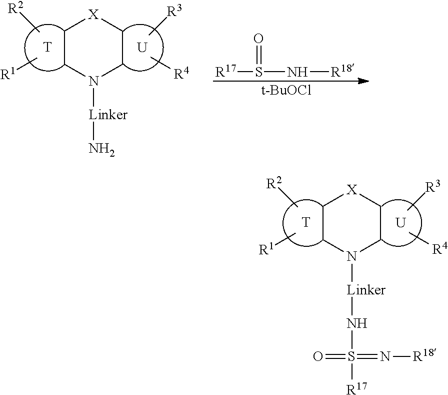

A suitably substituted chlorosulfonimine formed in situ by chlorinating a sulfinamide using t-butyl hypochlorite can be coupled to an intermediate amine 2 in a base-catalyzed reaction to form sulfonimidamide (I) or protected sulfonimidamide (I). A suitable base may be e.g. t-BuOCl, TEA, or mixtures thereof.

Suitably substituted chlorosulfonimines can be formed using any method known to a person skilled in the art or they may be acquired from commercial sources.

R^{18′} may be R¹⁸ as defined for compound of formula (I) e.g. methyl, or a protection group, e.g. methyl-2,4-dimethoxyphenyl, 9-fluorenylmethyl carbamate (Fmoc), t-butyl carbamate (Boc), benzyl carbamate (Cbz), triphenylmethyl (trityl), to mention only a few suitable examples. R¹⁷ may be as defined for compound of formula (I).

### Removal of Protecting Group

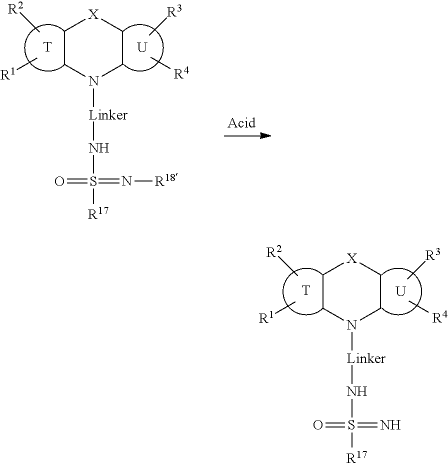

Where needed, the protection group may be removed in an acid-catalyzed process using trifluoroacetic acid (TFA) to yield the final product I.

### Chiral Compounds

In one embodiment, the compounds synthesized using the methods described herein may contain one or more chiral carbon atoms, giving rise to two or more isomers. In one embodiment, the product formed in any of the reactions described may be a racemate.

If a racemate is formed, the isomers making up the racemate may be separated using any suitable method for chiral resolution known to a person skilled in the art. Suitable methods for chiral resolution include, but are not limited to, supercritical fluid chromatography (SFC), chiral HPCL, crystallization, derivatization, or any combination thereof.

In one embodiment, separation of the isomers formed in one or more separate reactions may require forming a derivative prior to chiral resolution. A non-limiting example of derivatization is protecting one or more functional groups present in a compound using known protecting groups (such as esters, amides, carbamates, ethers, etc.), followed by separation of the isomers by a suitable method. The desired compound is finally obtained through removal of the protecting group.

### Purification

A racemic mixture of products following racemization during the final reaction step may be separated into its constituent enantiomers using any suitable method know to a person skilled in the art. As a non-limiting example, racemic mixture may be separated into enantiomers using supercritical fluid chromatography (SFC).

The present disclosure relates also to a pharmaceutical composition comprising a compound according to one or more embodiments described in this specification, for example a compound of formula I, an enantiomer, a diastereomer, a tautomer or a pharmaceutically acceptable salt thereof, and pharmaceutically acceptable carrier.

In an embodiment, the pharmaceutical composition comprises a compound of formula (I) or a pharmaceutically acceptable salt thereof, together with one or more pharmaceutically carriers thereof and optionally one or more other therapeutic ingredients.

The carrier(s) may be “acceptable” in the sense of being compatible with the other ingredients of the formulation and not deleterious to the recipient thereof.

The pharmaceutical compositions may be manufactured in any manner known in the art, e.g., by means of conventional mixing, dissolving, granulating, dragee-making, levigating, emulsifying, encapsulating, entrapping or compression processes. “Pharmaceutically acceptable carrier” may refer to an excipient, carrier or adjuvant that can be administered to a patient, together with at least one therapeutic compound, and which does not destroy the pharmacological activity thereof and is generally safe, non-toxic and neither biologically nor otherwise undesirable when administered in doses sufficient to deliver a therapeutic amount of the compound.

The compounds according to one or more embodiments disclosed in this specification may be modulators of PP2A. The compounds described herein may exhibit anti-proliferative effects and may be useful as monotherapy in cancer treatment and/or in the treatment of other indications described in this specification. Additionally, they can be used in combination with other drugs to restore sensitivity to chemotherapy, targeted therapies, or immunotherapy where resistance has developed.

As used herein, the term “modulate” means to increase or decrease the activity of PP2A. In an embodiment, compounds according to one or more embodiments disclosed in this specification may increase the activity of specific PP2A holoenzymes while decreasing the activity of other PP2A heterotrimers.

PP2A enzymes may be involved in the regulation of cell transcription, cell cycle, and viral transformation. Many viruses, including cytomegalovirus, parainfluenza, DNA tumor viruses, and HIV-1, utilize different approaches to exploit PP2A in order to modify, control, or inactivate cellular activities of the host. Therefore, the compounds according to one or more embodiments disclosed in this specification may further be used in a method for treating a viral infection in a patient by administering to the patient a therapeutically effective amount of a compound according to one or more embodiments disclosed in this specification. Examples of viruses that may cause viral infections to be treated include, but are not limited to: a polyomavirus, such as John Cunningham Virus (JCV), Simian virus 40 (SV40), or BK Virus (BKV); influenza, Human Immunodeficiency Virus type 1 (HIV-1), Human Papilloma Virus (HPV), adenovirus, Epstein-Barr Virus (EBV), Hepatitis C Virus (HCV), Molluscum contagiosum virus (MCV); Human T-lymphotropic virus type 1 HTLV-1), Herpes Simplex Virus type 1 (HSV-1), cytomegalovirus (CMV), hepatitis B virus, Bovine papillomavirus (BPV-1), human T-cell lymphotropic virus type 1, Japanese encephalitis virus, respiratory syncytial virus (RSV), and West Nile virus.

The compounds or pharmaceutical compositions according to one or more embodiments disclosed in this specification may further be used in a method for treating a betacoronavirus infection in a patient by administering to the patient a therapeutically effective amount of a compound or pharmaceutical composition according to one or more embodiments disclosed in this specification.

The compounds according to one or more embodiments disclosed in this specification may further be used in the preventing of a betacoronavirus infection in a patient by administering to the patient a prophylactically effective amount of a compound or pharmaceutical composition according to one or more embodiments disclosed in this specification.

The compounds according to one or more embodiments disclosed in this specification may be used for the manufacture of a medicament for the treatment or prophylaxis of a betacoronavirus infection.

In an embodiment, the compounds or the pharmaceutical composition may further comprise or be administered in combination with one or more other antiviral agents including, but not limited to, oseltamivir phosphate, zanamivir or Virazole®, Remdesivir, Vidarabine, Acyclovir, Ganciclovir, Valganciclovir, Valacyclovir, Cidofovir, Famciclovir, Ribavirin, Amantadine, Rimantadine, Interferon, Oseltamivir, Palivizumab, Rimantadine, Zanamivir, nucleoside-analog reverse transcriptase inhibitors (NRTI) such as Zidovudine, Didanosine, Zalcitabine, Stavudine, Lamivudine and Abacavir, non-nucleoside reverse transcriptase inhibitors (NNRTI) such as Nevirapine, Delavirdine and Efavirenz, protease inhibitors such as Saquinavir, Ritonavir, Indinavir, Nelfinavir, Amprenavir, and other known antiviral compounds and preparations.

In an embodiment, the compounds or the pharmaceutical compositions may be co-administered with one or more antiviral agents. By “co-administered” is meant simultaneous administration in the same formulation or in two different formulations via the same or different routes or sequential administration by the same or different routes. By “sequential” administration is meant a time difference of from seconds, minutes, hours or days between the administration of the two or more separate compounds. The compounds or the pharmaceutical compositions of the present invention may be administered in any order.

In an embodiment betacoronavirus is selected from the group consisting of Severe Acute Respiratory Syndrome coronavirus SARS-CoV, Middle East Respiratory Syndrome MERS-CoV, and severe acute respiratory syndrome coronavirus 2 (SARS-CoV-2; originally known as nCoV-2019).

In an embodiment betacoronavirus is SARS-CoV.

In an embodiment betacoronavirus is SARS-CoV-2.

Serine/Threonine phosphatases, including PP2A may be involved in modulation of synaptic plasticity. Decreased PP2A activity is associated with maintenance of Long Term Potentiation (LTP) of synapses, thus treatment PP2A modulators such as those described here may reverse synaptic LTP. Psychostimulant drugs of abuse such as cocaine and methamphetamine are associated with deleterious synaptic LTP, which may underlie the pathology of addiction and relapse therefore PP2A modulators described here may be useful as treatments for psychostimulant abuse.

A compound according to one or more embodiments disclosed in this specification, for example a compound of formula (I), an enantiomer, a diastereomer, a tautomer or a pharmaceutically acceptable salt thereof, for use as a medicament is disclosed.

Use of a compound according to one or more embodiments disclosed in this specification, for example a compound of formula (I), an enantiomer. a diastereomer, a tautomer or a pharmaceutically acceptable salt thereof, in the manufacture of a medicament for preventing or treating a disease or condition in a patient is disclosed.

In an embodiment, the compound according to one or more embodiments disclosed in this specification, for example a compound of formula (I), an enantiomer, a diastereomer, a tautomer or a pharmaceutically acceptable salt thereof, is for use in preventing or treating a disease or condition ameliorated by the modulation of PP2A.

In an embodiment, the disease or condition is selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease and cardiac hypertrophy. In an embodiment, the disease is cancer.

In an embodiment, a compound of formula (I), an enantiomer, a diastereomer, a tautomer or a pharmaceutically acceptable salt thereof, is for use in the manufacture of a medicament for preventing or treating a disease or condition ameliorated by the modulating of PP2A.

The present application relates also to a method of preventing or treating a disease or condition by comprising administering to a patient a therapeutically effective amount of the compound according to one or more embodiments disclosed in this specification, for example a compound of formula (I), an enantiomer, a diastereomer, a tautomer or a pharmaceutically acceptable salt thereof.

In an embodiment, the disease or condition is ameliorated by the modulation of PP2A.

In an embodiment, the disease for treatment with the compound of formula (I) is identified as having or determined to have a suppressed, disordered or inhibited PP2A activity.

In an embodiment, a compound according to one or more embodiments disclosed in this specification, for example a compound of formula (I), an enantiomer, a diastereomer, a tautomer or a pharmaceutically acceptable salt thereof, is for use in the manufacture of a medicament for preventing or treating a disease or condition ameliorated by the modulating of PP2A.

In an embodiment, the patient in need of a treatment of a disease is administered a therapeutically effective amount of the compound according to one or more embodiments disclosed in this specification, for example a compound of formula (I), an enantiomer, a diastereomer, a tautomer or a pharmaceutically acceptable salt thereof.

In an embodiment, a method of treating cancer in a patient having a tumor that expresses PP2A comprises administering to the patient a therapeutically effective amount of a compound of formula (I), an enantiomer, a diastereomer, a tautomer or a pharmaceutically acceptable salt thereof.

In an embodiment, provided is a method for treating a malignant solid tumor in a patient in need thereof, comprising administering an effective amount of a compound or pharmaceutical composition provided herein to the patient. In certain embodiment, the malignant solid tumor is a carcinoma. In certain embodiments, the malignant solid tumor is a lymphoma. In certain embodiments, the malignant solid tumor is a sarcoma.

In some embodiments, cancer is of bladder, blood, bone, bone marrow, brain, breast, colon, esophagus, gastrointestine, gum, head, kidney, pancreas, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant or spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; gianT-cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast-cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast-cell leukemia; megakaryoblastic leukemia; myeloid sarcoma; and hairy cell leukemia.

In some embodiments, the autoimmune disease is colitis, multiple sclerosis, arthritis, rheumatoid arthritis, osteoarthritis, juvenile arthritis, psoriatic arthritis, acute pancreatitis, chronic pancreatitis, diabetes, insulin-dependent diabetes mellitus (IDDM or type I diabetes), insulitis, inflammatory bowel disease, Crohn's disease, ulcerative colitis, autoimmune hemolytic syndromes, autoimmune hepatitis, autoimmune neuropathy, autoimmune ovarian failure, autoimmune orchitis, autoimmune thrombocytopenia, reactive arthritis, ankylosing spondylitis, silicone implant associated autoimmune disease, Sjogren's syndrome, systemic lupus erythematosus (SLE), vasculitis syndromes (e.g., giant-cell arteritis, Behcet's disease & Wegener's granulomatosis), vitiligo, secondary hematologic manifestation of autoimmune diseases (e.g., anemias), drug-induced autoimmunity, Hashimoto's thyroiditis, hypophysitis, idiopathic thrombocytic pupura, metal-induced autoimmunity, myasthenia gravis, pemphigus, autoimmune deafness (e.g., Meniere's disease), Goodpasture's syndrome, Graves' disease, HIV-related autoimmune syndromes and Gullain-Barre disease.

In some embodiments, neurodegenerative disease is selected from the group consisting of Alzheimer's disease, Parkinson's disease, amyotrophic lateral sclerosis (ALS) with frontotemporal dementia, inclusion body myopathy, frontotemporal dementia (IBMPFD), frontotemporal lobar degeneration, synucleopathies, Huntington's disease, amyloidopathies, angiopathies, tauopathies and Lewy bodies dementia.

As used herein, the term “Neurodegenerative disease” has its general meaning in the art and refers to diseases with neurodegeneration which is the progressive loss of structure or function of neurons, including death of neurons. Many neurodegenerative diseases including amyotrophic lateral sclerosis, Parkinson's, Alzheimer's, and Huntington's occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously. Alzheimer's disease (AD) is characterized by amyloid deposits, intracellular neurofibrillary tangles, tau hyperphosphorylation, neuronal loss and a decline in cognitive function.

In certain instances, it may be appropriate to administer at least one of the compounds of formula (I) (an enantiomer, a diastereomer, a tautomer or a pharmaceutically acceptable salt thereof) in combination with another therapeutic agent. By way of example only, if one of the side effects experienced by a patient upon receiving one of the compounds herein for the treatment of cancer is nausea, then it may be appropriate to administer an antiemetic agent in combination. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for cancer involving administration of one of the compounds described herein, increased therapeutic benefit may result by also providing the patient with another therapeutic agent for cancer. In any case, regardless of the disease, disorder or condition being treated, the overall benefit experienced by the patient may simply be additive of the two therapeutic agents or the patient may experience a synergistic benefit.

The instant compounds may be particularly useful in combination with therapeutic and/or anti-cancer agents. Thus, the present disclosure provides a combination of compounds of Formula (I) are used in a combination with therapeutic and/or anti-cancer agents for simultaneous, separate or sequential administration. The compounds of formula (I) and the other anticancer agent can act additively or synergistically. A synergistic combination of the present compounds and another anticancer agent might allow the use of lower dosages of one or both of these agents and/or less frequent dosages of one or both of the instant compounds and other anticancer agents and/or to administer the agents less frequently can reduce any toxicity associated with the administration of the agents to a patient without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

The therapeutic agent and/or anti-cancer agent can be administered according to therapeutic protocols well known in the art. It will be apparent to those skilled in the art that the administration of the therapeutic agent and/or anti-cancer agent can be varied depending on the disease being treated and the known effects of the anti-cancer agent on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

In one embodiment, the compounds according to one or more embodiments disclosed in this specification, for example compounds of formula I, may be administered in combination with one or more agent selected from aromatase inhibitors, anti-estrogens, antiprogesterons, anti-androgens, or gonadorelin agonists, anti-inflammatory agents, antihistamines, anti-cancer agent, inhibitors of angiogenesis, topoisomerase 1 and 2 inhibitors, microtubule active agents, alkylating agents, antineoplastic, antimetabolite, dacarbazine (DTIC), platinum containing compound, lipid or protein kinase targeting agents, protein or lipid phosphatase targeting agents, anti-angiogenic agents, agents that induce cell differentiation, bradykinin 1 receptor and angiotensin II antagonists, cyclooxygenase inhibitors, heparanase inhibitors, lymphokines or cytokine inhibitors, bisphosphanates, rapamycin derivatives, antiapoptotic pathway inhibitors, apoptotic pathway agonists, PPAR agonists, HSP90 inhibitor, smoothened antagonist, inhibitors of Ras isoforms, telomerase inhibitors, protease inhibitors, metalloproteinase inhibitors, aminopeptidase inhibitors, immunomodulators, therapeutic antibody and a protein kinase inhibitor, e.g., a tyrosine kinase or serine/threonine kinase inhibitor.

In another embodiment is provided a combination of a compound of formula I and an anti-cancer agent for simultaneous, separate or sequential administration.

A person of ordinary skill in the art would be able to discern which combinations of agents would be useful based on the particular characteristics of the drugs and the cancer involved. Classes of such agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs), agents that interfere with cell cycle checkpoints, PARP inhibitors, HDAC inhibitors, Smo antagonists (HH inhibitors), HSP90 inhibitors, CYP17 inhibitors, 3rd generation AR antagonists, JAK inhibitors e.g. Ruxolitinib (trade name Jakafi, and BTK kinase inhibitors.

Anticancer agents suitable for use in the combination therapy with compounds as disclosed herein include, but are not limited to:

1)alkaloids and natural product drugs, including, microtubule inhibitors (e.g., Vincristine, Vinblastine, and Vindesine, and vinorelbine etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-161, and Teniposide [VM261, etc.), and agents that target topoisomerase I (e.g., Camptothecin, topotecan (Hycamtin) and Irinotecan [CPT-11], rubitecan (Orathecin) etc.);

2) covalent DNA-binding agents [alkylating agents], including, nitrogen mustards (e.g., Mechloretharnine, chlormethine, Chlorambucil, Cyclophosphamide, estramustine (Emcyt, Estracit), ifosfamide, Ifosphamide, melphalan (Alkeran) etc.); alkyl sulfonates like Busulfan [Myleran], nitrosoureas (e.g., Carmustine or BCNU (bis-chloroethylnitrosourea), fotemustine Lomustine, and Semustine, streptozocin etc.), and other alkylating agents (e.g., Dacarbazine, procarbazine ethylenimine/methylmelamine, thriethylenemelamine (TEM), triethylene thiophosphoramide (thiotepa), hexamethylmelamine (HMM, altretamine), and Mitocycin, uramustine etc.) including Temozolomide (brand names Temodar and Temodal and Temcad), altretamine (also hexalen) and mitomycin; and

3) noncovalent DNA-binding agents [antitumor antibiotics], including nucleic acid inhibitors (e.g., Dactinomycin [Actinomycin Dl, etc.), anthracyclines (e.g., Daunorubicin [Daunomycin, and Cerubidine], Doxorubicin [Adrianycin], epirubicin (Ellence), and Idarubicin [Idamycin], valrubicin (Valstar) etc.), anthracenediones (e.g., anthracycline analogues, such as, [Mitoxantrone], etc.), bleomycins (Blenoxane), etc., amsacrine and plicamycin (Mithramycin), dactinomycin, mitomycin C.

In certain embodiments, a patient with cancer is treated with a combination of a compound formula (I) and radiation therapy. In certain embodiments, the method comprises administering to a patient with cancer a therapeutically effective amount of a compound of the disclosure, and adjunctively treating the patient with an effective amount of radiation therapy. In certain embodiments, the compound is administered to the patient in need thereof prior to, concurrently with, or subsequent to the treatment with radiation.

As used herein, the term “increase” or the related terms “increased,” “enhance” or “enhanced” may refer to a statistically significant increase, and the terms “decreased,” “suppressed,” or “inhibited” to a statistically significant decrease. For the avoidance of doubt, an increase generally refers to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control, baseline, or prior-in-time value. Inhibition generally refers to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease over the control value.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single formulation (e.g., a capsule or injection) having a fixed ratio of active ingredients or in multiple, separate dosage forms for each active ingredient. In addition, such administration also encompasses use of each type of therapeutic agent in a sequential manner. In either case, the treatment regimen will provide beneficial effects of the drug combination in treating the conditions or disorders described herein.

The terms “effective amount” or “therapeutically effective amount” as used herein, refer to a sufficient amount of at least one compound being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer cell. In certain instances, an “effective amount” for therapeutic uses is the amount of the composition comprising a compound as set forth herein required to provide a clinically significant decrease in a disease. An appropriate “effective” amount in any individual case is determined using any suitable technique, such as a dose escalation study.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to an individual at risk of developing a particular disease, to an individual reporting one or more of the physiological symptoms of a disease, or to an individual at risk of reoccurrence of the disease.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the compounds and compositions described herein are administered orally.

As used herein, the term “patient” means all mammals including humans. Examples of patients include humans, cows, dogs, cats, goats, sheep, pigs, and rabbits. In some embodiments, the patient is a human.

The pharmaceutical formulations may include those suitable for oral, parenteral (including subcutaneous, intradermal, intramuscular, intravenous, intraarticular, and intramedullary), intraperitoneal, transmucosal, transdermal, intranasal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of formula (I) or a pharmaceutically acceptable salt, ester, amide, solvate, or enantiomer or diastereomer or tautomer thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds of formula (I) suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds of formula (I) may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, a compound of formula (I) may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

In an embodiment, compounds as disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds of formula (I) may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds disclosed herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Intranasal delivery, in particular, may be useful for delivering compounds to the CNS. It had been shown that intranasal drug administration is a noninvasive method of bypassing the blood-brain barrier (BBB) to deliver neurotrophins and other therapeutic agents to the brain and spinal cord. Delivery from the nose to the CNS occurs within minutes along both the olfactory and trigeminal neural pathways. Intranasal delivery occurs by an extracellular route and does not require that drugs bind to any receptor or undergo axonal transport. Intranasal delivery also targets the nasal associated lymphatic tissues (NALT) and deep cervical lymph nodes. In addition, intranasally administered therapeutics are observed at high levels in the blood vessel walls and perivascular spaces of the cerebrovasculature. Using this intranasal method in animal models, researchers have successfully reduced stroke damage, reversed Alzheimer's neurodegeneration, reduced anxiety, improved memory, stimulated cerebral neurogenesis, and treated brain tumors.

In an embodiment, unit dosage formulations are those containing an effective dose or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds according to one or more embodiments disclosed in this specification may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compound which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compound of formula (I) can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

The description below discloses some embodiments in such a detail that a person skilled in the art is able to utilize the invention based on the disclosure. Not all steps of the embodiments are discussed in detail, as many of the steps will be obvious for the person skilled in the art based on this specification.

### EXAMPLES

### Abbreviations

### PE: Petroleum Ether

### EA: Ethyl Acetate

### DMF: N,N-dimethylformamide

### THF: Tetrahydrofuran

### DCM: Dichloromethane

TFA: Trifluoroacetic acid

### TEA: Triethylamine

DIEA: N-ethyl-N-isopropylpropan-2-amine
DIPEA: N-ethyl-N-isopropylpropan-2-amine
NMMO: 4-methylmorpholine 4-oxide

### ACN: Acetonitrile

### SFC: Supercritical Fluid Chromatography

### TPP: Triphenylphosphine

mCPBA/m-CPBA: meta-chloroperbenzoic acid

### DMAP: N, N-dimethylaminopyridine

KHMDS: potassium hexamethyldisilazan

TABLE 3
Structures of target compounds.

| Target compound | Structure |
|---|---|
| 2 (T2) compound 37 N-[3-(3-chloro-10,11- dihydro-5H-di- benzo[b,f]azepin-5- yl)propyl]-4-(trifluoro- methoxy) benzenesulfonimidamide | 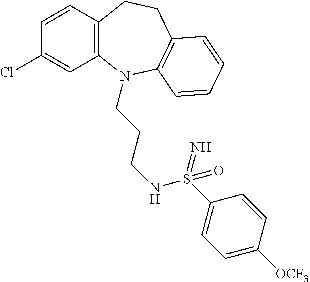 [SMILES: N=S(=O)(NCCCN1c2ccccc2CCc2ccc(Cl)cc21)c1ccc(OC(F)(F)F)cc1] |
| | 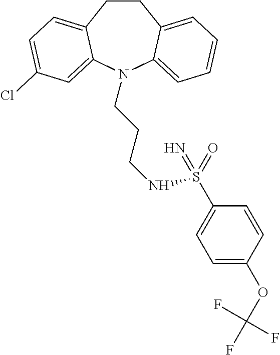 [SMILES: N=[S@](=O)(NCCCN1c2ccccc2CCc2ccc(Cl)cc21)c1ccc(OC(F)(F)F)cc1] |
| | 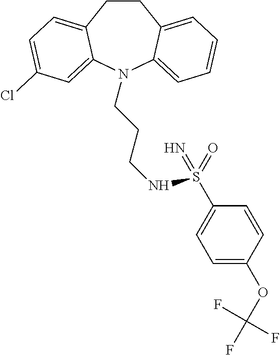 [SMILES: N=[S@@](=O)(NCCCN1c2ccccc2CCc2ccc(Cl)cc21)c1ccc(OC(F)(F)F)cc1] |
| 3 (T3) compound 38 N′-methyl-N-[3-(3-chloro- 10,11- dihydro-5H- dibenzo[b,f]azepin-5- yl)propyl]-4-(trifluoro- methoxy) benzenesulfonimidamide | 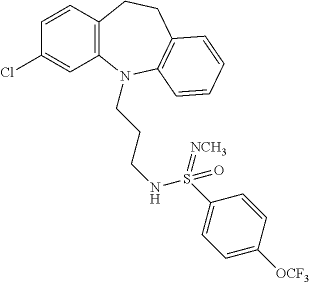 |
| 5 (T5) N-[(2R)-3-(10,11-dihydro- 5H-dibenzo[b,f]azepin-5- yl)-2-hydroxypropyl]-4- (trifluoromethoxy) ben- zenesulfonimidamide | 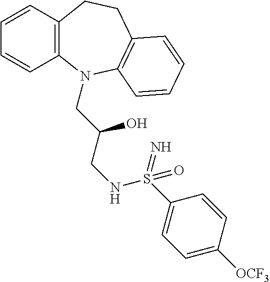 [SMILES: N=S(=O)(NC[C@H](O)CN1c2ccccc2CCc2ccccc21)c1ccc(OC(F)(F)F)cc1] |
| | 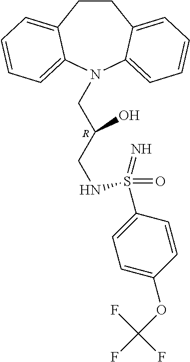 |
| | 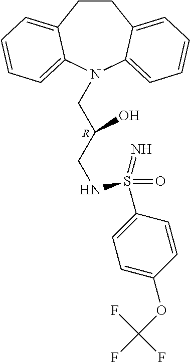 |
| 6 (T6) N′-methyl-N-[(2R)-3- (10,11-dihydro-5H-di- benzo[b,f]azepin-5-yl)- 2-hydroxypropyl]-4- (trifluoromethoxy) benzenesulfonimidamide | 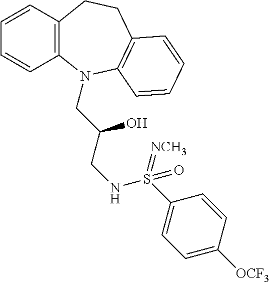 |
| | 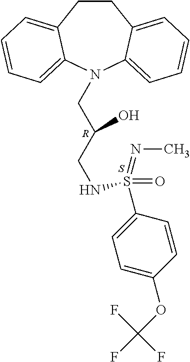 |
| | 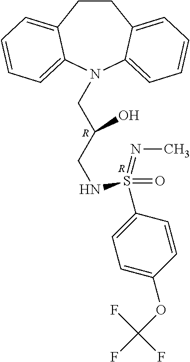 |
| 8 (T8) N-[(1S,2S,3R)-3-(10,11- dihydro-5H-di- benzo[b,f]azepin-5-yl)-2- hydroxycyclohexyl]-4- (trifluoromethoxy)ben- zene-1-sulfonimidoamide | 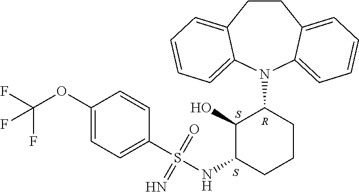 |
| 9 (T9) N-[(1S,2S,3R)-3-(10,11- dihydro-5H-di- benzo[b,f]azepin-5-yl)-2- hydroxycyclohexyl]-N′- methyl-4-(trifluoromethoxy) benzene-1-sulfonimidoamide | 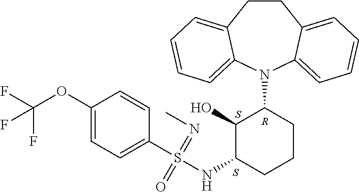 |
| 11 (T11) N-[2-hydroxy-3-(10H- phenoxazin-10-yl)cyclo- hexyl]-4-(trifluoromethoxy) benzene-1-sulfonimidoamide | 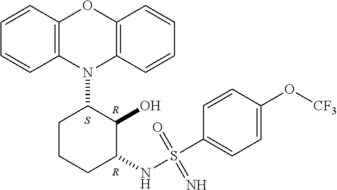 |
| | 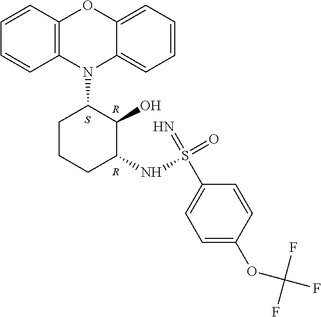 |
| | 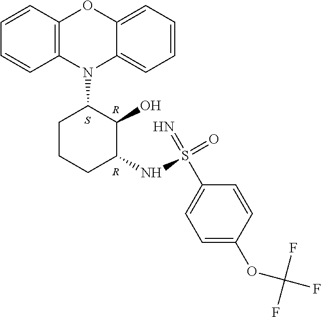 |
| 12 (T12) N-[2-hydroxy-3-(10H- phenoxazin-10-yl)cyclo- hexyl]-N′-methyl-4- (trifluoromethoxy) benzene-1- sulfonimidoamide | 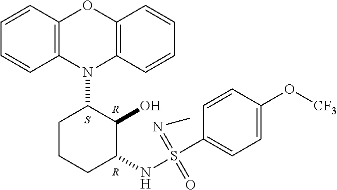 |
| N′-((1R,2R,3S)-2-hydroxy- 3-(10H-phenoxazin-10- yl)cyclohexyl)- N-methyl-4-(trifluoro- methoxy) benzenesulfonimidoamide | 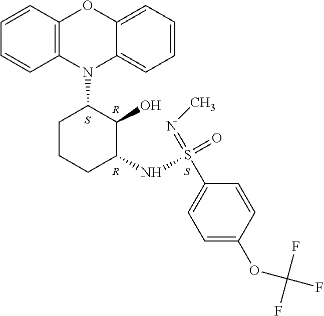 |
| N′-((1R,2R,3S)-2-hydroxy- 3-(10H-phenoxazin-10- yl)cyclohexyl)- N-methyl-4-(trifluoro- methoxy) benzenesulfonimidoamide | 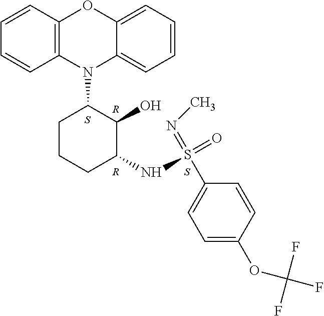 |
| 14 (T14) N-[(3R,4R,5S)-4-hydroxy- 5-(10H-phenoxazin-10- yl)oxan-3-yl]-4- (trifluoromethoxy)benzene-1- sulfonimidoamide | 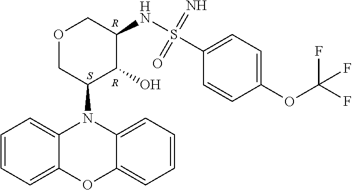 |
| | 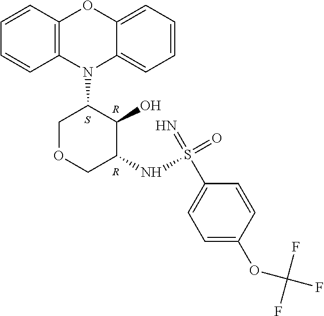 |
| | 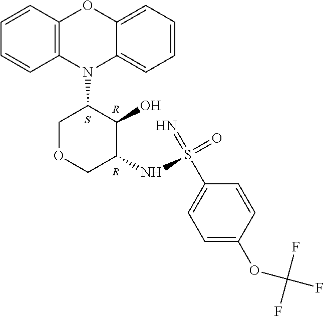 |
| 15 (T15) N-[(3R,4R,5S)-4-hydroxy- 5-(10H-phenoxazin-10- yl)oxan-3-yl]-N′-methyl- 4-(trifluoromethoxy)- benzene-1-sulfonimidoamide | 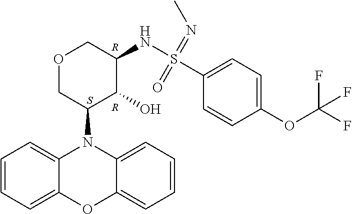 |
| N′-((3R,4R,5S)-4-hydroxy- 5-(10H-phenoxazin-10- yl)tetrahydro- 2H-pyran-3-yl)-N-methyl- 4-(trifluoromethoxy) benzenesulfonimidoamide | 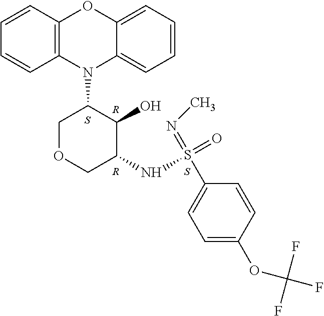 |
| N′-((3R,4R,5S)-4-hydroxy- 5-(10H-phenoxazin-10- yl)tetrahydro- 2H-pyran-3-yl)-N-methyl- 4-(trifluoromethoxy) benzenesulfonimidoamide | 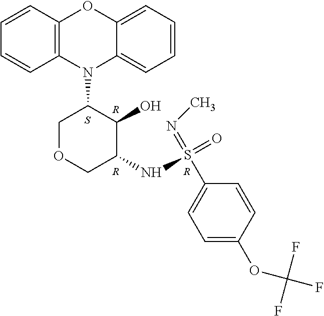 |
| 17 (T17) N-[(3R,4R,5S)-5-(3,6- dichloro-9H-carbazol-9- yl)-4-hydroxyoxan-3-yl]- 4-(trifluoromethoxy) benzene-1-sulfonimidoamide | 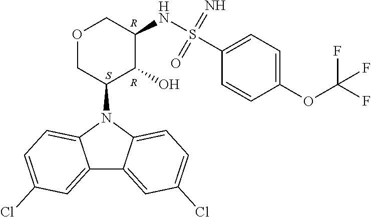 |
| | 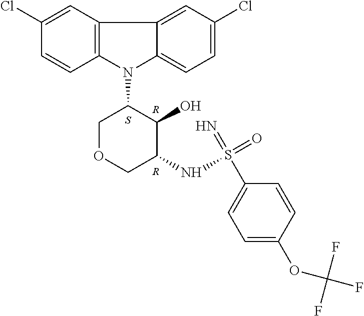 |
| | 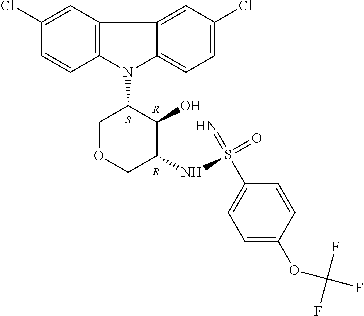 |
| 18 (T18) N-[(3R,4R,5S)-5-(3,6- dichloro-9H-carbazol-9- yl)-4-hydroxyoxan-3-yl]- N′-methyl-4-(trifluoro- methoxy)benzene-1- sulfonimidoamide | 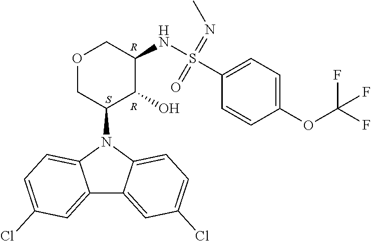 |
| N′-((3R,4R,5S)-5-(3,6- dichloro-9H-carbazol-9- yl)-4-hydroxytetrahydro- 2H-pyran-3-yl)-N-methyl- 4-(trifluoromethoxy) benzenesulfonimidoamide | 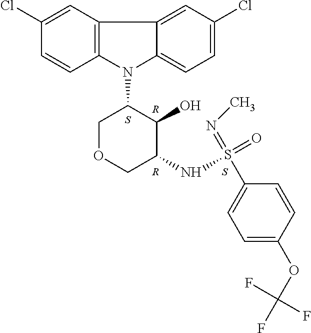 |
| N′-((3R,4R,5S)-5-(3,6- dichloro-9H-carbazol-9- yl)-4-hydroxytetrahydro- 2H-pyran-3-yl)-N-methyl- 4-(trifluoromethoxy) benzenesulfonimidoamide | 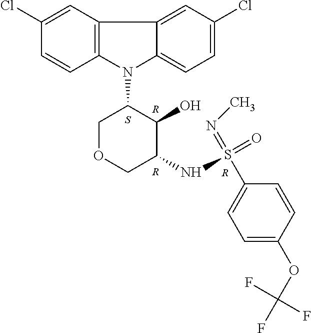 |
| 15A (T15A) | 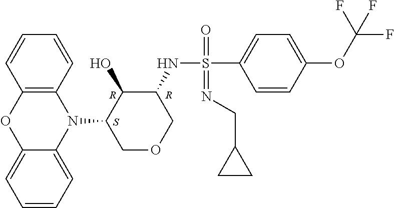 |
| N-(cyclopropylmethyl)-N′- ((3R,4R,5S)-4-hydroxy-5- (10H-phenoxazin-10- yl)tetrahydro-2H-pyran-3- yl)-4-(trifluoromethoxy) benzenesulfonimidoamide | 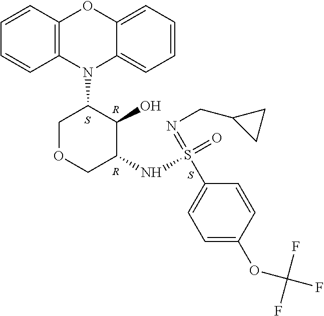 |
| N-(cyclopropylmethyl)-N′- ((3R,4R,5S)-4-hydroxy-5- (10H-phenoxazin-10- yl)tetrahydro-2H-pyran-3- yl)-4-(trifluoromethoxy) benzenesulfonimidoamide | 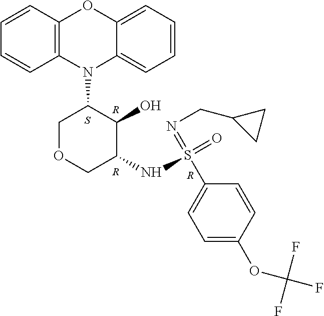 |
| 15C (T15C) N-((3R,4R,5S)-4-hydroxy- 5-(10H-phenoxazin-10- yl)tetrahydro-2H-pyran-3- yl)-N′-propyl-4- (trifluoromethoxy) benzenesulfonimidoamide | 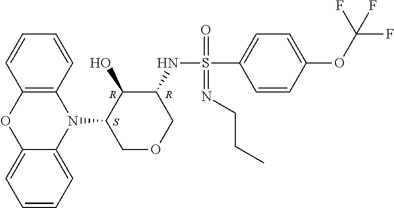 |
| N′-((3R,4R,5S)-4-hydroxy- 5-(10H-phenoxazin-10- yl)tetrahydro-2H-pyran-3- yl)-N-propyl-4- (trifluoromethoxy) benzenesulfonimidoamide | 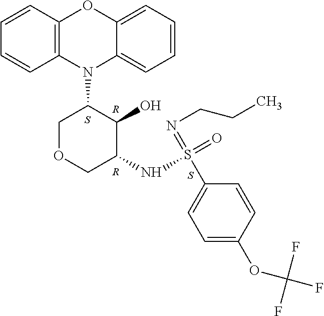 |
| N′-((3R,4R,5S)-4-hydroxy- 5-(10H-phenoxazin-10- yl)tetrahydro-2H-pyran-3- yl)-N-propyl-4- (trifluoromethoxy) benzenesulfonimidoamide | 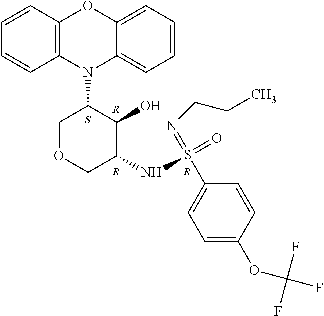 |
| 18A (T18A) N-(cyclopropylmethyl)-N′- ((3R,4R,5S)-5-(3,6- dichloro-9H-carbazol-9-yl)-4- hydroxytetrahydro-2H- pyran-3-yl)-4-(trifluoro- methoxy) benzenesulfonimidoamide | 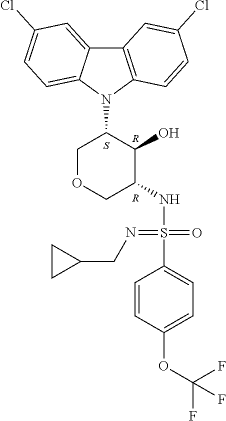 |
| N-(cyclopropylmethyl)-N′- ((3R,4R,5S)-5-(3,6- dichloro-9H-carbazol-9-yl)-4- hydroxytetrahydro-2H- pyran-3-yl)-4-(trifluoro- methoxy) benzenesulfonimidoamide | 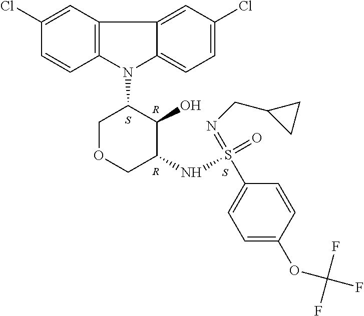 |
| N-(cyclopropylmethyl)-N′- ((3R,4R,5S)-5-(3,6- dichloro-9H-carbazol-9-yl)-4- hydroxytetrahydro-2H- pyran-3-yl)-4-(trifluoro- methoxy) benzenesulfonimidoamide | 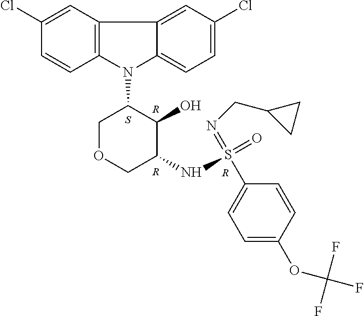 |
| 18C (T18C) N-((3R,4R,5S)-5-(3,6- dichloro-9H-carbazol-9- yl)-4-hydroxytetrahydro- 2H-pyran-3-yl)-N′-propyl- 4-(trifluoromethoxy) benzenesulfonimidoamide | 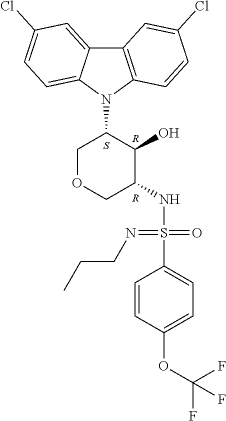 |
| N′-((3R,4R,5S)-5-(3,6- dichloro-9H-carbazol-9- yl)-4-hydroxytetrahydro- 2H-pyran-3-yl)-N-propyl- 4-(trifluoromethoxy) benzenesulfonimidoamide | 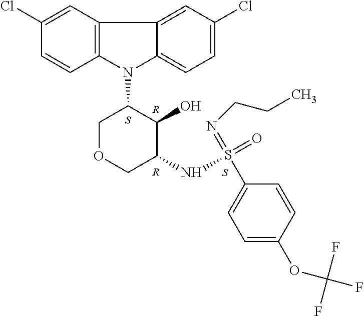 |
| N′-((3R,4R,5S)-5-(3,6- dichloro-9H-carbazol-9- yl)-4-hydroxytetrahydro- 2H-pyran-3-yl)-N-propyl- 4-(trifluoromethoxy) benzenesulfonimidoamide | 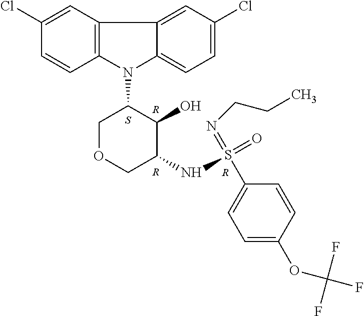 |

TABLE 4
Structures of control compounds.

| Control compound | Structure |
|---|---|
| T1 (CAS 1423077-49-9) | 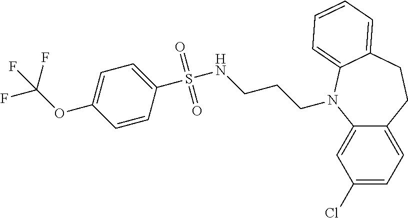 [SMILES: O=S(=O)(NCCCN1c2ccccc2CCc2ccc(Cl)cc21)c1ccc(OC(F)(F)F)cc1] |
| T4 (CAS 1423077-95-5) | 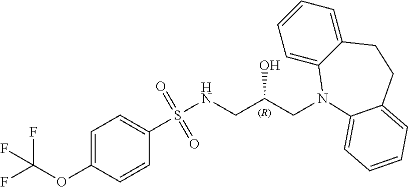 |
| T7 (CAS 1809068-70-9) | 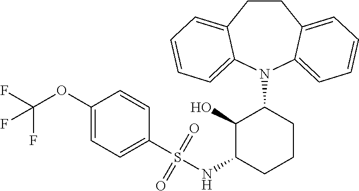 [SMILES: O=S(=O)(N[C@H]1CCC[C@@H](N2c3ccccc3CCc3ccccc32)[C@@H]1O)c1ccc(OC(F)(F)F)cc1] |
| T10 (CAS 1809427-19-7) | 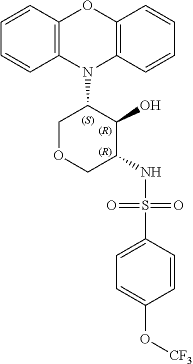 |
| T13 (CAS 2088956-23-2) | 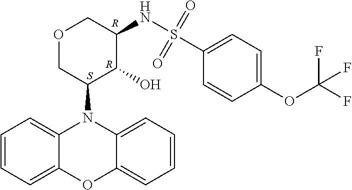 |
| T16 (CAS 2088956-21-0) | 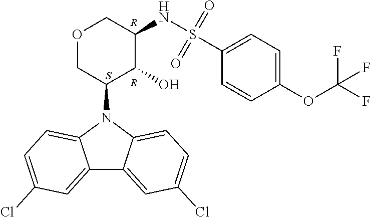 |

### Example 1—Synthesis of T1 (CAS 1423077-49-9)

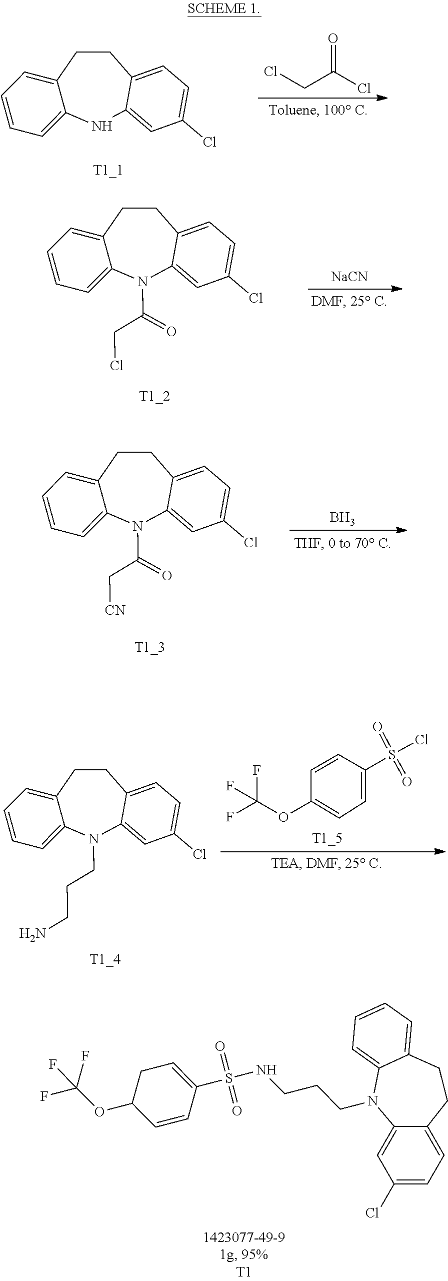

### Synthesis of Compound T1_2:

To a solution of compound T1_1 (50 g, 217.7 mmol) in Toluene (500 mL) was added 2-chloroacetyl chloride (25.8 g, 228.6 mmol) with stirring, then the reaction mixture was heated to 100° C. for 18 h. TLC showed that the reaction has been completed. The mixture was cooled to 25° C., concentrated under reduced pressure to remove most of the solvent. The residue was suspended in PE (100 mL), the white precipitate had formed that was collected by filtration, then it was dried at 40° C. under reduced pressure to afford compound T1_2 as an off-white solid (51 g, 76%).

### Synthesis of Compound T1_3:

To a solution of compound T1_2 (60 g, 196 mmol) in DMF (600 mL) was added NaCN (25.8 g, 205.8 mmol) with stirring, then the reaction mixture was stirred at 25° C. for 15 h. TLC showed that the reaction has been completed. The mixture was poured into ice-water (2000 mL), the white precipitate had formed that was collected by filtration, then it was dried at 40° C. under reduced pressure to afford compound T1_3 as an off-white solid (40 g, 69%).

### Synthesis of Compound T1_4:

To a solution of compound T1_3 (20 g, 67.4 mmol) in THF (300 mL) was added BH₃-Methyl sulfide complex (10 mol/L in THF, 27 mL) dropwise at 0° C., then the reaction mixture was heated to 70° C. for 5 h. TLC showed that the reaction has been completed. The result mixture was cooled to 0° C., a solution of aqueous HCl (1 mol/L, 50 mL) was added dropwise, it was stirred for an additional 1.5 h, and then heated to 70° C. for 1 h. The mixture was cooled to 25° C., EtOAc (100) was added, the organic layer was washed with water (80 mL×3), the aqueous layer was adjusted with aqueous NaOH (4 mol/L) to pH >8, the mixture was extracted with EA (100 mL×3), the combined organic layers was concentrated in vacuo, the residue was purified by column chromatography on silica gel (DCM:MeOH=30:1) to give compound T1_4 as a yellow oil (5.6 g, 30%).

### Synthesis of Compound T1:

To a solution of compound T1_4 (2.0 g, 7.0 mmol) and TEA (1.5 g, 14.6 mmol) in DMF (20 mL) was added T1_5 (2.73 g, 10.5 mmol) at 0° C., then the reaction mixture was stirred at 25° C. for 15 h. TLC showed that the reaction has been completed. The mixture was poured into water (40 mL), extracted with DCM (30 mL×2), the combined organic layers was washed with brine (20 mL×3), dried over Na₂SO₄, concentrated under reduced pressure, the residue was purified by column chromatography on silica gel (PE to PE:EA=5:1) to give T1 as a white solid (7.3 g, 73%).

### Example 2—Synthesis of Target 2 (T2)

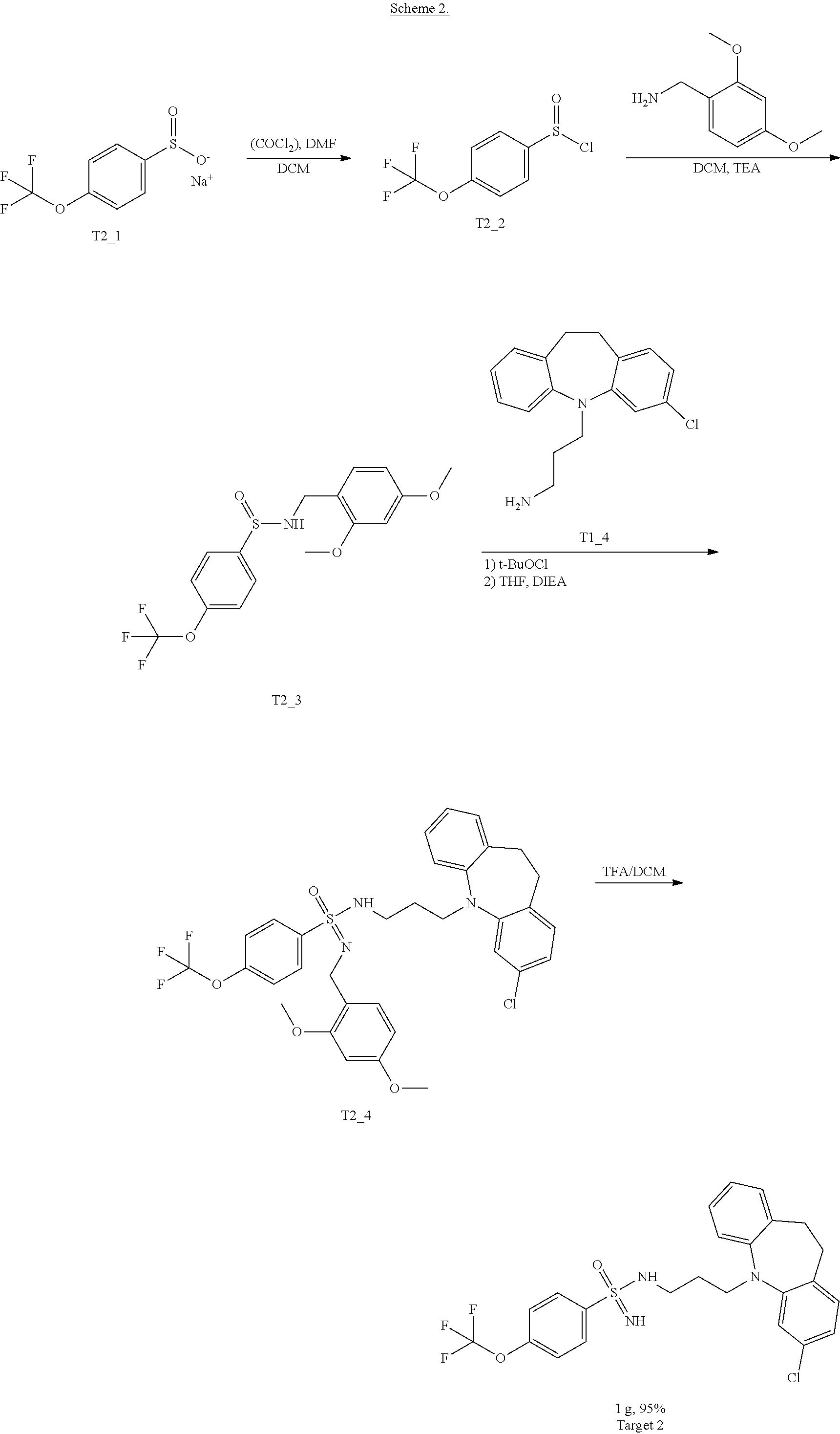

### Synthesis of Compound T2_1:

To a mixture of compound T1_5 (100 g, 383.7 mmol), Na₂SO₃ (106.4 g, 844 mmol) and NaHCO₃ (70.8 g, 844 mmol) in water (1000 mL) was stirred for 18 h at 65° C. under Ar atmosphere. The mixture was concentrated to remove water below 60° C. under vacuum with oil pump. Then the residue was stirred with MeOH (1000 mL) for 2 h at 15° C. to 25° C., the reaction mixture was filtered and the filtrate was concentrated to afford compound T2_1 as white solid (105 g, 95%).

### Synthesis of Compound T2_3:

To a mixture of compound T2_1 (20 g, 80.6 mmol) and DMF (0.2 mL) in DCM (200 mL) was added (COCl)₂ (15.4 g, 121.3 mmol) below 20° C. The mixture was stirred for 1 h after the addition, then the reaction mixture was concentrated under vacuum to give crude compound T2_2. The crude compound T2_2 was dissolved with another 200 mL of DCM and then (2,4-dimethoxyphenyl) methanamine (20.2 g, 121.3 mmol) and TEA (24.6 g, 241.8 mmol) was added into the mixture. The mixture was stirred for 18 h at 15 to 25° C. The reaction was quenched with water (100 mL) and separated to collect organic phase, the aqueous was extracted with another DCM (100 mL), combined organic phase and washed with brine (20 mL), dried over Na₂SO₄, concentrated and purified by column chromatography on silica gel (PE/EA=10/1 to PE/EA=5/1) to give compound T2_3 as white solid (13.6 g, 45%).

### Synthesis of Compound T2_4:

A mixture of T2_3 (12.5 g, 33.3 mmol) and t-BuOCl (4.2 g, 35 mmol) in CCL₄ (150 mL) was stirred for 1 h at 0° C. (in dark). The reaction mixture was concentrated with oil pump below 5° C. to remove CCl₄. The residue was dissolved into THF (100 mL), then compound T1_4 (10 g, 35 mmol) and DIPEA (12.9 g, 99.9 mmol) was added at 0° C., then the mixture was stirred for 18 h at 15° C. to 25° C. The reaction was quenched with water (100 mL) and extracted with EA (100 mL×3), combined the organic phase and washed with brine (20 mL), dried over Na₂SO₄, purified by column chromatography on silica gel (PE/EA=5/1 to PE/EA=3/1) to afford compound T2_4 as white solid (7 g, 32%).

### Synthesis of Compound Target 2 (T2):

Compound T2_4 (5 g, 7.57 mmol) was dissolved into DCM (50 mL) at 10° C. to 20° C., followed by TFA (50 mL), the mixture was stirred for 24 h at 10 to 20° C. The pH of reaction was adjusted with sat. NaHCO₃ aqueous until 7 to 8. The resulting mixture was filtered and filtrate was extracted with DCM (100 mL×3), combined the organic phase and washed with brine (50 mL), dried over Na₂SO₄, concentrated and purified by column chromatography on silica gel (PE/EA=5/1 to PE/EA=2/1) to afford Target 2 as white solid (1.1 g, 28%). ¹H NMR (400 MHz, CDCl₃) δ 7.93 (2H, d, J=8.8 Hz), 7.28-7.26 (2H, m), 7.18-7.13 (2H, m), 7.13-6.99 (4H, m), 6.92-6.90 (1H, m), 3.72 (2H, t, J=6.4, 6.0 Hz), 3.07-2.99 (6H, m), 1.74-1.71 (2H, m).

### Example 3—Synthesis of Target 3 (T3)

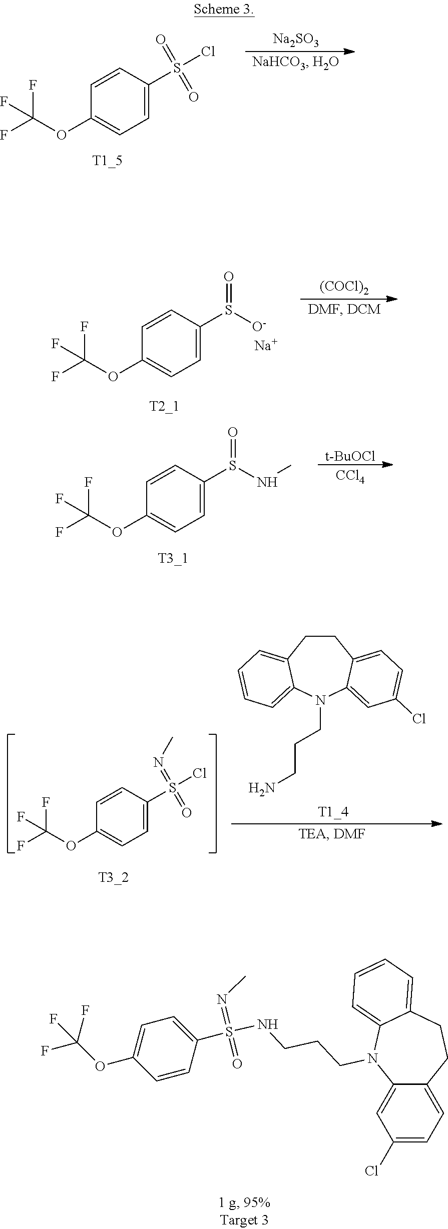

### Synthesis of Compound T3_1:

To a mixture of T2_1 (10.3 g, 30.3 mmol) and DMF (0.15 mL) in DCM (100 mL) was added (COCl)₂ (6.1 g, 48.36 mmol) below 20° C. The mixture was stirred for 1 h after the addition, then the reaction mixture was concentrated under vacuum to give crude compound T2_2. The crude compound T2_2 was dissolved with DCM (100 mL) and then MeNH₂ (2M in THF, 19 g, 40 mmol) and DIEA (7.8 g, 60.45 mmol) was added into the mixture. The mixture was stirred for 18 h at 15 to 25° C. The reaction was quenched with water (100 mL) and separated to collect organic phase, the aqueous phase was extracted with another DCM (100 mL), combined organic phase and washed with brine (30 mL), dried over Na₂SO₄, concentrated and purified by chromatography on silica gel (PE/EA=10/1) to give compound T3_1 as yellow oil (3.5 g, 32%).

### Synthesis of Compound Target 3 (T3):

A mixture of compound T3_1 (10 g, 41.8 mmol) and t-BuOCl (10.9 g, 43.9 mmol) in CCl₄ (100 mL) was stirred for 1 h at 0° C. (in dark). The reaction mixture was concentrated with oil pump below 5° C. to remove CCl₄. The residue was dissolved into THF (100 mL), then compound T1_4 (12 g, 43.9 mmol) and DIPEA (16.2 g, 125.4 mmol) was added at 0° C., then the mixture was stirred for 18 h at 15° C. to 25° C. The reaction was quenched with water (100 mL) and extracted with EA (100 mL×2), combined the organic phase and washed with brine (50 mL), dried over Na₂SO₄, purified by column chromatography on silica gel (PE/EA=10/1) to afford Target 3 as off-white solid (1.2 g, 6%). ¹H NMR (400 MHz, CDCl₃) δ 7.89 (2H, d, J=8.8 Hz), 7.29-7.27 (2H, m), 7.17-7.08 (4H, m), 7.01-6.97 (2H, m), 6.70-6.87 (1H, m), 3.86-3.82 (2H, m), 3.15-3.08 (6H, m), 2.60 (3H, s), 1.84-1.81 (2H, m).

### Example 4—Synthesis of T4 (CAS 1423077-95-5)

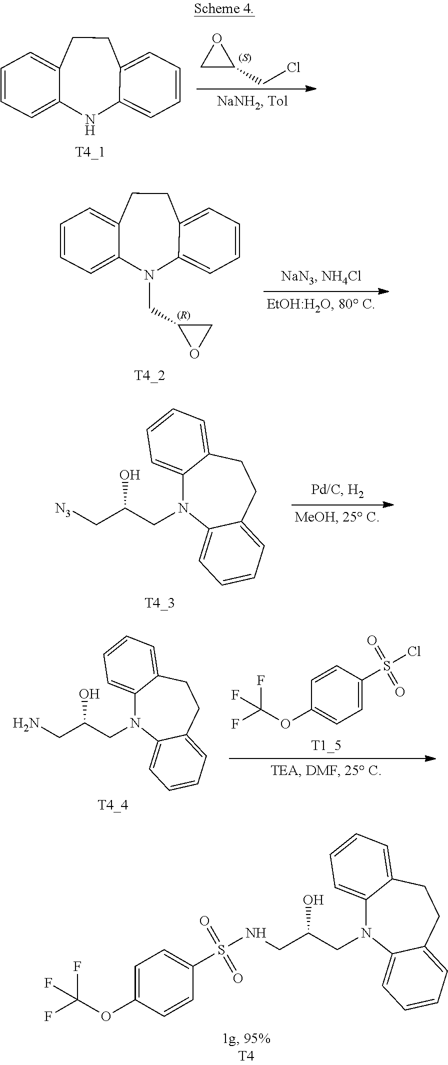

### Synthesis of Compound T4_2:

To a solution of compound T4_1 (50 g, 256.1 mmol) and (S)-2-(chloromethyl) oxirane (47.4 g, 512.1 mmol) in Toluene (500 mL) was added NaNH₂ (25.0 g, 640.2 mmol) in small portions with stirring, then the reaction mixture was refluxed for 15 h. TLC showed that the reaction has been completed. The mixture was cooled to 25° C., the organic layer was washed with brine (100 mL×3), then the organic layer was concentrated in vacuo, the residue was purified by column chromatography on silica gel (PE:EA=20:1) to give compound T4_2 as a yellow oil (30 g, 46%).

### Synthesis of Compound T4_3:

A suspension of compound T4_2 (30 g, 119.4 mmol), NaN₃ (11.6 g, 179.1 mmol) and NH₄Cl (8.3 g, 155.2 mmol) in EtOH and water (4:1, 300 mL) was stirred at 80° C. for 15 h. TLC showed that the reaction has been completed. The mixture was cooled to 25° C. and concentrated in vacuo. The residue was dissolved in EA (100 mL), the organic layer was washed with brine (20 mL×3), then it was concentrated to give compound T4_3 as a yellow oil (22 g, 63%) which was used for next step without further purification.

### Synthesis of Compound T4_4:

To a solution of compound T4_3 (24 g, 119.4 mmol) in MeOH (240 mL) was added Pd/C (6.0 g, 10 wt %) at 25° C. under N₂. The suspension was degassed under vacuum and purged with H₂ several times, and then the mixture was stirred under H₂ (16 psi, 1.1 bar) at 25° C. for 15 h. The mixture was filtered and the filtrate was concentrated to give compound T4_4 as a white solid (18 g, 82%) which was used for next step without further purification.

### Synthesis of Compound T4:

To a solution of compound T4_4 (2.5 g, 9.32 mmol) and TEA (1.41 g, 14.0 mmol) in DMF (25 mL) was added Compound T1_5 (9.32 g, 10.5 mmol) at 0° C., then the reaction mixture was stirred at 25° C. for 15 h. TLC showed that the reaction has been completed. The mixture was poured into water (40 mL), extracted with DCM (30 mL×2), the combined organic layers was washed with brine (20 mL×3), dried over Na₂SO₄, concentrated under reduced pressure, the residue was purified by column chromatography on silica gel (PE:EA=5:1) to give Target 4 as a white solid (1.5 g, 32%).

### Example 5—Synthesis of Target 5 (T5)

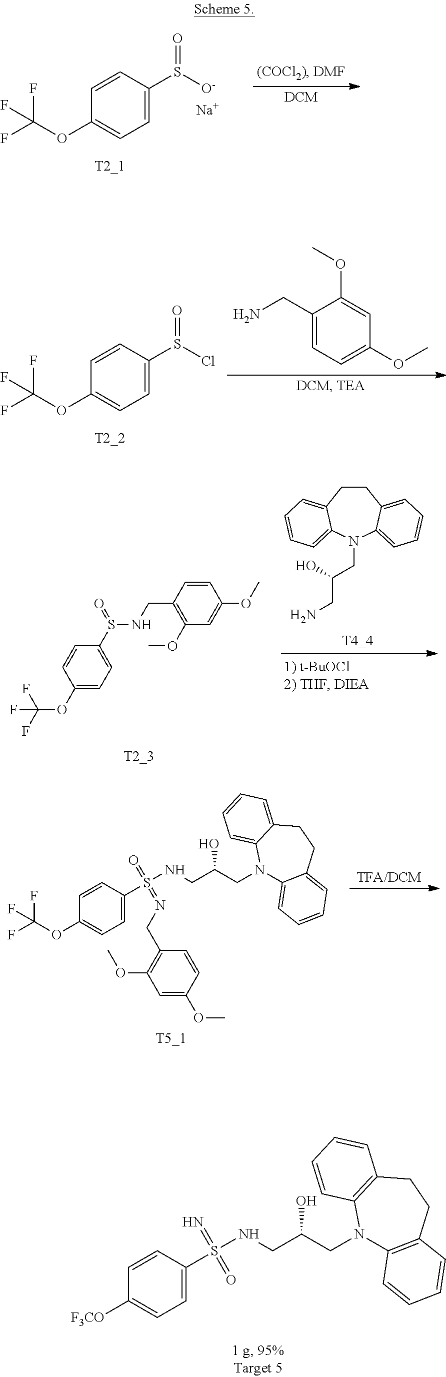

### Synthesis of Compound T5_1:

A mixture of T2_3 (3 g, 8 mmol, Example 2) and t-BuOCl (1 g, 8.4 mmol) in of CCL₄ (50 mL) was stirred for 1 h at 0° C. (in dark). The reaction mixture was concentrated with oil pump below 5° C. to remove CCl₄. The residue was dissolved into THF (100 mL), then compound T4_4 (2.3 g, 8.4 mmol) and DIPEA (3.1 g, 24 mmol) was added at 0° C., then the mixture was stirred for 18 h at 15° C. to 25° C. The reaction was quenched with water (50 mL) and extracted with EA (100 mL×2), the organic phase was combined and washed with brine (50 mL), dried over Na₂SO₄, purified by column chromatography on silica gel (PE/EA=5/1 to PE/EA=2/1) to afford compound T5_1 as white solid (2 g, 37%).

### Synthesis of Compound Target 5 (T5):

T5_1 (1 g, 1.5 mmol) was dissolved into DCM (10 mL) at 10° C. to 20° C., followed by of TFA (5 mL), the mixture was stirred for 24 h at 10° C. to 20° C. The pH of reaction was adjusted with sat. aqueous NaHCO₃ until 7 to 8. The result mixture was filtered and the filtrate was extracted with DCM (10 mL×3), the organic phase was combined and washed with brine (10 mL), dried over Na₂SO₄, concentrated and purified by column chromatography on silica gel (PE/EA=5/1 to PE/EA=1/1) to afford target 5 as white solid (120 mg, 16%). ¹H NMR (400 MHz, CDCl₃) δ 7.99-7.96 (2H, m), 7.26-7.24 (2H, m), 7.17-7.12 (4H, m), 7.08-7.05 (2H, m), 7.01-6.97 (2H, m), 3.89-3.67 (4H, m), 3.28-3.22 (1H, m), 3.16 (4H, s), 3.04-2.93 (1H, m), 2.63-2.62 (1H, br).

### Example 6—Synthesis of Target 6 (T6)

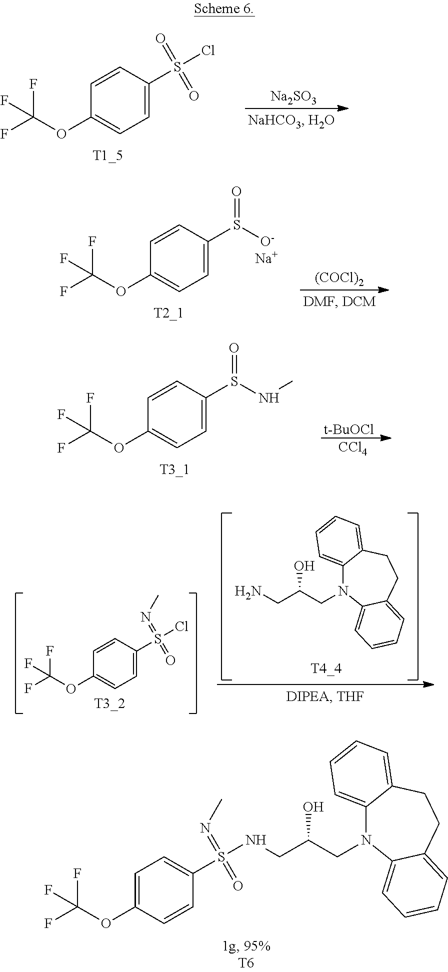

### Synthesis of Compound T3_1:

To a mixture of compound T2_1 (10.3 g, 30.3 mmol, Example 2) and DMF (0.15 mL) in DCM (100 mL) was added (COCl)₂ (6.1 g, 48.36 mmol) below 20° C. The mixture was stirred for 1 h after the addition, then the reaction mixture was concentrated under vacuum to give crude T3_1. The crude compound T2_2 was dissolved with DCM (100 mL) and then MeNH₂ (2M in THF, 19 g, 40 mmol) and DIEA (7.8 g, 60.45 mmol) was added into the mixture. The mixture was stirred for 18 h at 15 to 25° C. The reaction was quenched with water (100 mL) and separated to collect organic phase, the aqueous phase was extracted with DCM (100 mL), organic phase was combined and washed with brine, dried over Na₂SO₄, concentrated and purified by chromatography on silica gel (PE/EA=10/1) to give compound T3_1 as yellow oil (3.5 g 32%).

### Synthesis of Compound Target 6 (T6):

A mixture of T3_1 (10 g, 41.8 mmol) and t-BuOCl (10.9 g, 43.9 mmol) in 100 mL of CCl₄ was stirred for 1 h at 0° C. (in dark). The reaction mixture was concentrated with oil pump below 5° C. to remove CCl₄. The residue was dissolved into THF (150 mL), then compound T4_4 (11.7 g, 43.9 mmol) and DIPEA (16.2 g, 125.4 mmol) was added at 0° C., then the mixture was stirred for 18 h at 15 to 25° C. The reaction was quenched with water (100 mL) and extracted with EA (100 mL×2), the organic phase was combined and washed with brine (50 mL), dried over Na₂SO₄, concentrated under reduced pressure, the residue was purified by chromatography on silica gel (PE/EA=10/1) to afford Target 6 as off-white solid (1.1 g, 5%). ¹H NMR (400 MHz, CDCl₃) δ 7.88 (2H, t, J=8.4, 8.0 Hz), 7.28-7.25 (3H, m), 7.13-7.09 (6H, m), 6.96-6.92 (2H, m), 3.93-3.78 (3H, m), 3.43-3.19 (2H, m), 3.16 (4H, s), 2.57 (1H, d, J=17.6 Hz).

### Example 7—Synthesis of T10 (CAS 1809427-19-7)

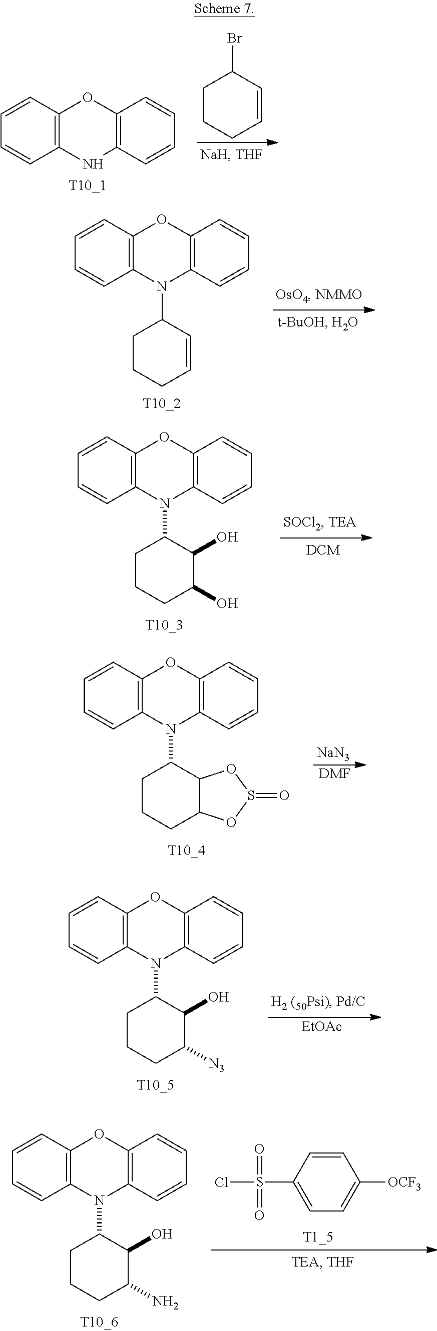

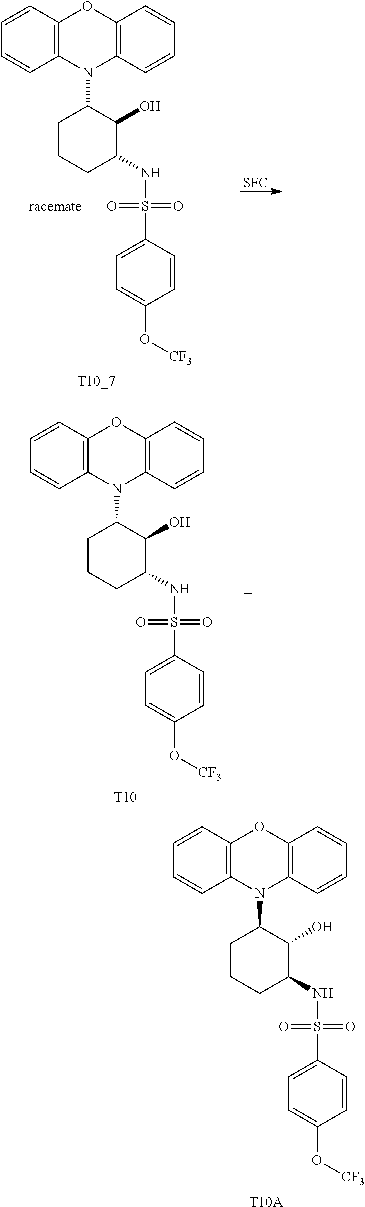

### Synthesis of Compound T10_2:

NaH (20.4 g, 510 mmol) was added into DMF (310 mL) at 25° C., then a solution of compound T10_1 (85.0 g, 464 mmol) in DMF (200 mL) was added into the mixture by drop wise. The mixture was stirred at 0° C. for 1 h, then 3-bromocyclohex-1-ene (89.7 g, 557 mmol) was added into the reaction by drop wise at 0° C., the mixture was stirred at 25° C. for 3 h after addition. TLC (petroleum ether/ethyl acetate=10/1, Rf=0.35) indicated compound T10_1 was consumed completely. The reaction mixture was quenched by NH₄Cl (400 mL) at 0° C. and filtered, the filter cake was purified by trituration with ACN (700 mL) to afford compound T10_2 (125 g, crude) as a light yellow solid, which was used for next step without further purification.

### Synthesis of Compound T10_3:

To a mixture of compound T10_2 (250 g, 949 mmol), NMMO (122 g, 1040 mmol), t-BuOH (1.50 L) and H₂O (150 mL) was added OsO₄ (4.00 g) at 25° C., the mixture was stirred at 25° C. for 18 h. The reaction mixture was quenched by addition saturated sodium bisulfite (200 mL) and stirred for 1 h. The reaction mixture was concentrated under reduced pressure to remove solvent to give a residue. The crude product was purified by trituration with ACN (2.00 L) to give compound T10_3 (230 g, crude) as an off-white solid, which was used for next step without further purification.

### Synthesis of Compound T10_4:

To a solution of compound T10_3 (230 g, 770 mmol) in DCM (1.10 L) was added TEA (235 g, 2.32 mol) at 0° C., followed by a solution of SOCl₂ (101 g, 850 mmol) in DCM (220 mL) was added into the reaction mixture by drop wise at 0° C., the mixture was stirred for 3 h at 25° C. Water (60 mL) was charged into the mixture and pH was adjusted to 9 with saturated aqueous Na₂CO₃ (300 mL). Ethyl acetate (500 mL) and H₂O (250 mL) were charged into the mixture, the mixture was filtered and the filtrate was washed with brine (250 mL), dried over Na₂SO₄, concentrated to give crude which was triturated with ACN (500 mL) at 25° C. for 60 min to obtain compound T10_4 as a white solid (220 g, 83%).

### Synthesis of Compound T10_5:

To a solution of compound T10_4 (100 g, 291 mmol) in DMF (1.00 L) was added NaN₃ (55.4 g, 853 mmol), then the mixture was heated at 120° C. for 12 h. The reaction mixture was quenched by saturation sodium carbonate (1.00 L) and extracted with EtOAc (2.00 L×3). The combined organic layers were washed with water (2.00 L), brine (2.00 L), dried over Na₂SO₄, concentrated to give compound T10_5 (90 g, crude) as a brown oil, which was used for next step directly.

### Synthesis of Compound T10_6:

To a solution of compound T10_5 (99.0 g, 307 mmol) in EtOAc (1.00 L) was added Pd/C (9.9 g, 10 wt %) at 25° C. under N₂. The suspension was degassed under vacuum and purged with H₂ several times, and then the mixture was stirred under H₂ (50 psi, 3.4 bar) at 25° C. for 48 h. The reaction mixture was filtered and the filtrate was concentrated. The residue was purified by triturating with ACN (300 mL) to afford compound T10_6 as a white solid (25 g, 26%).

### Synthesis of Compound T10_7:

A solution of compound T10_6 (50.0 g, 169 mmol) in THF (400 mL) was added TEA (17.0 g, 169 mmol) and compound T1_5 (52.8 g, 202 mmol) at 0° C., the mixture was stirred for 12 h at 25° C. Ethyl acetate (150 mL) and H₂O (75.0 mL) were charged into the mixture. Then the mixture filtered and the filtrate was washed with brine (50.0 mL), dried over Na₂SO₄, the organic phase was concentrated, the residue was triturated with acetonitrile at 25° C. for 60 min to give compound T10_7 as a white solid (25.0 g, 28%).

SFC for Compound T10_7:

25 g of compound T10_7 was taken for SFC, which afforded T10 (9.2 g) as a white solid and T10A (9.2 g) as a white solid.

### Example 8—Western Blotting

Cell protein was isolated with lysis buffer from CoIP kit (Life Technologies 14321D) with added 100 mM NaCl and Roche Protease and Phosphatase inhibitors. Isolated protein was quantified, normalized via Bio-Rad assay (Bio-Rad), run on a 12% SDS-PAGE (Invitrogen, Life Technologies), and transferred onto nitrocellulose membranes (Bio-Rad). The membrane was blocked with 5% nonfat milk (LabScientific Inc.) in Tris-buffered saline-Tween 20 buffer. For methyl-PP2A-C antibody, membrane was blocked with 3% nonfat milk in Phosphate-buffered saline-Tween 20 buffer. Membranes were probed with anti-phospho c-MYC s62 (Abcam), total c-MYC (Cell Signaling), methyl-PP2A-C, total PP2A-C(Abcam), cleaved PARP (Cell Signaling) and vinculin (Santa Cruz). Primary antibodies were probed with either goat anti-mouse (Abcam, Cambridge, United Kingdom) or donkey anti-rabbit (GE Healthcare, Little Chalfont, United Kingdom) conjugated to horseradish peroxidase and imaged and quantified using the Bio-Rad ChemiDoc XRS chemiluminescence imager and software. All values were normalized to vinculin or GAPDH and expressed as fold change relative to control.

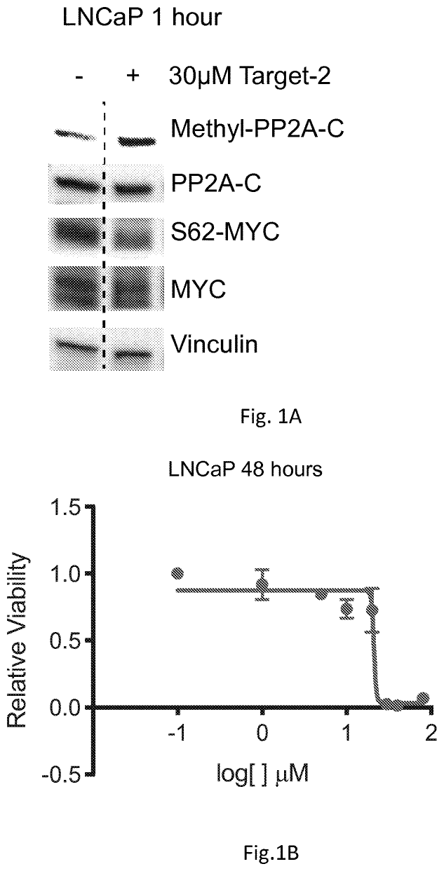

Western blot of Methyl-PP2A-C, total PP2A-C, S62-MYC, total MYC, and Vinculin in LNCaP cells treated with vehicle control or 30 μM Target-2 and harvested at 1 hour is shown in FIG. 1A.

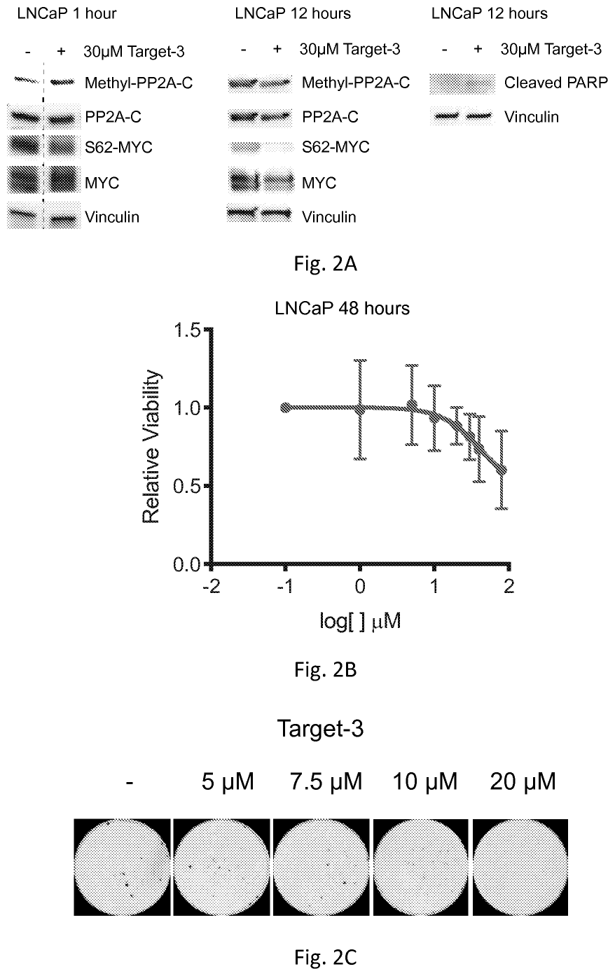

Western blots of Methyl-PP2A-C, total PP2A-C, S62-MYC, total MYC, Vinculin and cleaved PARP in LNCaP cells treated with vehicle control or 30 μM Target-3 and harvested at 1 hour or 12 hours are shown in FIG. 2A.

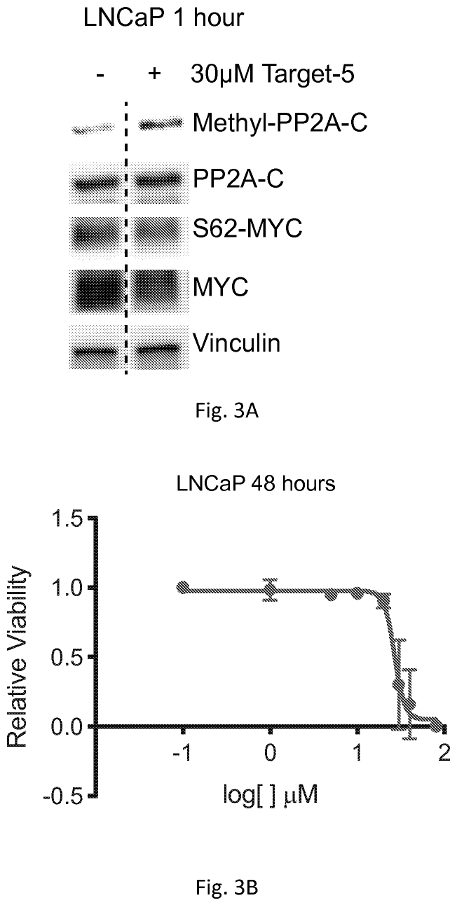

Western blot of Methyl-PP2A-C, total PP2A-C, S62-MYC, total MYC, and Vinculin in LNCaP cells treated with vehicle control or 30 μM Target-5 and harvested at 1 hour is shown in FIG. 3A.

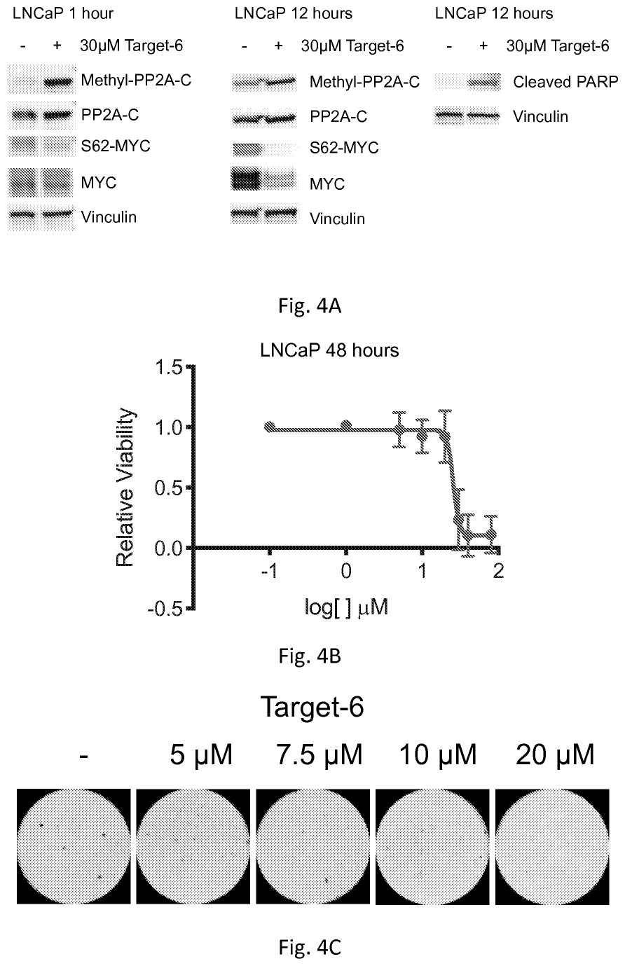

Western blots of Methyl-PP2A-C, total PP2A-C, S62-MYC, total MYC, Vinculin and cleaved PARP in LNCaP cells treated with vehicle control or 30 μM Target-6 and harvested at 1 hour or 12 hours are shown in FIG. 4A.

### Example 9—Cell Viability Assay and Human Pregnane X Receptor (PXR, NR1I2) Activation Assay

Cell line LNCaP was used for this assay. LNCaP cells were plated in 96-well plates at a density of 5000 cells per well. After 24 hours of plating, cells were treated with increasing doses of compound ranging from 1 μM to 80 μM. Relative cell numbers were analyzed after 48 hours using a 3-(4,5-dimethylthiazol-2-yl)-5-(3-carboxymethoxyphenyl)-2-(4-sulfophenyl)-2H-tetrazolium (MTS) assay (Promega) according to the manufacturer's directions.

LNCaP cells were treated with increasing doses of Target compound and cell viability was measured at 48 hours by MTS analysis.

The cell viability data was analyzed with GraphPad Prism software. The values were log transformed and analyzed with nonlinear regression (curve-fit) using log(inhibitor) vs. response variable slope (four parameters) and constraining the bottom to equal zero. The results are provided in Table 5.

The measured Cell viability for LNCaP cells treated with Target-2, Target-3, Target-5 and Target-6 is shown in FIGS. 1B, 2B, 3B, and 4B, respectively.

PXR is a nuclear hormone receptor involved in the transcriptional regulation of a number of metabolic enzymes (e.g. CYP3A4) and transporters (e.g. PGP). Its activation by xenobiotic substances leads to upregulation of those metabolic enzymes and transporters and consequent removal of those substances from systemic circulation. It is not uncommon to find that drugs activate the PXR receptor with problematic consequences. PXR activation by a drug can results in increased metabolism of concomitantly administered drugs that are cleared by, for example, CYP3A4 rendering them less effective (negative drug-drug interaction). In addition, if the drug itself is metabolized by, for example, CYP3A4, then there can be reduced exposure of the drug on repeated administration (auto-induction). It is thus considered beneficial to eliminate or at least minimize PXR activation if drug candidates. For drug series where PXR activation is significant, PXR activation is generally treated as a key criterion for optimization and advancement (V. Chu et al., Drug Metab. Dispos. 37, 1339 (2009).

PXR activation assay was performed according to manufacturer's instructions (Puracyp, Inc, Catalog #DPX2-96-001). The results are provided in Table 6.

TABLE 5
Cell viability assay data for compounds.

| Compound | Structure | GI₅₀ (μM) |
|---|---|---|
| T1 | 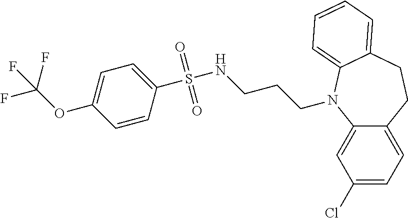 [SMILES: O=S(=O)(NCCCN1c2ccccc2CCc2ccc(Cl)cc21)c1ccc(OC(F)(F)F)cc1] | Not determined |
| T2 | 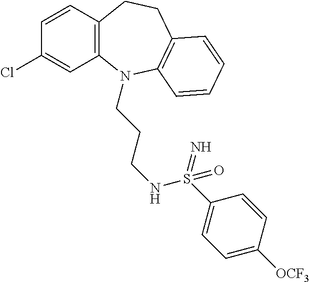 [SMILES: N=S(=O)(NCCCN1c2ccccc2CCc2ccc(Cl)cc21)c1ccc(OC(F)(F)F)cc1] | Not determined |
| T3 | 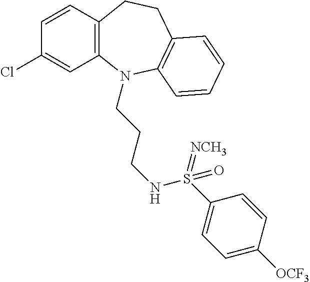 | Not determined |
| T4 | 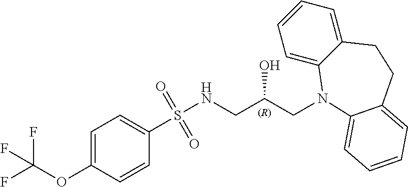 | Not determined |
| T5d1 | 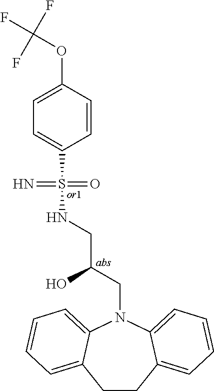 [SMILES: N=[S@@](=O)(NC[C@H](O)CN1c2ccccc2CCc2ccccc21)c1ccc(OC(F)(F)F)cc1] | 25.83 |
| T5d2 | 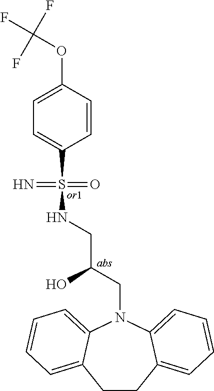 [SMILES: N=[S@](=O)(NC[C@H](O)CN1c2ccccc2CCc2ccccc21)c1ccc(OC(F)(F)F)cc1] | 20.2 |
| T6d1 | 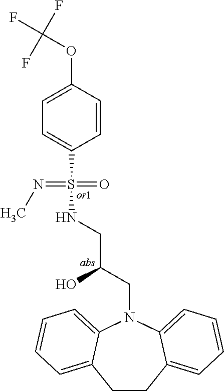 [SMILES: CN=[S@@](=O)(NC[C@H](O)CN1c2ccccc2CCc2ccccc21)c1ccc(OC(F)(F)F)cc1] | 22.7 |
| T6d2 | 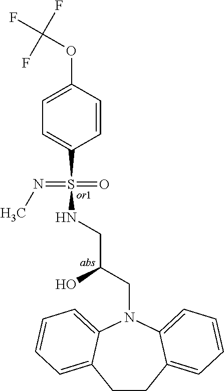 [SMILES: CN=[S@](=O)(NC[C@H](O)CN1c2ccccc2CCc2ccccc21)c1ccc(OC(F)(F)F)cc1] | 24.47 |
| T7 | 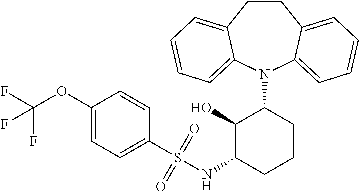 [SMILES: O=S(=O)(N[C@H]1CCC[C@@H](N2c3ccccc3CCc3ccccc32)[C@@H]1O)c1ccc(OC(F)(F)F)cc1] | Not determined |
| T8 | 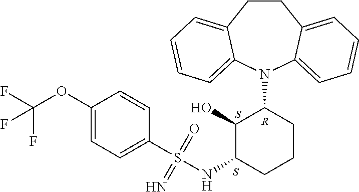 | Not determined |
| T9 | 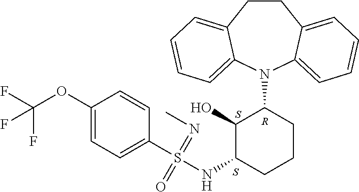 | Not determined |
| T10 (DT-061) | 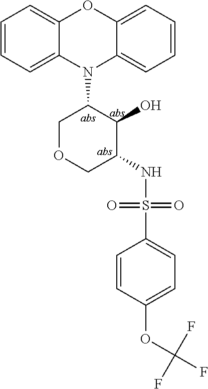 | 10.68 |
| T11-1 | 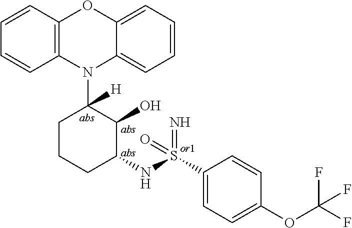 | 16.71 |
| T11-2 | 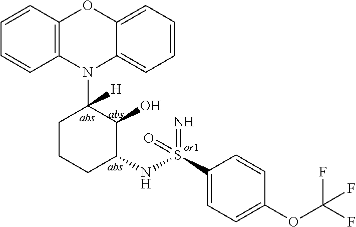 | 16.75 |
| T13 | 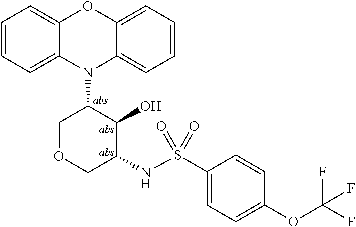 | 14.2 |
| T14-1 | 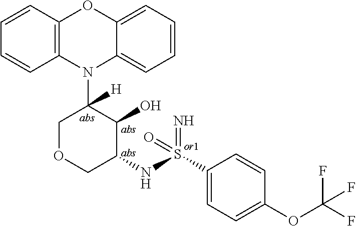 | 25.63 |
| T14-2 | 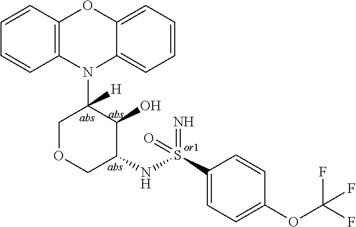 | 25.59 |
| T15-1 | 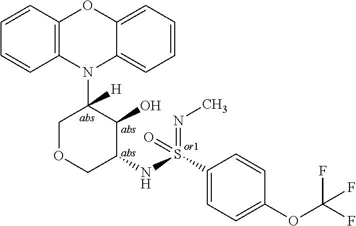 | 16.65 |
| T15-2 | 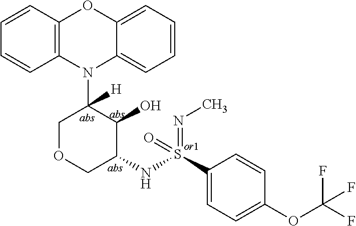 | 23.61 |
| T15A | 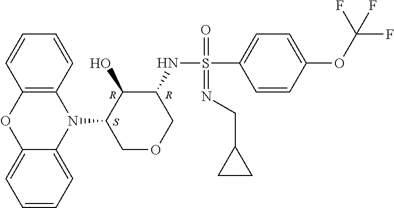 | 9.54 |
| T15C | 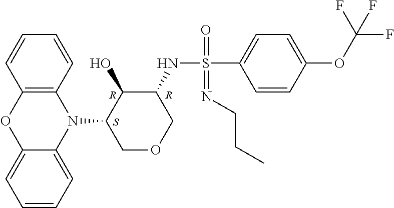 | 55.38 |
| T16 | 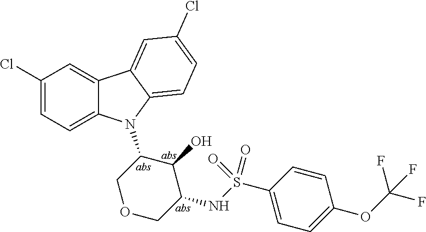 [SMILES: O=S(=O)(N[C@@H]1COC[C@H](n2c3ccc(Cl)cc3c3cc(Cl)ccc32)[C@H]1O)c1ccc(OC(F)(F)F)cc1] | 8.202 |
| T17-1 | 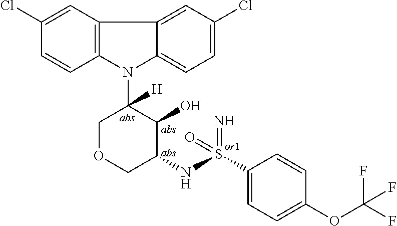 | 10.22 |
| T17-2 | 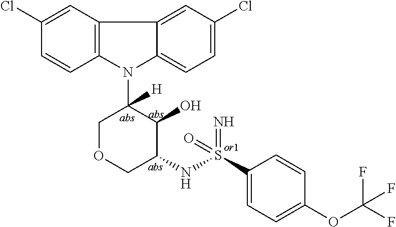 | 9.64 |
| T18-1 | 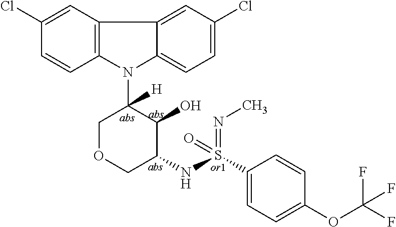 | 8.53 |
| T18-2 | 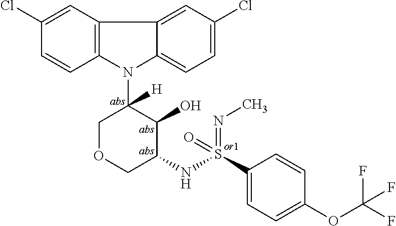 | 12.22 |

TABLE 6
PXR activation assay data for compounds. Concentration
observed is at 10 μM.

| | Maximum fold activation | Percent positive |
|---|---|---|
| Compound | observed (Average) | control response |
| T10 (DT-061) | 17.3 | 89.8 |
| T11-1 | 4.57 | 24.2 |
| T11-2 | 4.82 | 26 |
| T13 | 11 | 57 |
| T14-1 | 6.21 | 35.4 @30 μM |
| T14-2 | 4.12 | 21.2 |
| T15-1 | 3.89 | 19.6 |
| T15-2 | 7.15 | 41.8 |
| T16 | 3.93 | 16.7 |
| T17-1 | 2.43 | 9.75 |
| T17-2 | 2.45 | 9.88 |
| T18-1 | 1.79 | 5.37 |
| T18-2 | 2.36 | 9.23 |

### Example 10—Colony Formation Assay

LNCaP cells were plated at a low density in 6-well plates. After 48 hours, cells were treated with increasing concentrations of Targets for 3 weeks. Cells were fixed and stained with 1% crystal violet solution. Quantification was performed through the cell counter function on ImageJ.

Colony formation assay of LNCaP cells treated with 5, 7.5, 10, and 20 μM Target-3 for 3 weeks is shown in FIG. 2C.

Colony formation assay of LNCaP cells treated with 5, 7.5, 10, and 20 μM Target-6 for 3 weeks is shown in FIG. 4C.

### Example 11—Screening of Active Compounds for Prevention and/or Treatment of Betacorovirus Infection

Drug screen, data analysis, SARS-CoV-2, SARS-CoV, or MERS viral infections, pseudovirus fusion/entry assays and in vivo infections of the compounds of the present invention can be performed using Vero E6 cells, for example, as described in Stuart Weston S et al. (2020) Broad anti-coronaviral activity of FDA approved drugs against SARS-CoV-2 in vitro and SARS-CoV in vivo. bioRxiv 2020.03.25.008482; doi: https://doi.org/10.1101/2020.03.25.008482 or as described in Dyall J. et al. (2014) Repurposing of Clinically Developed Drugs for Treatment of Middle East Respiratory Syndrome Coronavirus Infection. Antimicrobial Agents and Chemotherapy July 2014, 58 (8) 4885-4893; DOI: 10.1128/AAC.03036-14.

### Example 12—Synthesis of T8

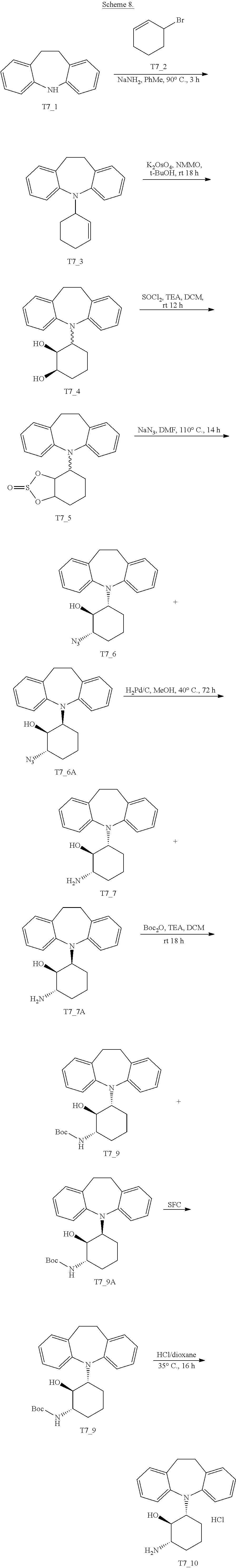

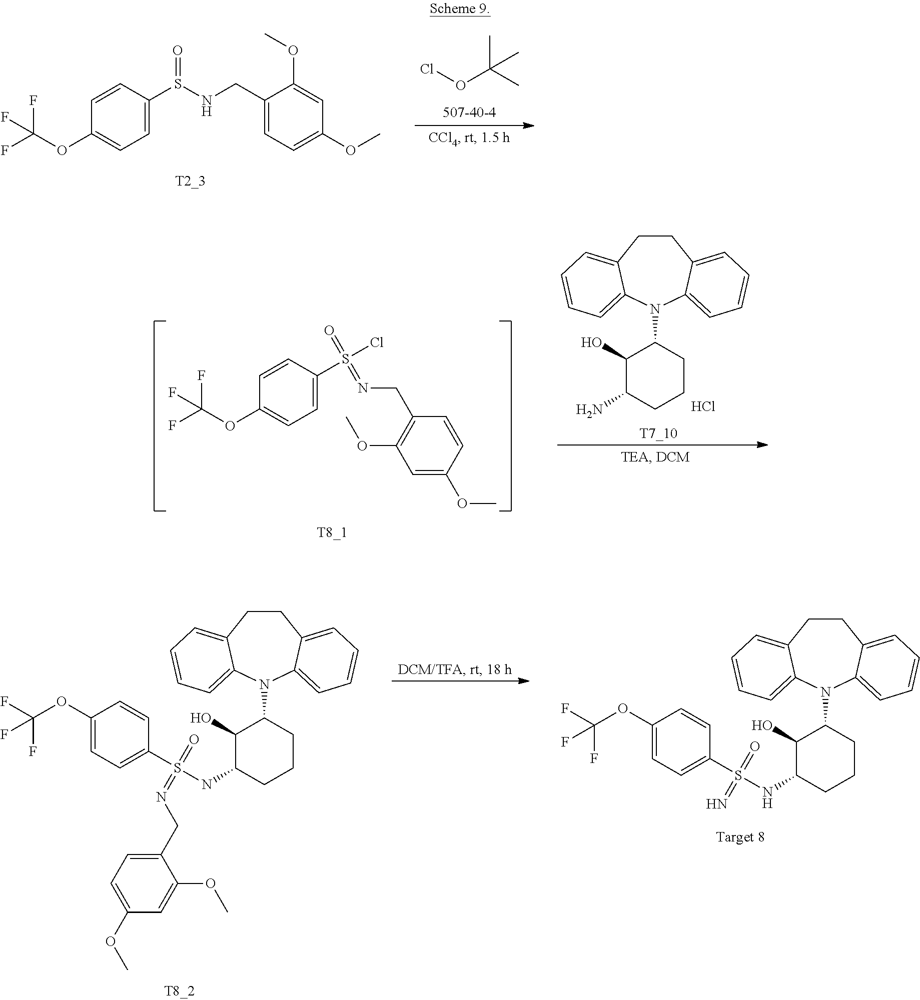

### Synthesis of Compound T7_3:

To a solution of compound T7_1 (200 g, 1.02 mol) in Toluene (4 L) was added NaNH₂ (59.9 g, 1.54 mol) and T7_2 (173.2 g, 1.08 mol) with stirring, then the reaction mixture was heated to 90° C. for 18 h. TLC showed that the reaction has been completed. The mixture was cooled to 25° C. and poured into saturated NH₄Cl aqueous (2 L). The organic layer was separated, the organic layer was dried over Na₂SO₄, then it was filtered and concentrated under reduced pressure. The residue was purified by column chromatography on silica gel (PE) to afford compound T7_3 as white solid (200 g, 70.9%). ¹H NMR (400 MHz, CDCl₃) δ 7.33-7.29 (d, J=15.0 Hz, 2H), 7.14-7.09 (m, 4H), 6.97-6.94 (m, 2H), 6.38-6.35 (m, 1H), 5.88-5.83 (m, 1H), 4.60-4.57 (m, 1H), 3.22-3.09 (m, 2H), 3.07-3.04 (m, 2H), 2.02-2.03 (m, 2H), 1.93-1.90 (m, 1H), 1.80-1.76 (m, 1H), 1.72-1.60 (m, 2H).

### Synthesis of Compound T7_4:

To a solution of compound T7_3 (200 g, 727.3 mmol) and NMMO (127.3 g, 1.09 mol) in t-BuOH (1.5 L) and H₂O (0.15 L) was added K₂OsO₄.2H₂O (5.0 g, 13.6 mmol) in one portion at 25° C. The mixture was stirred at 25° C. for 18 h. TLC showed that the reaction has been completed. The reaction mixture was quenched by saturated Na₂SO₃ aqueous (1 L), then extracted with EtOAc (1.5 L×3). The combined organic layers were dried over Na₂SO₄, then it was filtered and concentrated under reduced pressure to afford T7_4 as brown oil (200 g, 89%). ¹H NMR (400 MHz, CDCl₃) δ 7.24-7.22 (s, 1H), 7.15-7.09 (m, 4H), 7.00-6.97 (m, 2H), 4.23-4.16 (m, 2H), 3.73-3.71 (m, 1H), 2.86-2.85 (d, J=2.4 Hz, 1H), 2.33 (s, 1H), 2.17-2.11 (m, 1H), 1.84-1.80 (m, 1H), 1.64-1.60 (m, 1H), 1.49-1.30 (m, 3H).

### Synthesis of Compound T7_5:

To a solution of compound T7_4 (200 g, 646.6 mmol) and TEA (196 g, 1.94 mmol) in DCM (1.5 L) was added SOCl₂ (84.6 g, 711 mmol) dropwise while keeping internal temperature below 5° C. After the addition, it was stirred for 12 h at 25° C. LCMS showed that the reaction has been completed, so the resulting mixture was washed with saturated Na₂CO₃ aqueous (500 mL) and brine (1 L). The organic phase was dried over Na₂SO₄, then it was filtered and concentrated under reduced pressure to afford compound T7_5 as brown oil (180 g, 78%). LCMS: M/Z (M+H)⁺ 356.2.

Synthesis of Compound rac-T7_6 (Mixture of T7_6 and T7_6A):

A suspension of compound T7_5 (180 g, 506 mmol) and NaN₃ (98.79 g, 1.52 mol) in DMF (1.8 L) was stirred at 110° C. for 16 h. LCMS showed that the reaction has been completed, so the reaction mixture was poured into saturated Na₂CO₃ aqueous (5.4 L), it was extracted with EtOAc (1.5 L×3). The combined organic layers were washed with brine (1.5 L×3), dried over Na₂SO₄, then it was filtered and concentrated under reduced pressure, the residue was purified by column chromatography on silica gel (EA:PE=1:10) to afford compound rac-T7_6 as brown oil (145 g, 85.6%). LCMS: M/Z (M+H)⁺ 335.2.

Synthesis of Compound rac-T7_7 (Mixture of T7_7 and T7_7A):

A mixture of rac-T7_6 (145 g, 431 mmol) and Pd/C (10%, 14.5 g) in MeOH (2 L) was stirred at 40° C. under hydrogen atmosphere for 72 h. TLC showed that the reaction has been completed, it was filtered and filtrate was concentrated under reduced pressure to obtain the crude rac-T7_7 as brown solid (100 g, 75%) which was directly used for next step without further purification.

Synthesis of Compound rac-T7_9 (Mixture of T7_9 and T7_9A):

To a solution of rac-T7_7 (44 g, 142.66 mmol) and TEA (21.65 g, 214 mmol) and DCM (500 mL) was added (Boc)₂O (37.4 g, 171.2 mmol) dropwise at 25° C. The resulting mixture was stirred at 25° C. for 18 h. TLC showed that the reaction has been completed, then it was poured into water (500 mL), the organic layer was separated, the aqueous was extracted with DCM (500 mL), the combined organic layers was washed with brine (300 mL), dried over Na₂SO₄, it was filtered and concentrated under reduced pressure, then the residue was purified by column chromatography on silica gel (PE:EA=10:1) to afford rac-T7_9 (55 g, 94%) as white solid which was separate by chiral SFC (SFC-7; ChiralPak IC, 300×50 mm I.D., 10 μm, CO2/Methanol-0.1% NH₃H₂O=60%/40%; 200 mL/min) to afforded T7_9 (20 g, 36%, 98.3% ee.) as off-white solid. ¹H NMR (400 MHz, CDCl₃) δ 7.56 (br s, 2H), 7.30-7.12 (m, 6H), 4.65 (s, 1H), 3.80-3.75 (m, 1H), 3.38-3.66 (m, 4H), 2.85 (s, 1H), 2.19-2.16 (m, 2H), 1.99-1.96 (m, 2H), 1.69-1.64 (m, 2H), 1.52 (s, 9H), 1.42-1.34 (m, 1H), 1.24-1.03 (m, 2H). LCMS: M/Z (M+H)⁺ 409.2.

### Synthesis of Compound T7_10:

To a solution of T7_9 (20 g) in dioxane (100 mL) was added HCl/dioxane (5M, 100 mL) dropwise at 25° C. The reaction mixture was stirred at 25° C. for 16 h. TLC showed that the reaction has been completed, the resulting mixture was concentrated under reduced pressure to afford T7_10 as white solid (14 g, 83%). ¹H NMR (400 MHz, CD₃OD) δ 7.47-6.98 (m, 8H), 3.74-3.66 (m, 2H), 3.52-3.47 (t, J₁=9.6 Hz, J₂=10 Hz, 1H), 3.10-3.04 (m, 2H), 2.26-2.20 (m, 1H), 1.97-1.93 (m, 1H), 1.78-1.73 (m, 1H), 1.47-1.35 (m, 1H), 1.30-1.19 (m, 3H). LCMS: M/Z (M+H)⁺ 309.2.

### Synthesis of Compound T8_2:

To a solution of T2_3 (10 g, 26.57 mmol; see Example 2) in CCl₄ (100 mL) was added tert-butyl hypochlorite (3.2 g, 29.2 mmol) dropwise at 0° C. The mixture was stirred at 0° C. for 1 h (kept in dark). TLC showed that the reaction has been completed, the solvent was removed by reduce pressure under 5° C. to afford crude product T8_1 (10 g).

To a solution of T7_10 (8.2 g, 20.96 mmol) and TEA (11.2 g, 110.5 mmol) in THF (80 mL) was added a solution of the crude product T8_1 in THF (20 mL) dropwise at 0° C. After addition, the mixture was stirred at 25° C. for 18 h. TLC showed that the reaction has been completed, it was poured into water (50 mL), extracted with EtOAc (40 mL×3), the combined organic layers were washed with brine (50 mL), dried over Na₂SO₄, then it was filtered and concentrated under reduced pressure, then the residue purified by column chromatography on silica gel (PE:EA=20:1) to afford T8_2 as white solid (4.6 g 31%). ¹H NMR (400 MHz, CDCl₃) δ 7.92-7.87 (m, 2H), 7.77-7.40 (m, 0.5H), 7.50 (s, 1H), 7.32-7.26 (m, 2.5H), 7.10-6.93 (m, 7H), 6.33-6.26 (m, 2H), 4.21-4.16 (m, 2H), 4.04-4.08 (m, 8H), 3.45-3.24 (m, 3H), 2.14-2.11 (m, 1H), 1.89-1.85 (m, 1H), 1.58-1.56 (m, 1H), 1.31-1.20 (m, 3H).

### Synthesis of Compound Target 8:

To a solution of T8_2 (4.6 g, 6.75 mmol) in DCM (50 mL) was added TFA (25 mL) dropwise at 25° C. The mixture was stirred at 25° C. for 18 h. TLC showed that the reaction has been completed, saturated NaHCO₃ aqueous was added to adjust pH >7.5, then extracted with DCM (20 mL×3), the combined organic layers were washed with brine (10 mL), dried over Na₂SO₄, it was filtered and concentrated under reduced pressure, then the residue was purified by column chromatography on silica gel (PE:EA=10:1 to 2:1) to afford Target 8 as white solid (1.1 g, 11%). ¹H NMR (400 MHz, CD₃OD) δ 8.05-8.03 (d, J=8.4 Hz, 2H), 7.42-7.40 (d, J=8.4 Hz, 2H), 7.09-7.04 (m, 4H), 6.93 (s, 2H), 3.71-3.66 (m, 2H), 3.37-3.35 (m, 1H), 3.30-2.86 (m, 3H), 2.11-2.08 (m, 1H), 1.51-1.16 (m, 6H). LCMS: M/Z (M+H)⁺ 532.2.

### Example 13—Synthesis of Target 9 (T9)

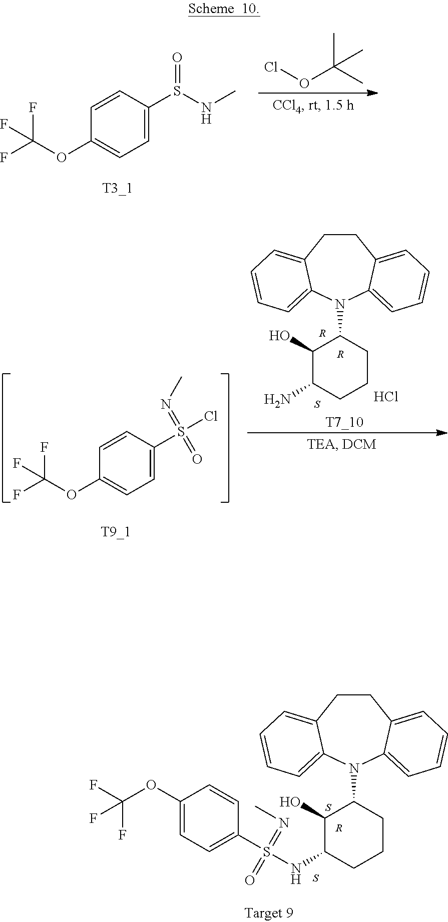

### Synthesis of Compound Target 9:

To a solution of T3_1 (4 g, 16.07 mmol; see Example 3) in CCl₄ (100 mL) was added tert-butyl hypochlorite (1.83 g, 16.8 mmol) dropwise at 0° C. The mixture was stirred at 0° C. for 1 h (in dark). TLC showed that the reaction has been completed, the solvent was removed by reduce pressure under 5° C. to afford crude product T9_1 (4 g).

To a solution of T7_10 (3.4 g, 24.1 mmol) and TEA (5.6 g, 55.8 mmol) in THF (40 mL) was added a solution of the crude product T9_1 in THF (20 mL) dropwise at 0° C. After addition, the mixture was stirred at 25° C. for 18 h. TLC showed that the reaction has been completed, water (50 mL) was added, extracted with EA (40 mL×3), the combined organic layers were washed with brine (50 mL), dried over Na₂SO₄, it was filtered and concentrated under reduced pressure, then the residue was purified by column chromatography on silica gel (PE:EA=10:1 to 5:1) to afford Target 9 as white solid (1.3 g 24%). ¹H NMR (400 MHz, CD₃OD) δ 8.09-8.04 (m, 2H), 7.48-7.45 (m, 4H), 7.11-7.05 (m, 4H), 6.93 (s, 2H), 3.68 (s, 1H), 3.41-3.30 (m, 2H), 2.62-2.55 (m, 3H), 2.18-2.15 (m, 1H), 1.89 (bs, 1H), 1.62-1.60 (m, 1H), 1.29-1.26 (m, 3H). LCMS: M/Z (M+H)⁺ 546.2.

### Example 14—Synthesis of Target 11 (T11)

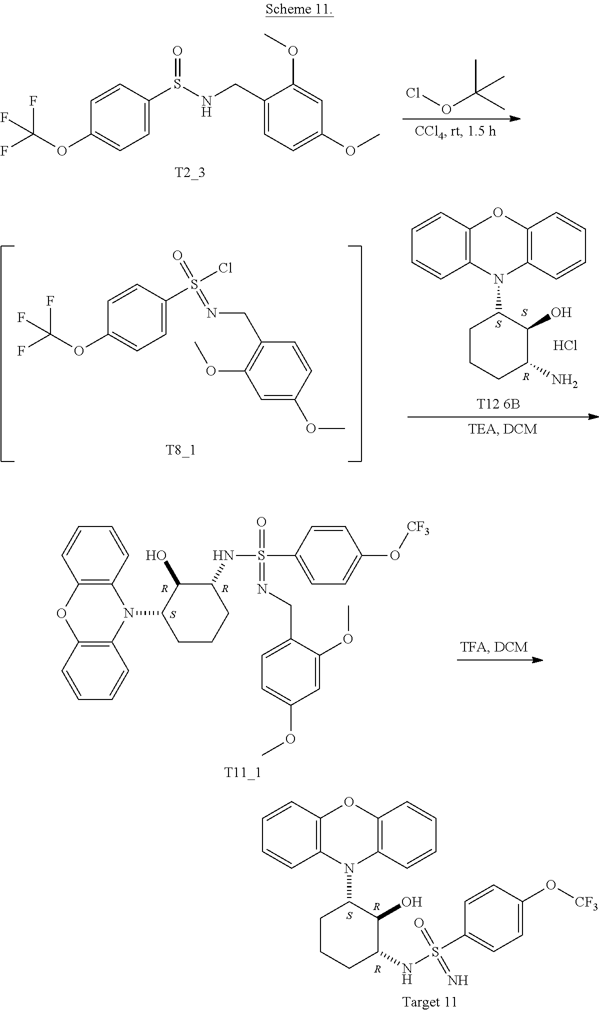

### Synthesis of Compound T11_1:

To a solution of T2_3 (10 g, 26.57 mmol; see Example 2) in CCl₄ (100 mL) was added tert-butyl hypochlorite (3.2 g, 29.2 mmol) dropwise at 0° C., the mixture was stirred at 0° C. for 1 h (in dark). TLC showed that the reaction has been completed, the solvent was removed by reduce pressure under 5° C. to afford crude T8_1 (10 g).

To a solution of T12_6B (8.2 g, 22.27 mmol, Example 15) and TEA (13.5 g, 133.9 mmol) in THF (70 mL) was added a solution of the crude T8_1 in THF (20 mL) dropwise at 0° C. After addition, the mixture was stirred at 25° C. for 18 h. TLC showed that the reaction has been completed, the reaction mixture was poured into water (50 mL), extracted with EA (40 mL×3), the combined organic layers was washed with brine (50 mL), dried over Na₂SO₄, it was filtered and concentrated under reduced pressure, then the residue was purified by column chromatography on silica gel (PE:EA=20:1) to afford T11_1 as white solid (4.5 g 27%). ¹H NMR (400 MHz, CDCl₃) δ 7.86-7.84 (d, J=8.8 Hz, 2H), 7.76-7.74 (d, J=8.8 Hz, 1H), 7.26-7.16 (m, 2H), 7.02-6.98 (m, 2H), 6.89-6.75 (m, 7H), 6.29-6.25 (m, 2H), 4.06 (s, 2H), 3.97-3.84 (m, 2H), 3.84 (s, 3H), 3.68 (s, 3H), 3.49-2.88 (m, 2H), 1.96-1.92 (m, 2H), 1.83-1.77 (m, 2H), 1.77-1.54 (m, 5H), 1.41-1.21 (m, 4H).

### Synthesis of Compound Target 11:

To a solution of T11_1 (4.5 g, 5.78 mmol) in 50 mL of DCM was added TFA (25 mL) dropwise at 25° C., The mixture was stirred at 25° C. for 18 h. TLC showed that the reaction has been completed, saturated aqueous NaHCO₃ aqueous added to adjust pH >7.5, then extracted with DCM (20 mL×3), the combined organic layers was washed with brine (10 mL), dried over Na₂SO₄, it was filtered and concentrated under reduced pressure, then the residue was purified by column chromatography on silica gel (PE:EA=40:1) to afford Target 11 as white solid (1.1 g, 31%). ¹H NMR (400 MHz, CD₃OD) δ 8.12-8.09 (d, J=8.8 Hz, 2H), 7.44-7.42 (d, J=8.4 Hz, 2H), 6.99-6.98 (m, 2H), 6.96-6.86 (m, 2H), 6.85-6.80 (m, 2H), 6.78-6.72 (m, 2H), 3.87-3.67 (m, 3H), 3.44-3.38 (m, 2H), 3.14-3.12 (s, 1H), 2.03 (s, 1H), 1.92-1.64 (m, 4H), 1.31-1.29 (m, 2H).

### Example 15—Synthesis of Target 12 (T12)

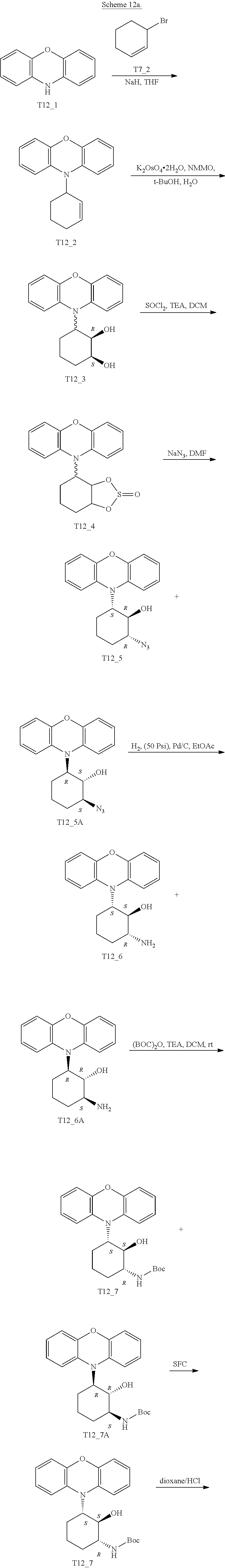

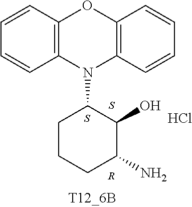

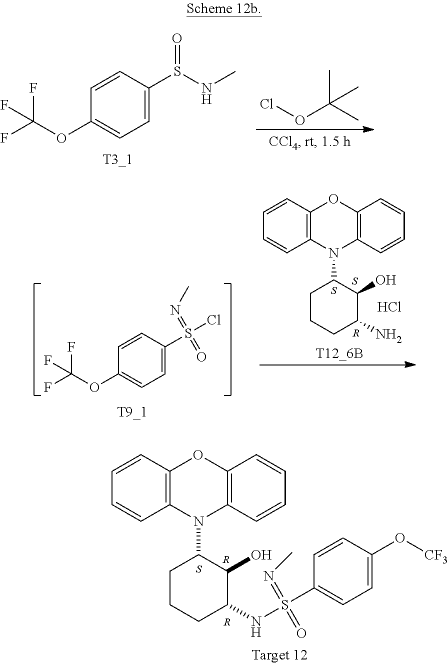

### Synthesis of Compound T12_2:

To a mixture of NaH (60%, 24 g) in THF (100 mL) was added T12_1 (100 g, 545.8 mmol) in small portions while keeping internal temperature below 0° C. The mixture was stirred for 1 h at the same temperature after the addition, then T7_2 (105.48 g, 656.0 mmol) was added at 0° C. The reaction mixture was stirred at 25° C. for 3 h. TLC showed that the reaction has been completed, it was poured into saturated NH₄Cl aqueous (100 mL), then it was filtered, the filter cake was purified by triturating with ACN (700 mL) to afford T12_2 as white solid (100 g, 69.9%). ¹H NMR (400 MHz, CDCl₃) δ 6.87-6.72 (m, 8H), 5.97-5.94 (m, 1H), 5.87-5.84 (m, 1H), 4.62-4.59 (m, 1H), 2.25-2.20 (m, 3H), 2.18-2.01 (m, 2H), 1.85-1.80 (m, 1H), 0.98-0.91 (m, 1H).

### Synthesis of Compound T12_3:

To a solution of T12_2 (150 g, 569.5 mmol) and NMMO (115 g, 854.2 mmol) in t-BuOH (1 L) and water (100 mL) was added K₂OsO₄.2H₂O (25.5 g, 56.95 mmol) in one portion at 25° C. The mixture was stirred for 16 h at 25° C. TLC showed that the reaction has been completed, the reaction mixture was quenched by addition saturated sodium bisulfite aqueous (200 mL) and stirred for 1 h. Then filtered, the filter cake was purified by triturating with ACN (700 mL) to afford T12_3 as white solid (130 g, 75%). ¹H NMR (400 MHz, CDCl₃) δ 6.97-6.84 (m, 8H), 4.26 (s, 1H), 3.97-3.92 (m, 1H), 3.81-3.76 (m, 1H), 3.24 (s, 1H), 2.53 (s, 1H), 1.99-1.92 (m, 3H), 1.74-1.67 (m, 2H), 1.57-1.50 (m, 1H), 1.44-1.25 (m, 3H).

### Synthesis of Compound T12_4:

To a solution of T12_3 (130 g, 445.9 mmol) and TEA (135.3 g, 1.34 mol) in DCM (1 L) was added SOCl₂ (58.3 g, 490.5 mmol) dropwise while keeping the internal temperature below 0° C. After addition, the mixture was stirred at 25° C. for 3 h. TLC showed that the reaction has been completed, the reaction mixture was poured into water (500 mL), extracted with DCM (200 mL×2), the combined organic layers was washed with saturated Na₂CO₃ aqueous (100 mL×3) and brine (100 mL×3), dried over Na₂SO₄, concentrated under reduced pressure, and the residue was purified by triturating with ACN (700 mL) to afford T12_4 as white solid (94 g, 59%). ¹H NMR (400 MHz, CDCl₃), δ 6.94-6.82 (m, 7H), 6.78-6.76 (m, 1H), 5.29-5.25 (m, 1H), 5.01-4.97 (m, 0.5H), 4.82-4.80 (m, 0.5H), 4.45-4.39 (m, 0.5H), 3.52-3.46 (m, 0.5H), 2.42-2.36 (m, 1H), 2.16-2.00 (m, 1H), 1.96-1.71 (m, 4H).

Synthesis of Compound rac-T12_5 (Mixture of T12_5 and T12_5A):

A suspension of T12_4 (90 g, 261.2 mmol) and NaN₃ (50.9 g, 783.5 mmol) in DMF (1 L) heated to 100° C. for 18 h. TLC showed that the reaction has been completed, the reaction mixture was quenched by saturated Na₂SO₃ aqueous (1 L), extracted with EtOAc (2 L), the organic layer was washed with brine (200 mL), dried over Na₂SO₄, and it was filtered and concentrated under reduced pressure to afford crude product rac-T12_5 as a yellow solid (75 g, 89%). ¹H NMR (400 MHz, CDCl₃), δ 6.95-6.85 (m, 8H), 3.86-3.81 (m, 1H), 3.43-3.27 (m, 3H), 2.00-1.95 (m, 2H), 1.79-1.76 (m, 1H), 1.60-1.52 (m, 2H), 1.35-1.21 (m, 2H).

Synthesis of Compound rac-T12_6 (Mixture of T12_6 and T12_6A):

A suspension of rac-T12_5 (75 g, 232.9 mmol) and Pd/C (15 g, 10 wt %) in EtOAc (2 L) was stirred at 25° C. under hydrogen (50 psi, 3.4 bar) for 48 h. TLC showed that the reaction has been completed, the reaction mixture was filtered, the filtrate was concentrated, and the residue was purified by triturating with ACN (700 mL) to afford rac-T12_6 as white solid (25 g, 37%). ¹H NMR (400 MHz, CDCl₃), δ 6.95-6.81 (m, 8H), 3.64-3.59 (m, 1H), 3.37-3.30 (m, 1H), 2.73-2.67 (m, 1H), 1.96-1.93 (m, 1H), 1.86-1.80 (m, 1H), 1.73-1.60 (m, 2H), 1.39-1.21 (m, 1H), 1.21-1.11 (m, 1H).

### Synthesis of Compound T12_7:

To a solution of rac-T12_6 (24 g, 84.4 mmol) and TEA (12.7 g, 126.1 mmol) in DCM (500 mL) was added (BOC)₂O (21.9 g, 100.7 mmol) dropwise at 25° C. The mixture was stirred at 25° C. for 18 h. TLC showed that the reaction has been completed, the reaction mixture was added water (500 mL), the organic layer was separated, the aqueous layer was extracted with DCM (500 mL), the combined organic layers were washed with brine (500 mL), dried over Na₂SO₄, it was filtered and concentrated under reduced pressure, then the residue was purified by column chromatography on silica gel (PE:EA=10:1) to afford rac-T12_7 as yellow solid (30 g, 85%) which was separated by chiral SFC (SFC-7; ChiralPak AS, 300×50 mm I.D., 10 μm; CO₂/Ethanol (0.1% NH₃.H₂O)=65%/35%; 200 mL/min) to afforded T12_7 (14 g, 47% yield, 99.9% ee). ¹H NMR (400 MHz, CDCl₃), δ 6.97-6.82 (m, 8H), 4.67 (br s, 1H), 3.73-3.67 (m, 1H), 3.53-3.48 (m, 2H), 3.40-3.34 (m, 1H), 2.07-1.95 (m, 2H), 1.75-1.71 (m, 1H), 1.61-1.57 (m, 2H), 1.38 (s, 9H), 1.37-1.16 (m, 3H).

### Synthesis of Compound T12_6B:

To a solution of T12_7 (14 g, 35.31 mmol) in dioxane (20 mL) was added HCl/dioxane (5 M, 100 mL) dropwise at 25° C. The mixture was stirred at 25° C. for 1 h. TLC showed that the reaction has been completed, MTBE (300 mL) was added, then it was filtered to afford T12_6B as a white solid (10 g, 92%). ¹H NMR (400 MHz, CDCl₃), δ 8.03 (s, 3H), 6.98-6.96 (m, 2H), 6.92-6.88 (m, 2H), 6.82-6.75 (m, 4H), 4.09-4.04 (t, J₁=1.6 Hz, J₂=19.6 Hz, 1H), 3.64-3.57 (m, 7H), 2.96 (bs, 1H), 1.95-1.93 (m, 2H), 1.93-1.84 (m, 1H), 1.84-1.71 (m, 1H), 1.48-1.43 (m, 2H), 1.23 (s, 1H).

### Synthesis of Compound Target 12:

To a solution of T3_1 (3.0 g, 12.68 mmol) in CCl₄ (40 mL) was added tert-butyl hypochlorite (1.37 g, 12.06 mmol) dropwise at 0° C. The mixture was stirred at 0° C. for 1 h (in dark). TLC showed that the reaction has been completed, the solvent was removed by reduce pressure below 5° C. to afford T9_1 (3.0 g, crude).

To a solution of T12_6B (3.9 g, 19.76 mmol) and TEA (5.27 g, 52.65 mmol) in THF (40 mL) was added a solution of the crude T9_1 (3.0 g) in THF (20 mL) dropwise at 0° C. After addition, the mixture was stirred at 25° C. for 18 h. TLC showed that the reaction has been completed, water (50 mL) was added, extracted with EtOAc (40 mL), the organic layer was washed with brine (50 mL), dried over Na₂SO₄, it was filtered and concentrated under reduced pressure, then purified by column chromatography on silica gel (PE:EA=7:1) to afford Target 12 as white solid (1.1 g, 23%). ¹H NMR (400 MHz, CD₃OD), δ 8.09-8.05 (m, 2H), 7.48-7.44 (m, 2H), 7.02-6.99 (m, 2H), 6.92-6.87 (m, 2H), 6.82-6.78 (m, 2H), 6.75-6.72 (m, 2H), 3.95-3.84 (m, 1H), 3.55-3.42 (m, 1H), 3.24-3.21 (m, 1H), 2.59-2.55 (m, 3H), 2.03-1.75 (m, 4H), 1.46-1.30 (m, 3H).

### Example 16—Synthesis of Target 14

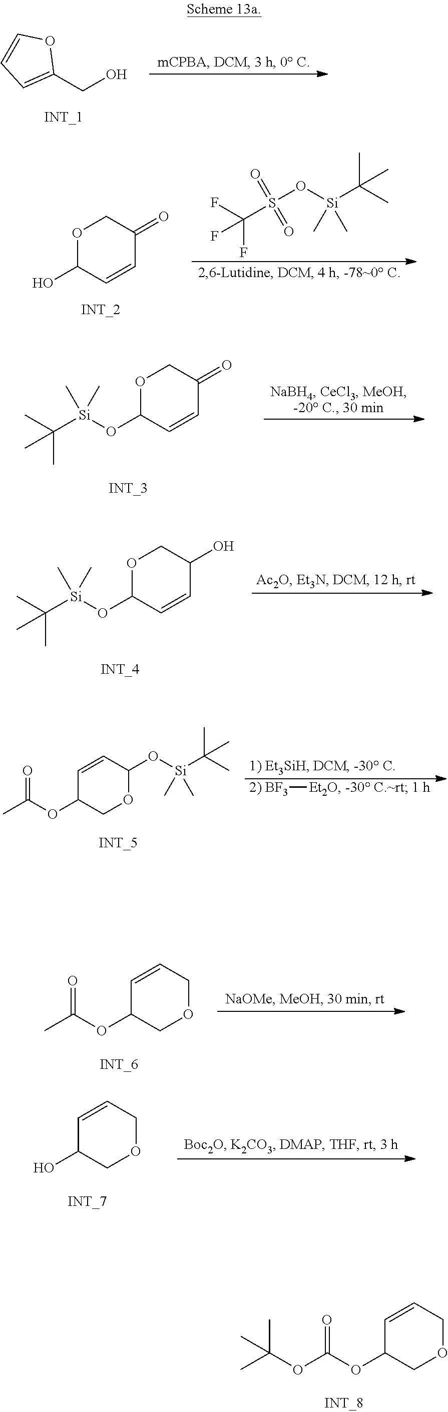

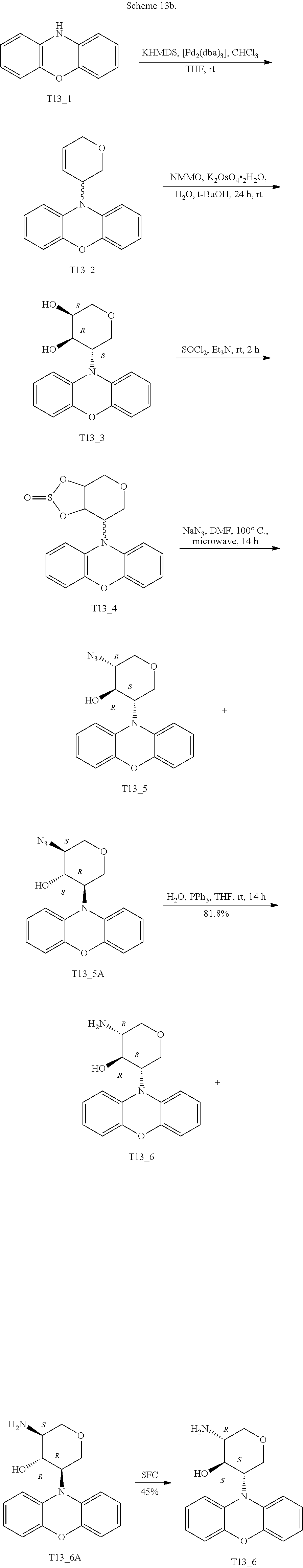

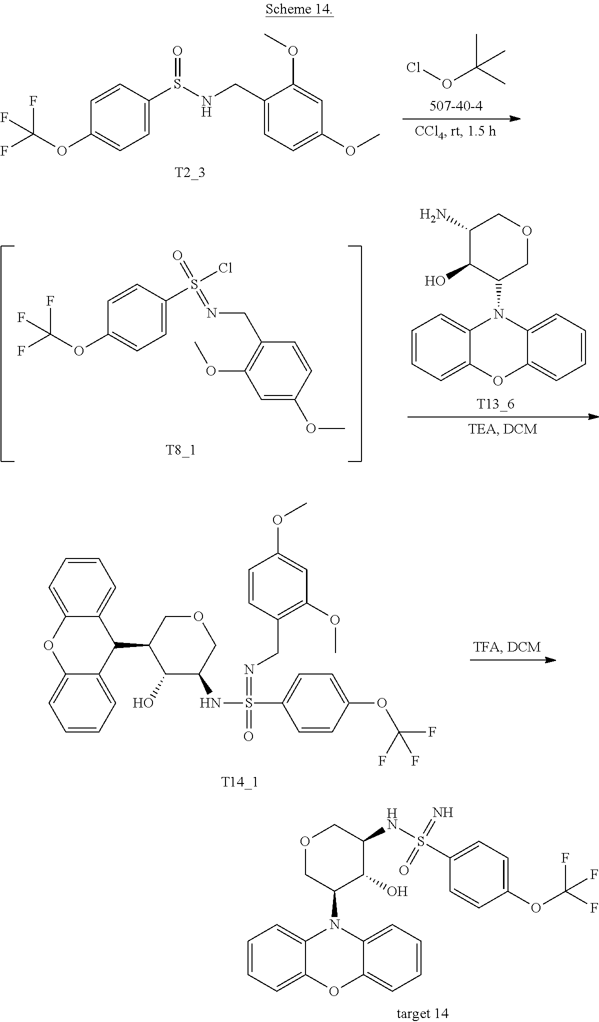

### Synthesis of Compound INT_2:

To a solution of m-CPBA (2 kg, 9.85 mol) in DCM (6 L) was added a solution of INT_1 (800 g, 8.15 mol) in DCM (800 mL) dropwise while keeping internal temperature below 10° C. After the addition, the mixture was warmed to 25° C. and stirred at 25° C. for 16 h. TLC showed that the reaction has been completed, the resulting mixture was filtered and filter cake was washed with DCM (2 L). The filtrate was concentrated under reduce pressure, the residue was purified by column chromatography on silica gel (PE:EA=7:1 to PE:EA=1:1) to afford INT_2 as a brown solid (500 g, 56.8%). ¹H NMR (400 MHz, CDCl₃), δ 7.01-6.98 (q, J₁=2.8 Hz, J₂=10.4 Hz, 1H), 6.20-6.18 (d, J=10.4 Hz, 1H), 5.66 (d, J=2.8 Hz, 1H), 4.62-4.57 (d, J=17.2 Hz, 1H), 4.18 4.14 (d, J=16.8 Hz, 1H).

### Synthesis of Compound INT_3:

To a solution of INT_2 (500 g, 4.37 mol) and 2,6-Lutidine (657.7 g, 5.2 mol) in DCM (2.5 L) was added t-butyldimethylsilyltrifluoromethanesulfonate (3.1 kg, 5.2 mol) dropwise under N₂ at −78° C. The reaction mixture was slowly warmed to 0° C. over 4 h. TLC showed that the reaction has been completed, the reaction was quenched by addition of water (1 L). The organic phase was washed with 10% citric acid aqueous (1 L) and brine (1 L). The organic layer was dried over Na₂SO₄, it was filtered and concentrated under reduced pressure, the residue was purified by column chromatography on silica gel (PE:EA=50:1) to afford INT_3 as a yellow oil (650 g, 74%). ¹H NMR (400 MHz, CDCl₃), δ 6.92-6.88 (q, J₁=3.2, J₂=10.4 Hz, 1H), 6.13-6.10 (d, J=10.4 Hz, 1H), 5.57-5.56 (d, J=3.2 Hz, 1H), 4.56-4.52 (d, J=16.8 Hz, 2H), 4.13-4.09 (d, J=16.8 Hz, 1H), 0.95 (s, 9H), 0.20 (s, 6H).

### Synthesis of Compound INT_4:

To a suspension of INT_3 (650 g, 2.8 mol) and CeCl₃.7H₂O (1.06 kg, 2.8 mol) in MeOH was added NaBH₄ (85.2 g, 3.2 mol) in small portions while keeping internal temperature below −20° C. The mixture was stirred at −20° C. for 30 min, TLC showed that the reaction has been completed, the resulting mixture was quenched with acetone (1.4 L) and stirred at 20° C. for 1 h. The solvent was removed under reduced pressure. The residue was dissolved with DCM (3 L) and eluted on silica gel pie to afford INT_4 as yellow oil (500 g, 76%). ¹H NMR (400 MHz, CDCl₃), δ 6.00-5.97 (q, J₁=2.8 Hz, J₂=2.8 Hz 10 Hz, 1H), 5.81-5.78 (m, 1H), 4.30-4.29 (m, 1H), 4.22-4.16 (m, 1H), 3.82-3.80 (m, 2H), 1.87-1.85 (d, J=9.2 Hz, 1H), 0.96 (s, 9H), 0.18 (d, J=2.0 Hz, 1H).

### Synthesis of Compound INT_5:

To a solution of INT_4 (500 g, 2.1 mol) and TEA (438.3 g, 4.4 mol) in DCM (4 L) was added acetic anhydride (332.2 g, 3.3 mol) dropwise at 25° C. The reaction mixture was stirred at 25° C. for 15 h. TLC showed that the reaction has been completed, MeOH (150 mL) was added and the mixture was stirred at 25° C. for 30 min before adding water (1.5 L). The organic layer was washed with water (1 L×2) and brine (1 L), dried over Na₂SO₄, concentrated under reduced pressure, then purified by column chromatography on silica gel (PE:EA=10:1) to afford INT_5 as a pale yellow oil (500 g, 84.5%). ¹H NMR (400 MHz, CDCl₃), δ 5.90-5.88 (m, 2H), 5.31-5.26 (m, 2H), 3.89-3.87 (m, 2H), 2.11 (s, 3H), 0.95 (s, 9H), 0.17 (d, J=8 Hz, 6H).

### Synthesis of Compound INT_6:

INT_5 (500 g, 1.84 mol) was dissolved in DCM (2.5 L), placed under a nitrogen atmosphere, and cooled to −30° C. (dry ice/acetone).

Triethylsilane (428.2 g, 9.2 mol) was added slowly via dropping funnel, followed by BF₃-Et₂O (318.8 g, 2.21 mol) was added dropwise at −30° C. The reaction mixture was kept under nitrogen and slowly warmed to 25° C. After 1 h, TLC showed that the reaction has been completed, the reaction mixture was quenched by dropped into saturated sodium bicarbonate aqueous (1 L). The organic layer was washed with water (1 L×2) and brine (1 L), dried over Na₂SO₄, it was filtered and concentrated under reduced pressure to give crude product INT_6 (500 g) that was directly used in the next step without further purification. ¹H NMR (400 MHz, CDCl₃), δ 6.12-6.08 (m, 1H), 5.98-5.93 (m, 1H), 5.12-5.10 (m, 1H), 4.27-4.22 (m, 1H), 4.14-4.09 (m, 1H), 3.97-3.93 (m, 1H), 3.85-3.80 (m, 1H), 2.12 (s, 3H).

### Synthesis of Compound INT_7:

To a solution of INT_6 (500 g crude, 1.84 mol) in MeOH (2.5 L) was added 30% sodium methoxide in methanol (149.2 g, 2.8 mol) at 25° C. The solution was stirred at 25° C. for 16 h, TLC showed that the reaction has been completed, the mixture was concentrated below 20° C. The residue was purified by column chromatography on silica gel (PE:EA=10:1 to 1:1) to afford INT_7 as light yellow oil (100 g, 53% in two steps). ¹H NMR (400 MHz, CDCl₃), δ 6.03-5.94 (m, 2H), 4.21-4.17 (m, 1H), 4.12-4.11 (m, 1H), 4.07 (m, 1H), 4.01-4.00 (m, 1H), 3.89-3.76 (m, 1H).

### Synthesis of Compound INT_8:

To a solution of INT_7 (100 g, 1.0 mol) in THF (1.0 L) was added n-BuLi (437 mL, 1.09 mol) dropwise while keeping internal temperature below −60° C. After addition, the reaction was stirred at −60° C. for 1 h. Then it was warmed to −40° C., Boc₂O (236 g, 1.18 mol) in THF (500 mL) was added into the mixture below −40° C. After addition, the mixture was stirred at −40° C. for 4 h. TLC showed that the reaction has been completed, the reaction was quenched by saturated NH₄Cl aqueous (2 L). Extracted with EtOAc (500 mL×2), the combined organic layers were washed with brine (500 mL), dried over NaSO₄, it was filtered and concentrated under reduced pressure, then the residue was purified by column chromatography on silica gel (PE EA=10:1) to afford INT_8 as light yellow solid (165 g, 81.9%). ¹H NMR (400 MHz, CDCl₃), δ 6.12-6.09 (m, 1H), 6.02-5.98 (m, 1H), 4.96-4.94 (m, 1H), 4.28-4.22 (m, 1H), 4.15-4.13 (m, 1H), 4.10-3.89 (m, 1H), 3.89-3.85 (m, 1H), 1.53 (s, 9H).

### Synthesis of Compound T13_2:

To a solution of T13_1 (100 g, 545.8 mmol) in THF (625 mL) was added a solution of KHMDS in THF (655 mL, 655 mmol, 1 mol/L) dropwise below 10° C. After addition, the mixture was stirred at 25° C. for 1 h to afford mixture A.

A suspension of INT_8 (131 g, 655 mmol) and Pd₂dba₃.CHCl₃ (28.3 g, 27.3 mmol) in THF (1.5 L) was degassed with Argon three times, then TPP (21.5 g, 81.9 mmol) was added in one portion and the mixture was stirred at 25° C. for 1 h under Argon atmosphere. Then the mixture A was added dropwise while keeping internal temperature below 20° C. After addition, the reaction mixture was stirred for 16 h at 25° C. TLC showed that the reaction has been completed, the mixture was poured into water (1 L), then it was extracted with EtOAc (500 mL×2), the combined organic layers was washed with brine (500 mL), dried over Na₂SO₄, it was filtered and concentrated under reduced pressure, the residue was purified by column chromatography on silica gel (PE:EA=50:1 to 40:1) to afford T13_2 as a light yellow solid (110 g, 76%). ¹H NMR (400 MHz, CDCl₃) δ 6.86-6.78 (m, 8H), 6.05-6.03 (m, 2H), 4.65-4.64 (m, 1H), 4.35-4.11 (m, 4H).

### Synthesis of Compound T13_3:

To a solution of T13_2 (110 g, 410.45 mmol) and NMMO (83.3 g, 615.7 mmol) in t-BuOH (2.0 L) and water (80 mL) was added K₂OsO₄.2H₂O (4.1 g, 11.1 mmol) in one portion at 25° C. The reaction mixture was stirred for 16 h at 25° C. TLC showed that the reaction has been completed, the resulting mixture was quenched by addition of water (1.5 L) and stirred for 1 h. Then filtered, the filter cake was dried at 55° C. for 48 h to afford T13_3 as grey solid (101 g, 83%). ¹H NMR (400 MHz, DMSO-d₆) δ 6.96-6.95 (m, 2H), 6.92-6.90 (m, 2H), 6.80-6.75 (m, 4H), 4.02-4.00 (m, 1H), 4.00-3.95 (m, 2H), 3.83-3.74 (m, 3H), 3.59-3.56 (m, 1H).

### Synthesis of Compound T13_4:

To a solution of T13_3 (101 g, 336.75 mmol) and TEA (102.2 g, 1.0 mol) in DCM (1 L) was added SOCl₂ (44.1 g, 370.4 mmol) dropwise at 0° C. The mixture was stirred at 25° C. for 3 h. TLC showed that the reaction has been completed, the reaction mixture was poured into water (500 mL), extracted with DCM (1 L×2), the combined organic layers was washed with brine (500 mL), dried over Na₂SO₄, it was filtered and concentrated under reduced pressure, then the residue was purified by column chromatography on silica gel (PE:EA=40:1) to afford T13_4 as yellow solid (80 g, 69%). ¹H NMR (400 MHz, CDCl₃) δ 6.98-6.88 (m, 6H), 6.78-6.76 (m, 2H), 5.46-5.42 (q, J₁=5.6 Hz, J₂=9.2 Hz, 1H), 5.10-5.09 (m, 1H), 4.51 4.48 (d, J=14.4 Hz, 1H), 4.26-4.22 (dd, J₁=4.8 Hz, J₂=11.6 Hz, 1H), 3.99-3.95 (dd, J₁=2.4 Hz, J₂=14.4 Hz, 1H), 3.84-3.78 (m, 1H), 3.72-3.67 (m, 1H).

Synthesis of Compound rac-T13_5 (Mixture of T13_5 and T13_5A):

A suspension of T13_4 (80 g, 232.3 mmol) and NaN₃ (42.7 g, 705 mmol) in DMF (700 mL) was heated to 110° C. for 18 h. TLC showed that the reaction has been completed, the reaction mixture was poured into water (2 L), extracted with EtOAc (500 mL×3), the combined organic layers were washed with brine (500 mL), dried over Na₂SO₄, it was filtered and concentrated under reduced pressure, then the residue was purified by column chromatography on silica gel (PE:EA=50:1 to DCM) to afford rac-T13_5 as yellow solid (60 g, 79.6%). ¹H NMR (400 MHz, CDCl₃) δ 6.91-6.82 (m, 9H), 4.12-4.03 (m, 2H), 3.99-3.97 (m, 1H), 3.96-3.91 (m, 3H), 3.59-3.50 (m, 1H), 3.10-2.80 (m, 1H).

### Synthesis of Compound T13_6:

A suspension of rac-T13_5 (60 g, 185 mmol) in EtOAc (2 L) was added Pd/C (12 g, 10 wt %) was stirred at 25° C. under hydrogen atmosphere for 48 h. TLC showed that the reaction has been completed, acetonitrile (2.5 L) was added and stirred for 1 h. The reaction mixture was filtered, the filtrate was concentrated to afford rac-T13_6 as off-white solid (40 g, 72.7%). Rac-T13_6 was purified by chiral SFC (SFC-7; ChiralPak AD, 300×50 mm I.D., 10 μm, CO₂/Methanol-0.1% NH₃H₂O=150%; 200 mL/min) to afford T13_6 as off-white solid (16.5 g, 41.3%, 98.3% ee).

### Synthesis of Compound T14_1:

To a solution of T2_3 (10 g, 26.57 mmol; see Example 2) in CCl₄ (100 mL) was added tert-butyl hypochlorite (3.2 g, 29.2 mmol) dropwise at 0° C. The mixture was stirred at 0° C. for 1 h (in dark). The solvent was removed by reduced pressure under 5° C. to afford crude product T8_1 (10 g).

To a solution of T13_6 (6.6 g, 22.15 mmol) and TEA (6.6 g, 65 mmol) in THF (70 mL) was added a solution of the crude T8_1 in THF (20 mL) dropwise while keeping internal temperature below 0° C. After addition, the mixture was stirred at 25° C. for 18 h. TLC showed that the reaction has been completed, water (50 mL) was added, extracted with EtOAc (40 mL×3), the combined organic layers were washed with brine (50 mL), dried over Na₂SO₄, it was filtered and concentrated under reduced pressure, then the residue was purified by column chromatography on silica gel (PE:EA=10:1 to 5:1) to afford T14_1 as white solid (4.8 g 29%). ¹H NMR (400 MHz, CDCl₃) δ 7.87-7.82 (m, 2H), 7.31-7.16 (m, 2H), 6.99-6.82 (m, 9H), 6.33-6.25 (m, 2H), 4.21-4.01 (m, 5H), 3.82-3.66 (m, 9H), 3.44-3.35 (m, 1H).

### Synthesis of Compound Target 14:

To a solution of T14_1 (4.8 g, 7.05 mmol) in DCM (50 mL) was added TFA (25 mL) dropwise at 25° C. The mixture was stirred at 25° C. for 18 h. TLC showed that the reaction has been completed, saturated NaHCO₃ aqueous was added to adjust pH >7.5, then extracted with DCM (20 mL×3), the combined organic layers were washed with brine (20 mL), dried over Na₂SO₄, it was filtered and concentrated under reduced pressure, then the residue was purified by column chromatography on silica gel (PE:EA=10:1 to 2:1) to afford Target 14 as white solid (1.15 g, 32%). ¹H NMR (400 MHz, CD₃OD) δ 8.14-8.11 (m, 2H), 7.47-7.45 (d, J=8.4, 2H), 6.99-6.85 (m, 6H), 6.80-6.78 (m, 2H), 4.11-4.04 (m, 2H), 3.81-3.63 (m, 3H), 3.26 (br s, 2H).

### Example 17—Synthesis of Target 15 (T15)

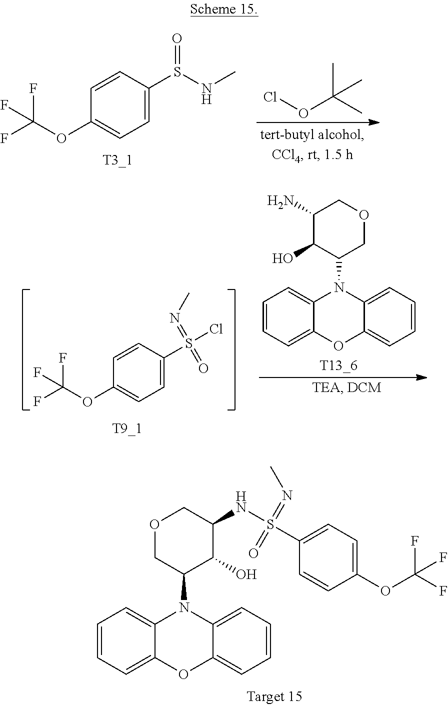

### Synthesis of Compound Target 15:

To a solution of T3_1 (5.0 g, 20.76 mmol; see Example 3) in CCl₄ (50 mL) was added tert-butyl hypochlorite (24.1 g, 22.48 mmol) dropwise at 0° C., The mixture was stirred at 0° C. for 1 h (in dark). The solvent was removed by reduced pressure under 5° C. to afford crude T9_1 (5 g).

To a solution of T13_6 (5.2 g, 17.3 mmol) and TEA (5.5 g, 54.2 mmol) in THF (40 mL) was added a solution of the crude T9_1 in THF (10 mL) dropwise at 0° C. After addition, the mixture was stirred at 25° C. for 18 h. TLC showed that the reaction has been completed, water (50 mL) was added, extracted with EtOAc (40 mL×3), the combined organic layers were washed with brine (50 mL), dried over Na₂SO₄, it was filtered and concentrated under reduced pressure, then the residue was purified by column chromatography on silica gel (PE:EA=10:1 to 5:1) to afford Target 15 as white solid (3.5 g 13.4%). ¹H NMR (400 MHz, CDCl₃) δ 8.01-8.00 (m, 2H), 7.39-7.30 (m, 2H), 6.98-6.82 (m, 8H), 4.22-4.17 (m, 3H), 4.11-3.94 (m, 2H), 3.83-3.53 (m, 2H), 2.69-2.64 (d, J=22.4 Hz, 3H). LCMS: M/Z (M+H)⁺ 536.1.

### Example 18—Synthesis of Target 17

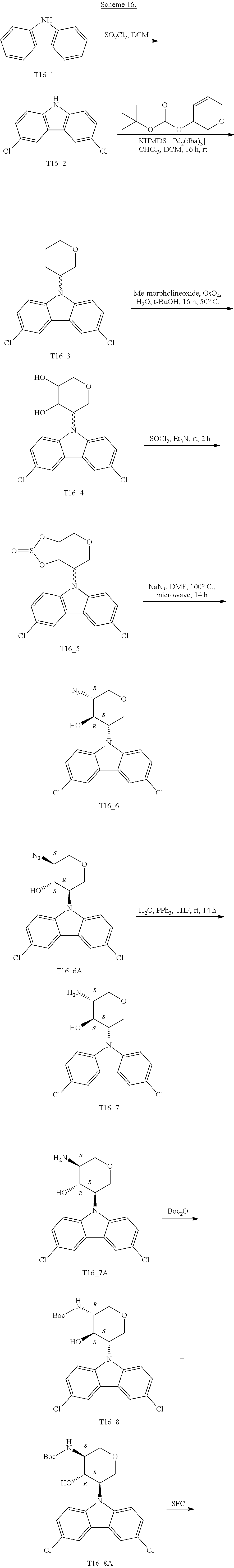

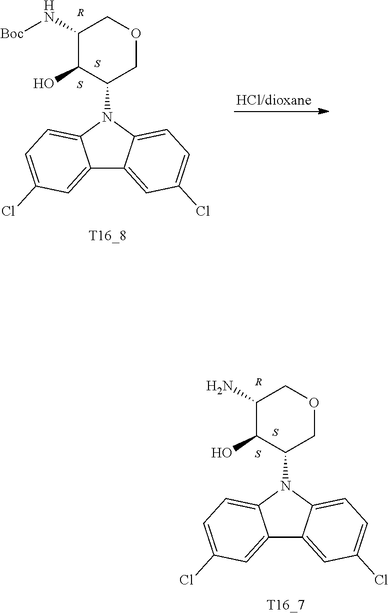

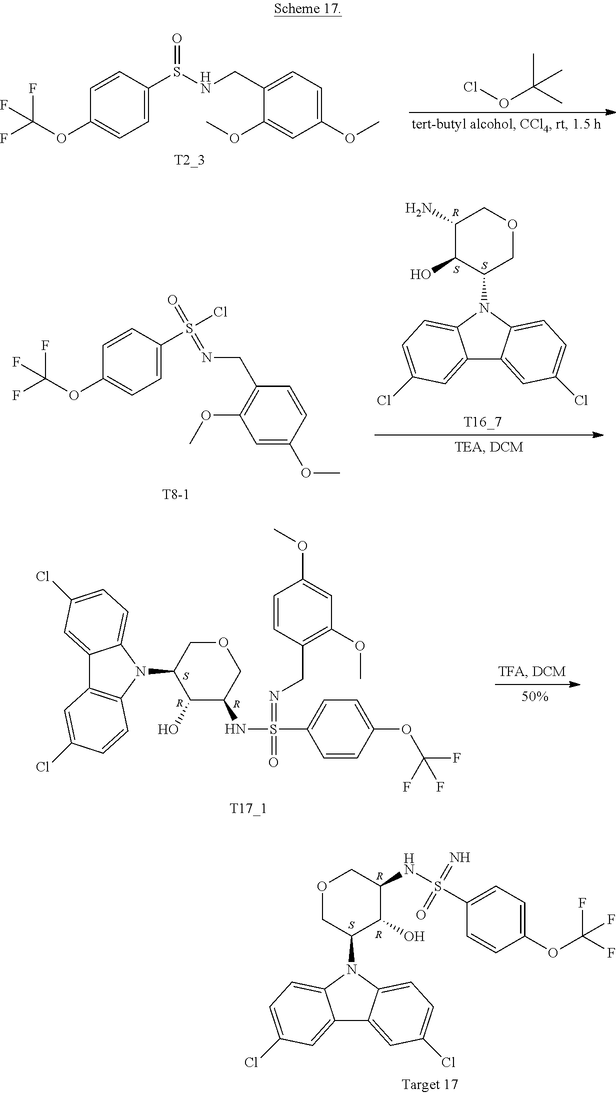

### Synthesis of Compound T16_2:

To a solution of T16_1 (100 g, 598.06 mmol) in CH₂Cl₂ (1 L) were added SO₂Cl₂ (96 mL, 1.2 mol) was added dropwise while keeping internal temperature below 2° C. After addition, the reaction mixture was stirred for 4 h at 25° C. TLC showed that the reaction has been completed, the precipitate was filtered, filtered cake was washed with CH₂Cl₂ (100 mL) and dried to afford T16_2 as white solid (60 g, 42%); ¹H NMR (400 MHz, DMSO-d₆) δ 11.60 (s, 1H), 8.32-8.31 (d, J=4 Hz, 2H), 7.55-7.53 (d, J=8.8 Hz, 2H), 7.46 7.43 (dd, J₁=8.8 Hz, J₂=4 Hz, 2H).

### Synthesis of Compound T16_3:

To a solution of T16_2 (100 g, 425.5 mmol) in THF (2 L) was added a solution of KHMDS in THF (508.27 mL, 510.6 mmol, 1 mol/L) dropwise while keeping internal temperature below 10° C. The mixture was warmed to 25° C. for 1 h to afford mixture A.

A suspension of INT_8 (101.77 g, 510.6 mmol) and Pd₂dba₃.CHCl₃ (21.92 g, 21.275 mmol) in THF (1 L) was degassed with Argon for three times, then TPP (16.74 g, 63.825 mmol) was added in one portion and the mixture was stirred for 1 h at 25° C. under Argon atmosphere. Then the mixture A was added dropwise while keeping the internal temperature below 20° C. After addition, the reaction mixture was stirred at 25° C. for 16 h. LCMS showed that the reaction has been completed, the mixture was poured into water (1 L), then it was extracted with EtOAc (1 L×2), the combined organic layers were washed with brine (300 mL), dried over Na₂SO₄, it was filtered and concentrated under reduced pressure, then the residue was purified by column chromatography on silica gel (PE:EA=50:1 to 40:1) to afford T16_3 as red solid (85 g, 63%); LCMS: M/Z (M+Na)⁺ 340.1.

### Synthesis of Compound T16_4:

To a solution of T16_3 (85 g, 268.1 mmol) and NMMO (33.13 g, 210.84 mmol) in t-BuOH (850 mL) and water (85 mL) was added K₂OsO₄-2H₂O (6.91 g, 18.8 mmol) in one portion at 25° C. The reaction mixture was heated to 50° C. for 16 h. LCMS showed that the reaction has been completed, it was poured into water (1.5 L), extracted with DCM (1 L×3). The combined organic layers were washed with brine (500 mL), dried over MgSO₄, filtered, and concentrated under reduced pressure to afford T16_4 as grey solid (60 g, 90%). LCMS: M/Z (M+H)⁺ 352.0.

### Synthesis of Compound T16_5:

To a solution of T16_4 (60 g, 170.45 mmol) and TEA (51.74 g, 511.35 mmol) in DCM (600 mL) was added SOCl₂ (60.85 g, 511.35 mmol) dropwise while keeping internal temperature below 0° C. The mixture was stirred at 25° C. for 3 h. LCMS showed that the reaction has been completed, it was quenched by pouring into water (500 mL), extracted with DCM (200 mL×2), the combined organic layers were washed with brine (100 mL), dried over Na₂SO₄, filtered, and concentrated under reduced pressure, the residue was purified by column chromatography on silica gel (PE:EA=2:1) to afford T16_5 as a yellow solid (60 g, 89%). LCMS: M/Z (M+H)⁺ 398.0.

### Synthesis of Compound T16_6:

A suspension of T16_5 (60 g, 150.6 mmol) and NaN₃ (29.4 g, 452.2 mmol) in DMF (600 mL) was heated to 110° C. for 18 h. LCMS showed that the reaction has been completed, it was quenched by pouring into water (2 L), extracted with EA (500 mL×3), the combined organic layers were washed with brine (500 mL), dried over Na₂SO₄, filtered, and concentrated under reduced pressure to afford T16_6 as yellow solid (54 g, 95%) which was directly used for the next step without further purification. LCMS: M/Z (M+H)⁺ 377.0.

Synthesis of Compound rac-T16_7 (Mixture of T16_7 and T16_7A):

A solution of T16_6 (54 g, 143.2 mmol) in THF (540 mL) and H₂O (5.4 mL) was added PPh₃ (43.15 g, 164.68 mmol) in small portions at 0° C. After addition, it was stirred at 25° C. for 15 h. LCMS showed that the reaction has been completed, it was concentrated to dryness, the residue was purified by column chromatography on silica gel (DCM:MeOH=10:1) to afford rac-T16_7 as white solid (45 g, 90%). ¹H NMR (400 MHz, DMSO-d₆) δ 8.39-8.34 (dd, J₁=2 Hz, J₂=16.4 Hz, 2H), 7.90-7.88 (d, J=9.2 Hz, 1H), 7.75-7.73 (d, J=8.8 Hz, 1H), 7.51-7.44 (m, 2H), 5.10-5.09 (d, J=5.6 Hz, 1H), 4.62-4.57 (m, 1H), 4.21-4.14 (m, 2H), 3.91-3.86 (m, 2H), 3.43-3.36 (m, 1H), 2.54-2.51 (m, 1H). LCMS: M/Z (M+H)⁺ 351.1.

### Synthesis of Compound T16_8:

A solution of rac-T16_7 (45 g, 127.8 mmol) and TEA (26.58 mL, 191.8 mmol) in THF (450 mL), was added Boc₂O (33.45 g, 153.36 mmol) dropwise at 25° C. The reaction mixture was stirred at 25° C. for 16 h. LCMS showed that the reaction has been completed, it was quenched by pouring into water (450 mL), extracted with DCM (400 mL×3), the combined organic layers were washed with brine (300 mL), dried over Na₂SO₄ and concentrated under reduced pressure to afford rac-T16_8 (50 g) which was separated by SFC (SFC-10; Chiralpak AD-3 150×4.6 mm I.D., 3 μm; 40% of ethanol (0.05% DEA) in CO₂; 2.5 mL/min) to afford T16_8 as light yellow solid (22 g, 48.89%). ¹H NMR (400 MHz, CDCl₃) δ 8.05-8.02 (m, 2H), 7.48-7.30 (m, 4H), 4.71-4.58 (m, 3H), 4.28-4.22 (m, 2H), 4.18-4.13 (m, 1H), 4.09-3.88 (m, 1H), 3.54-3.14 (m, 1H), 2.08 (br s, 1H), 1.47 (s, 9H). LCMS: M/Z (M-56)⁺ 397.1.

### Synthesis of Compound T16_7:

To a solution of T16_8 (22 g, 48.9 mmol) in dioxane (100 mL) was added HCl/dioxane (200 mL) dropwise at 25° C. The mixture was stirred at 25° C. for 2 h. LCMS showed that the reaction has been completed, it was concentrated to afford the T16_7 as white solid (16.5 g, 87.3%). ¹H NMR (400 MHz, DMSO-d₆) δ 8.39-8.34 (dd, J₁=2 Hz, J₂=16.4 Hz, 2H), 7.90-7.88 (d, J=9.2 Hz, 1H), 7.75-7.73 (d, J=8.8 Hz, 1H), 7.50-7.44 (m, 2H), 5.10-5.09 (d, J=5.6 Hz, 1H), 4.62-4.57 (m, 1H), 4.21-4.14 (m, 2H), 3.91-3.86 (m, 2H), 3.43-3.18 (m, 1H), 2.88-2.82 (m, 1H). LCMS: M/Z (M+H)+351.

### Synthesis of Compound T17_1:

To a solution of T2_3 (7.7 g, 17 mmol; see Example 2) in CCl₄ (60 mL) was added tert-butyl hypochlorite (2.6 g, 23.94 mmol) dropwise at 0° C. The mixture was stirred at 0° C. for 1 h (in dark). TLC showed that the reaction has been completed, the solvent was removed by reduce pressure under 5° C. to afford crude product T8_1 (7.7 g, crude).

To a solution of T16_7 (6 g, 16.78 mmol) and TEA (8.34 g, 82.4 mmol) in THF (40 mL) was added a solution of T8_1 in THF (20 mL) was added into the mixture at 0° C. After addition, it was stirred at 25° C. for 18 h. LCMS showed that the reaction has been completed, water (80 mL) was added, extracted with EtOAc (50 mL×3), the combined organic layers were washed with brine (30 mL), dried over Na₂SO₄, it was filtered and concentrated under reduced pressure, then the residue was purified by column chromatography on silica gel (PE:EA=10:1 to 5:1) to afford T17_1 as white solid (8 g, 64.8%). LCMS: M/Z (M+H)⁺ 724.2, 726.2.

### Synthesis of Compound Target 17:

To a solution of T17_1 (4 g, 11.05 mmol) in DCM (40 mL) was added TFA (20 mL) dropwise at 25° C. The mixture was stirred at 25° C. for 16 h. LCMS showed that the reaction has been completed, saturated NaHCO₃ aqueous was added to adjust pH >7.5, then it was extracted with DCM (100 mL×3), the combined organic layers were washed with brine (50 mL), dried over Na₂SO₄, filtered and concentrated under reduced pressure, then the residue was purified by column chromatography on silica gel (PE:EA=8:1 to 4:1) to afford Target 17 as white solid (1.2 g, 40%). ¹H NMR (400 MHz, CD₃OD) δ 8.15-8.10 (m, 4H), 7.76-7.74 (d, J=8.8 Hz, 1H), 7.61-7.59 (d, J=8.8 Hz, 1H), 7.46-7.44 (m, 4H), 4.67-4.63 (m, 1H), 4.50-4.48 (m, 1H), 4.27 (m, 1H), 3.97-3.93 (m, 2H), 3.52 (m, 1H), 3.35-3.33 (br s, 2H). LCMS: M/Z (M+H)⁺ 574.1.

### Example 19—Synthesis of Target 18 (T18)

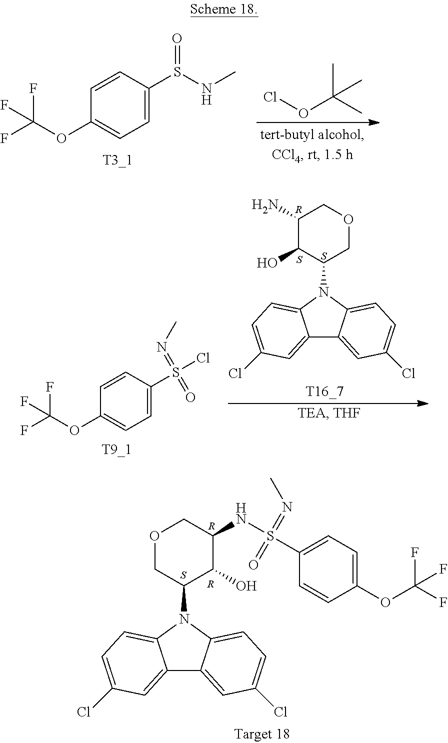

### Synthesis of Compound Target 18:

To a solution of T3_1 (4 g, 16.27 mmol; see Example 3) in CCl₄ (30 mL) was added tert-butyl hypochlorite (2.1 g, 43.9 mmol) dropwise at 0° C., the mixture was stirred at 0° C. for 1 h (in dark). LCMS showed that the reaction has been completed, the solvent was removed by reduced pressure under 5° C. to afford crude product T9_1 (4.0 g).

To a solution of T16_7 (3.9 g, 11.1 mmol) and TEA (5.7 g, 55.5 mmol) in THF (40 mL) was added a solution of the T9_1 in THF (10 mL) dropwise at 0° C. After addition, the mixture was stirred at 25° C. for 18 h. LCMS showed that the reaction has been completed, water (50 mL) was added, extracted with EtOAc (40 mL×3), the combined organic layers were washed with brine (50 mL), dried over Na₂SO₄, filtered and concentrated under reduced pressure, then the residue was purified by prep-HPLC to afford Target 18 as white solid (1.1 g, 29%). ¹H NMR (400 MHz, CD₃OD) δ 8.16-8.08 (m, 4H), 7.82-7.77 (m, 1H), 7.68-7.61 (m, 1H), 7.51-7.42 (m, 4H), 4.89-4.67 (m, 1H), 4.56-4.46 (m, 1H), 4.36-4.29 (m, 1H), 4.14-4.11 (m, 1H), 4.02-3.96 (m, 1H), 3.76-3.33 (m, 2H), 2.61 (s, 3H). LCMS: M/Z (M+H)⁺ 588.1.

### Example 20—Chiral Separation Method Parameters for Targets

### 1. Chiral Separation Method for Compound Target 5:

Instrument: MG II preparative SFC(SFC-14)

### Column: ChiralPak IC, 250×30 mm I.D., 10 μm

Mobile phase: A for CO₂ and B for Ethanol (0.1% NH₃H₂O)

### Gradient: B 40%

Flow rate: 80 mL/min
Back pressure: 100 bar
Column temperature: 38° C.

### Wavelength: 220 nm

Cycle time: ˜2 min
Sample preparation: Compound was dissolved in ˜50 ml methanol/DCM
Injection: 2 ml per injection.
Work up: After separation, the fractions were evaporated to dryness using a rotary evaporator (bath temperature 40° C.) to get the desired isomers.

Spectrogram of diastereomer 1: ¹H NMR (400 MHz, CDCl₃) δ 7.97 7.95 (d, J=8.8 Hz, 2H), 7.30-7.27 (d, J=10.8 Hz, 2H), 7.15-7.13 (m, 4H), 7.09-7.07 (d, J=7.2 Hz, 2H), 7.01-7.00 (m, 2H), 3.85-3.75 (m, 3H), 3.26-3.22 (dd, J=2.8 Hz, J=13.2 Hz, 1H), 3.16 (s, 4H), 3.03-2.98 (dd, J=6 Hz, J=13.2 Hz, 1H). LCMS: M/Z (M+H)⁺ 492.2.

Spectrogram of diastereomer 2: ¹H NMR (400 MHz, CDCl₃) δ 8.10 8.09 (d, J=8.8 Hz, 2H), 7.42-7.40 (m, 2H), 7.29-7.24 (m, 4H), 7.19-7.13 (d, J=8 Hz, 2H), 7.13-7.09 (t, J=7.2 Hz, J=14.4 Hz, 2H), 4.02-3.93 (m, 2H), 3.86-3.81 (m, 1H), 3.39-3.35 (m, 1H), 3.27 (s, 4H), 3.09-3.04 (q, J=6.8 Hz, J=13.6 Hz, 1H). LCMS: M/Z (M+H)⁺ 492.2.

### 2. Chiral Separation Method for Compound Target 6:

Instrument: MG II preparative SFC (SFC-14)

### Column: ChiralPak IC, 250×30 mm I.D., 10 μm

Mobile phase: A for CO₂ and B for Ethanol (0.1% NH₃H₂O)

### Gradient: B 40%

Flow rate: 80 mL/min
Back pressure: 100 bar
Column temperature: 38° C.

### Wavelength: 220 nm

Cycle time: ˜2 min
Sample preparation: Compound was dissolved in ˜30 ml methanol
Injection: 1 ml per injection.
Work up: After separation, the fractions were evaporated to dryness using a rotary evaporator (bath temperature 40° C.) to get the desired isomers.

Spectrogram of diastereomer 1: ¹H NMR (400 MHz, CDCl₃) δ 7.93-7.90 (m, 2H), 7.32-7.30 (m, 2H), 7.17-7.15 (m, 6H), 6.99-6.95 (m, 2H), 3.97-3.93 (m, 2H), 3.85-3.83 (m, 1H), 3.38-3.20 (m, 6H), 2.59 (s, 3H). LCMS: M/Z (M+H)⁺ 506.2.

Spectrogram of diastereomer 2: ¹H NMR (400 MHz, CDCl₃) δ 7.95-7.93 (m, 2H), 7.30 (s, 2H), 7.18-7.14 (m, 6H), 7.01-6.97 (m, 2H), 3.97-3.94 (m, 3H), 3.49-3.45 (m, 1H), 3.23-3.18 (m, 5H), 2.64 (s, 3H). LCMS: M/Z (M+H)⁺ 506.2.

### 3. Chiral Separation Method for Compound Target 11:

Instrument: MG 1H preparative SFC(SFC-14)

### Column: ChiralCel OX, 250×30 mm I.D., 5 μm

Mobile phase: A for CO₂ and B for Ethanol

### Gradient: B 30%

Flow rate: 40 mL/min
Back pressure: 100 bar
Column temperature: 38° C.

### Wavelength: 254 nm

Cycle time: ˜4.7 min
Sample preparation: Compound was dissolved in ˜20 ml methanol/DCM
Injection: 1 ml per injection.
Work up: After separation, the fractions were evaporated to dryness using a rotary evaporator (bath temperature 40° C.) to get the desired isomers.

Spectrogram of diastereomer 1: ¹H NMR (400 MHz, CD₃OD) δ 8.16-8.13 (m, 2H), 7.48-7.46 (d, J=8.8 Hz, 2H), 7.03-7.01 (d, J=8 Hz, 2H), 6.94-6.92 (m, 2H), 6.91-6.90 (m, 2H), 6.87-6.82 (m, 2H), 3.85-3.83 (m, 1H), 3.51-3.40 (m, 1H), 3.35-3.34 (m, 1H), 1.96-1.67 (m, 4H), 1.34 (s, 2H).

LCMS: M/Z (M+H)⁺ 520.1.

Spectrogram of diastereomer 2: ¹H NMR (400 MHz, CD₃OD) δ 8.15-8.13 (m, 2H), 7.47-7.45 (d, J=8.4 Hz, 2H), 7.01-7.00 (m, 2H), 6.93-6.90 (m, 2H), 6.86-6.82 (m, 2H), 6.77-6.75 (m, 2H), 3.90-3.85 (t, J=9.2 Hz, J=19.2 Hz, 1H), 3.48-3.40 (m, 1H), 3.35-3.17 (m, 1H), 1.93-1.67 (m, 4H), 1.33 (s, 2H).

LCMS: M/Z (M+H)⁺ 520.2.

### 4. Chiral Separation Method for Compound Target 12:

Instrument: MG H preparative SFC(SFC-14)

### Column: ChiralPak AD, 250×30 mm I.D., 5 μm

Mobile phase: A for CO₂ and B for Ethanol

### Gradient: B 50%

Flow rate: 40 mL/min
Back pressure: 100 bar
Column temperature: 38° C.

### Wavelength: 220 nm

Cycle time: ˜3.85 min
Sample preparation: Compound was dissolved in 30 ml methanol/DCM
Injection: 3 ml per injection.
Work up: After separation, the fractions were evaporated to dryness using a rotary evaporator (bath temperature 40° C.) to get the desired isomers.

Spectrogram of diastereomer1: ¹H NMR (400 MHz, CD₃OD) δ 8.13 8.11 (d, J=8.0 Hz, 2H), 7.50-7.48 (d, J=8.8 Hz, 2H), 7.06-7.04 (d, J=8.0 Hz, 2H), 6.95-6.91 (m, 2H), 6.86-6.82 (m, 2H), 6.78-6.76 (m, 2H), 4.01-3.97 (t, J=9.2 Hz, J=19.2 Hz, 1H), 3.56-3.34 (m, 2H), 2.62 (s, 3H), 2.07-1.34 (m, 7H).

LCMS: M/Z (M+H)⁺ 534.2.

Spectrogram of diastereomer2: ¹H NMR (400 MHz, CD₃OD) δ 8.11-8.08 (m, 2H), 7.51-7.29 (d, J=10.0 Hz, 2H), 7.04-7.02 (d, J=7.6 Hz, 2H), 6.94-6.89 (m, 2H), 6.86-6.81 (m, 2H), 6.77-6.75 (m, 2H), 3.92-3.89 (t, J=8.8 Hz, J=10.0 Hz, 1H), 3.48-3.47 (m, 1H), 3.35-3.25 (m, 1H), 2.58 (s, 3H), 1.98-1.78 (m, 4H), 1.33 (m, 3H).

LCMS: M/Z (M+H)⁺ 534.2.

### 5. Chiral Separation Method for Compound Target 14:

Instrument: MG H preparative SFC (SFC-22)

### Column: ChiralPak AD, 250×30 mm I.D., 10 μm

Mobile phase: A for CO₂ and B for Ethanol

### Gradient: B 40%

Flow rate: 80 mL/min
Back pressure: 100 bar
Column temperature: 38° C.

### Wavelength: 220 nm

Cycle time: ˜5 min
Sample preparation: Compound was dissolved in ˜40 ml methanol/DCM
Injection: 1 ml per injection.
Work up: After separation, the fractions were evaporated to dryness using a rotary evaporator (bath temperature 40° C.) to get the desired isomers.

Spectrogram of diastereomer1: ¹H NMR (400 MHz, CD₃OD) δ 8.13-8.11 (m, 2H), 7.46-7.44 (d, J=8.8 Hz, 2H), 6.99-6.84 (m, 6H), 6.79-6.77 (dd, J=1.6 Hz, J=9.2 Hz, 2H), 4.12-4.03 (m, 2H), 3.85-3.76 (m, 2H), 3.67-3.60 (m, 1H), 3.35-3.23 (m, 2H).

LCMS: M/Z (M+H)⁺ 522.1

Spectrogram of diastereomer2: ¹H NMR (400 MHz, CD₃OD) δ 8.13-8.11 (m, 2H), 7.46-7.44 (d, J=8.8 Hz, 2H), 6.99-6.84 (m, 6H), 6.79-6.77 (dd, J=1.6 Hz, J=9.2 Hz, 2H), 4.12-4.03 (m, 2H), 3.81-3.76 (m, 2H), 3.63-3.62 (m, 1H), 3.35-3.26 (m, 2H).

LCMS: M/Z (M+H)⁺ 522.2

### 6. Chiral Separation Method for Compound Target 15:

Instrument: MG 1H preparative SFC(SFC-14)

### Column: ChiralPak IG, 250×30 mm I.D., 5 μm

Mobile phase: A for CO₂ and B for Ethanol

### Gradient: B 35%

Flow rate: 70 mL/min
Back pressure: 100 bar
Column temperature: 38° C.

### Wavelength: 220 nm

Cycle time: ˜7 min
Sample preparation: Compound was dissolved in 30 ml methanol/DCM
Injection: 3 ml per injection.
Work up: After separation, the fractions were evaporated to dryness using a rotary evaporator (bath temperature 40° C.) to get the desired isomers.

Spectrogram of diastereomer1: ¹H NMR (400 MHz, CD₃OD) δ 8.11-8.09 (m, 2H), 7.48-7.46 (d, J=8.0 Hz, 2H), 6.97-6.93 (m, 2H), 6.89-6.88 (m, 2H), 6.87-6.85 (m, 2H), 6.80-6.78 (m, 2H), 4.18-4.14 (m, 2H), 4.01-3.97 (m, 1H), 3.91-3.85 (m, 1H), 3.77-3.71 (m, 1H), 3.58-3.51 (m, 1H), 3.35-328 (m, 1H), 2.59 (s, 3H).

LCMS: M/Z (M+H)⁺ 536.2.

Spectrogram of diastereomer2: ¹H NMR (400 MHz, CD₃OD) δ 8.19-8.17 (m, 2H), 7.62-7.60 (d, J=8.0 Hz, 2H), 7.13-7.04 (m, 4H), 7.00-6.96 (m, 2H), 6.92-6.89 (m, 2H), 4.25-4.13 (m, 2H), 4.11-4.01 (m, 1H), 3.99-3.95 (m, 1H), 3.85-3.77 (m, 1H), 3.47-3.45 (m, 2H), 2.59 (s, 3H).

LCMS: M/Z (M+H)⁺ 536.2.

### 7. Chiral Separation Method for Compound Target 17:

Instrument: MG 1H preparative SFC(SFC-14)

### Column: ChiralPak IC, 250×30 mm I.D., 5 μm

Mobile phase: A for CO₂ and B for Ethanol

### Gradient: B 40%

Flow rate: 80 mL/min
Back pressure: 100 bar
Column temperature: 38° C.

### Wavelength: 254 nm

Cycle time: ˜3.5 min
Sample preparation: Compound was dissolved in ˜120 ml methanol/DCM
Injection: 3 ml per injection.
Work up: After separation, the fractions were evaporated to dryness using a rotary evaporator (bath temperature 40° C.) to get the desired isomers.

Spectrogram of diastereomer1: ¹H NMR (400 MHz, CD₃OD) δ 8.17-8.09 (m, 4H), 7.77-7.64 (m, 2H), 7.48-7.44 (m, 4H), 4.72 (br s, 1H), 4.53-4.48 (m, 1H), 4.30-4.29 (m, 1H), 3.99-3.93 (m, 2H), 3.81-3.35 (m, 2H).

LCMS: M/Z (M+H)⁺ 574.1.

Spectrogram of diastereomer2: ¹H NMR (400 MHz, CD₃OD) δ 8.16-8.01 (m, 4H), 7.76-7.74 (d, J=8.8 Hz, 1H), 7.61-7.58 (m, 1H), 7.46-7.44 (m, 4H), 4.70-4.63 (m, 1H), 4.51-4.46 (m, 1H), 4.30-4.25 (m, 1H), 4.03-3.93 (m, 2H), 3.77-3.34 (m, 2H).

LCMS: M/Z (M+H)⁺ 574.1.

### 8. Chiral Separation Method for Compound Target 18:

Instrument: MG 1H preparative SFC(SFC-14)

### Column: ChiralCel OX, 250×30 mm I.D., 5 μm

Mobile phase: A for CO₂ and B for Ethanol

### Gradient: B 25%

Flow rate: 60 mL/min
Back pressure: 100 bar
Column temperature: 38° C.° C.

### Wavelength: 220 nm

Cycle time: ˜12.5 min
Sample preparation: Compound was dissolved in ˜10 ml methanol/DCM
Injection: 3.5 ml per injection.
Work up: After separation, the fractions were evaporated to dryness using a rotary evaporator (bath temperature 40° C.) to get the desired isomers.

Spectrogram of diastereomer1: ¹H NMR (400 MHz, CD₃OD) δ 8.16-8.09 (m, 4H), 7.83-7.80 (d, J=8.8 Hz, 1H), 7.68-7.66 (d, J=9.2 Hz, 1H), 7.50-7.44 (m, 4H), 4.79-4.72 (m, 1H), 4.57-4.52 (t, J=8.8 Hz, J=19.2 Hz, 1H), 4.36-4.30 (t, J=11.6 Hz, J=22.8 Hz, 1H), 4.16-4.12 (m, 1H), 4.03-3.98 (m, 1H), 3.79-3.73 (m, 1H), 3.35-3.34 (m, 1H), 2.61 (s, 3H).

LCMS: M/Z (M+H)⁺ 588.1.

Spectrogram of diastereomer2: ¹H NMR (400 MHz, CD₃OD) δ 8.14-8.08 (m, 4H), 7.80-7.78 (d, J=8.8 Hz, 1H), 7.64-7.61 (d, J=9.2 Hz, 1H), 7.51-7.43 (m, 4H), 4.71-4.69 (m, 1H), 4.52 4.47 (t, J=8.4, J=18.8, 1H), 4.35-4.30 (t, J=11.2 Hz, J=22.8 Hz, 1H), 4.14-4.12 (m, 1H), 4.01-3.97 (m, 1H), 3.64-3.62 (m, 2H), 2.60 (s, 3H).

LCMS: M/Z (M+H)⁺ 588.1.

### Example 21—Synthesis of Target 15A (T15A)

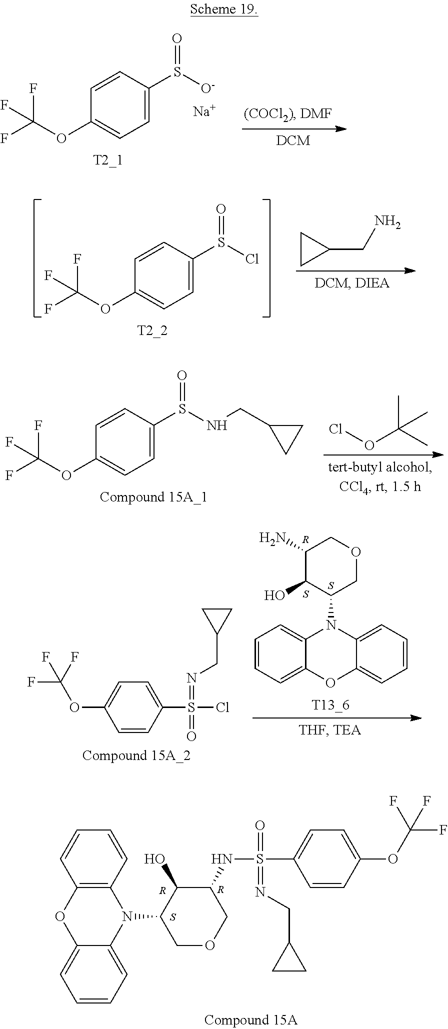

### Synthesis of Compound 15A_1

Oxalyl chloride (3.1 g, 24.2 mmol) was added to a mixture of T2_1 (5.0 g, 20.2 mmol) and DMF (0.1 mL) in DCM (50 mL) with temperature being set below 20° C. The mixture was stirred for 1 h after the addition, the resulting mixture was concentrated under vacuum to give crude compound T2_2. The crude compound T2_2 was dissolved in DCM (50 mL) and cyclopropylmethanamine (1.8 g, 25.2 mmol) and DIEA (7.8 g, 60.5 mmol) was added into the mixture. The mixture was stirred for 18 h at 15 to 25° C. The reaction was quenched with water (50 mL) and separated to collect the organic phase. We repeated this procedure by adding DCM (50 mL) to the remaining aqueous phase and collected the organic. Combined organic phase was washed with brine (30 mL), dried over Na₂SO₄, concentrated, and purified by column chromatography on silica gel (PE/EA=10/1) to give Compound 15A_1 as off-white solid (1.6 g, 28%). ¹H NMR (400 MHz, CDCl₃) δ 7.80-7.77 (m, 2H), 7.38-7.36 (d, J=8.4 Hz, 2H), 4.26-4.23 (m, 1H), 2.97-2.91 (m, 1H), 2.74-2.67 (m, 1H), 1.03-0.97 (m, 1H), 0.57-0.53 (m, 2H), 0.22-0.11 (m, 2H).

### Synthesis of Compound 15A

To a solution of Compound 15A_1 (170 mg, 0.6 mmol) in CCl₄ (5 mL) was added tert-butyl hypochlorite (97 mg, 0.9 mmol) dropwise at 0° C. The mixture was stirred at 0° C. for 1 h (in the dark). TLC showed that the reaction had been completed, the solvent was removed by reduce pressure under 5° C. to afford crude product Compound 15A 2 (170 mg).

To a solution of T13_6 (120 mg, 0.4 mmol) and TEA (182 mg, 1.8 mmol) in THF (5 mL) was added a solution of crude product Compound 15A 2 in THF (2 mL) dropwise at 0° C. After addition, the mixture was stirred at 25° C. for 18 h. After TLC showed that the reaction had been completed, the mixture was poured into water (10 mL), extracted with EtOAc (10 mL) three times, the combined organic layers were washed with brine (10 mL), dried over Na₂SO₄, filtered, and concentrated under reduced pressure. The residue was purified by column chromatography on silica gel (PE:EA=5:1) to afford Compound 15A as pale purple solid (70 mg, 30%). ¹H NMR (400 MHz, CD₃OD) δ 8.17-8.14 (m, 2H), 7.59-7.52 (m, 2H), 7.03-6.93 (m, 4H), 6.91-6.85 (m, 2H), 6.81-6.78 (m, 2H), 4.20-4.08 (m, 2H), 4.06-3.93 (m, 1H), 3.88-3.79 (m, 1H), 3.77-3.44 (m, 2H), 3.44-3.38 (m, 1H), 3.03-2.94 (m, 2H), 0.98-0.89 (m, 1H), 0.54-0.44 (m, 2H), 0.24-0.15 (m, 2H). LCMS: M/Z (M+H)+576.2.

### Example 22—Synthesis of Target 15C (T15C)

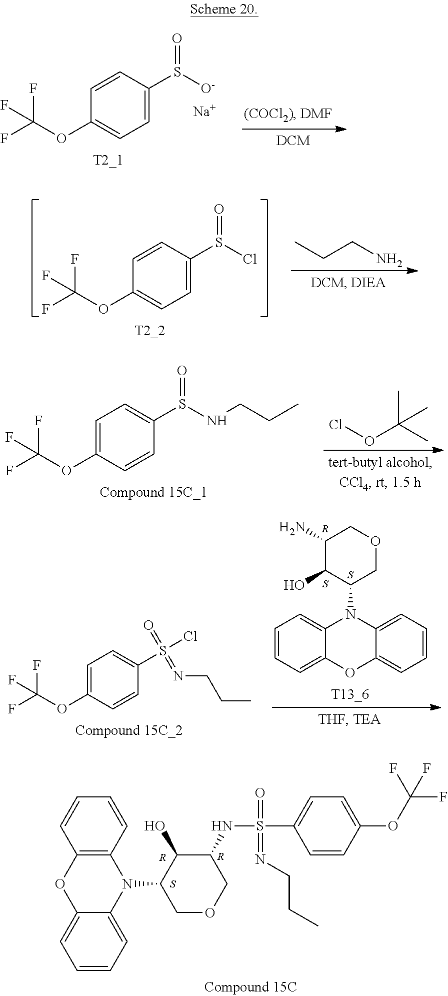

### Synthesis of Compound 15C_1

To a mixture of T2_1 (5.0 g, 20.2 mmol) and DMF (0.1 mL) in DCM (50 mL) was added Oxalyl chloride (3.1 g, 24.2 mmol) below 20° C. The mixture was stirred for 1 h after the addition, the resulting mixture was concentrated under vacuum to give crude compound T2_2. Crude compound T2_2 was dissolved in DCM (50 mL) and then propan-1-amine (1.5 g, 25.2 mmol) and DIEA (7.8 g, 60.5 mmol) were added into the mixture. The mixture was stirred for 18 h at 15 to 25° C. Then the reaction was quenched with water (50 mL) and separated to collect the organic phase, the remaining aqueous phase was extracted with DCM (50 mL) another time. The combined organic phase was washed with brine (30 mL), dried over Na₂SO₄, concentrated, and purified by column chromatography on silica gel (PE/EA=10/1) to give Compound 15C_1 as off-white solid (1.7 g, 31%). ¹H NMR (400 MHz, CDCl₃) δ 7.81-7.77 (m, 2H), 7.39-7.37 (m, 2H), 4.16-4.12 (m, 1H), 4.15-3.09 (m, 1H), 2.86-2.79 (m, 1H), 1.60-1.55 (m, 2H), 0.96-0.92 (t, J=8.4, 14.8 Hz, 3H).

### Synthesis of Compound 15C

To a solution of Compound 15A_1 (160 mg, 0.6 mmol) in CCl₄ (5 mL) was added tert-butyl hypochlorite (97 mg, 0.9 mmol) dropwise at 0° C. The mixture was stirred at 0° C. for 1 h (keep it in dark). After TLC showed that the reaction had been completed, the solvent was removed by reducing pressure under 5° C. to afford crude product Compound 15C_2 (160 mg).

To a solution of T13_6 (120 mg, 0.4 mmol) and TEA (182 mg, 1.8 mmol) in THF (5 mL) was added a solution of the crude product Compound 15C_2 in THF (2 mL) dropwise at 0° C. After addition, the mixture was stirred at 25° C. for 18 h. After TLC showed that the reaction had been completed, it was poured into water (10 mL), extracted with EtOAc (10 mL) three times, the combined organic layers were washed with brine (10 mL), dried over Na₂SO₄, then it was filtered and concentrated under reduced pressure, then the residue was purified by column chromatography on silica gel (PE:EA=5:1) to afford Compound 15C as pale purple solid (55 mg, 25%). ¹H NMR (400 MHz, CD₃OD) δ 8.16-8.13 (m, 2H), 7.59-7.52 (m, 2H), 7.03-6.77 (m, 8H), 4.21-3.93 (m, 3H), 3.88-3.79 (m, 1H), 3.77-3.57 (m, 1.5H), 3.41-3.39 (m, 1H), 3.34-3.32 (m, 0.5H), 3.11-2.96 (m, 2H), 1.63-1.49 (m, 2H), 0.97-0.88 (m, 3H). LCMS: M/Z (M+H)⁺ 564.2.

The embodiments described hereinbefore may be used in any combination with each other. Several of the embodiments may be combined together to form a further embodiment. A product, a method or a use, to which the invention is related, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

It is obvious to a person skilled in the art that with the advancement of technology, the basic idea of the invention may be implemented in various ways. The invention and its embodiments are thus not limited to the examples described above; instead they may vary within the scope of the claims.

1. A compound of formula (I): 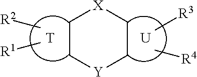
wherein:
X is absent, direct bond, —S—, —(CH₂CH₂)—, —CH═CH—; —O—, —CH₂O—, —OCH₂—, —C(═O)NR^{D}—, or —N(R^{D})C(═O)—;
R^{D} is selected from hydrogen and (C₁-C₆)alkyl;
Y is selected from the group consisting of:
—N(R⁵)—, —N(R⁵)—S(O)₂—, —CH₂—N(R⁵)—CH₂—, —C(R¹⁴)(R⁵)—, —C(═R^{5a})— and —C(R^{A}R^{B}),
R^{A} and R^{B} together with the C atom to which they are attached form a three to six membered aliphatic carbocycle or a heterocycle which is substituted with Z and attached at the C atom as a spiro ring, and the carbocycle or the heterocycle which is substituted with Z is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;
T is a benzene ring or a five- or six-membered heteroaromatic ring;
U is a benzene ring or a five- or six-membered heteroaromatic ring;
R¹, R², R³, and R⁴ are independently selected from the group consisting of: H, halo, —N₃, —NR⁶R⁷, (C₁-C₆)alkyl, (C₁-C₆)haloalkyl, —OR⁶, —C(O)R⁶, —OC(O)R⁶, —C(O)NR⁶R⁷, —C(O)OR⁶, —SR⁶, —SO₂R⁶, and —SO₂NR⁶R⁷, —CF₃, and —CN, nitro, (C₁-C₆)haloalkoxy, (C₁-C₆)haloalkylthio, (C₁-C₆)haloalkylthio, —CHC(═O)O(C₁-C₆)alkyl;
R⁵ is —(CR¹⁵R¹⁶)ₚ-Q_{q}-(CR¹⁵R¹⁶)ₙ₋ₚ—Z or 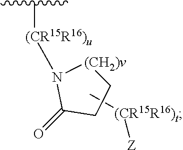
R^{5a} is ═CR¹⁴(CR¹⁵R¹⁶)ₚ-Q_{q}-(CR¹⁵R¹⁶)ₘ₋ₚ—Z;
Q is selected from —O—, —NR^{14′}—, —C(O)—, —S—, —S(O)— and —S(O)₂—;
R⁶ and R⁷ are independently selected from the group consisting of: H and (C₁-C₆)alkyl;
R¹⁴ is hydrogen or (C₁-C₆)alkyl;
R^{14′} is hydrogen or optionally substituted (C₁-C₆)alkyl, (C₃-C₇)cycloalkyl, aryl, or heteroaryl; —SO₂R⁸; —SO₂NR⁸R⁹; —C(═O)R¹⁰; —C(═O)OR¹⁰; or —C(═O)NR⁸R⁹; wherein said substituents on the (C₁-C₆)alkyl, (C₃-C₇)cycloalkyl, aryl, or heteroaryl are selected from the group consisting of hydroxy, halogen, cyano, nitro, amino, (C₁-C₆)alkylamino, (C₁-C₆)dialkylamino, (C₁-C₆)acylamino, (C₁-C₆)alkylsulfonyl, (C₁-C₆)alkylthio, (C₁-C₆)alkyl, (C₃-C₇)cycloalkyl, (C₁-C₆)haloalkyl, (C₁-C₆)haloalkoxy, and (C₁-C₆)alkoxy,
R⁸ and R⁹ are independently selected in each instance from hydrogen, (C₁-C₆)alkyl, aryl, and arylalkyl, wherein said aryl or the aryl of the arylalkyl is optionally substituted with hydroxy, halogen, cyano, nitro, amino, (C₁-C₆)alkylamino, (C₁-C₆)dialkylamino, (C₁-C₆)acylamino, (C₁-C₆)alkylsulfonyl, (C₁-C₆)alkylthio, (C₁-C₆)alkyl, (C₁-C₆)haloalkyl, (C₁-C₆)haloalkoxy, or (C₁-C₆)alkoxy;
R¹⁰ is selected from hydrogen, optionally substituted (C₁-C₆)alkyl, or optionally substituted aryl, wherein said optional substituents are selected from the group consisting of (C₁-C₆)alkyl, OR⁶, NH₂, NHMe, N(Me)₂, and heterocycle;
R¹⁵ and R¹⁶ in each occurrence are selected independently from the group consisting of from H, OH, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, or, taken together, two of R¹⁴, R^{14′}, R¹⁵ and R¹⁶ may form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;
m is an integer from 1 to 3;
n is an integer from 2 to 4,
p is zero, 1 or 2 q is zero or 1:
with the proviso that when p is 2, m is not 1;
t is zero, 1 or 2;
u is zero, 1 or 2:
v is 1, 2 or 3;
Z is —NHS((O)(NR¹⁸))R¹⁷;
R¹⁷ is selected from phenyl and monocyclic heteroaryl, said phenyl and monocyclic heteroaryl optionally substituted with one or two substituents selected independently from the group consisting of OH, halogen, cyano, nitro, amino, (C₁-C₆)alkylamino, (C₁-C₆)dialkylamino, (C₁-C₆)acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O)(C₁-C₆)alkyl, —C(═O)H, (C₁-C₆)hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇)cycloalkyl, (C₁-C₆)alkenyl, (C₁-C₆)alkynyl or aryl;
R¹⁸ is H or (C₁-C₆)alkyl, wherein said alkyl is optionally substituted with (C₃-C₇)cycloalkyl group;
or an enantiomer, a diastereomer, a tautomer or a pharmaceutically acceptable salt thereof.

2. A compound according to claim 1, or an enantiomer, a diastereomer, a tautomer or a pharmaceutically acceptable salt thereof, wherein T is a benzene ring, U is a benzene ring and Y is —N(R⁵)— and the compound is of formula (IA) 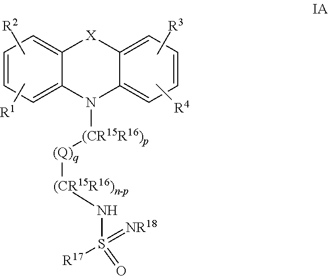
wherein
R¹, R², R³, and R⁴ are as defined in claim 1;
X is as defined in claim 1;
R¹⁵ and R¹⁶ in each occurrence are selected independently from the group consisting of H, OH, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, or, R¹⁵ and R¹⁶ taken together may form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;
Q, n, p and q are as defined in claim 1;
R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆)acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O)(C₁-C₆)alkyl, —C(═O)H, (C₁-C₆)hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇)cycloalkyl, (C₁-C₆)alkenyl, (C₁-C₆)alkynyl or aryl;
R⁸ is as defined in claim 1.

3. A compound according to claim 1, or an enantiomer, a diastereomer, a tautomer or a pharmaceutically acceptable salt thereof, wherein two of R¹⁵ and/or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, said compound being of formula (IA-1) or (IA-2): 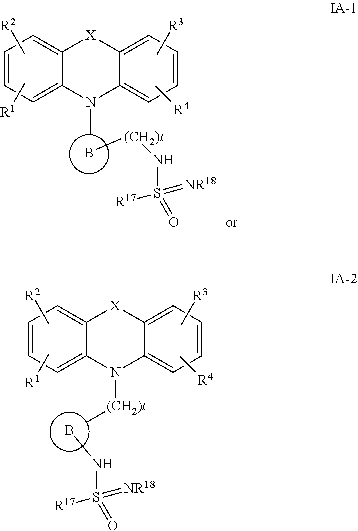
wherein
t is zero, 1 or 2.

4. A compound according to anyone of claim 1, or an enantiomer, a diastereomer, a tautomer or a pharmaceutically acceptable salt thereof, wherein
a) B is a five-membered ring as set forth in formula: 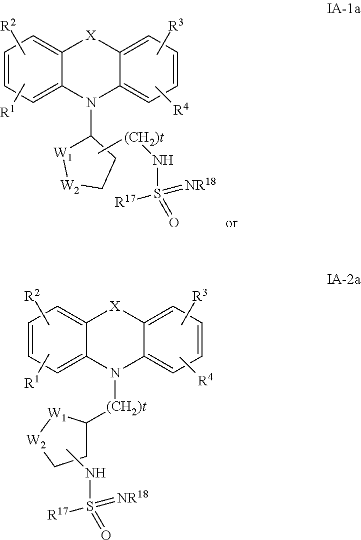
wherein:
W₁ and W₂ are both —CH₂—; or
one of W₁ and W₂ is selected from a group consisting of —O—, —NR^{14′}—, —C(O)—, —S—, —S(O)— or —S(O)₂ and the other is —CH₂—; or
one of W₁ and W₂ is —CH(OH)— and the other is —CH₂—;
or
b) B is a six-membered ring as set forth in formula: 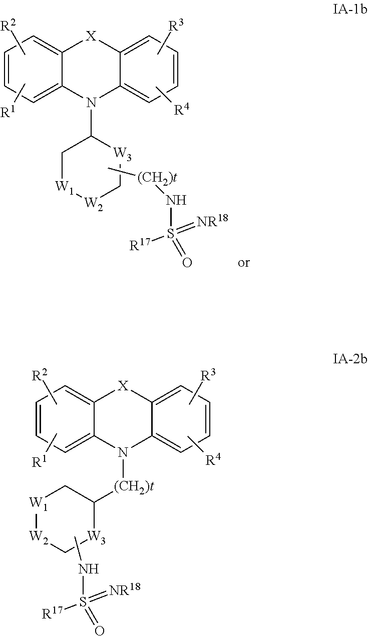
wherein:
all of W₁, W₂ and W₃ are —CH₂—; or
one of W₁, W₂ and W₃ is —O—, —NR^{14′}—, —C(O)—, —S—, —S(O)— or —S(O)₂ and the other two are —CH₂—; or
one of W₁, W₂ and W₃ is —O—, —NR^{14′}—, —C(O)—, —S—, —S(O)— or —S(O)₂ and the other two are —CH₂— and —CH(OH)—; or
one of W₁, W₂ and W₃ is —CH(OH)— and the other two are —CH₂.

5. A compound according to claim 1, or an enantiomer, a diastereomer, a tautomer or a pharmaceutically acceptable salt thereof, wherein p and q are both zero, R¹⁵ is H and R¹⁶ is selected from H or OH, and the compound is of formula (IA-3) or (IA-3′): 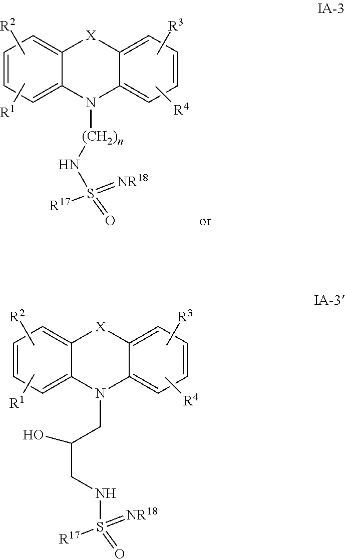
wherein
n is 2, 3 or 4.

6. A compound according to claim 1, or an enantiomer, a diastereomer, a tautomer or a pharmaceutically acceptable salt thereof, wherein T is a benzene ring, U is a benzene ring and Y is —C(R¹⁴)(R⁵)—, R¹⁴ is H, and the compound is of formula (IB) 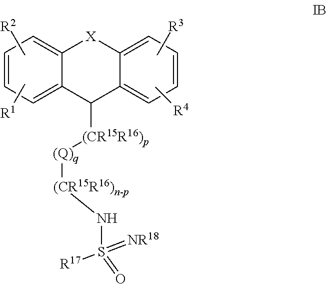
wherein
R¹, R², R³, and R⁴ are as defined in claim 1;
X is as defined in claim 1;
R¹⁵ and R¹⁶ in each occurrence are selected independently from the group consisting of H, OH, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, or, R¹⁵ and R¹⁶ taken together may form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;
Q, n, p and q are as defined in claim 1;
R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆)acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O)(C₁-C₆)alkyl, —C(═O)H, (C₁-C₆)hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇)cycloalkyl, (C₁-C₆)alkenyl, (C₁-C₆)alkynyl or aryl;
R¹⁸ is as defined in claim 1.

7. A compound according to claim 1, or an enantiomer, a diastereomer, a tautomer or a pharmaceutically acceptable salt thereof, wherein two of R¹⁵ and/or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, said compound being of formula IB-1 or IB-2: 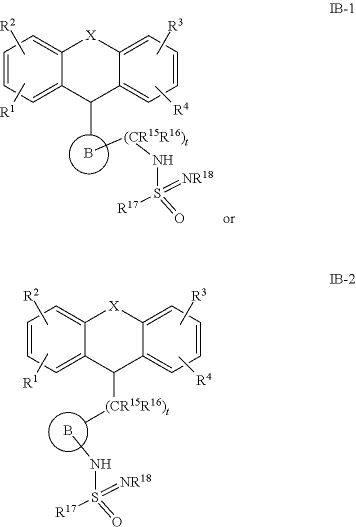
wherein
t is zero, 1 or 2.

8. A compound according to claim 1 or an enantiomer, a diastereomer, a tautomer or a pharmaceutically acceptable salt thereof, wherein
a) B is a five-membered ring as set forth in formula: 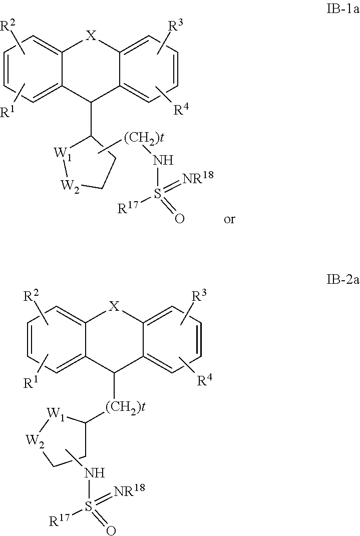
wherein:
W₁ and W₂ are both —CH₂—; or
one of W₁ and W₂ is selected from a group consisting of —O—, —NR^{14′}—, —C(O)—, —S—, —S(O)— or —S(O)₂ and the other is —CH₂—; or
one of W₁ and W₂ is —CH(OH)— and the other is —CH₂—;
or
b) B is a six-membered ring as set forth in formula: 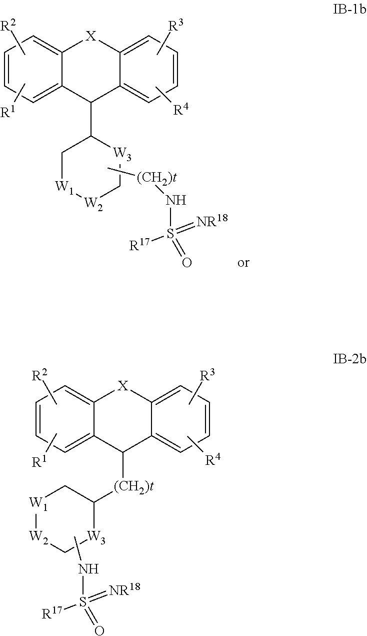
wherein:
all of W₁, W₂ and W₃ are —CH₂—; or
one of W₁, W₂ and W₃ is —O—, —NR^{14′}—, —C(O)—, —S—, —S(O)— or —S(O)₂ and the other two are —CH₂—; or
one of W₁, W₂ and W₃ is —O—, —NR^{14′}—, —C(O)—, —S—, —S(O)— or —S(O)₂ and the other two are —CH₂— and —CH(OH)—; or
one of W₁, W₂ and W₃ is —CH(OH)— and the other two are —CH₂.

9. A compound according to claim 1, or an enantiomer, a diastereomer, a tautomer or a pharmaceutically acceptable salt thereof, wherein p and q are both zero, R¹⁵ is H and R¹⁶ is selected from H and OH, and the compound is of formula (IB-3) or (IB-3′): 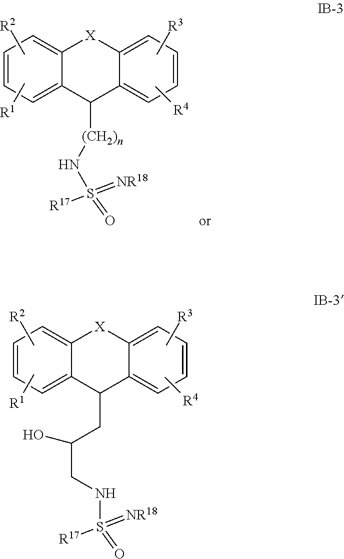
wherein
n is 2, 3 or 4.

10. A compound according to claim 1, or an enantiomer, a diastereomer, a tautomer or a pharmaceutically acceptable salt thereof, wherein p is zero, q is 1, and wherein two of R¹⁵ and/or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy.

11. A compound according to claim 1, or an enantiomer, a diastereomer, a tautomer or a pharmaceutically acceptable salt thereof, wherein T is a benzene ring, U is a benzene ring, Y is —C(═R^{5a})— and R¹⁴ is hydrogen, and the compound is of formula (IC) 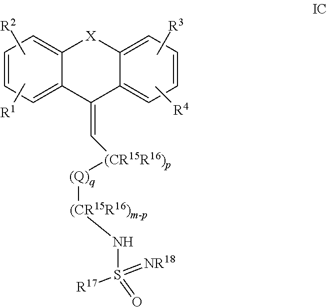
R¹, R², R³, and R⁴ are as defined in claim 1;
X is as defined in claim 1;
R¹⁵ and R¹⁶ in each occurrence are selected independently from the group consisting of H, OH, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, or, R¹⁵ and R¹⁶ taken together may form a three to seven membered non-aromatic carbocycle or heterocycle wherein said three to seven membered carbocycle or heterocycle is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy;
Q, m, p and q are as defined in claim 1;
R¹⁷ is selected from phenyl, pyridinyl, thiazolyl, furanyl, thiophenyl, pyrrolyl, thienyl, each optionally substituted with one or two substituents selected independently from the group consisting of OH, halogen, cyano, nitro, amino, (C₁-C₆)alkylamino, (C₁-C₆)dialkylamino, (C₁-C₆)acylamino, (C₁-C₆)alkylsulfonyl, (C₁-C₆)alkylthio, (C₁-C₆)alkyl, (C₁-C₆)haloalkyl, (C₁-C₆)haloalkoxy, and (C₁-C₆)alkoxy, —C(═O)(C₁-C₆)alkyl, —C(═O)H, (C₁-C₆)hydroxyalkyl, (C₁-C₆)haloalkylthio, N₃; —NR⁶C(O)OR^{6′}, —OC(O)R⁶, —C(O)NR⁶R⁷, —C(O)OR⁶, and —SO₂NR⁶R⁷; and R^{6′} is selected from (C₁-C₆)alkyl, (C₃-C₇)cycloalkyl, (C₁-C₆)alkenyl, (C₁-C₆)alkynyl or aryl;
R¹⁸ is as defined in claim 1.

12. A compound according to claim 1, or an enantiomer, a diastereomer, a tautomer or a pharmaceutically acceptable salt thereof, wherein two of R¹⁵ and/or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, said compound being of formula:

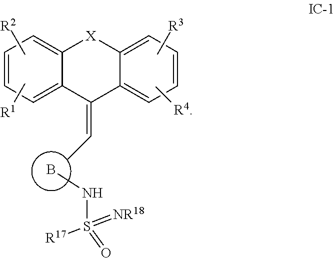

13. A compound according to claim 1, or an enantiomer, a diastereomer, a tautomer or a pharmaceutically acceptable salt thereof, wherein T is a benzene ring, U is a benzene ring, Y is —C(═R^{5a})—, —R¹⁴ and one R¹⁵ or R¹⁶ taken together form a three to seven membered non-aromatic carbocycle or heterocycle B, wherein said three to seven membered carbocycle or heterocycle B is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, said compound being of formula, said compound being of formula: 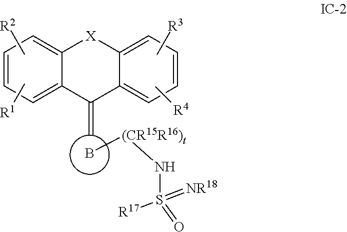
wherein
t is zero, 1 or 2.

14. A compound according to claim 1, or an enantiomer, a diastereomer, a tautomer or a pharmaceutically acceptable salt thereof, wherein
a) B is a five-membered ring as set forth in formula: 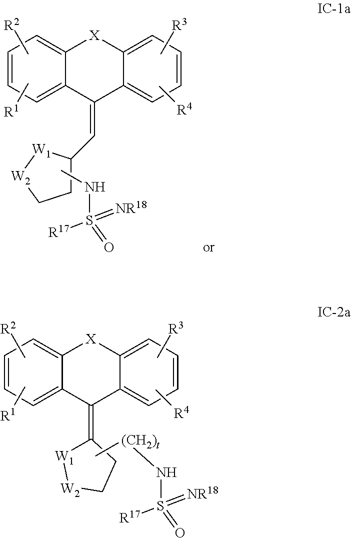
wherein:
W₁ and W₂ are both —CH₂—; or
one of W₁ and W₂ is selected from a group consisting of —O—, —NR^{14′}—, —C(O)—, —S—, —S(O)— or —S(O)₂ and the other is —CH₂—; or
one of W₁ and W₂ is —CH(OH)— and the other is —CH₂—;
or
b) B is a six-membered ring as set forth in formula: 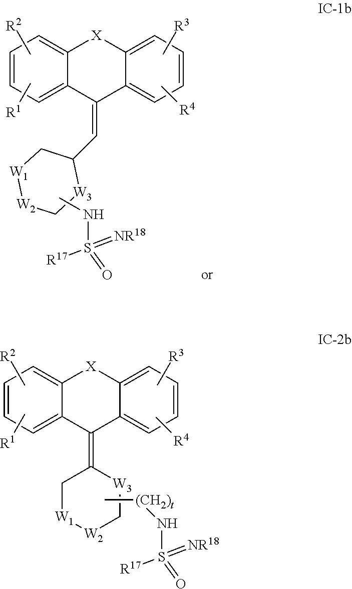
wherein:
all of W₁, W₂ and W₃ are —CH₂—; or
one of W₁, W₂ and W₃ is —O—, —NR^{14′}—, —C(O)—, —S—, —S(O)— or —S(O)₂ and the other two are —CH₂—; or
one of W₁, W₂ and W₃ is —O—, —NR^{14′}—, —C(O)—, —S—, —S(O)— or —S(O)₂ and the other two are —CH₂— and —CH(OH)—; or
one of W₁, W₂ and W₃ is —CH(OH)— and the other two are —CH₂.

15. A compound according to claim 1, or an enantiomer, a diastereomer, a tautomer or a pharmaceutically acceptable salt thereof wherein T is a benzene ring, U is a benzene ring, Y is —C(R^{A}R^{B}), and R^{A} and R^{B} together with the C atom which they are attached form a three to six membered aliphatic carbocycle or heterocycle A which is substituted with Z, and said carbocycle or heterocycle A which is substituted with Z is optionally substituted with one or two substituents selected independently from the group consisting of OH, F, cyano, amino, (C₁-C₆)alkylamino, (C₁-C₆)dialkylamino, (C₁-C₆)alkyl, (C₁-C₆)haloalkyl, (C₁-C₆)haloalkoxy, and (C₁-C₆)alkoxy, said compound being of formula (ID)

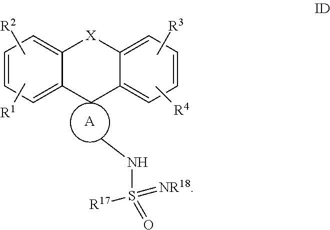

16. A compound according to claim 1, where R¹⁸ is hydrogen, methyl, propyl, or methylcyclopropyl.

17. A compound according to claim 1 selected from: 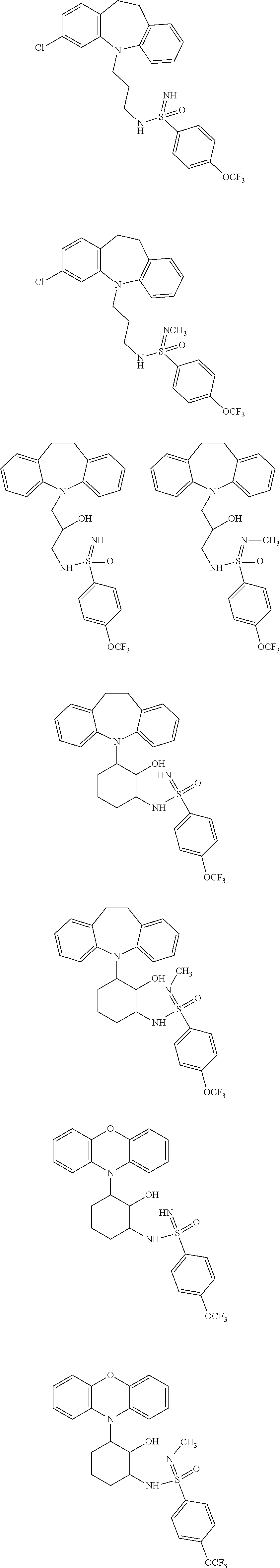 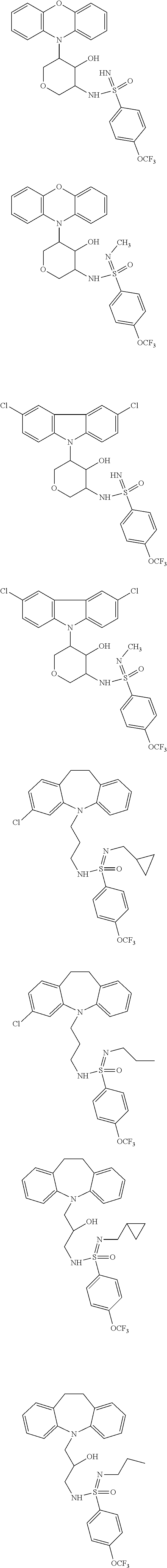 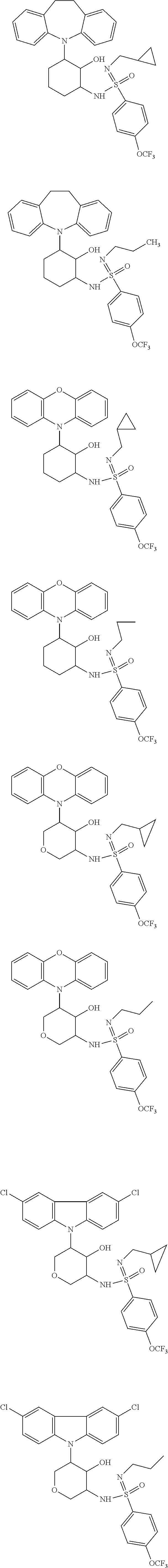
an enantiomer, a diastereomer, a tautomer or a pharmaceutically acceptable salt thereof.

18. A compound according to claim 1 selected from:

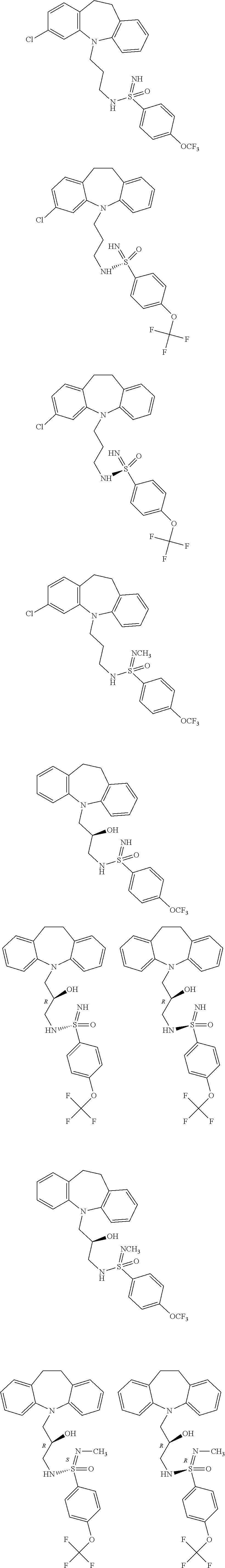

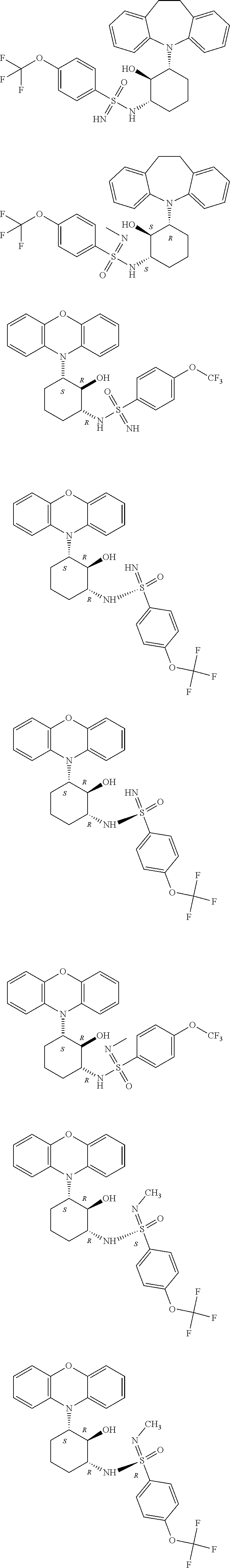

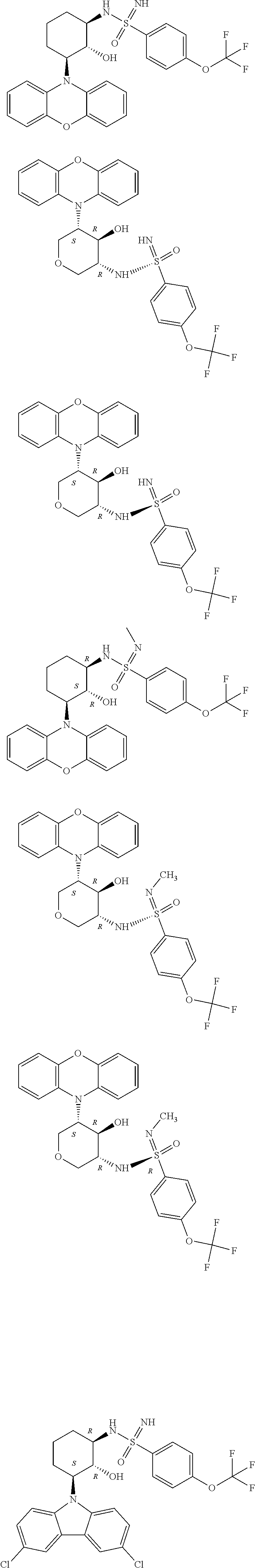

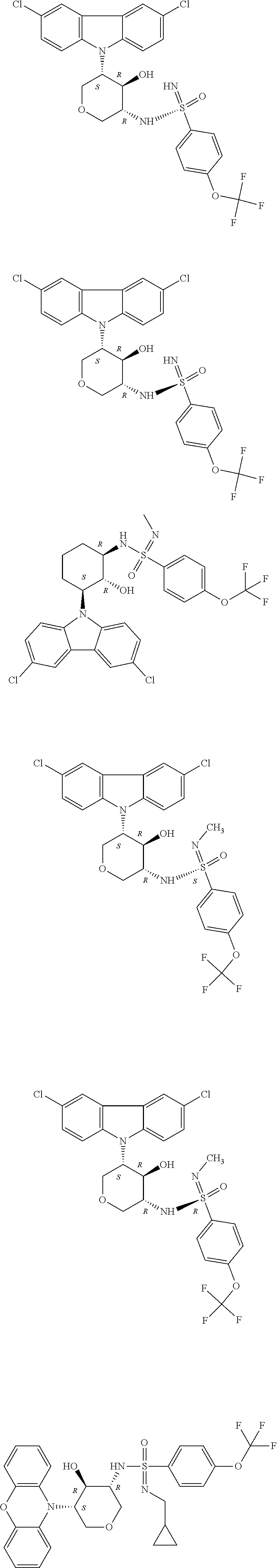

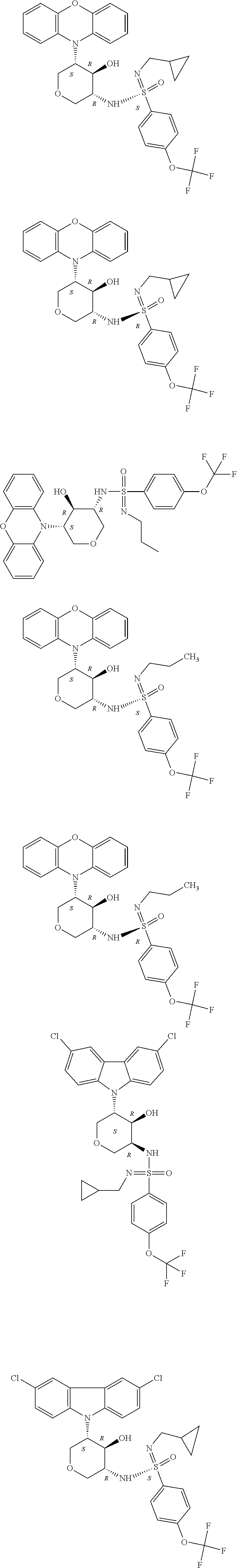

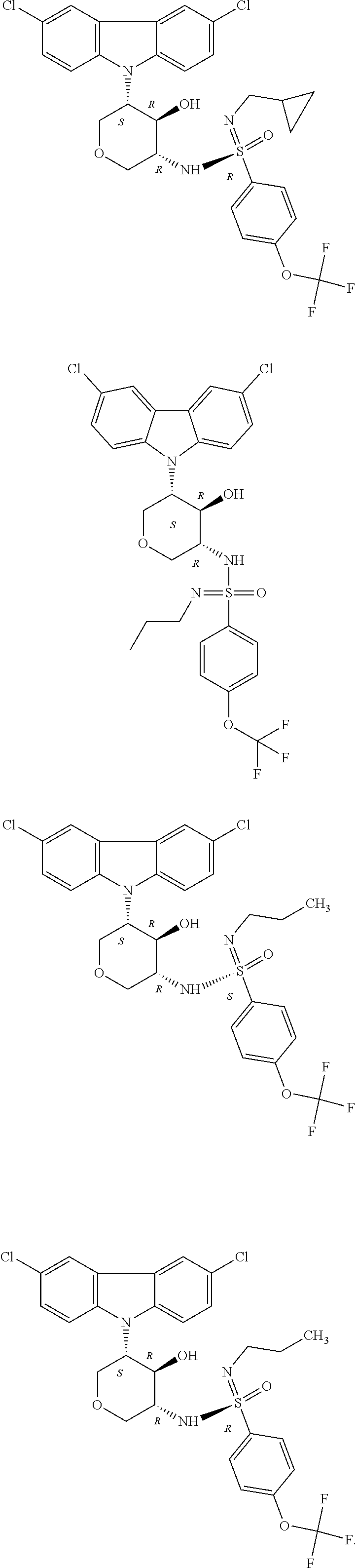

19. A pharmaceutical composition comprising a compound according to claim 1, an enantiomer, a diastereomer, a tautomer or a pharmaceutically acceptable salt thereof, and pharmaceutically acceptable carrier.

20-23. (canceled)

24. A method of preventing or treating a disease or condition by comprising administering to a patient a therapeutically effective amount of a compound, an enantiomer, a diastereomer, a tautomer or a pharmaceutically acceptable salt thereof according to claim 1.

25. The method of claim 24, wherein the disease or condition is selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, betacoronavirus infection, heart failure, neurodegenerative disease and cardiac hypertrophy.

26. The method of claim 24, wherein the disease is cancer.
